(19) United States
(12) Patent Application Publication
(10) Pub. No.: US 2023/0305027 A1
(43) Pub. Date: Sep. 28, 2023

(54) METHODS FOR TREATING EXACERBATIONS OF INFLAMMATORY RESPIRATORY DISEASES

(71) Applicants: Genentech, Inc., South San Francisco, CA (US); Hoffmann-La Roche Inc., Little Falls, NJ (US)

(72) Inventors: Qingling LI, San Jose, CA (US); Margaret NEIGHBORS, San Jose, CA (US); Carrie Melissa ROSENBERGER, San Francisco, CA (US); Wendy Noel SANDOVAL, Millbrae, CA (US); Gaik Wei TEW, Belmont, CA (US); Sha ZHU, Welwyn Garden City (GB); Arindam CHAKRABARTI, San Ramon, CA (US); Michele Anne GRIMBALDESTON, Menlo Park, CA (US)

(21) Appl. No.: 18/103,679

(22) Filed: Jan. 31, 2023

Related U.S. Application Data
(63) Continuation of application No. PCT/US2021/044064, filed on Jul. 30, 2021.
(60) Provisional application No. 63/059,887, filed on Jul. 31, 2020.

(51) Int. Cl. G01N 33/92

(52) U.S. Cl. CPC G01N 33/92; G01N 2405/04; G01N 2800/52; G01N 2800/122; G01N 2800/12

(57) ABSTRACT

Provided herein are therapeutic methods for the treatment of exacerbations of inflammatory respiratory diseases, including chronic obstructive pulmonary disease (COPD), idiopathic pulmonary fibrosis (IPF), and asthma. In particular, the invention provides methods for patient selection, diagnosis, and treatment. Also provided herein are methods for preparing and analyzing lysophosphatidic acid (LPA) samples.

### FIELD OF THE INVENTION

Provided herein are therapeutic methods for the treatment of exacerbations of inflammatory respiratory diseases, including chronic obstructive pulmonary disease (COPD), idiopathic pulmonary fibrosis (IPF), and asthma. In particular, the invention provides methods for patient selection, diagnosis, and treatment. Also provided herein are methods for preparing and analyzing lysophosphatidic acid (LPA) samples.

### BACKGROUND

Exacerbations of inflammatory respiratory diseases, including chronic obstructive pulmonary disease (COPD), idiopathic pulmonary fibrosis (IPF), and asthma, have a profound effect on disease progression. In COPD, the frequency and severity of exacerbations are associated with increased rate of lung function decline and worse health-related quality of life, and fewer than half of patients survive for a further five years after a severe exacerbation.

Exacerbations are heterogenous events, as the interactions between exacerbation triggers and host inflammatory responses are complex. Accordingly, studies have failed to identify consistent blood biomarkers associated with COPD exacerbation.

Thus, there exists an unmet need for methods of predicting whether patients having inflammatory respiratory diseases are likely to experience an exacerbation, as well as methods of treating such patients.

### SUMMARY OF THE INVENTION

In one aspect, the disclosure features a method for identifying, diagnosing, and/or predicting whether a patient having chronic obstructive pulmonary disease (COPD) may have an increased risk for an exacerbation, the method comprising measuring a level of one or more of lysophosphatidic acid (LPA)16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies, diagnoses, and/or predicts the patient as one who is at an increased risk for an exacerbation.

In another aspect, the disclosure features a method for identifying, diagnosing, and/or predicting whether a patient having COPD may benefit from a treatment comprising an agent that reduces exacerbations, the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies, diagnoses, and/or predicts the patient as one who may benefit from a treatment comprising an agent that reduces exacerbations.

In another aspect, the disclosure features a method of selecting a therapy for a patient having COPD, the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies the patient as one who may benefit from a treatment comprising an agent that reduces exacerbations.

In some aspects, the patient has a level of one or more of LPA16:0, LPA18:0, LPA18:1, LPA18:2, and LPA20:4 in the sample that is below a reference level and the method further comprises administering to the patient an effective amount of an agent that reduces exacerbations.

In another aspect, the disclosure features a method of treating a patient having COPD, the method comprising (a) measuring a level of one or more of LPA16:0, LPA18:0, LPA18:1, LPA18:2, and LPA20:4 in a sample from the patient, wherein the level of one or more of LPA16:0, LPA18:0, LPA18:1, LPA18:2, and LPA20:4 in the sample is below a reference level; and (b) administering an effective amount of an agent that reduces exacerbations to the patient.

In another aspect, the disclosure features a method of treating a patient having COPD and having a level of one or more of LPA16:0, LPA18:0, LPA18:1, LPA18:2, and LPA20:4 in a sample from the patient that is below a reference level comprising administering an effective amount of an agent that reduces exacerbations to the patient.

In another aspect, the disclosure features a method of treating a patient having COPD, the method comprising administering to the patient an effective amount of an agent that reduces exacerbations, wherein the level of one or more of LPA16:0, LPA18:0, LPA18:1, LPA18:2, and LPA20:4 in a sample from the patient has been determined to be below a reference level.

In another aspect, the disclosure features a method of reducing exacerbations in a patient having COPD, the method comprising (a) measuring a level of one or more of LPA16:0, LPA18:0, LPA18:1, LPA18:2, and LPA20:4 in a sample from the patient, wherein the level of one or more of LPA16:0, LPA18:0, LPA18:1, LPA18:2, and LPA20:4 in the sample is below a reference level; and (b) administering an effective amount of an agent that reduces exacerbations to the patient.

In another aspect, the disclosure features a method of reducing exacerbations in a patient having COPD and having a level of one or more of LPA16:0, LPA18:0, LPA18:1, LPA18:2, and LPA20:4 in a sample from the patient that is below a reference level comprising administering an effective amount of an agent that reduces exacerbations to the patient.

In another aspect, the disclosure features a method of reducing exacerbations in a patient having COPD, the method comprising administering to the patient an effective amount of an agent that reduces exacerbations, wherein the level of one or more of LPA16:0, LPA18:0, LPA18:1, LPA18:2, and LPA20:4 in a sample from the patient has been determined to be below a reference level.

In another aspect, the disclosure features a method of identifying a patient suitable for administration with an agent that treats COPD or an agent that reduces exacerbations of COPD, the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies the patient as one who is suitable for administration with an agent that treats COPD or an agent that reduces exacerbations of COPD.

In another aspect, the disclosure features a method of monitoring the response of a patient having COPD to a treatment comprising an agent that reduces exacerbations, the method comprising (a) measuring the level of one or more of LPA16:0, LPA18:0, LPA18:1, LPA18:2, and LPA20:4 in a sample obtained from the patient at a time point following the administration of a first dose of the treatment comprising the agent that reduces exacerbations; and (b) comparing the level of one or more of LPA16:0, LPA18:0, LPA18:1, LPA18:2, and LPA20:4 in the sample to a reference level, thereby monitoring the response of the patient to the treatment comprising an agent that reduces exacerbations.

In some aspects, a level of one or more of LPA16:0, LPA18:0, LPA18:1, LPA18:2, and LPA20:4 in a sample that is above a reference level indicates that the patient is responding to the agent that reduces exacerbations.

In another aspect, the method further comprises administering at least a second dose of the agent that reduces exacerbations to a patient for whom a level of one or more of LPA16:0, LPA18:0, LPA18:1, LPA18:2, and LPA20:4 in the sample is above a reference level.

In another aspect, the disclosure features a method of enrolling a patient suitable for a clinical study, the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies the patient as one who is suitable for the clinical study. In some aspects, the method further comprises enrolling the patient who has been identified as suitable for the clinical study in the clinical study.

In some aspects, the sample is a whole blood sample, a plasma sample, a serum sample, or a combination thereof.

In some aspects, the sample is a bronchoalveolar lavage fluid (BALF) sample.

In some aspects, the sample is an archival sample, a fresh sample, or a frozen sample.

In some aspects, the sample is a serum sample.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4.

In some aspects, the reference level is a pre-assigned level of one or more of LPA16:0, LPA18:0, LPA18:1, LPA18:2, and LPA20:4.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level is a level of one or more of LPA16:0, LPA18:0, LPA18:1, LPA18:2, and LPA20:4 in a reference population.

In some aspects, the level of one or more of LPA16:0, LPA18:0, LPA18:1, and LPA18:2 in the sample is at or below the 33^{rd} percentile of levels of LPA16:0, LPA18:0, LPA18:1, or LPA18:2, respectively, in the reference population.

In some aspects, the level of LPA20:4 in the sample is at or below the 67^{th} percentile of levels of LPA20:4 in the reference population.

In some aspects, the reference population is a population of patients having COPD. In some aspects, the COPD is stage II, stage III, or stage IV COPD.

In some aspects, the patient has experienced at least one exacerbation in the prior 12 months.

In some aspects, the benefit comprises an extension in the patient's time to an exacerbation compared to treatment without the agent that reduces exacerbations.

In some aspects, the exacerbation is an increase in one or more of dyspnea, cough, sputum volume, sputum purulence, fatigue, trouble sleeping, headache when waking up, confusion, and hypoxemia.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is an agent disclosed in the GLOBAL INITIATIVE FOR CHRONIC OBSTRUCTIVE LUNG DISEASE™ (GOLD) Pocket Guide to COPD Diagnosis, Management, and Prevention (2020 Edition).

In some aspects, the agent that reduces exacerbations is approved by a regulatory health agency for reducing, controlling, or maintaining exacerbations. In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

In some aspects, the patient is male.

In another aspect, the disclosure features use of an agent that reduces exacerbations in a patient having a level of one or more of LPA16:0, LPA18:0, LPA18:1, LPA18:2, and LPA20:4 in a sample from the patient that is below a reference level in the manufacture of a medicament for the treatment of COPD.

In another aspect, the disclosure features use of an agent that reduces exacerbations in a patient having a level of one or more of LPA16:0, LPA18:0, LPA18:1, LPA18:2, and LPA20:4 in a sample from the patient that is below a reference level in the manufacture of a medicament for reducing exacerbations of COPD.

In some aspects, the sample is a whole blood sample, a plasma sample, a serum sample, or a combination thereof.

In some aspects, the sample is a BALF sample.

In some aspects, the sample is an archival sample, a fresh sample, or a frozen sample.

In some aspects, the sample is a serum sample.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4.

In some aspects, the reference level is a pre-assigned level of one or more of LPA16:0, LPA18:0, LPA18:1, LPA18:2, and LPA20:4.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level is a level of one or more of LPA16:0, LPA18:0, LPA18:1, LPA18:2, and LPA20:4 in a reference population.

In some aspects, the level of one or more of LPA16:0, LPA18:0, LPA18:1, and LPA18:2 in the sample is at or below the 33^{rd} percentile of levels of LPA16:0, LPA18:0, LPA18:1, or LPA18:2 respectively, in the reference population.

In some aspects, the level of LPA20:4 in the sample is at or below the 67^{th} percentile of levels of LPA20:4 in the reference population.

In some aspects, the reference population is a population of patients having COPD.

In some aspects, the patient has COPD. In some aspects, the COPD is stage II, stage III, or stage IV COPD.

In some aspects, the patient has experienced at least one exacerbation in the prior 12 months.

In some aspects, the exacerbation is an increase in one or more of dyspnea, cough, sputum volume, sputum purulence, fatigue, trouble sleeping, headache when waking up, confusion, and hypoxemia.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is an agent disclosed in the GLOBAL INITIATIVE FOR CHRONIC OBSTRUCTIVE LUNG DISEASE™ (GOLD) Pocket Guide to COPD Diagnosis, Management, and Prevention (2020 Edition).

In some aspects, the agent that reduces exacerbations is approved by a regulatory health agency for reducing, controlling, or maintaining exacerbations. In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

In some aspects, the patient is male.

In another aspect, the disclosure features an agent that reduces exacerbations for use in the treatment of a patient having COPD and having a level of one or more of LPA16:0, LPA18:0, LPA18:1, LPA18:2, and LPA20:4 in a sample from the patient that is below a reference level.

In another aspect, the disclosure features an agent that reduces exacerbations for use in the treatment of a patient having COPD, wherein the level of one or more of LPA16:0, LPA18:0, LPA18:1, LPA18:2, and LPA20:4 in a sample from the patient has been determined to be below a reference level.

In some aspects, the sample is a whole blood sample, a plasma sample, a serum sample, or a combination thereof.

In some aspects, the sample is a BALF sample.

In some aspects, the sample is an archival sample, a fresh sample, or a frozen sample.

In some aspects, the sample is a serum sample.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4.

In some aspects, the reference level is a pre-assigned level of one or more of LPA16:0, LPA18:0, LPA18:1, LPA18:2, and LPA20:4.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level is a level of one or more of LPA16:0, LPA18:0, LPA18:1, LPA18:2, and LPA20:4 in a reference population.

In some aspects, the level of one or more of LPA16:0, LPA18:0, LPA18:1, and LPA18:2 in the sample is at or below the 33^{rd} percentile of levels of LPA16:0, LPA18:0, LPA18:1, or LPA18:2 respectively, in the reference population.

In some aspects, the level of LPA20:4 in the sample is at or below the 67^{th} percentile of levels of LPA20:4 in the reference population.

In some aspects, the reference population is a population of patients having COPD.

In some aspects, the patient has COPD. In some aspects, the COPD is stage II, stage III, or stage IV COPD.

In some aspects, the patient has experienced at least one exacerbation in the prior 12 months.

In some aspects, the exacerbation is an increase in one or more of dyspnea, cough, sputum volume, sputum purulence, fatigue, trouble sleeping, headache when waking up, confusion, and hypoxemia.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is an agent disclosed in the GLOBAL INITIATIVE FOR CHRONIC OBSTRUCTIVE LUNG DISEASE™ (GOLD) Pocket Guide to COPD Diagnosis, Management, and Prevention (2020 Edition).

In some aspects, the agent that reduces exacerbations is approved by a regulatory health agency for reducing, controlling, or maintaining exacerbations. In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

In some aspects, the patient is male.

In another aspect, the disclosure features a method for identifying, diagnosing, and/or predicting whether a patient having COPD may have an increased risk for an exacerbation, the method comprising measuring a level of one or more of LPC, sphingomyelins, and ceramides in a sample from the patient, wherein a level of LPC in the sample that is below a reference level and/or a level of one or both of sphingomyelins and ceramides in the sample that is above a reference level identifies, diagnoses, and/or predicts the patient as one who is at an increased risk for an exacerbation.

In some aspects, the LPC is LPC(16:0) or LPC(18:2).

In some aspects, the patient has a level of a LPC in the sample that is below a reference level and/or a level of one or both of sphingomyelins and ceramides in the sample that is above a reference level and the method further comprises administering to the patient an effective amount of an agent that reduces exacerbations.

In some aspects, the sample is a whole blood sample, a plasma sample, a serum sample, or a combination thereof.

In some aspects, the sample is a BALF sample.

In some aspects, the sample is an archival sample, a fresh sample, or a frozen sample.

In some aspects, the sample is a serum sample.

In some aspects, the level of one or more of LPC, sphingomyelins, and ceramides is a baseline level of one or more of LPC, sphingomyelins, and ceramides.

In some aspects, the reference level is a pre-assigned level of one or more of LPC, sphingomyelins, and ceramides.

In some aspects, the reference level for LPC is between about 227 nmol/mL to about 277 nmol/mL. In some aspects, the reference level for LPC is about 252 nmol/mL.

In some aspects, the reference level for sphingomyelins is between about 448 nmol/mL to about 548 nmol/mL. In some aspects, the reference level for sphingomyelins is about 498 nmol/mL.

In some aspects, the ceramide is hexosylceramide (HCER).

In some aspects, the reference level for HCER is between about 6.1 nmol/mL to about 7.5 nmol/mL. In some aspects, the reference level for HCER is about 6.8 nmol/mL.

In some aspects, the ceramide is lactosylceramide (LCER) In some aspects, the reference level for LCER is between about 4.3 nmol/mL to about 5.3 nmol/mL. In some aspects, the reference level for LCER is about 4.8 nmol/mL.

In some aspects, the reference level is a level of one or more of LPC, sphingomyelins, and ceramides in a reference population.

In some aspects, the ceramide is LCER or HCER.

In some aspects, the level of LPC in the sample is at or below the 33^{rd} percentile of levels of LPC in the reference population and/or the levels of sphingomyelins, LCER, and/or HCER are at or above the 67^{th} percentile of levels of sphingomyelins, LCER, or HCER, respectively, in the reference population.

In some aspects, the reference population is a population of patients having COPD.

In another aspect, the disclosure features a method for predicting the time to next exacerbation for a patient having COPD who has experienced at least one exacerbation in the prior 12 months, the method comprising measuring a level of one or both of LPA18:0 and LPA18:2 in a sample from the patient, wherein a level of one or both of LPA18:0 and LPA18:2 in the sample that is above a reference level identifies the patient as one who may have an increased time to next exacerbation.

In some aspects, the patient has a level of one or both of LPA18:0 and LPA18:2 in the sample that is above a reference level and the method further comprises maintaining the treatment regimen of the patient and/or reducing monitoring of the patient.

In some aspects, the sample is a whole blood sample, a plasma sample, a serum sample, or a combination thereof.

In some aspects, the sample is a BALF sample.

In some aspects, the sample is an archival sample, a fresh sample, or a frozen sample.

In some aspects, the sample is a serum sample.

In some aspects, the level of one or both of LPA18:0 and LPA18:2 is a baseline level of one or both of LPA18:0 and LPA18:2.

In some aspects, the reference level is a pre-assigned level of one or both of LPA18:0 and LPA18:2.

In some aspects, the reference level for LPA18:0 is between about 0.03 μM to about 0.05 μM. In some aspects, the reference level for LPA18:0 is about 0.04 μM.

In some aspects, the reference level for LPA18:2 is between about 0.68 μM to about 0.84 μM. In some aspects, the reference level for LPA18:2 is about 0.76 μM.

In some aspects, the reference level is a level of one or both of LPA18:0 and LPA18:2 in a reference population.

In some aspects, the level of one or both of LPA18:0 and LPA18:2 in the sample is at or above the 67^{th} percentile of levels of LPA18:0 or LPA18:2, respectively, in the reference population.

In some aspects, the increased time to next exacerbation is an increase of at least 100 days.

In some aspects, the COPD is stage II, stage III, or stage IV COPD.

In some aspects, the exacerbation is an increase in one or more of dyspnea, cough, sputum volume, sputum purulence, fatigue, trouble sleeping, headache when waking up, confusion, and hypoxemia.

In some aspects, the patient is male.

In another aspect, the disclosure features a method for identifying, diagnosing, and/or predicting whether a patient may have an increased risk of COPD, the method comprising measuring a level of one or both of LPA18:0 and LPA18:1 in a sample from the patient, wherein a level of one or both of LPA18:0 and LPA18:1 in the sample that is above a reference level identifies, diagnoses, and/or predicts the patient as one who has an increased risk of an inflammatory respiratory disease.

In some aspects, the sample is a whole blood sample, a plasma sample, a serum sample, or a combination thereof.

In some aspects, the sample is a BALF sample.

In some aspects, the sample is an archival sample, a fresh sample, or a frozen sample.

In some aspects, the sample is a serum sample.

In some aspects, the level of one or both of LPA18:0 and LPA18:1 is a baseline level of one or both of LPA18:0 and LPA18:1.

In some aspects, the reference level is a pre-assigned level of one or both of LPA18:0 and LPA18:1.

In some aspects, the reference level is a level of one or both of LPA18:0 and LPA18:1 in a reference population.

In some aspects, the reference population is a population of patients not having an inflammatory respiratory disease.

In some aspects, the level of one or both of LPA18:0 and LPA18:1 in the sample is at least 4.6-fold higher than the average level of one or both of LPA18:0 and LPA18:1, respectively, in the reference population.

In some aspects, the reference level for LPA18:0 is between about 0.01 nmol/mL to about 0.035 nmol/mL.

In some aspects, the reference level for LPA18:0 is 0.025 nmol/mL.

In some aspects, the reference level for LPA18:1 is between about 0.05 nmol/mL to about 0.17 nmol/mL.

In some aspects, the reference level for LPA18:1 is 0.11 nmol/mL.

In some aspects, the COPD is stage II, stage III, or stage IV COPD.

In some aspects, the sample is from a fasted patient.

In another aspect, the disclosure features a method for identifying, diagnosing, and/or predicting whether a patient having idiopathic pulmonary fibrosis (IPF) may have an increased risk for an exacerbation or respiratory hospitalization, the method comprising measuring a level of one or more of lysophosphatidic acid (LPA)16:0, LPA18:1, LPA18:2, and LPA20:4 in a sample from the patient, wherein a level of one or more of LPA16:0, LPA18:1, LPA18:2, and LPA20:4 in the sample that is at or above a reference level identifies, diagnoses, and/or predicts the patient as one who is at an increased risk for an exacerbation or respiratory hospitalization.

In another aspect, the disclosure features a method for identifying, diagnosing, and/or predicting whether a patient having IPF may benefit from a treatment comprising an agent that reduces exacerbations, the method comprising measuring a level of one or more of LPA16:0, LPA18:1, LPA18:2, and LPA20:4 in a sample from the patient, wherein a level of one or more of LPA16:0, LPA18:1, LPA18:2, and LPA20:4 in the sample that is at or above a reference level identifies, diagnoses, and/or predicts the patient as one who may benefit from a treatment comprising an agent that reduces exacerbations.

In another aspect, the disclosure features a method of selecting a therapy for a patient having IPF, the method comprising measuring a level of one or more of LPA16:0, LPA18:1, LPA18:2, and LPA20:4 in a sample from the patient, wherein a level of one or more of LPA16:0, LPA18:1, LPA18:2, and LPA20:4 in the sample that is at or above a reference level identifies the patient as one who may benefit from a treatment comprising an agent that reduces exacerbations.

In some aspects, the patient has a level of one or more of LPA16:0, LPA18:0, LPA18:2, and LPA20:4 in the sample that is at or above a reference level and the method further comprises administering to the patient an effective amount of an agent that reduces exacerbations.

In another aspect, the disclosure features a method of treating a patient having IPF, the method comprising (a) measuring a level of one or more of LPA16:0, LPA18:1, LPA18:2, and LPA20:4 in a sample from the patient, wherein the level of one or more of LPA16:0, LPA18:1, LPA18:2, and LPA20:4 in the sample is at or above a reference level; and (b) administering an effective amount of an agent that reduces exacerbations to the patient.

In another aspect, the disclosure features a method of treating a patient having IPF and having a level of one or more of LPA16:0, LPA18:1, LPA18:2, and LPA20:4 in a sample from the patient that is at or above a reference level comprising administering an effective amount of an agent that reduces exacerbations to the patient.

In another aspect, the disclosure features a method of treating a patient having IPF, the method comprising administering to the patient an effective amount of an agent that reduces exacerbations, wherein the level of one or more of LPA16:0, LPA18:1, LPA18:2, and LPA20:4 in a sample from the patient has been determined to be at or above a reference level.

In some aspects, the sample is a whole blood sample, a plasma sample, a serum sample, or a combination thereof. In some aspects, the sample is an archival sample, a fresh sample, or a frozen sample. In some aspects, the sample is a serum sample.

In some aspects, the level of one or more of LPA16:0, LPA18:1, LPA18:2, and LPA20:4 is a baseline level of one or more of LPA16:0, LPA18:1, LPA18:2, and LPA20:4.

In some aspects, the reference level is a pre-assigned level of one or more of LPA16:0, LPA18:1, LPA18:2, and LPA20:4.

In some aspects, (a) the patient is female and the reference level for LPA16:0 is between about 0.207 μM to about 0.247 μM; or (b) the patient is male and the reference level for LPA16:0 is between about 0.153 μM to about 0.193 μM. In some aspects, (a) the patient is female and the reference level for LPA16:0 is about 0.227 μM; or (b) the patient is male and the reference level for LPA16:0 is about 0.173 μM.

In some aspects, (a) the patient is female and the reference level for LPA18:1 is between about 0.082 μM to about 0.122 μM; or (b) the patient is male and the reference level for LPA18:1 is between about 0.078 μM to about 0.118 μM. In some aspects, (a) the patient is female and the reference level for LPA18:1 is about 0.102 μM; or (b) the patient is male and the reference level for LPA18:1 is about 0.098 μM.

In some aspects, (a) the patient is female and the reference level for LPA18:2 is between about 0.388 μM to about 0.428 μM; or (b) the patient is male and the reference level for LPA18:2 is between about 0.339 μM to about 0.379 μM. In some aspects, (a) the patient is female and the reference level for LPA18:2 is about 0.408 μM; or (b) the patient is male and the reference level for LPA18:2 is about 0.359 μM.

In some aspects, (a) the patient is female and the reference level for LPA20:4 is between about 0.100 μM to about 0.140 μM; or (b) the patient is male and the reference level for LPA20:4 is between about 0.110 μM to about 0.150 μM. In some aspects, (a) the patient is female and the reference level for LPA20:4 is about 0.120 μM; or (b) the patient is male and the reference level for LPA20:4 is about 0.130 μM.

In some aspects, the reference level is a level of one or more of LPA16:0, LPA18:1, LPA18:2, and LPA20:4 in a reference population.

In some aspects, the level of one or more of LPA16:0, LPA18:1, and LPA18:2 in the sample is at or above the median of levels of LPA16:0, LPA18:1, or LPA18:2, respectively, in the reference population.

In some aspects, the reference population is a population of patients having IPF.

In some aspects, the reference population is a population of patients not having IPF.

In some aspects. the level of one or more of LPA16:0, LPA18:1, LPA18:2, and LPA20:4 in the sample is at least two-fold greater than the reference level.

In some aspects, the benefit comprises an extension in the patient's time to an exacerbation compared to treatment without the agent that reduces exacerbations.

In some aspects, the exacerbation is an acute respiratory deterioration. In some aspects, the acute respiratory deterioration is dyspnea. In some aspects, the acute respiratory deterioration is not caused by pneumothorax, cancer, heart failure, fluid overload, or pulmonary embolism.

In some aspects, the acute respiratory deterioration is associated with a new radiologic abnormality. In some aspects, the radiologic abnormality is bilateral ground-glass opacification/consolidation.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof.

In some aspects, the agent that reduces exacerbations is nintedanib or pirfenidone.

In some aspects, the agent that reduces exacerbations is approved by a regulatory health agency for reducing, controlling, or maintaining exacerbations. In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

In another aspect, the disclosure features a method for identifying, diagnosing, and/or predicting whether a patient having idiopathic pulmonary fibrosis (IPF) may have an increased risk of death, the method comprising measuring a level of one or both of triglyceride (TG)48:4-FA12:0 and TG48:4-FA18:2 in a sample from the patient, wherein a level of one or both of TG48:4-FA12:0 and TG48:4-FA18:2 in the sample that is below a reference level identifies, diagnoses, and/or predicts the patient as one who is at an increased risk of death.

In another aspect, the disclosure features a method for identifying, diagnosing, and/or predicting whether a patient having IPF may benefit from a treatment comprising an agent that reduces exacerbations, the method comprising measuring a level of one or both of TG48:4-FA12:0 and TG48:4-FA18:2 in a sample from the patient, wherein a level of one or both of TG48:4-FA12:0 and TG48:4-FA18:2 in the sample that is below a reference level identifies, diagnoses, and/or predicts the patient as one who may benefit from a treatment comprising an agent that reduces exacerbations.

In another aspect, the disclosure features a method of selecting a therapy for a patient having IPF, the method comprising measuring a level of one or both of TG48:4-FA12:0 and TG48:4-FA18:2 in a sample from the patient, wherein a level of one or both of TG48:4-FA12:0 and TG48:4-FA18:2 in the sample that is below a reference level identifies the patient as one who may benefit from a treatment comprising an agent that reduces exacerbations.

In some aspects, the patient has a level of one or both of TG48:4-FA12:0 and TG48:4-FA18:2 in the sample that is below a reference level and the method further comprises administering to the patient an effective amount of an agent that reduces exacerbations.

In another aspect, the disclosure features a method of treating a patient having IPF, the method comprising: (a) measuring a level of one or both of TG48:4-FA12:0 and TG48:4-FA18:2 in a sample from the patient, wherein the level of one or both of TG48:4-FA12:0 and TG48:4-FA18:2 in the sample is below a reference level; and (b) administering an effective amount of an agent that reduces exacerbations to the patient.

In another aspect, the disclosure features a method of treating a patient having IPF and having a level of one or both of TG48:4-FA12:0 and TG48:4-FA18:2 in a sample from the patient that is below a reference level comprising administering an effective amount of an agent that reduces exacerbations to the patient.

In another aspect, the disclosure features a method of treating a patient having IPF, the method comprising administering to the patient an effective amount of an agent that reduces exacerbations, wherein the level of one or both of TG48:4-FA12:0 and TG48:4-FA18:2 in a sample from the patient has been determined to be below a reference level.

In some aspects, the sample is a whole blood sample, a plasma sample, a serum sample, or a combination thereof.

In some aspects, the sample is an archival sample, a fresh sample, or a frozen sample.

In some aspects, the sample is a serum sample.

In some aspects, the level of one or both of TG48:4-FA12:0 and TG48:4-FA18:2 is a baseline level of one or both of TG48:4-FA12:0 and TG48:4-FA18:2.

In some aspects, the reference level is a pre-assigned level of one or both of TG48:4-FA12:0 and TG48:4-FA18:2.

In some aspects, (a) the patient is female and the reference level for TG48:4-FA12:0 is between about 0.800 μM to about 0.840 μM; or (b) the patient is male and the reference level for TG48:4-FA12:0 is between about 1.166 μM to about 1.206 μM. In some aspects, (a) the patient is female and the reference level for TG48:4-FA12:0 is about 0.820 μM; or (b) the patient is male and the reference level for TG48:4-FA12:0 is about 1.186 μM.

In some aspects, (a) the patient is female and the reference level for TG48:4-FA18:2 (μM) is between about 1.587 μM to about 1.627 μM; or (b) the patient is male and the reference level for TG48:4-FA18:2 (μM) is between about 2.153 μM to about 2.193 μM. In some aspects, (a) the patient is female and the reference level for TG48:4-FA18:2 (μM) is about 1.607 μM; or (b) the patient is male and the reference level for TG48:4-FA18:2 is about 2.173 μM.

In some aspects, the reference level is a level of one or both of TG48:4-FA12:0 and TG48:4-FA18:2 in a reference population.

In some aspects, the level of one or both of TG48:4-FA12:0 and TG48:4-FA18:2 in the sample is below the median of levels of TG48:4-FA12:0 or TG48:4-FA18:2, respectively, in the reference population.

In some aspects, the reference population is a population of patients having IPF.

In some aspects, the reference population is a population of patients not having IPF.

In some aspects, the level of one or both of TG48:4-FA12:0 or TG48:4-FA18:2 in the sample is at least two-fold less than the reference level.

In some aspects, the benefit comprises an extension in the patient's time to death compared to treatment without the agent that reduces exacerbations.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof.

In some aspects, the agent that reduces exacerbations is nintedanib or pirfenidone.

In some aspects, the agent that reduces exacerbations is approved by a regulatory health agency for reducing, controlling, or maintaining exacerbations.

In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

In another aspect, the disclosure features a method for predicting the time to exacerbation or respiratory hospitalization for a patient having IPF, the method comprising measuring a level of one or more of LPA16:0, LPA18:1, LPA20:4, LPA22:4, TG48:4-FA12:0, and TG48:4-FA18:2 in a sample from the patient, wherein (a) a level of one or more of LPA16:0, LPA18:1, LPA20:4, and LPA22:4 in the sample that is at or above a reference level or (b) a level of one or both of TG48:4-FA12:0 and TG48:4-FA18:2 in the sample that is below a reference level identifies the patient as one who may have a decreased time to exacerbation or respiratory hospitalization.

In some aspects, the patient has (a) a level of one or more of LPA16:0, LPA18:1, LPA20:4, and LPA22:4 in the sample that is at or above a reference level or (b) a level of one or both of TG48:4-FA12:0 and TG48:4-FA18:2 in the sample that is below a reference level and the method further comprises administering to the patient an effective amount of an agent that reduces exacerbations.

In some aspects, the sample is a whole blood sample, a plasma sample, a serum sample, or a combination thereof.

In some aspects, the sample is an archival sample, a fresh sample, or a frozen sample.

In some aspects the sample is a serum sample.

In some aspects, the level of one or more of LPA16:0, LPA18:1, LPA20:4, LPA22:4, TG48:4-FA12:0, and TG48:4-FA18:2 is a baseline level of one or more of LPA16:0, LPA18:1, LPA20:4, LPA22:4, TG48:4-FA12:0, and TG48:4-FA18:2. In some aspects, the reference level is a pre-assigned level of one or more of LPA16:0, LPA18:1, LPA20:4, LPA22:4, TG48:4-FA12:0, and TG48:4-FA18:2.

In some aspects, (a) the patient is female and the reference level for LPA16:0 is between about 0.207 μM to about 0.247 μM; or (b) the patient is male and the reference level for LPA16:0 is between about 0.153 μM to about 0.193 μM. In some aspects, (a) the patient is female and the reference level for LPA16:0 is about 0.227 μM; or (b) the patient is male and the reference level for LPA16:0 is about 0.173 μM.

In some aspects, (a) the patient is female and the reference level for LPA18:1 is between about 0.082 μM to about 0.122 μM; or (b) the patient is male and the reference level for LPA18:1 is between about 0.078 μM to about 0.118 μM. In some aspects, (a) the patient is female and the reference level for LPA18:1 is about 0.102 μM; or (b) the patient is male and the reference level for LPA18:1 is about 0.098 μM.

In some aspects, (a) the patient is female and the reference level for LPA20:4 is between about 0.100 μM to about 0.140 μM; or (b) the patient is male and the reference level for LPA20:4 is between about 0.110 μM to about 0.150 μM. In some aspects, (a) the patient is female and the reference level for LPA20:4 is about 0.120 μM; or (b) the patient is male and the reference level for LPA20:4 is about 0.130 μM.

In some aspects, (a) the patient is female and the reference level for LPA22:4 is between about 0.100 μM to about 0.140 μM; or (b) the patient is male and the reference level for LPA22:4 is between about 0.110 μM to about 0.150 μM. In some aspects, (a) the patient is female and the reference level for LPA22:4 is about 0.120 μM; or (b) the patient is male and the reference level for LPA22:4 is about 0.130 μM.

In some aspects, (a) the patient is female and the reference level for TG48:4-FA12:0 is between about 0.800 μM to about 0.840 μM; or (b) the patient is male and the reference level for TG48:4-FA12:0 is between about 1.166 μM to about 1.206 μM. In some aspects, (a) the patient is female and the reference level for TG48:4-FA12:0 is about 0.820 μM; or (b) the patient is male and the reference level for TG48:4-FA12:0 is about 1.186 μM.

In some aspects, (a) the patient is female and the reference level for TG48:4-FA18:2 (μM) is between about 1.587 μM to about 1.627 μM; or (b) the patient is male and the reference level for TG48:4-FA18:2 (μM) is between about 2.153 μM to about 2.193 μM. In some aspects, (a) the patient is female and the reference level for TG48:4-FA18:2 (μM) is about 1.607 μM; or (b) the patient is male and the reference level for TG48:4-FA18:2 is about 2.173 μM.

In some aspects, the reference level is a level of one or more of LPA16:0, LPA18:1, LPA20:4, LPA22:4, TG48:4-FA12:0, and TG48:4-FA18:2 in a reference population.

In some aspects, (a) the level of one or more of LPA16:0, LPA18:1, LPA20:4, and LPA22:4 in the sample is at or above the median of levels of LPA16:0, LPA18:1, LPA20:4, or LPA22:4, respectively, in the reference population or (b) the level of one or both of TG48:4-FA12:0 and TG48:4-FA18:2 in the sample is at or below the median of levels of TG48:4-FA12:0 or TG48:4-FA18:2, respectively, in the reference population.

In some aspects, the reference population is a population of patients having IPF.

In some aspects, the reference population is a population of patients not having IPF.

In some aspects, (a) the level of one or more of LPA16:0, LPA18:1, LPA20:4, and LPA22:4 in the sample is at least two-fold greater than the reference level or (b) the level of one or both of TG48:4-FA12:0 or TG48:4-FA18:2 in the sample is at least two-fold less than the reference level.

In some aspects, the exacerbation is an acute respiratory deterioration.

In some aspects, the acute respiratory deterioration is dyspnea.

In some aspects, the acute respiratory deterioration is not caused by pneumothorax, cancer, heart failure, fluid overload, or pulmonary embolism.

In some aspects, the acute respiratory deterioration is associated with a new radiologic abnormality.

In some aspects, the radiologic abnormality is bilateral ground-glass opacification/consolidation.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof.

In some aspects, the agent that reduces exacerbations is nintedanib or pirfenidone.

In some aspects, the agent that reduces exacerbations is approved by a regulatory health agency for reducing, controlling, or maintaining exacerbations.

In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

In another aspect, the disclosure features a method for identifying, diagnosing, and/or predicting whether a patient having idiopathic pulmonary fibrosis (IPF) may have an increased risk for deterioration in a measure of lung health, the method comprising: (a) measuring a level of one or more of LPA16:0, LPA16:1, LPA18:1, LPA18:2, and LPA20:4 in a sample from the patient, wherein the measure of lung health is diffusing capacity of carbon monoxide (DLCO) and a level of one or more of LPA16:0, LPA16:1, LPA18:1, LPA18:2, and LPA20:4 in the sample that is at or above a reference level identifies, diagnoses, and/or predicts the patient as one who is at an increased risk for deterioration of DLCO; (b) measuring a level of LPA22:4 in a sample from the patient, wherein the measure of lung health is ground glass opacity in the whole lung and a level of LPA22:4 in the sample that is at or above a reference level identifies, diagnoses, and/or predicts the patient as one who is at an increased risk for increased ground glass opacity in the whole lung; or (c) measuring a level of one or more of LPA16:0, LPA16:1, LPA18:0, LPA18:1, LPA18:2, and LPA20:4 in a sample from the patient, wherein the measure of lung health is fibrosis in lower zones of the lung and a level of one or more of LPA16:0, LPA16:1, LPA18:0, LPA18:1, LPA18:2, and LPA20:4 in the sample that is at or above a reference level identifies, diagnoses, and/or predicts the patient as one who is at an increased risk for fibrosis in lower zones of the lung.

In some aspects, (a) the patient is female and the reference level for LPA16:0 is between about 0.207 μM to about 0.247 μM; or (b) the patient is male and the reference level for LPA16:0 is between about 0.153 μM to about 0.193 μM. In some aspects, (a) the patient is female and the reference level for LPA16:0 is about 0.227 μM; or (b) the patient is male and the reference level for LPA16:0 is about 0.173 μM.

In some aspects, (a) the patient is female and the reference level for LPA16:1 is between about 0.101 ratio-to-standard (rts) to about 0.141 rts; or (b) the patient is male and the reference level for LPA16:1 is between about 0.058 rts to about 0.098 rts. In some aspects, (a) the patient is female and the reference level for LPA16:1 is about 0.121 rts; or (b) the patient is male and the reference level for LPA16:1 is about 0.078 rts.

In some aspects, (a) the patient is female and the reference level for LPA18:0 is between about 0.007 μM to about 0.047 μM; or (b) the patient is male and the reference level for LPA18:0 is between about 0.003 μM to about 0.043 μM. In some aspects, (a) the patient is female and the reference level for LPA18:0 is about 0.027 μM; or (b) the patient is male and the reference level for LPA18:0 is about 0.023 μM.

In some aspects, (a) the patient is female and the reference level for LPA18:1 is between about 0.082 μM to about 0.122 μM; or (b) the patient is male and the reference level for LPA18:1 is between about 0.078 μM to about 0.118 μM. In some aspects, (a) the patient is female and the reference level for LPA18:1 is about 0.102 μM; or (b) the patient is male and the reference level for LPA18:1 is about 0.098 μM.

In some aspects, (a) the patient is female and the reference level for LPA18:2 is between about 0.388 μM to about 0.428 μM; or (b) the patient is male and the reference level for LPA18:2 is between about 0.339 μM to about 0.379 μM. In some aspects, (a) the patient is female and the reference level for LPA18:2 is about 0.408 μM; or (b) the patient is male and the reference level for LPA18:2 is about 0.359 μM.

In some aspects, (a) the patient is female and the reference level for LPA20:4 is between about 0.100 μM to about 0.140 μM; or (b) the patient is male and the reference level for LPA20:4 is between about 0.110 μM to about 0.150 μM. In some aspects, (a) the patient is female and the reference level for LPA20:4 is about 0.120 μM; or (b) the patient is male and the reference level for LPA20:4 is about 0.130 μM.

In some aspects, (a) the patient is female and the reference level for LPA22:4 is between about 0.009 rts to about 0.049 rts; or (b) the patient is male and the reference level for LPA22:4 is between about 0.011 rts to about 0.051 rts. In some aspects, (a) the patient is female and the reference level for LPA22:4 is about 0.029 rts; or (b) the patient is male and the reference level for LPA22:4 is about 0.031 rts.

In another aspect, the disclosure features a method for preparing an LPA fraction from a patient useful for analyzing LPA species involved in an inflammatory respiratory disease, the method comprising (a) providing a serum sample or a BALF sample from the patient, wherein the serum sample has a volume of between about 5 μL to about 20 μL; and (b) extracting LPA from the serum sample in (a) using an extraction buffer comprising citric acid and disodium phosphate, wherein the extraction buffer does not result in the hydrolysis of the choline group from other lysophospholipids in the serum sample.

In some aspects, the method further comprises (c) separating the LPA species from the fraction of LPA extracted in (b).

In some aspects, the extraction buffer comprises between about 27-33 mM citric acid and between about 36-44 mM disodium phosphate. In some aspects, the extraction buffer comprises about 30 mM citric acid and 40 mM disodium phosphate. In some aspects, the extraction buffer does not comprise hydrochloric acid.

In some aspects, the separating in (c) is by liquid chromatography. In some aspects, the liquid chromatography is high performance liquid chromatography (HPLC). In some aspects, the HPLC is performed using a reverse-phase column. In some aspects, the reverse-phase column is a C18 column.

In another aspect, the disclosure features an LPA fraction from a patient produced by a method comprising (a) providing a serum sample from the patient, wherein the serum sample has a volume of between about 5 μL to about 20 μL; and (b) extracting LPA from the serum sample in (a) using an extraction buffer comprising citric acid and disodium phosphate, wherein the extraction buffer does not result in the hydrolysis of the choline group from other lysophospholipids in the serum sample.

In another aspect, the disclosure features a purified LPA species produced by a method comprising (a) providing a serum sample from a patient, wherein the serum sample has a volume of between about 5 μL to about 20 μL; (b) extracting LPA from the serum sample in (a) using an extraction buffer comprising citric acid and disodium phosphate, wherein the extraction buffer does not result in the hydrolysis of the choline group from other lysophospholipids in the serum sample; and (c) separating the LPA species from the fraction of LPA extracted in (b).

In another aspect, the disclosure features (a) method for analyzing the LPA fraction of claim 175, the method comprising separating the LPA species from the LPA fraction. In some aspects, the method further comprises analyzing the separated LPA species.

In another aspect, the disclosure features a method for analyzing an LPA species in a serum sample from a patient, the method comprising (a) providing a serum sample from the patient, wherein the serum sample has a volume of between about 5 μL to about 20 μL; (b) extracting LPA from the serum sample in (a) using an extraction buffer comprising citric acid and disodium phosphate, wherein the extraction buffer does not result in the hydrolysis of the choline group from other lysophospholipids in the serum sample; (c) separating the LPA species from the fraction of LPA extracted in (b); and (d) analyzing the separated LPA species produced in (c).

In some aspects, the analyzing is by mass spectrometry. In some aspects, the mass spectrometry is performed using a negative ionization mode. In some aspects, the limit of detection (LOD) for the LPA species is less than 0.008 pmol/μL serum. In some aspects, the LOD for the LPA species is between 0.002 pmol/μL and 0.008 pmol/μL serum. In some aspects, the LOD for the LPA species is less than 0.002 pmol/μL serum. In some aspects, the absolute recovery of the LPA species from the sample is between 82% and 110%.

In some aspects, the LPA species are one or more of LPA14:0, LPA16:0, LPA16:1, LPA18:0, LPA18:1, LPA18:2, LPA20:4, and LPA 22:4. In some aspects, the LPA species are one or more of LPA16:0, LPA18:0, LPA18:1, LPA18:2, and LPA20:4.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a scatter plot showing levels of LPA 16:0 detected by LC-MS/MS following extraction with two different sample preparation buffers. LPA: lysophosphatidic acid. Sodium buffer: 40 mM Na₂HP₄, 20 mM citric acid. LPA was extracted from serum of healthy donors. P-values shown were calculated by Student's t-test.

FIG. 1B is a scatter plot showing levels of LPA 18:0 detected by LC-MS/MS following extraction with two different sample preparation buffers. P-values shown were calculated by Student's t-test.

FIG. 1C is a scatter plot showing levels of LPA 18:1 detected by LC-MS/MS following extraction with two different sample preparation buffers. P-values shown were calculated by Student's t-test.

FIG. 1D is a scatter plot showing levels of LPA 18:2 detected by LC-MS/MS following extraction with two different sample preparation buffers. P-values shown were calculated by Student's t-test.

FIG. 1E is a scatter plot showing levels of LPA 20:4 detected by LC-MS/MS following extraction with two different sample preparation buffers. P-values shown were calculated by Student's t-test.

FIG. 1F is a scatter plot showing levels of LPA 16:0 detected by LC-MS/MS following extraction with two different sample preparation buffers. Disodium buffer: 40 mM Na2HPO4, 30 mM citric acid.

FIG. 1G is a scatter plot showing levels of LPA 18:0 detected by LC-MS/MS following extraction with two different sample preparation buffers. Disodium buffer: 40 mM Na2HPO4, 30 mM citric acid. P values were calculated by nonparametric Mann-Whitney u test.

FIG. 1H is a scatter plot showing levels of LPA 18:1 detected by LC-MS/MS following extraction with two different sample preparation buffers. Disodium buffer: 40 mM Na2HPO4, 30 mM citric acid. P values were calculated by nonparametric Mann-Whitney u test.

FIG. 1I is a scatter plot showing levels of LPA 18:2 detected by LC-MS/MS following extraction with two different sample preparation buffers. Disodium buffer: 40 mM Na2HPO4, 30 mM citric acid. P values were calculated by nonparametric Mann-Whitney u test.

FIG. 1J is a scatter plot showing levels of LPA 20:4 detected by LC-MS/MS following extraction with two different sample preparation buffers. Disodium buffer: 40 mM Na2HPO4, 30 mM citric acid. P values were calculated by nonparametric Mann-Whitney u test.

FIG. 1K is a scatter plot showing levels of LPA 17:0 detected by LC-MS/MS following extraction with two different sample preparation buffers. Disodium buffer: 40 mM Na2HPO4, 30 mM citric acid. P values were calculated by nonparametric Mann-Whitney u test.

FIG. 2A is a line graph showing stability of LPA species in a −80° C. freezer as concentration (μM) of the LPA species at timepoints over 35 days. LPA species were extracted from quality control (QC) samples.

FIG. 2B is a line graph showing stability of LPA species in extraction buffer in a 15° C. autosampler as the measured peak area of the LPA species at timepoints over 55 hours. LPA species were extracted from quality control (QC) samples.

FIG. 3A is a box and whisker plot showing concentrations of LPA 16:0 (log 2[μM]) in serum samples from healthy subjects and patients having chronic obstructive pulmonary disease (COPD). P-values shown were calculated by Student's t-test.

FIG. 3B is a box and whisker plot showing concentrations of LPA 18:0 (log 2[μM]) in serum samples from healthy subjects and patients having COPD. P-values shown were calculated by Student's t-test.

FIG. 3C is a box and whisker plot showing concentrations of LPA 18:1 (log 2[μM]) in serum samples from healthy subjects and patients having COPD. P-values shown were calculated by Student's t-test.

FIG. 3D is a box and whisker plot showing concentrations of LPA 18:2 (log 2[μM]) in serum samples from healthy subjects and patients having COPD. P-values shown were calculated by Student's t-test.

FIG. 3E is a box and whisker plot showing concentrations of LPA 20:4 (log 2[μM]) in serum samples from healthy subjects and patients having COPD. P-values shown were calculated by Student's t-test.

FIG. 4A is a box and whisker plot showing concentrations of LPA 16:0 (log 2[μM]) in serum samples from female (F) and male (M) patients having COPD. Univariate p values shown were calculated from nonparametric Mann-Whitney u test on the logarithm scale of LPA concentration. P values from multivariable analysis and q values from FDR adjustment are shown in Table 5.

FIG. 4B is a box and whisker plot showing concentrations of LPA 18:0 (log 2[μM]) in serum samples from female (F) and male (M) patients having COPD. P-values shown were calculated by Student's t-test. Univariate p values shown were calculated from nonparametric Mann-Whitney u test on the logarithm scale of LPA concentration. P values from multivariable analysis and q values from FDR adjustment are shown in Table 5.

FIG. 4C is a box and whisker plot showing concentrations of LPA 18:1 (log 2[μM]) in serum samples from female (F) and male (M) patients having COPD. P-values shown were calculated by Student's t-test. Univariate p values shown were calculated from nonparametric Mann-Whitney u test on the logarithm scale of LPA concentration. P values from multivariable analysis and q values from FDR adjustment are shown in Table 5.

FIG. 4D is a box and whisker plot showing concentrations of LPA 18:2 (log 2[μM]) in serum samples from female (F) and male (M) patients having COPD. P-values shown were calculated by Student's t-test. Univariate p values shown were calculated from nonparametric Mann-Whitney u test on the logarithm scale of LPA concentration. P values from multivariable analysis and q values from FDR adjustment are shown in Table 5.

FIG. 4E is a box and whisker plot showing concentrations of LPA 20:4 (log 2[μM]) in serum samples from female (F) and male (M) patients having COPD. P-values shown were calculated by Student's t-test. Univariate p values shown were calculated from nonparametric Mann-Whitney u test on the logarithm scale of LPA concentration. P values from multivariable analysis and q values from FDR adjustment are shown in Table 5.

FIG. 5A is a box and whisker plot showing LPA 16:0 levels in serum samples from female and male patients with and without chronic bronchitis (CB). No: patients without CB; Yes: patients with CB. The univariate analysis p values shown were calculated from the nonparametric Mann-Whitney u test on the logarithm scale of LPA concentration. P values from multivariable analysis and q values from FDR adjustment are shown in Table 5.

FIG. 5B is a box and whisker plot showing LPA 18:0 levels in serum samples from female and male patients with and without CB. No: patients without CB; Yes: patients with CB. The univariate analysis p values shown were calculated from the nonparametric Mann-Whitney u test on the logarithm scale of LPA concentration. P values from multivariable analysis and q values from FDR adjustment are shown in Table 5.

FIG. 5C is a box and whisker plot showing LPA 18:1 levels in serum samples from female and male patients with and without CB. No: patients without CB; Yes: patients with CB. The univariate analysis p values shown were calculated from the nonparametric Mann-Whitney u test on the logarithm scale of LPA concentration. P values from multivariable analysis and q values from FDR adjustment are shown in Table 5.

FIG. 5D is a box and whisker plot showing LPA 18:2 levels in serum samples from female and male patients with and without CB. No: patients without CB; Yes: patients with CB. The univariate analysis p values shown were calculated from the nonparametric Mann-Whitney u test on the logarithm scale of LPA concentration. P values from multivariable analysis and q values from FDR adjustment are shown in Table 5.

FIG. 5E is a box and whisker plot showing LPA 20:4 levels in serum samples from female and male patients with and without CB. No: patients without CB; Yes: patients with CB. The univariate analysis p values shown were calculated from the nonparametric Mann-Whitney u test on the logarithm scale of LPA concentration. P values from multivariable analysis and q values from FDR adjustment are shown in Table 5.

FIG. 6A is a box and whisker plot showing LPA 16:0 levels in serum samples from female and male COPD patients from North and South America or the rest of the world. NSA: North and South America; Others: rest of the world. P-values shown were calculated by Student's t-test.

FIG. 6B is a box and whisker plot showing LPA 18:0 levels in serum samples from female and male COPD patients from North and South America or the rest of the world. NSA: North and South America; Others: rest of the world. P-values shown were calculated by Student's t-test.

FIG. 6C is a box and whisker plot showing LPA 18:1 levels in serum samples from female and male COPD patients from North and South America or the rest of the world. NSA: North and South America; Others: rest of the world. P-values shown were calculated by Student's t-test.

FIG. 6D is a box and whisker plot showing LPA 18:2 levels in serum samples from female and male COPD patients from North and South America or the rest of the world. NSA: North and South America; Others: rest of the world. P-values shown were calculated by Student's t-test.

FIG. 6E is a box and whisker plot showing LPA 20:4 levels in serum samples from female and male COPD patients from North and South America or the rest of the world. NSA: North and South America; Others: rest of the world. P-values shown were calculated by Student's t-test.

FIG. 7A is a box and whisker plot showing LPA 16:0 levels in serum samples from healthy subjects from the small cohort study who were smokers (S) or non-smokers (NS).

FIG. 7B is a box and whisker plot showing LPA 18:0 levels in serum samples from healthy subjects from the small cohort study who were smokers or non-smokers.

FIG. 7C is a box and whisker plot showing LPA 18:1 levels in serum samples from healthy subjects from the small cohort study who were smokers or non-smokers.

FIG. 7D is a box and whisker plot showing LPA 18:2 levels in serum samples from healthy subjects from the small cohort study who were smokers or non-smokers.

FIG. 7E is a box and whisker plot showing LPA 20:4 levels in serum samples from healthy subjects from the small cohort study who were smokers or non-smokers.

FIG. 8A is a box and whisker plot showing LPA16:0 levels in baseline serum samples from female and male COPD patients who were current or former smokers.

FIG. 8B is a box and whisker plot showing LPA18:0 levels in baseline serum samples from female and male COPD patients who were current or former smokers.

FIG. 8C is a box and whisker plot showing LPA18:1 levels in baseline serum samples from female and male COPD patients who were current or former smokers.

FIG. 8D is a box and whisker plot showing LPA18:2 levels in baseline serum samples from female and male COPD patients who were current or former smokers.

FIG. 8E is a box and whisker plot showing LPA20:4 levels in baseline serum samples from female and male COPD patients who were current or former smokers.

FIG. 9A is a box and whisker plot showing LPA16:0 levels in baseline serum samples from female and male COPD patients who were underweight or had normal weight (15<BMI<25), were overweight (OV), (25<BMI<30), or were obese (BMI>30).

FIG. 9B is a box and whisker plot showing LPA18:0 levels in baseline serum samples from female and male COPD patients who were underweight or had normal weight (15<BMI<25), were overweight (OV), (25<BMI<30), or were obese (BMI>30).

FIG. 9C is a box and whisker plot showing LPA18:1 levels in baseline serum samples from female and male COPD patients who were underweight or had normal weight (15<BMI<25), were overweight (OV), (25<BMI<30), or were obese (BMI>30).

FIG. 9D is a box and whisker plot showing LPA18:2 levels in baseline serum samples from female and male COPD patients who were underweight or had normal weight (15<BMI<25), were overweight (OV), (25<BMI<30), or were obese (BMI>30).

FIG. 9E is a box and whisker plot showing LPA20:4 levels in baseline serum samples from female and male COPD patients who were underweight or had normal weight (15<BMI<25), were overweight (OV), (25<BMI<30), or were obese (BMI>30).

FIG. 10A is a scatter plot showing correlation (Pearson) of levels of LPA16:0 to age of female (F) patients.

FIG. 10B is a scatter plot showing correlation (Pearson) of levels of LPA18:0 to age of female (F) patients.

FIG. 10C is a scatter plot showing correlation (Pearson) of levels of LPA18:1 to age of female (F) patients.

FIG. 10D is a scatter plot showing correlation (Pearson) of levels of LPA18:2 to age of female (F) patients.

FIG. 10E is a scatter plot showing correlation (Pearson) of levels of LPA20:4 to age of female (F) patients.

FIG. 10F is a scatter plot showing correlation (Pearson) of levels of LPA16:0 to age of male (M) patients.

FIG. 10G is a scatter plot showing correlation (Pearson) of levels of LPA18:0 to age of male (M) patients.

FIG. 10H is a scatter plot showing correlation (Pearson) of levels of LPA18:1 to age of male (M) patients.

FIG. 10I is a scatter plot showing correlation (Pearson) of levels of LPA18:2 to age of male (M) patients.

FIG. 10J is a scatter plot showing correlation (Pearson) of levels of LPA20:4 to age of male (M) patients.

FIG. 11 is a set of scatter plots showing correlation of levels of the LPA species LPA16:0, LPA18:0, LPA18:1, LPA18:2, and LPA20:4 with measures of lung function: FEV₁, FVC, and FEV₁/FVC. Pearson's r- and p-values are shown.

FIG. 12 is a chart showing correlation values (Spearman's rho) for correlation of levels of the LPA species LPA16:0, LPA18:0, LPA18:1, LPA18:2, and LPA20:4 with one another and with levels of the biomarkers monocytes, neutrophils, eosinophils, platelets, fibrinogen, and immunoglobulin E (IgE) at baseline in serum samples from placebo patients from the NCT02546700 clinical trial.

FIG. 13 is a chart showing the risk of exacerbation by baseline biomarker in male COPD patients. Each baseline biomarker profile was fitted to a multivariate logistic regression model adjusted for the following covariates: number of exacerbations within the last 12 months, smoking status, geographical region, bronchodilator response, and baseline COPD medications. An odds ratio above 1 denotes a higher risk of exacerbation in patients with blood eosinophils ≥200 cells/μl compared to <200 cells/μl; with chronic bronchitis compared to no chronic bronchitis (CB_SGRQ: chronic bronchitis as identified using St. George's Respiratory Questionnaire for COPD); with fibrinogen ≥3.5 g/L compared to <3.5 g/L; or with a level of the LPA species LPA20:4, LPA18:1, LPA16:0, LPA18:2, or LPA18:0 that is in the lowest tertile of values for the LPA species compared to highest tertile. Q value is the false-discovery-rate-adjusted p value. Line arrows denote censored confidence intervals.

FIG. 14 is a set of graphs showing the adjusted exacerbation rate (per patient per year) over 24 weeks by baseline biomarker profile and gender. L=lowest-; M=mid-; H=highest tertile of LPA levels for the respective LPA species. Adjusted exacerbation rates are estimates from a Quasi-Poisson regression model adjusted for the following covariates in addition to log_{(patient-years)} as an offset: number of exacerbations within the last 12 months, smoking status, geographical region, bronchodilator response, and baseline COPD medications. P-values compare L to M or H subgroup. *p<0.05; **p<0.01. N=number of patients.

FIG. 15A is a set of Kaplan-Meier curves showing the percentage of male patients who have not had an exacerbation over time stratified by L=lowest-; M=mid-; H=highest tertile of baseline concentrations of LPA16:0. The baseline biomarker profile was fitted to a Cox proportional hazards regression model adjusted for the following covariates: number of exacerbations within the last 12 months, smoking status, geographical region, bronchodilator response, and baseline COPD medications. P-values show the comparison among the three LPA16:0 subgroups.

FIG. 15B is a set of Kaplan-Meier curves showing the percentage of male patients who have not had an exacerbation over time stratified by L=lowest-; M=mid-; H=highest tertile of baseline concentrations of LPA18:0. The baseline biomarker profile was fitted to a Cox proportional hazards regression model as described for FIG. 15A.

FIG. 15C is a set of Kaplan-Meier curves showing the percentage of male patients who have not had an exacerbation over time stratified by L=lowest-; M=mid-; H=highest tertile of baseline concentrations of LPA18:1. The baseline biomarker profile was fitted to a Cox proportional hazards regression model as described for FIG. 15A.

FIG. 15D is a set of Kaplan-Meier curves showing the percentage of male patients who have not had an exacerbation over time stratified by L=lowest-; M=mid-; H=highest tertile of baseline concentrations of LPA18:2. The baseline biomarker profile was fitted to a Cox proportional hazards regression model as described for FIG. 15A.

FIG. 15E is a set of Kaplan-Meier curves showing the percentage of male patients who have not had an exacerbation over time stratified by L=lowest-; M=mid-; H=highest tertile of baseline concentrations of LPA20:4. The baseline biomarker profile was fitted to a Cox proportional hazards regression model as described for FIG. 15A.

FIG. 16 is a Venn diagram showing the overlap of lipid species with unadjusted p-value <0.05 when compared between LPA-low versus LPA-high subgroups for each lipid species in men.

FIG. 17 is a Venn diagram showing the overlap of lipid species with unadjusted p-value <0.05 when compared between LPA-low versus LPA-high subgroups for each lipid species in women.

FIG. 18A is a set of box plots showing the baseline concentration of each LPA species (μM) stratified by gender. **p<0.005; ***p<0.001 Student t-test.

FIG. 18B is a set of box plots showing the baseline concentration of each LPA species (μM) stratified by statin use. **p<0.005; ***p<0.001 Student t-test.

FIG. 18C is a set of box plots showing the baseline concentration of each LPA species (μM) stratified by whether the patient has chronic bronchitis (CB_SGRQ: chronic bronchitis as identified using St. George's Respiratory Questionnaire for COPD). **p<0.005; ***p<0.001 Student t-test.

FIG. 19A is a heatmap showing overlap in low, mid, and high LPA species levels in men. Rows show LPA species and are coded by tertile cutoffs (μM) used to categorize patients into low (blue), mid (gray), and high (red) tertiles for levels of the LPA species. Each column represents a patient. Patients who were in the low or high tertile for all LPA species are indicated by brackets.

FIG. 19B is a heatmap showing overlap in low, mid, and high LPA species levels in women. Rows show LPA species and are coded by tertile cutoffs (μM) used to categorize patients into low (blue), mid (gray), and high (red) tertiles for levels of the LPA species. Each column represents a patient. Patients who were in the low or high tertile for all LPA species are indicated by brackets.

FIG. 20 is a chart showing the risk of exacerbation by baseline biomarker in female COPD patients. Each baseline biomarker profile was fitted to a multivariate logistic regression model adjusted for the following covariates: number of exacerbations within the last 12 months, smoking status, geographical region, bronchodilator response, and baseline COPD medications. An odds ratio above 1 denotes a higher risk of exacerbation in patients with blood eosinophils ≥200 cells/μl compared to <200 cells/μl; with chronic bronchitis compared to no chronic bronchitis (CB_SGRQ: chronic bronchitis as identified using St. George's Respiratory Questionnaire for COPD); with fibrinogen ≥3.5 g/L compared to <3.5 g/L; or with a level of the LPA species LPA20:4, LPA18:1, LPA16:0, LPA18:2, or LPA18:0 that is in the lowest tertile of values for the LPA species compared to highest tertile. Q value is the false-discovery-rate-adjusted p value.

FIG. 21A is a set of Kaplan-Meier curves showing the percentage of female patients who have not had an exacerbation overtime stratified by L=lowest-; M=mid-; H=highest tertile of baseline concentrations of LPA16:0. The baseline biomarker profile was fitted to a Cox proportional hazards regression model as described for FIG. 15A.

FIG. 21B is a set of Kaplan-Meier curves showing the percentage of female patients who have not had an exacerbation overtime stratified by L=lowest-; M=mid-; H=highest tertile of baseline concentrations of LPA18:0. The baseline biomarker profile was fitted to a Cox proportional hazards regression model as described for FIG. 15A.

FIG. 21C is a set of Kaplan-Meier curves showing the percentage of female patients who have not had an exacerbation overtime stratified by L=lowest-; M=mid-; H=highest tertile of baseline concentrations of LPA18:1. The baseline biomarker profile was fitted to a Cox proportional hazards regression model as described for FIG. 15A.

FIG. 21D is a set of Kaplan-Meier curves showing the percentage of female patients who have not had an exacerbation overtime stratified by L=lowest-; M=mid-; H=highest tertile of baseline concentrations of LPA18:2. The baseline biomarker profile was fitted to a Cox proportional hazards regression model as described for FIG. 15A.

FIG. 21E is a set of Kaplan-Meier curves showing the percentage of female patients who have not had an exacerbation over time stratified by L=lowest-; M=mid-; H=highest tertile of baseline concentrations of LPA20:4. The baseline biomarker profile was fitted to a Cox proportional hazards regression model as described for FIG. 15A.

FIG. 22 is a set of box plots showing the duration of exacerbations (in days) stratified by L=lowest-; M=mid-; H=highest tertile of baseline LPA levels for LPA16:0, LPA18:0, LPA18:1, LPA18:2, and LPA20:4 and gender. Kruskal-Wallis p-values are shown. N=number of exacerbation events.

FIG. 23A is a set of bar graphs showing differential expression of twelve classes of lipids between men in low and high baseline LPA species subgroups. Xaxes denote the average log 2 (analyte abundance in low/analyte abundance in high); values less than 0 indicate a decrease, and values greater than 0 an increase in low subgroup versus high subgroup patients. Green bars denote unadjusted p-value <0.05. CE: cholesteryl esters; CER: ceramides; DAG: diacylglycerols; DCER: dihydroceramides; HCER: hexosylceramides; LCER: lactosylceramides; LPC: lysophosphatidylcholines; LPE: lysophosphatidylethanolamines; PC: phosphatidylcholines; PE: phosphatidylethanolamines; SM: sphingomyelins; TAG: triacylglycerols.

FIG. 23B is a set of volcano plots showing lipid species in men in low and high baseline LPA species subgroups. X axes denote the average log₂ (analyte abundance in low/analyte abundance in high). Y axes indicates the −log₁₀ (unadjusted p-value). Colored circles denote unadjusted p-value <0.05; red circles denote lipid species with higher abundance in LPA low subgroups compared to LPA high subgroups; blue circles denote lipid species with lower abundance in LPA low subgroups compared to LPA high subgroups. Colored labels highlight species with greater fold change.

FIG. 24A is a set of bar graphs showing differential expression of twelve classes of lipids between women in low and high baseline LPA species subgroups. Xaxes denote the average log₂ (analyte abundance in low/analyte abundance in high); values less than 0 indicate a decrease, and values greater than 0 an increase in low subgroup versus high subgroup patients. Green bars denote unadjusted p-value <0.05; orange bars denote false discovery rate <0.1. CE: cholesteryl esters; CER: ceramides; DAG: diacylglycerols; DCER: dihydroceramides; HCER: hexosylceramides; LCER: lactosylceramides; LPC: lysophosphatidylcholines; LPE: lysophosphatidylethanolamines; PC: phosphatidylcholines; PE: phosphatidylethanolamines; SM: sphingomyelins; TAG: triacylglycerols.

FIG. 24B is a set of volcano plots showing lipid species in women in low and high baseline LPA species subgroups. Xaxes denote the average log₂ (analyte abundance in low/analyte abundance in high). Yaxes indicates the −log₁₀ (unadjusted p-value). Colored circles denote unadjusted p-value <0.05; red circles denote lipid species with higher abundance in LPA low subgroups compared to LPA high subgroups; blue circles denote lipid species with lower abundance in LPA low subgroups compared to LPA high subgroups. Colored labels highlight species with greater fold change or smaller p-value.

FIG. 25A is a plot showing the adjusted exacerbation rate (per patient per year) stratified by L=lowest-; M=mid-; H=highest tertile of baseline ceramide (CER) levels in female COPD patients.

FIG. 25B is a plot showing the adjusted exacerbation rate (per patient per year) stratified by L=lowest-; M=mid-; H=highest tertile of baseline CER levels in male COPD patients.

FIG. 26A is a plot showing the adjusted exacerbation rate (per patient per year) stratified by L=lowest-; M=mid-; H=highest tertile of baseline hydroxyceramide (HCER) levels in female COPD patients.

FIG. 26B is a plot showing the adjusted exacerbation rate (per patient per year) stratified by L=lowest-; M=mid-; H=highest tertile of baseline HCER levels in male COPD patients.

FIG. 27A is a plot showing the adjusted exacerbation rate (per patient per year) stratified by L=lowest-; M=mid-; H=highest tertile of baseline lactosylceramide (LCER) levels in female COPD patients.

FIG. 27B is a plot showing the adjusted exacerbation rate (per patient per year) stratified by L=lowest-; M=mid-; H=highest tertile of baseline LCER levels in male COPD patients.

FIG. 28A is a plot showing the adjusted exacerbation rate (per patient per year) stratified by L=lowest-; M=mid-; H=highest tertile of baseline lysophosphatidylcholine (LPC) levels in female COPD patients.

FIG. 28B is a plot showing the adjusted exacerbation rate (per patient per year) stratified by L=lowest-; M=mid-; H=highest tertile of baseline LPC levels in male COPD patients.

FIG. 29A is a plot showing the adjusted exacerbation rate (per patient per year) stratified by L=lowest-; M=mid-; H=highest tertile of baseline sphingomyelin (SM) levels in female COPD patients.

FIG. 29B is a plot showing the adjusted exacerbation rate (per patient per year) stratified by L=lowest-; M=mid-; H=highest tertile of baseline SM levels in male COPD patients.

FIG. 30 is a plot showing a multivariate linear regression model adjusted for age and sex used to assess the differences in lipid levels between healthy controls and IPF patients. The x-axis shows log₂ (analyte abundance in IPF/analyte abundance in healthy controls), and the y-axis shows −log₁₀ (adjusted p-value or false discovery rate of the multivariate regression). Yellow circles denote lipid species with false discovery rate <0.05; red circles denote lipid species with false discovery rate <0.05 and fold change >2.

FIG. 31A is a set of plots showing the results of univariate and multivariate linear regressions adjusted for age and sex used to assess the association of LPA16:0, LPA16:1, and LPA18:0 with baseline demographic or clinical measures in healthy patients.

FIG. 31B is a set of plots showing the results of univariate and multivariate linear regressions adjusted for age and sex used to assess the association of LPA16:0, LPA16:1, and LPA18:0 with baseline demographic or clinical measures in idiopathic pulmonary fibrosis (IPF) patients.

FIG. 31C is a chart showing correlation values (Spearman rho) for LPA and TG species in IPF patients.

FIG. 31D is a chart showing the association between LPA or TG species and protein biomarkers in multivariate linear regression analyses adjusted for age, sex and geographic region. −ve=negative association; F=female; M=males; 6 MWT=6-minute walk test distance in meter; rts=ratio-to-standard.

FIG. 32 is a set of scatter plots showing the results of multivariate linear regression models adjusted for the covariates age, sex, baseline FVC % pred, baseline DLCO % pred, and geographical region used to assess the association between baseline levels of the indicated LPA or TG species with DLCO % pred decline (over 52 weeks) calculated as slope. DLCO % pred=percentage of predicted diffusion capacity of carbon monoxide; FVC % pred=percentage of predicted forced vital capacity; rts=ratio to standard; uM=micromolar. **p<0.01; ***p<0.001.

FIG. 33 is a plot showing the risk of exacerbation or respiratory hospitalization over 52 weeks based on baseline lipid profile. Baseline lipid profile was fitted to a multivariate logistic regression model adjusted for the following covariates: age, sex, baseline FVC % pred, baseline DLCO % pred, and geographical region. An odd ratio above 1 denotes higher odds of exacerbation or respiratory hospitalization in patients with the higher levels (median) LPA compared to patients with lower levels of (<median) of LPA; or in patients with the lower levels (<median) TG compared to patients with higher levels of (≥median) of TG. Line arrows denote censored confidence intervals. DLCO % pred=percentage of predicted diffusion capacity of carbon monoxide; FVC % pred=percentage of predicted forced vital capacity.

FIG. 34 is a set of charts showing the probability of exacerbation or lack of respiratory hospitalization overtime based on levels of lipid biomarkers. Baseline LPA and TG profiles were fitted to a Cox proportional hazards regression model adjusted for the following covariates: age, sex, baseline FVC % pred, baseline DLCO % pred, and geographical region. Group (gr) 0=biomarker-low (<median); 1=biomarker-high (≥median). DLCO % pred=percentage of predicted diffusion capacity of carbon monoxide; FVC % pred=percentage of predicted forced vital capacity. #p<0.1; *p<0.05.

FIG. 35 is a plot showing the risk of mortality over 52 weeks based on baseline lipid profile. Baseline lipid profile was fitted to a multivariate logistic regression model adjusted for the following covariates: age, sex, baseline FVC % pred, baseline DLCO % pred, and geographical region. An odd ratio above 1 denotes higher odds of mortality in patients with the higher levels (<median) LPA compared to patients with lower levels of (<median) of LPA; or in patients with the lower levels (<median) TG compared to patients with higher levels of (≥median) of TG. Line arrows denote censored confidence intervals. DLCO % pred=percentage of predicted diffusion capacity of carbon monoxide; FVC % pred=percentage of predicted forced vital capacity.

FIG. 36 is a set of charts showing the probability of mortality over time based on levels of lipid biomarkers. Baseline TG profile was fitted to a Cox proportional hazards regression model adjusted for the following covariates: age, sex, baseline FVC % pred, baseline DLCO % pred, and geographical region. Group (gr) 0=biomarker-low (<median); 1=biomarker-high (≥median). DLCO % pred=percentage of predicted diffusion capacity of carbon monoxide; FVC % pred=percentage of predicted forced vital capacity. #p<0.1.

FIG. 37 is a set of scatter plots showing ground glass opacity change from baseline in whole lungs over 72 weeks and baseline lipid levels. A multivariate linear regression model adjusted for the following covariates: age, sex, baseline FVC % pred, baseline DLCO % pred, and geographical region, was used to assess the association between LPA and TG with ground glass opacity change from baseline. DLCO % pred=percentage of predicted diffusion capacity of carbon monoxide; FVC % pred=percentage of predicted forced vital capacity; rts=ratio to standard; uM=micromolar. *p<0.05.

FIG. 38 is a set of plots showing the proportion of radiographic changes (ground glass opacity (upper left panel), honeycombing (lower left panel), and fibrosis (right panel)) in the indicated regions of the lungs at screen visit and week 72. Median and interquartile ranges of the radiographic metrics are shown as boxplots, with grey lines connecting the individual patients. GGCAD=ground glass opacity; HCCAD=honeycombing; QLFCAD=fibrosis; SCRN=screen; wk=week.

FIG. 39A is a set of scatter plots showing fibrosis change from baseline in lower left zones of the lung over 72 weeks and baseline lipid levels. A multivariate linear regression model adjusted for the following covariates: age, sex, baseline FVC % pred, baseline DLCO % pred, and geographical region, was used to assess the association between LPA and TG with fibrosis change from baseline. DLCO % pred=percentage of predicted diffusion capacity of carbon monoxide; rts=ratio to standard; uM=micromolar. #p<0.1; *p<0.05; **p<0.01.

FIG. 39B is a set of scatter plots showing fibrosis change from baseline in lower right zones of the lung over 72 weeks and baseline lipid levels. A multivariate linear regression model adjusted for the following covariates: age, sex, baseline FVC % pred, baseline DLCO % pred, and geographical region, was used to assess the association between LPA and TG with fibrosis change from baseline. DLCO % pred=percentage of predicted diffusion capacity of carbon monoxide; rts=ratio to standard; uM=micromolar. #p<0.1; *p<0.05; **p<0.01.

FIG. 40 is a set of plots showing the level (log 2-transformed) of LPA16:0, LPA16:1, LPA18:1, LPA18:2, and LPA20:4 in IPF patients and healthy controls.

FIG. 41A is a set of plots showing levels of LPA16:0, LPA16:1 and LPA18:0 in female (F) and male (M) IPF patients and a plot showing a negative correlation between LPA18:0 level (log₂ transformed) and diffusing capacity of carbon monoxide (DLCO) at baseline.

FIG. 41B is a set of scatter plots showing correlation between levels of the indicated LPA species (log₂ transformed) and six-minute walk distance (6 MWD) in IPF patients at baseline in univariate or multivariate regression adjusted for age and sex.

FIG. 42 is a set of scatter plots showing correlation between levels of the indicated LPA species (log₂ transformed; μm or ratio to standard) and DLCO (slope: DLCO decline over 48 weeks) in male IPF patients.

FIG. 43 is a set of scatter plots showing correlation between levels of the indicated LPA species (log₂ transformed; μm or ratio to standard) and FVC (slope: FVC decline over 48 weeks) in male IPF patients.

FIG. 44 is a set of curves showing survival probability overtime in male IPF patients having levels of the indicated LPA species that are below a median level (gr=0) or greater than or equal to a median level (gr=1). Median cutoffs: LPA16:0-0.173 μM, LPA16:1-0.0780 ratio-to-standard; LPA18:1-0.0983 μM; LPA20:4-0.130 μM.

FIG. 45 is a set of scatter plots showing correlation between levels of the indicated LPA species (log 2 transformed; μm or ratio to standard) and increased ground glass opacity at week 72 (GGCAD_CHG) in male IPF patients.

FIG. 46 is a set of scatter plots showing correlation between levels of the indicated LPA species (log 2 transformed; μm or ratio to standard) and increased honeycombing at week 72 (HCCAD_CHG) in male IPF patients.

FIG. 47 is a set of scatter plots showing correlation between levels of the indicated LPA species (log 2 transformed; μm or ratio to standard) and increased interstitial lung disease (ILD) metric at week 72 (QILD_CHG) in male IPF patients.

FIG. 48A is a set of plots showing the level (log 2-transformed) of the indicated LPC species in IPF patients and healthy controls.

FIG. 48B is a set of plots showing the level (log 2-transformed) of the indicated LPC species in IPF patients and healthy controls.

FIG. 49 is a set of scatter plots showing correlation between levels of the indicated LPC species (log 2 transformed) and decline in FVC over 48 weeks in IPF patients.

FIG. 50 is a set of curves showing survival probability overtime in IPF patients having levels of the indicated LPC species that are below a median level (gr=0) or greater than or equal to a median level (gr=1). Median cutoffs: (μM): LPC15:0: 1.2696 (female), 1.3919 (male); LPC20:2: 0.7232 (female), 0.7513 (male); LPC22:0: 0.0854 (female), 0.0906 (male); LPC22:1: 0.0853 (female), 0.0888 (male).

FIG. 51A is a set of scatter plots showing correlation between levels of the indicated LPC species (log 2 transformed) and increased ground glass opacity at week 72 (GGCAD_CHG) in IPF patients.

FIG. 51B is a set of scatter plots showing correlation between levels of the indicated LPC species (log 2 transformed) and increased ground glass opacity at week 72 (GGCAD_CHG) in IPF patients.

FIG. 52 is a set of scatter plots showing correlation between levels of the indicated LPC species (log 2 transformed) and increased honeycombing at week 72 (HCCAD_CHG) in IPF patients.

FIG. 53 is a set of scatter plots showing correlation between levels of the indicated LPC species (log 2 transformed) and increased fibrosis at week 72 (QLFCAD_CHG) in IPF patients.

FIG. 54A is a set of scatter plots showing correlation between levels of the indicated LPC species (log 2 transformed) and increased ILD metric at week 72 (QILD_CHG) in IPF patients.

FIG. 54B is a set of scatter plots showing correlation between levels of the indicated LPC species (log 2 transformed) and increased ILD metric at week 72 (QILD_CHG) in IPF patients.

FIG. 55A is a set of plots showing the level (log 2-transformed) of the indicated LPE species in IPF patients and healthy controls.

FIG. 55B is a set of plots showing the level (log 2-transformed) of the indicated LPE species in IPF patients and healthy controls.

FIG. 56 is a scatter plot showing correlation between levels of the indicated LPE species (log 2 transformed) and decreased DLCO at week 48 in IPF patients.

FIG. 57 is a set of scatter plots showing correlation between levels of the indicated LPE species (log 2 transformed) and decline in FVC over 48 weeks in IPF patients.

FIG. 58 is a set of curves showing survival probability overtime in IPF patients having levels of the indicated LPE species that are below a median level (gr=0) or greater than or equal to a median level (gr=1). Median cutoffs: (μM): 0.0435 (female), 0.0422 (male).

FIG. 59 is a set of scatter plots showing correlation between levels of the indicated LPE species (log 2 transformed) and increased ground glass opacity at week 72 (GGCAD_CHG) in IPF patients.

FIG. 60 is a set of scatter plots showing correlation between levels of the indicated LPE species (log 2 transformed) and increased honeycombing at week 72 (HCCAD_CHG) in IPF patients.

FIG. 61 is a scatter plot showing correlation between levels of the indicated LPE species (log 2 transformed) and increased fibrosis at week 72 (QLFCAD_CHG) in IPF patients.

FIG. 62 is a set of scatter plots showing correlation between levels of the indicated LPE species (log 2 transformed) and increased ILD metric at week 72 (QILD_CHG) in IPF patients.

FIG. 63A is a pair of bar graphs showing sex (female (F)) or male (M)) and status (alive or dead) of patients for whom lipid analyses were performed.

FIG. 63B is a chart showing classes of lipids assessed in global lipid profiling.

FIG. 64 is a set of box-and-whisker plots showing levels of the indicated lipid species in patients having IPF and in healthy control patients.

FIG. 65 is a set of schematic diagrams showing the structures of LPA18:1, LPE18:1, and LPC18:1 and a chart showing the results of a lipid profiling analysis in patients having IPF compared to healthy control patients. Lipid species that were at significantly higher levels in the IPF patient samples (p<0.05, <0.01, <0.001, or <0.0001) are indicated by shade.

FIG. 66 is a set of box-and-whisker plots showing levels of the indicated ceramide (CE) species in patients having IPF and in healthy control patients.

FIG. 67A is a plot showing levels of phosphatidylcholine (PC) species in patient samples compared to healthy donors.

FIG. 67B is a set of box-and-whisker plots showing levels of PC species in patients having IPF and in healthy control patients.

FIG. 68 is a set of box-and-whisker plots showing levels of the indicated LPC species in IPF patients who were disease progressors (FP) or non-progressors (0).

FIG. 69 is a set of box-and-whisker plots showing levels of the indicated lipid species in IPF patients who had fibrosis (1) or did not have fibrosis (0).

FIG. 70 is a set of charts showing correlations between the indicated LPA, LPE, and LPC species and the indicated biomarkers. The direction of correlation is indicated by color.

FIG. 71 is a set of box-and-whisker plots showing levels of the indicated dihydroceramide (DCER) species in IPF patients who had fibrosis (1), did not have fibrosis (0), or were a mix between (1) and (0).

FIG. 72 is a set of box-and-whisker plots showing levels of the indicated phosphatidylcholine (PC) species in IPF patients who were disease progressors (1), were not disease progressors (0), or were a mix between (1) and (0).

FIG. 73 is a pie chart and a stacked bar chart showing correlation between PC species and the indicated biomarkers.

FIG. 74 is a set of box-and-whisker plots showing levels of the indicated phosphatidylethanolamine (PE) species in IPF patients who were disease progressors (1), were not disease progressors (0), or were a mix between (1) and (0).

FIG. 75 is a pie chart and a pair of stacked bar charts showing correlation between PE or PC species and the indicated biomarkers.

FIG. 76A is a chromatogram showing separation of the indicated LPA standards.

FIG. 76B is a pair of chromatograms showing levels of LPA14:0, LPA16:1 and LPA22:4 detected in healthy and COPD serum using theoretical multiple reactions monitoring (MRM) transitions.

FIG. 76C is a chromatogram showing levels and separation of LPG18:0, LP118:0, LPS18:0, LPC18:0 and LPE18:0 with LPA18:0 in serum.

FIG. 76D is a pair of extracted ion chromatograms showing levels of LPA18:0 and LPC18:0 from healthy and COPD serum.

FIG. 77A is a box and whisker plot showing concentrations of the indicated LPA species in serum samples from healthy subjects and patients having chronic obstructive pulmonary disease (COPD). Q values shown are from logistic regression analysis that were adjusted for age and gender on the logarithm scale of LPA concentration, and then adjusted by false-discovery-rate.

FIG. 77B is a PLS-DA score plot showing LPA species (Healthy controls versus COPD patients). PLS-DA model was validated using 7-fold internal cross-validation. Permutation test confirmed the robustness of the model (100 permutations).

### DETAILED DESCRIPTION OF THE INVENTION

### 1. Definitions

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.” In some embodiments, “about” may refer to ±15%, ±10%, ±5%, or ±1% as understood by a person of skill in the art.

It is understood that aspects of the invention described herein include “comprising,” “consisting,” and “consisting essentially of” aspects.

As used herein, the term “inflammatory respiratory disease” refers to a disease, disorder, or condition associated with inflammation in the respiratory tract, e.g., chronic obstructive pulmonary disease (COPD), idiopathic pulmonary fibrosis (IPF), asthma, interstitial lung disease (ILD), or cystic fibrosis.

As used herein, the term “exacerbation” refers to a worsening of one or more symptoms of an inflammatory respiratory disease (e.g., COPD, IPF, or asthma), e.g., a significant deterioration in a clinical measure that requires medical attention. An exacerbation of COPD may be one or more new or increased symptoms of COPD, e.g., an increase in breathlessness (dyspnea), cough, sputum volume, sputum purulence, fatigue, trouble sleeping, headache when waking up, confusion, or reduced oxygen level (hypoxemia), e.g., a new or increased symptom that lasts for at least two consecutive days and/or that leads to hospitalization and/or treatment with systemic corticosteroids and/or antibiotics. An exacerbation of IPF may be one or more new or increased symptoms of the IPF, e.g., acute respiratory deterioration (e.g., dyspnea), wherein the acute respiratory deterioration is not caused by pneumothorax, cancer, heart failure, fluid overload, or pulmonary embolism. The exacerbation of IPF may be associated with a new radiologic abnormality, e.g., bilateral ground-glass opacification/consolidation. An exacerbation of asthma may be an episode of one or more of progressively worsening shortness of breath, coughing, wheezing, and chest tightness. The episode may be acute or subacute. Duration of an exacerbation may be defined as, e.g., the duration for which the patient experiences symptoms and/or the number of days for which the patient is on systemic corticosteroids and/or antibiotics to treat the exacerbation. The exacerbation may be a severe exacerbation, e.g., an exacerbation requiring hospitalization.

As used herein, the term “agent that reduces exacerbations” refers to an agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in patients having an inflammatory respiratory disease. Such agents include agents that are used for the treatment of an inflammatory respiratory disease (e.g., maintenance medications), e.g., agents used for the treatment of COPD, IPF, and/or asthma, and agents that are used for the treatment of exacerbations of inflammatory respiratory diseases. Agents that reduce exacerbations include agents that have been approved by a regulatory health agency (e.g., the U.S. Food & Drug Administration (FDA), the European Medicines Agency (EMA), the Pharmaceuticals and Medical Devices Agency (PMDA), or the National Medical Products Administration (NMPA)) for reducing, controlling, or maintaining exacerbations. Exemplary agents that reduce exacerbations include, but are not limited to an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid (e.g., an inhaled corticosteroid (ICS) or an oral corticosteroid (OCS)), an antibiotic, an althpa-1 antitrypsin augmentation therapy, mepolizumab, benralizumab, or a combination thereof; an agent disclosed in the GLOBAL INITIATIVE FOR CHRONIC OBSTRUCTIVE LUNG DISEASE™ (GOLD) Pocket Guide to COPD Diagnosis, Management, and Prevention (2020 Edition); nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof; an inhaled short-acting beta₂-agonist (SABA), albuterol, bitolterol, levalbuterol, pirbuterol, a systemic SABA (e.g., epinephrine or terbutaline), an anticholinergic (e.g., ipratropium bromide), or a systemic corticosteroid (e.g., prednisone, methylprednisolone, or prednisolone).

As used herein, the term “efficacy” refers to the effectiveness of a therapy (e.g., a therapy comprising an agent that reduces exacerbations) in the treatment of a disease (e.g., an inflammatory respiratory disease, e.g., chronic obstructive pulmonary disease (COPD), idiopathic pulmonary fibrosis (IPF), or asthma). Efficacy may be assessed using, e.g., exacerbation rate, time to exacerbation (e.g., time to a first or subsequent exacerbation), severity of exacerbation, or duration of exacerbation. In some aspects, efficacy may be assessed using overall survival (OS). In some aspects, efficacy may be assessed using measures of lung function, e.g., spirometry, e.g., FEV₁ (forced expiratory volume in one second) or FVC (forced vital capacity). In some aspects, the patient has asthma or COPD and efficacy is assessed using FEV₁. In other aspects, the patient has IPF and efficacy is assessed using FVC.

“Individual response” or “response” can be assessed using any endpoint indicating a benefit to the individual, including, without limitation, (1) inhibition, to some extent, of disease progression (e.g., exacerbation or progression of an inflammatory respiratory disease), including slowing down or complete arrest; (2) relief, to some extent, of one or more symptoms associated with the disease or disorder (e.g., relief of symptoms of exacerbations of an inflammatory respiratory disease); (3) increase or extension in the length of survival, including overall survival and progression free survival (e.g., increase or extension in the length of time to exacerbation); (4) decreased duration and/or severity of exacerbations, (5) decreased mortality at a given point of time following treatment, and/or (6) improvement in one or more measures of lung function (e.g., FEV₁ or FVC).

An “effective response” of a patient or a patient's “responsiveness” to treatment with a medicament and similar wording refers to the clinical or therapeutic benefit imparted to a patient at risk for, or suffering from, a disease or disorder, such as an inflammatory respiratory disease. In some aspects, such benefit includes one or more of extending survival (including overall survival and/or progression-free survival (e.g., increase or extension in the length of time to an exacerbation)), reducing the rate, duration, and or severity of exacerbations, or improving signs or symptoms of the inflammatory respiratory disease (e.g., improving one or more measures of lung function, e.g., FEV₁ or FVC).

An “effective amount” of a compound, for example, an agent that reduces exacerbations or a composition (e.g., pharmaceutical composition) thereof, is at least the minimum amount required to achieve the desired therapeutic or prophylactic result, such as a measurable improvement or prevention of a particular disorder (e.g., an inflammatory respiratory disease (e.g., COPD, IPF, or asthma), or an exacerbation thereof). An effective amount herein may vary according to factors such as the disease state, age, sex, and weight of the patient, and the ability of the compound to elicit a desired response in the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “disorder” is any condition that would benefit from treatment including, but not limited to, chronic and acute disorders or diseases including those pathological conditions which predispose the mammal to the disorder in question. In one aspect, the disorder is an inflammatory respiratory disease, e.g., COPD, IPF, or asthma.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the patient being treated, and can be performed either for prophylaxis or during the course of clinical pathology. Desirable effects of treatment include, but are not limited to, preventing occurrence or recurrence of disease, preventing exacerbations of disease, reducing the rate of exacerbations, reducing the duration of exacerbations, reducing the severity of exacerbations, reducing the risk of an exacerbation (e.g., a next exacerbation), alleviation of symptoms, diminishment of any direct or indirect pathological consequences of the disease, decreasing the rate of disease progression, amelioration or palliation of the disease state, and remission or improved prognosis. In some aspects, an agent that reduces exacerbations is used to reduce the frequency, duration, or severity of an exacerbation of an inflammatory respiratory disease, e.g., COPD, IPF, or asthma.

A “patient,” “subject,” or “individual” is a human. In certain aspects, the patient is male.

As used herein, “administering” is meant a method of giving a dosage of a compound (e.g., an agent that reduces exacerbations) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

The term “concomitant” or “concurrent” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, e.g., the frequency, duration, or severity of an exacerbation of an inflammatory respiratory disease.

### II. Methods for Preparing and Analyzing Lpa Fractions

Lysophosphatidic acids (LPAs) are phospholipid derivatives that can act as signaling mediators. Species of LPA (e.g., LPA14:0, LPA16:0, LPA16:1, LPA18:0, LPA8:1, LPA18:2, LPA20:4, LPA22:5, and LPA22:6) differ in length and fatty acid saturation.

LPAs are generated by multiple enzymes, including phospholipase C, phospholipase A1 (PLA1), and phospholipase A2 (PLA2), as well as lysophospholipase D (lysoPLD). Autotaxin (ATX), a lysoPLD family member, has been detected in various tissues and biofluids, such as plasma, serum, follicular fluid, saliva and malignant effusions. The majority of bioactive LPAs detected in blood and inflamed sites are generated by the autotaxin-lysophosphatidic acid (ATX-LPA) pathway: ATX, a secreted glycoprotein, functions primarily as lysophospholipase D to remove the choline moiety from lysophosphatidylcholine (LPC), thereby generating LPA.

LPAs generated through the ATX pathway are extracellular signaling molecules. LPAs bind to G-protein coupled receptors LPAR1-6 to regulate fibrosis; regulate lymphocyte homing; regulate platelet aggregation; promote proliferation of vascular smooth muscle cells and fibroblasts; and activate vascular endothelial cells, monocytes, and macrophages.

Previously used methods for extracting LPA species have used HCl in the extraction process; however, such methods have been shown to overestimate the levels of LPA species in samples. In the presence of strong acid, lysophospholipids such as lysophosphatidylcholines (LPCs) can be converted to LPA by hydrolysis of the choline group from the lysophospholipid (e.g., LPC species), thereby artificially increasing LPA levels in the sample. Onorato et al. (J. Lipid Res., 55: 1784-1796, 2014) showed that using 6N HCl to acidify samples can cause overestimation of LPA levels by about 10-fold.

LPAs are known to have critical functions in many pathophysiological contexts. The autotaxin-lysophosphatidic acid (ATX-LPA) pathway has been implicated in inflammatory lung conditions including COPD (Shea et al., Proc Am Thorac Soc, 9: 102-110, 2012; Magkrioti, World J Respirol, 3: 77, 2013).

Thus, the accurate measurement of LPAs as potential biomarkers is very important for diagnosis and prognosis of inflammatory respiratory diseases, e.g., COPD, IPF, or asthma.

A. Methods for Preparing LPA Fractions

i. LPA Fractions and Methods for Preparing Same

In some aspects, the disclosure features a method for preparing an LPA fraction from a patient useful for analyzing LPA species involved in an inflammatory respiratory disease, the method comprising a step (a) of providing a sample from the patient (e.g., a whole blood sample, a plasma sample, a serum sample, or a combination thereof; a bronchoalveolar lavage fluid (BALF) sample; or a urine sample), for example, a sample having a volume of between about 5 μL to about 20 μL, and a step (b) of extracting LPA from the sample in (a) using an extraction buffer comprising citric acid and disodium phosphate, wherein the extraction buffer does not result in the hydrolysis of the choline group from other lysophospholipids in the sample. The sample may be an archival sample, a fresh sample, or a frozen sample. In some aspects, the sample is a serum sample.

In some aspects, the disclosure features an LPA fraction from a patient produced by a method comprising a step (a) of providing a sample from the patient (e.g., a whole blood sample, a plasma sample, a serum sample, or a combination thereof; a bronchoalveolar lavage fluid (BALF) sample; or a urine sample), for example, a sample having a volume of between about 5 μL to about 20 μL; and a step (b) of extracting LPA from the sample in (a) using an extraction buffer comprising citric acid and disodium phosphate, wherein the extraction buffer does not result in the hydrolysis of the choline group from other lysophospholipids in the serum sample. The sample may be an archival sample, a fresh sample, or a frozen sample. In some aspects, the sample is a serum sample.

In some aspects, the LPA species are one or more of LPA14:0, LPA16:0, LPA16:1, LPA18:0, LPA18:1, LPA18:2, LPA20:4, and LPA 22:4, e.g., one or more of LPA16:0, LPA18:0, LPA18:1, LPA18:2, and LPA20:4.

ii. Samples

In some aspects, the sample (e.g., whole blood sample, plasma sample, serum sample, or combination thereof; BALF sample; or urine sample) has a volume of between about 0.5 μL and about 3 mL, e.g., has a volume of 3 mL or less, 2 mL or less, 1 mL or less, 600 μL or less, 500 μL or less, 100 μL or less, 50 μL or less, 20 μL or less, 19 μL or less, 18 μL or less, 17 μL or less, 16 μL or less, 15 μL or less, 14 μL or less, 13 μL or less, 12 μL or less, 11 μL or less, 10 μL or less, 9 μL or less, 8 μL or less, 7 μL or less, 6 μL or less, 5 μL or less, 4 μL or less, 3 μL or less, 2 μL or less, 1 μL or less, or 0.5 μL or less. In some aspects, the sample has a volume of about 0.5 μL to about 100 μL, e.g., has a volume of about 1 μL to about 80 μL, about 1 μL to about 50 μL, about 5 μL to about 30 μL, or about 5 μL to about 20 μL. In some aspects, the sample has a volume of about 5 μL to about 100 μL, e.g., has a volume of about 10 μL to about 80 μL, about 10 μL to about 50 μL, about 15 μL to about 30 μL, or about 15 μL to about 25 μL. In some aspects, the sample has a volume of about 5 μL to about 600 μL, e.g., has a volume of about 10 μL to about 500 μL, about 20 μL to about 400 μL, about 50 μL to about 300 μL, or about 100 μL to about 200 μL. In some aspects, the sample has a volume of 20 μL.

In some aspects, the sample is a serum sample having a volume of between about 0.5 μL and about 3 mL, e.g., has a volume of 3 mL or less, 2 mL or less, 1 mL or less, 500 μL or less, 100 μL or less, 50 μL or less, 20 μL or less, 19 μL or less, 18 μL or less, 17 μL or less, 16 μL or less, 15 μL or less, 14 μL or less, 13 μL or less, 12 μL or less, 11 μL or less, 10 μL or less, 9 μL or less, 8 μL or less, 7 μL or less, 6 μL or less, 5 μL or less, 4 μL or less, 3 μL or less, 2 μL or less, 1 μL or less, or 0.5 μL or less. In some aspects, the serum sample has a volume of about 0.5 μL to about 100 μL, e.g., has a volume of about 1 μL to about 80 μL, about 1 μL to about 50 μL, about 5 μL to about 30 μL, or about 5 μL to about 20 μL. In some aspects, the serum sample has a volume of about 5 μL to about 100 μL, e.g., has a volume of about 10 μL to about 80 μL, about 10 μL to about 50 μL, about 15 μL to about 30 μL, or about 15 μL to about 25 μL. In some aspects, the serum sample has a volume of about 5 μL to about 600 μL, e.g., has a volume of about 10 μL to about 500 μL, about 20 μL to about 400 μL, about 50 μL to about 300 μL, or about 100 μL to about 200 μL. In some aspects, the serum sample has a volume of 20 μL.

In some aspects, the sample is collected from a fasted patient, e.g., a patient who has fasted for at least 4 hours, at least 6 hours, at least 8 hours, at least 10 hours, at least 12 hours, or more than 12 hours prior to the collection of the sample.

iii. Extraction Buffers

In some aspects, the extraction buffer of step (b) comprises about 25 mM to about 35 mM citric acid, e.g., comprises about 26 mM-34 mM, 27 mM-33 mM, 28 mM-32 mM, 29 mM-31 mM, 29.5 mM-30.5 15 mM, or 29.9 mM to 30.1 mM citric acid, e.g., comprises about 25 mM, 26 mM, 27 mM, 28 mM, 29 mM, 29.1 mM, 29.2 mM, 29.3 mM, 29.4 mM, 29.5 mM, 29.6 mM, 29.7 mM, 29.8 mM, 29.9 mM, 30.0 mM, 30.1 mM, 30.2 mM, 30.3 mM, 30.4 mM, 30.5 mM, 30.6 mM, 30.7 mM, 30.8 mM, 30.9 mM, 31 mM, 32 mM, 33 mM, 34 mM, or 35 mM citric acid. In some aspects, the extraction buffer of step (b) comprises about 27 mM to about 33 mM citric acid.

In some aspects, the extraction buffer comprises about 35 mM to about 45 mM disodium phosphate, e.g., comprises about 36 mM-44 mM, 37 mM-43 mM, 38 mM-42 mM, 39 mM-41 mM, 39.5 mM-40.5 mM, or 39.9 mM to 40.1 mM disodium phosphate, e.g., comprises about 35 mM, 36 mM, 37 mM, 38 mM, 39 mM, 39.1 mM, 39.2 mM, 39.3 mM, 39.4 mM, 39.5 mM, 39.6 mM, 39.7 mM, 39.8 mM, 39.9 mM, 40.0 mM, 40.1 mM, 40.2 mM, 40.3 mM, 40.4 mM, 40.5 mM, 40.6 mM, 40.7 mM, 40.8 mM, 40.9 mM, 41 mM, 42 mM, 43 mM, 44 mM, or 45 mM disodium phosphate. In some aspects, the extraction buffer of step (b) comprises about 30 mM citric acid and 40 mM disodium phosphate.

In some aspects, the extraction buffer does not comprise hydrochloric acid.

B. Methods for Separating LPA Species from LPA Fractions

In some aspects, the methods described herein further comprise a step (c) of separating the LPA species from the fraction of LPA extracted in step (b), e.g., separating one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4) from the fraction of LPA extracted in step (b). In some aspects, the separating in (c) is by liquid chromatography, e.g., high performance liquid chromatography (HPLC). The HPLC may be performed using a reverse-phase column, e.g., a C18 column.

In some aspects, the disclosure features a method for analyzing an LPA fraction produced using the methods described herein, the method comprising separating the LPA species (e.g., separating one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4) from the LPA fraction, e.g., separating the species using liquid chromatography, e.g., HPLC (e.g., HPLC performed using a reverse-phase column, e.g., a C18 column).

In some aspects, the disclosure features a purified LPA species produced by a method comprising a step (a) of providing a sample (e.g., a whole blood sample, a plasma sample, a serum sample, or a combination thereof; a bronchoalveolar lavage fluid (BALF) sample; or a urine sample) from a patient, wherein the sample has a volume of between about 5 μL to about 20 μL; a step (b) of extracting LPA from the serum in (a) using an extraction buffer comprising citric acid and disodium phosphate, wherein the extraction buffer does not result in the hydrolysis of the choline group from other lysophospholipids in the sample; and a step (c) of separating the LPA species from the fraction of LPA extracted in (b), e.g., separating the species using liquid chromatography, e.g., HPLC (e.g., HPLC performed using a reverse-phase column, e.g., a C18 column). The separated LPA species may be one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4). In some aspects, the sample is a serum sample.

C. Methods for Analyzing LPA Species

In some aspects of the methods described herein, the methods further comprise a step (d) of analyzing the separated LPA species produced in step (c), e.g., analyzing the identity, amount, and/or level of the LPA species in the sample, e.g., analyzing the identity, amount, and/or level of one or more LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., analyzing one, two three, four, five, six, seven, or all eight of LPA14:0, LPA16:0, LPA16:1, LPA18:0, LPA18:1, LPA18:2, LPA20:4, and LPA 22:4 or analyzing one, two, three, four, or all five of LPA16:0, LPA18:0, LPA18:1, LPA18:2, and LPA20:4).

In some aspects, the disclosure features a method for analyzing an LPA species in a serum sample from a patient, the method comprising (a) providing a sample from the patient (e.g., a whole blood sample, a plasma sample, a serum sample, or a combination thereof; a bronchoalveolar lavage fluid (BALF) sample; or a urine sample; e.g., a serum sample as described in Section IIA(ii)); (b) extracting LPA from the sample in (a) using an extraction buffer comprising citric acid and disodium phosphate (e.g., an extraction buffer as described in Section IIA(iii)), wherein the extraction buffer does not result in the hydrolysis of the choline group from other lysophospholipids in the sample; (c) separating the LPA species from the fraction of LPA extracted in (b) (e.g., separating the LPA species as described in Section IIB); and (d) analyzing the separated LPA species produced in (c).

In some aspects, the analyzing is by mass spectrometry, e.g., mass spectrometry performed using a negative ionization mode.

In some aspects, the limit of detection (LOD) for the LPA species is less than 0.05 pmol/μL serum, e.g., less than 0.01 pmol/μL serum, 0.009 pmol/μL serum, 0.008 pmol/μL serum, 0.007 pmol/μL serum, 0.006 pmol/μL serum, 0.005 pmol/μL serum, 0.004 pmol/μL serum, 0.003 pmol/μL serum, 0.002 pmol/μL serum, 0.001 pmol/μL serum, or less than 0.0001 pmol/μL serum. In some aspects, the LOD for the LPA species is less than 0.008 pmol/μL serum. In some aspects, the LOD for the LPA species is less than 0.002 pmol/μL serum. In some aspects, the LOD for the LPA species is between 0.0001 and 0.05 pmol/μL serum, e.g., between 0.001 and 0.01 pmol/μL serum, between 0.001 and 0.009 pmol/μL serum, or between 0.002 pmol/μL and 0.008 pmol/μL serum.

In some aspects, the absolute recovery of the LPA species from the sample is between 75% and 125%, e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124% or 125%, e.g., between 80% and 110%, between 82% and 110%, or between 82% and 102%. In some aspects, the absolute recovery of the LPA species from the sample is between 82% and 110%.

### III. Diagnostic and Therapeutic Methods

A. Diagnostic Methods for COPD and Asthma

i. LPA Biomarkers for Increased Risk of Exacerbation

In some aspects, the disclosure features a method for identifying, diagnosing, and/or predicting whether a patient having an inflammatory respiratory disease (e.g., a respiratory disease described in Section IIIE herein, e.g., COPD or asthma) may have an increased risk for an exacerbation (e.g., an exacerbation of the respiratory disease as described in Section IIIE herein), the method comprising measuring a level of one or more of lysophosphatidic acid (LPA)16:0, LPA18:0, LPA18:1, LPA18:2, and LPA20:4 in a sample from the patient (e.g., a baseline level of the one or more LPA species), wherein a level of one or more of LPA16:0, LPA18:0, LPA18:1, LPA18:2, and LPA20:4 in the sample that is below a reference level identifies the patient as one who is at an increased risk for an exacerbation; diagnoses the patient as one who is at an increased risk for an exacerbation; or predicts that the patient is one who is at an increased risk for an exacerbation. In some aspects, the inflammatory respiratory disease is COPD. In some aspects, the inflammatory respiratory disease is asthma and the method comprises measuring a level of one or more of LPA16:0, LPA18:0, and LPA18:2, e.g., one, two, or all three of LPA16:0, LPA18:0, and LPA18:2) in the sample from the patient (e.g., a baseline level of one or more of LPA16:0, LPA18:0, and LPA18:2).

In some aspects, the disclosure features a method for identifying, diagnosing, and/or predicting whether a patient having an inflammatory respiratory disease may benefit from a treatment comprising an agent that reduces exacerbations (e.g., an agent described in Section IIIG herein), the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies the patient as one who may benefit from a treatment comprising an agent that reduces exacerbations. In some aspects, the inflammatory respiratory disease is COPD. In some aspects, the inflammatory respiratory disease is asthma and the method comprises measuring a level of one or more of LPA16:0, LPA18:0, and LPA18:2, e.g., one, two, or all three of LPA16:0, LPA18:0, and LPA18:2) in the sample from the patient (e.g., a baseline level of one or more of LPA16:0, LPA18:0, and LPA18:2).

In some aspects, the disclosure features a method of selecting a therapy for a patient having an inflammatory respiratory disease (e.g., a respiratory disease described in Section IIIE herein, e.g., COPD or asthma), the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies the patient as one who may benefit from a treatment comprising an agent that reduces exacerbations. In some aspects, the inflammatory respiratory disease is COPD. In some aspects, the inflammatory respiratory disease is asthma and the method comprises measuring a level of one or more of LPA16:0, LPA18:0, and LPA18:2, e.g., one, two, or all three of LPA16:0, LPA18:0, and LPA18:2) in the sample from the patient (e.g., a baseline level of one or more of LPA16:0, LPA18:0, and LPA18:2).

In some aspects, the benefit comprises an extension in the patient's time to an exacerbation compared to treatment without an agent that reduces exacerbations, e.g., an extension of at least one day, at least two days, at least three days, at least four days, at least five days, at least six days, at least one week, at least two weeks, at least three weeks, at least one month, at least two months, at least three months, at least four months, at least five months, at least six months, at least one year, or more than one year in the patient's time to a first exacerbation or time to a next exacerbation.

In some aspects, the benefit comprises a reduction in the duration of an exacerbation in the patient compared to treatment without the agent that reduces exacerbations, e.g., a reduction of at least 10 minutes, at least 20 minutes, at least 30 minutes, at least 1 hour, at least 2 hours, at least 6 hours, at least 12 hours, at least one day, at least two days, at least three days, at least four days, at least five days, at least six days, at least one week, at least two weeks, at least three weeks, at least one month, at least two months, at least three months, at least four months, at least five months, at least six months, at least one year, or more than one year in the duration of an exacerbation. In some aspects, the reduction in the duration of an exacerbation is a reduction of at least one day, at least two days, at least three days, at least four days, at least five days, at least six days, at least one week, at least two weeks, at least three weeks, at least one month, at least two months, at least three months, at least four months, at least five months, or at least six months in the duration of an exacerbation.

In some aspects, the benefit comprises a reduction in the frequency and/or duration of hospitalization. In some aspects, the benefit comprises a reduction in the use of a therapeutic agent, e.g., a systemic corticosteroid or an antibiotic.

In some aspects, the disclosure features a method of identifying a patient suitable for administration with an agent that treats an inflammatory respiratory disease (e.g., a respiratory disease described in Section IIIE herein, e.g., COPD or asthma) or an agent that reduces exacerbations of an inflammatory respiratory disease (e.g., an agent described in Section IIIG herein), the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level identifies the patient as one who is suitable for administration with an agent that treats an inflammatory respiratory disease or an agent that reduces exacerbations of an inflammatory respiratory disease. In some aspects, the inflammatory respiratory disease is COPD. In some aspects, the inflammatory respiratory disease is asthma and the method comprises measuring a level of one or more of LPA16:0, LPA18:0, and LPA18:2, e.g., one, two, or all three of LPA16:0, LPA18:0, and LPA18:2) in the sample from the patient (e.g., a baseline level of one or more of LPA16:0, LPA18:0, and LPA18:2).

In some aspects, the patient has a level of one or more of LPA16:0, LPA18:0, LPA18:1, LPA18:2, and LPA20:4 (e.g., a level of one, two, three, four, or all five of LPA16:0, LPA18:0, LPA18:1, LPA18:2, and LPA20:4) in the sample that is below a reference level, and the method further comprises administering to the patient an effective amount of an agent that reduces exacerbations (e.g., an agent described in Section IIIG herein). In some aspects, the inflammatory respiratory disease is asthma, the patient has a level of one or more of LPA16:0, LPA18:0, and LPA18:2, (e.g., a level of one, two, or all three of LPA16:0, LPA18:0, LPA18:2) in the sample that is below a reference level, and the method further comprises administering to the patient an effective amount of an agent that reduces exacerbations (e.g., an agent described in Section IIIG herein).

The sample may be, e.g., a whole blood sample, a plasma sample, a serum sample, or a combination thereof; a bronchoalveolar lavage fluid (BALF) sample; or a urine sample. The sample may be, e.g., an archival sample, a fresh sample, or a frozen sample. In some aspects, the sample is a serum sample.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4, e.g., a level that is measured when the patient is not experiencing an exacerbation, e.g., has recovered from an exacerbation or has never experienced an exacerbation.

In some aspects, the level of one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4) in the sample from the patient is below a reference level.

In some aspects, the reference level is a pre-assigned level of one or more of LPA16:0, LPA18:0, LPA18:1, LPA18:2, and LPA20:4.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level of LPA16:0, LPA18:0, LPA18:1, or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:0, LPA18:1, or LPA18:2 levels, respectively, in the reference population.

In some aspects, the reference level of LPA16:0, LPA18:0, LPA18:1, or LPA18:2 is the 33^{rd} percentile of LPA16:0, LPA18:0, LPA18:1, or LPA18:2 levels, respectively, in the reference population.

In some aspects, the level of LPA16:0, LPA18:0, LPA18:1, or LPA18:2 in the sample from the patient (e.g., the baseline level of LPA16:0, LPA18:0, LPA18:1, or LPA18:2 in the patient) is at or below the 33^{rd} percentile of LPA16:0, LPA18:0, LPA18:1, or LPA18:2 levels in the reference population, e.g., is at or below the 32^{nd} percentile, 31^{st} percentile, 30^{th} percentile, 29^{th} percentile, 28^{th} percentile, 27^{th} percentile, 26^{th} percentile, 25^{th} percentile, 24^{th} percentile, 23^{rd} percentile, 22^{nd} percentile, 21^{st} percentile, 20^{th} percentile, 19^{th} percentile, 18^{th} percentile, 17^{th} percentile, 16^{th} percentile, 15^{th} percentile, 14^{th} percentile, 13^{th} percentile, 12^{th} percentile, 11^{th} percentile, 10^{th} percentile, 9^{th} percentile, 8^{th} percentile, 7^{th} percentile, 6^{th} percentile, 5^{th} percentile, 4^{th} percentile, 3^{rd} percentile, 2^{nd} percentile, or 1^{st} percentile of LPA16:0, LPA18:0, LPA18:1, LPA18:2, or LPA20:4 levels, respectively, in the reference population.

In some aspects, the reference level of LPA20:4 is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA20:4 levels, in the reference population.

In some aspects, the reference level of LPA20:4 is the 67^{th} percentile of LPA20:4 levels in the reference population.

In some aspects, the level of LPA20:4 in the sample from the patient (e.g., the baseline level of LPA20:4 in the patient) is at or below the 67^{th} percentile of LPA20:4 levels in the reference population, e.g., is at or below the 66^{th} percentile, 65^{th} percentile, 64^{th} percentile, 63^{rd} percentile, 62^{nd} percentile, 61^{st} percentile, 60^{th} percentile, 59^{th} percentile, 58^{th} percentile, 57^{th} percentile, 56^{th} percentile, 55^{th} percentile, 54^{th} percentile, 53^{rd} percentile, 52^{nd} percentile, 51^{st} percentile, 50^{th} percentile, 49^{th} percentile, 48^{th} percentile, 47^{th} percentile, 46^{th} percentile, 45^{th} percentile, 44^{th} percentile, 43^{rd} percentile, 42^{nd} percentile, 41^{st} percentile, 40^{th} percentile, 39^{th} percentile, 38^{th} percentile, 37^{th} percentile, 36^{th} percentile, 35^{th} percentile, 34^{th} percentile, 33^{rd} percentile, 32^{nd} percentile, 31^{st} percentile, 30^{th} percentile, 29^{th} percentile, 28^{th} percentile, 27^{th} percentile, 26^{th} percentile, 25^{th} percentile, 24^{th} percentile, 23^{rd} percentile, 22^{nd} percentile, 21^{st} percentile, 20^{th} percentile, 19^{th} percentile, 18^{th} percentile, 17^{th} percentile, 16^{th} percentile, 15^{th} percentile, 14^{th} percentile, 13^{th} percentile, 12^{th} percentile, 11^{th} percentile, 10^{th} percentile, 9^{th} percentile, 8^{th} percentile, 7^{th} percentile, 6^{th} percentile, 5^{th} percentile, 4^{th} percentile, 3^{rd} percentile, 2^{nd} percentile, or 1^{st} percentile of LPA20:4 levels in the reference population.

ii. LPA Biomarkers for Decreased Risk of Exacerbation of COPD and Asthma

In some aspects, the disclosure features a method for predicting the time to next exacerbation for a patient having an inflammatory respiratory disease (e.g., a respiratory disease described in Section IIIE herein, e.g., COPD or asthma) who has experienced at least one exacerbation in the prior 12 months, the method comprising measuring a level of one or both of LPA18:0 and LPA18:2 in a sample from the patient, wherein a level of one or both of LPA18:0 and LPA18:2 in the sample that is above a reference level identifies the patient as one who may have an increased time to next exacerbation. In some aspects, the inflammatory respiratory disease is COPD.

In some aspects, the patient has a level of one or both of LPA18:0 and LPA18:2 in the sample that is above a reference level and the method further comprises maintaining the treatment regimen of the patient and/or reducing monitoring of the patient.

In some aspects, the increased time to next exacerbation is an increase of at least 1 day, at least 2 days, at least 3 days, at least 4 days, at least 5 days, at least 6 days, at least 7 days, at least 8 days, at least 9 days, at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 100 days, at least 110 days, at least 120 days, at least 130 days, at least 140 days, at least 150 days, at least 160 days, at least 170 days, at least 180 days, at least 190 days, at least 200 days, at least 210 days, at least 220 days, at least 230 days, at least 240 days, at least 250 days, at least 260 days, at least 270 days, at least 280 days, at least 290 days, at least 300 days, at least one year, or more than one year, e.g., 1-20 days, 20-40 days, 40-60 days, 60-80 days, 80-100 days, 100-120 days, 120-140 days, 140-180 days, 180-200 days, 200-220 days, 220-240 days, 240-260 days, 260-280 days, 280-300 days, 300-320 days, 320-340 days, 340-360 days, 360-380 days, 380-400 days, 400-420 days, 420-440 days, 440-460 days, 460-480 days, 480-500 days, 500-520 days, 520-540 days, 540-560 days, 560-580 days, or 580-600 days. In some aspects, the increased time to next exacerbation is an increase of at least 100 days.

The sample may be, e.g., a whole blood sample, a plasma sample, a serum sample, or a combination thereof; a bronchoalveolar lavage fluid (BALF) sample; or a urine sample. The sample may be, e.g., an archival sample, a fresh sample, or a frozen sample. In some aspects, the sample is a serum sample.

In some aspects, the level of one or both of LPA18:0 and LPA18:2 is a baseline level of one or both of LPA18:0 and LPA18:2, e.g., a level that is measured when the patient is not experiencing an exacerbation.

In some aspects, the reference level is a pre-assigned level of one or both of LPA18:0 and LPA18:2.

In some aspects, the reference level is a level of one or one or both of LPA18:0 and LPA18:2 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPA18:0 is between about 0.03 μM to about 0.05 μM. In some aspects, the reference level for LPA18:0 is about 0.04 μM.

In some aspects, the reference level for LPA18:2 is between about 0.68 μM to about 0.84 μM. In some aspects, the reference level for LPA18:2 is about 0.76 μM.

In some aspects, the reference level of LPA18:0 or LPA18:2 is the 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA18:0 or LPA18:2 levels, respectively, in the reference population.

In some aspects, the reference level of LPA18:0 or LPA18:2 is the 67^{th} percentile of LPA16:0, LPA18:0 or LPA18:2 levels, respectively, in the reference population.

In some aspects, the reference level for LPA18:0 is between about 0.01 μM to about 0.035 μM. In some aspects, the reference level for LPA18:0 is about 0.025 μM.

In some aspects, the reference level for LPA18:2 is between about 0.42 μM to about 0.53 μM. In some aspects, the reference level for LPA18:2 is about 0.48 μM.

In some aspects, the reference level of LPA18:0 or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA18:0 or LPA18:2 levels, respectively, in the reference population.

In some aspects, the reference level of LPA18:0 or LPA18:2 is the 33^{rd} percentile of LPA16:0, LPA18:0 or LPA18:2 levels, respectively, in the reference population.

In some aspects, the level of LPA18:0 or LPA18:2 in the sample from the patient (e.g., the baseline level of LPA18:0 or LPA18:2 in the sample from the patient) is at or above the 67th percentile of LPA18:0 or LPA18:2 levels in the reference population, e.g., is at or above the 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA18:0 or LPA18:2 levels, respectively, in the reference population.

iii. LPC, Sphingomyelin, and Ceramide Biomarkers for COPD and Asthma

In some aspects, the disclosure features a method for identifying, diagnosing, and/or predicting whether a patient having an inflammatory respiratory disease (e.g., a respiratory disease described in Section IIIE herein, e.g., COPD or asthma) may have an increased risk for an exacerbation, the method comprising measuring a level of one or more of LPC, sphingomyelins, and ceramides (e.g., hexosylceramide (HCER) or lactosylceramide (LCER)) in a sample from the patient, wherein a level of LPC in the sample that is below a reference level and/or a level of one or both of sphingomyelins and ceramides (e.g., HCER or LCER) in the sample that is above a reference level identifies, diagnoses, and/or predicts the patient as one who is at an increased risk for an exacerbation. In some aspects, the inflammatory respiratory disease is COPD.

In some aspects, the LPC is LPC(16:0) or LPC(18:2).

In some aspects, the disclosure features a method for identifying, diagnosing, and/or predicting whether a patient having an inflammatory respiratory disease may benefit from a treatment comprising an agent that reduces exacerbations (e.g., an agent described in Section IIIG herein), the method comprising measuring a level of one or more of LPC, sphingomyelins, and ceramides (e.g., HCER or LCER) in a sample from the patient, wherein a level of LPC in the sample that is below a reference level and/or a level of one or both of sphingomyelins and ceramides (e.g., HCER or LCER) in the sample that is above a reference level identifies, diagnoses, and/or predicts the patient as one who may benefit from a treatment comprising an agent that reduces exacerbations. In some aspects, the inflammatory respiratory disease is COPD.

In some aspects, the disclosure features a method of selecting a therapy for a patient having an inflammatory respiratory disease (e.g., a respiratory disease described in Section IIIE herein, e.g., COPD or asthma), the method comprising measuring a level of one or more of LPC, sphingomyelins, and ceramides in a sample from the patient, wherein a level of LPC in the sample that is below a reference level and/or a level of one or both of sphingomyelins and ceramides (e.g., HCER or LCER) in the sample that is above a reference level identifies the patient as one who may benefit from a treatment comprising an agent that reduces exacerbations. In some aspects, the inflammatory respiratory disease is COPD.

In some aspects, the benefit comprises an extension in the patient's time to an exacerbation compared to treatment without the agent that reduces exacerbations, e.g., an extension of at least one day, at least two days, at least three days, at least four days, at least five days, at least six days, at least one week, at least two weeks, at least three weeks, at least one month, at least two months, at least three months, at least four months, at least five months, at least six months, at least one year, or more than one year in the patient's time to first exacerbation or time to next exacerbation.

In some aspects, the benefit comprises a reduction in the duration of an exacerbation in the patient compared to treatment without the agent that reduces exacerbations, e.g., a reduction of at least 10 minutes, at least 20 minutes, at least 30 minutes, at least 1 hour, at least 2 hours, at least 6 hours, at least 12 hours, at least one day, at least two days, at least three days, at least four days, at least five days, at least six days, at least one week, at least two weeks, at least three weeks, at least one month, at least two months, at least three months, at least four months, at least five months, at least six months, at least one year, or more than one year in the duration of an exacerbation.

In some aspects, the disclosure features a method of identifying a patient suitable for administration with an agent that treats an inflammatory respiratory disease (e.g., a respiratory disease described in Section IIIE herein, e.g., COPD or asthma) or an agent that reduces exacerbations of an inflammatory respiratory disease (e.g., an agent described in Section IIIG herein), the method comprising measuring a level of one or more of LPC, sphingomyelins, and ceramides (e.g., HCER or LCER) in a sample from the patient, wherein a level of LPC in the sample that is below a reference level and/or a level of one or both of sphingomyelins and ceramides in the sample that is above a reference level identifies the patient as one who is suitable for administration with an agent that treats an inflammatory respiratory disease or an agent that reduces exacerbations of an inflammatory respiratory disease. In some aspects, the inflammatory respiratory disease is COPD.

In some aspects, the patient has a level of a LPC (e.g., LPC(16:0) or LPC(18:2)) in the sample that is below a reference level and/or a level of one or both of sphingomyelins and ceramides (e.g., HCER or LCER) in the sample that is above a reference level and the method further comprises administering to the patient an effective amount of an agent that reduces exacerbations.

In some aspects, the benefit comprises a reduction in the duration of an exacerbation in the patient compared to treatment without the agent that reduces exacerbations, e.g., a reduction of at least 10 minutes, at least 20 minutes, at least 30 minutes, at least 1 hour, at least 2 hours, at least 6 hours, at least 12 hours, at least 1 day, at least two days, at least three days, at least four days, at least five days, at least six days, at least one week, at least two weeks, at least three weeks, at least one month, at least two months, at least three months, at least four months, at least five months, at least six months, at least one year, or more than one year in the duration of an exacerbation.

The sample may be, e.g., a whole blood sample, a plasma sample, a serum sample, or a combination thereof. In some aspects, the sample is a BALF sample or a urine sample. The sample may be an archival sample, a fresh sample, or a frozen sample. In some aspects, the sample is a serum sample.

In some aspects, the level of one or more of LPC, sphingomyelins, and ceramides (e.g., HCER or LCER) is a baseline level of one or more of LPC (e.g., LPC(16:0) or LPC(18:2)), sphingomyelins, and ceramides, e.g., a level that is measured when the patient is not experiencing an exacerbation.

In some aspects, the reference level is a pre-assigned level of one or more of LPC (e.g., LPC(16:0) or LPC(18:2)), sphingomyelins, and ceramides.

In some aspects, the reference level is a level of one or more of LPC (e.g., LPC(16:0) or LPC(18:2)), sphingomyelins, and ceramides (e.g., HCER or LCER) in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPC (e.g., LPC(16:0) or LPC(18:2)) is between about 227 nmol/mL to about 277 nmol/mL. In some aspects, the reference level for LPC (e.g., LPC(16:0) or LPC(18:2)), is about 252 nmol/mL.

In some aspects, the reference level for sphingomyelins is between about 448 nmol/mL to about 548 nmol/mL. In some aspects, the reference level for sphingomyelins is about 498 nmol/mL.

In some aspects, the ceramide is hexosylceramide (HCER). In some aspects, the reference level for HCER is between about 6.1 nmol/mL to about 7.5 nmol/mL. In some aspects, the reference level for HCER is about 6.8 nmol/mL.

In some aspects, the ceramide is lactosylceramide (LCER). In some aspects, the reference level for LCER is between about 4.3 nmol/mL to about 5.3 nmol/mL. In some aspects, the reference level for LCER is about 4.8 nmol/mL. In some aspects, the reference level of LPC (e.g., LPC(16:0) or LPC(18:2)) is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPC (e.g., LPC(16:0) or LPC(18:2)), sphingomyelins, or ceramides levels, respectively, in the reference population.

In some aspects, the reference level of LPC (e.g., LPC(16:0) or LPC(18:2)) is the 33^{rd} percentile of LPC (e.g., LPC(16:0) or LPC(18:2)) levels in the reference population.

In some aspects, the reference level of sphingomyelins or ceramides (e.g., HCER or LCER) is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels, respectively, in the reference population.

In some aspects, the reference level of sphingomyelins or ceramides (e.g., HCER and/or LCER) is the 67^{th} percentile of sphingomyelins or ceramides levels, respectively, in the reference population.

In some aspects, the level of LPC (e.g., LPC(16:0) or LPC(18:2)) (e.g., the baseline level of LPC (e.g., LPC(16:0) or LPC(18:2)) in the patient) is at or below the 33^{rd} percentile of LPC (e.g., LPC(16:0) or LPC(18:2)) levels in the reference population, e.g., is at or below the 32^{nd} percentile, 31^{st} percentile, 30^{th} percentile, 29^{th} percentile, 28^{th} percentile, 27^{th} percentile, 26^{th} percentile, 25^{th} percentile, 24^{th} percentile, 23^{rd} percentile, 22^{nd} percentile, 21^{st} percentile, 20^{th} percentile, 19^{th} percentile, 18^{th} percentile, 17^{th} percentile, 16^{th} percentile, 15^{th} percentile, 14^{th} percentile, 13^{th} percentile, 12^{th} percentile, 11^{th} percentile, 10^{th} percentile, 9^{th} percentile, 8^{th} percentile, 7^{th} percentile, 6^{th} percentile, 5^{th} percentile, 4^{th} percentile, 3^{rd} percentile, 2^{nd} percentile, or 1^{st} percentile of LPC (e.g., LPC(16:0) or LPC(18:2)) levels in the reference population.

In some aspects, the level of sphingomyelins or ceramides (e.g., HCER and/or LCER) (e.g., the baseline level of sphingomyelins or ceramides in the patient) is at or above the 67^{th} percentile of sphingomyelins or ceramides levels in the reference population, e.g., is at or above the 68^{th} percentile, 69^{th} percentile, 70^{th} percentile, 71^{st} percentile, 72^{nd} percentile, 73^{rd} percentile, 74^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels in the reference population.

B. Methods of Treatment for COPD and Asthma

i. LPA Biomarkers for COPD and Asthma

In some aspects, the disclosure features a method of treating a patient having an inflammatory respiratory disease (e.g., a respiratory disease described in Section IIIE herein, e.g., COPD or asthma), the method comprising: (a) measuring a level of one or more of LPA16:0, LPA18:0, LPA18:1, LPA18:2, and LPA20:4 in a sample from the patient, wherein the level of one or more of LPA16:0, LPA18:0, LPA18:1, LPA18:2, and LPA20:4 in the sample is below a reference level; and (b) administering an effective amount of an agent that reduces exacerbations to the patient. In some aspects, the inflammatory respiratory disease is COPD or IPF. In some aspects, the inflammatory respiratory disease is asthma and the level of one or more of LPA16:0, LPA18:0, and LPA18:2, e.g., one, two, or all three of LPA16:0, LPA18:0, and LPA18:2) in the sample from the patient is below a reference level.

In some aspects, the disclosure features a method of treating a patient having an inflammatory respiratory disease (e.g., a respiratory disease described in Section IIIE herein, e.g., COPD or asthma) and having a level of one or more of LPA16:0, LPA18:0, LPA18:1, LPA18:2, and LPA20:4 in a sample from the patient that is below a reference level comprising administering an effective amount of an agent that reduces exacerbations to the patient. In some aspects, the inflammatory respiratory disease is asthma and the level of one or more of LPA16:0, LPA18:0, and LPA18:2, e.g., one, two, or all three of LPA16:0, LPA18:0, and LPA18:2) in the sample from the patient is below a reference level.

In some aspects, the disclosure features a method of treating a patient having an inflammatory respiratory disease (e.g., a respiratory disease described in Section IIIE herein, e.g., COPD or asthma), the method comprising administering to the patient an effective amount of an agent that reduces exacerbations, wherein the level of one or more of LPA16:0, LPA18:0, LPA18:1, LPA18:2, and LPA20:4 in a sample from the patient has been determined to be below a reference level. In some aspects, the inflammatory respiratory disease is asthma and the level of one or more of LPA16:0, LPA18:0, and LPA18:2, e.g., one, two, or all three of LPA16:0, LPA18:0, and LPA18:2) in a sample from the patient has been determined to be below a reference level.

In some aspects, the disclosure features a method of reducing exacerbations in a patient having an inflammatory respiratory disease (e.g., a respiratory disease described in Section IIIE herein, e.g., COPD or asthma), the method comprising (a) measuring a level of one or more of LPA16:0, LPA18:0, LPA18:1, LPA18:2, and LPA20:4 in a sample from the patient, wherein the level of one or more of LPA16:0, LPA18:0, LPA18:1, LPA18:2, and LPA20:4 in the sample is below a reference level; and (b) administering an effective amount of an agent that reduces exacerbations to the patient. In some aspects, the inflammatory respiratory disease is asthma and the level of one or more of LPA16:0, LPA18:0, and LPA18:2, e.g., one, two, or all three of LPA16:0, LPA18:0, and LPA18:2) in the sample from the patient is below a reference level.

In some aspects, the disclosure features a method of reducing exacerbations in a patient having an inflammatory respiratory disease (e.g., a respiratory disease described in Section IIIE herein, e.g., COPD or asthma) and having a level of one or more of LPA16:0, LPA18:0, LPA18:1, LPA18:2, and LPA20:4 in a sample from the patient that is below a reference level comprising administering an effective amount of an agent that reduces exacerbations to the patient. In some aspects, the inflammatory respiratory disease is asthma and the patient has a level of one or more of LPA16:0, LPA18:0, and LPA18:2, e.g., one, two, or all three of LPA16:0, LPA18:0, and LPA18:2) in a sample from the patient that is below a reference level.

In some aspects, the disclosure features a method of reducing exacerbations in a patient having an inflammatory respiratory disease (e.g., a respiratory disease described in Section IIIE herein, e.g., COPD or asthma), the method comprising administering to the patient an effective amount of an agent that reduces exacerbations, wherein the level of one or more of LPA16:0, LPA18:0, LPA18:1, LPA18:2, and LPA20:4 in a sample from the patient has been determined to be below a reference level. In some aspects, the inflammatory respiratory disease is asthma and the level of one or more of LPA16:0, LPA18:0, and LPA18:2, e.g., one, two, or all three of LPA16:0, LPA18:0, and LPA18:2) in a sample from the patient has been determined to be below a reference level.

In some aspects, the patient has a level of one or more of LPA16:0, LPA18:0, LPA18:1, LPA18:2, and LPA20:4 (e.g., a level of one, two, three, four, or all five of LPA16:0, LPA18:0, LPA18:1, LPA18:2, and LPA20:4) in the sample that is below a reference level.

The sample may be, e.g., a whole blood sample, a plasma sample, a serum sample, or a combination thereof; a bronchoalveolar lavage fluid (BALF) sample; or a urine sample. The sample may be, e.g., an archival sample, a fresh sample, or a frozen sample. In some aspects, the sample is a serum sample.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4, e.g., a level that is measured when the patient is not experiencing an exacerbation.

In some aspects, the level of one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4) in the sample from the patient is below a reference level.

In some aspects, the reference level is a pre-assigned level of one or more of LPA16:0, LPA18:0, LPA18:1, LPA18:2, and LPA20:4.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD), IPF, or asthma). In some aspects, the patient has experienced at least one exacerbation in the prior 12 months.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD), IPF, or asthma). In some aspects, the patient has experienced at least one exacerbation in the prior 12 months.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level of LPA16:0, LPA18:0, LPA18:1, or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:0, LPA18:1, or LPA18:2 levels, respectively, in the reference population.

In some aspects, the reference level of LPA16:0, LPA18:0, LPA18:1, or LPA18:2 is the 33^{rd} percentile of LPA16:0, LPA18:0, LPA18:1, or LPA18:2 levels, respectively, in the reference population.

In some aspects, the reference level of LPA20:4 is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA20:4 levels, in the reference population.

In some aspects, the reference level of LPA20:4 is the 67^{th} percentile of LPA20:4 levels in the reference population.

ii. Lipid Biomarkers for COPD and Asthma

In some aspects, the disclosure features a method of treating a patient having an inflammatory respiratory disease (e.g., a respiratory disease described in Section IIIE herein, e.g., COPD or asthma), the method comprising: (a) measuring a level of one or more of LPC, sphingomyelins, and ceramides (e.g., HCER and/or LCER) in a sample from the patient, wherein a level of LPC in the sample is below a reference level and/or a level of one or both of sphingomyelins and ceramides in the sample is above a reference level; and (b) administering an effective amount of an agent that reduces exacerbations to the patient. In some aspects, the inflammatory respiratory disease is COPD or IPF.

In some aspects, the disclosure features a method of treating a patient having an inflammatory respiratory disease (e.g., a respiratory disease described in Section IIIE herein, e.g., COPD or asthma) and having a level of LPC in the sample that is below a reference level and/or a level of one or both of sphingomyelins and ceramides (e.g., HCER or LCER) in the sample that is above a reference level comprising administering an effective amount of an agent that reduces exacerbations to the patient.

In some aspects, the disclosure features a method of treating a patient having an inflammatory respiratory disease (e.g., a respiratory disease described in Section IIIE herein, e.g., COPD or asthma), the method comprising administering to the patient an effective amount of an agent that reduces exacerbations, wherein a level of LPC in the sample that has been determined to be below a reference level and/or a level of one or both of sphingomyelins and ceramides (e.g., HCER or LCER) in the sample has been determined to be above a reference level.

In some aspects, the disclosure features a method of reducing exacerbations in a patient having an inflammatory respiratory disease (e.g., a respiratory disease described in Section IIIE herein, e.g., COPD or asthma), the method comprising (a) measuring a level one or more of LPC, sphingomyelins, and ceramides (e.g., HCER or LCER) in a sample from the patient, wherein a level of LPC in the sample is below a reference level and/or a level of one or both of sphingomyelins and ceramides in the sample is above a reference level; and (b) administering an effective amount of an agent that reduces exacerbations to the patient.

In some aspects, the disclosure features a method of reducing exacerbations in a patient having an inflammatory respiratory disease (e.g., a respiratory disease described in Section IIIE herein, e.g., COPD or asthma) and having a level of LPC in a sample from the patient that is below a reference level and/or a level of one or both of sphingomyelins and ceramides (e.g., HCER or LCER) in a sample from the patient that is above a reference level comprising administering an effective amount of an agent that reduces exacerbations to the patient.

In some aspects, the disclosure features a method of reducing exacerbations in a patient having an inflammatory respiratory disease (e.g., a respiratory disease described in Section IIIE herein, e.g., COPD or asthma), the method comprising administering to the patient an effective amount of an agent that reduces exacerbations, wherein a level of LPC in a sample from the patient has been determined to be below a reference level and/or a level of one or both of sphingomyelins and ceramides (e.g., HCER or LCER) in a sample from the patient has been determined to be above a reference level.

The sample may be, e.g., a whole blood sample, a plasma sample, a serum sample, or a combination thereof; a bronchoalveolar lavage fluid (BALF) sample; or a urine sample. The sample may be, e.g., an archival sample, a fresh sample, or a frozen sample. In some aspects, the sample is a serum sample.

In some aspects, the level of one or more of LPC, sphingomyelins, and ceramides is a baseline level of one or more of LPC, sphingomyelins, and ceramides, e.g., a level that is measured when the patient is not experiencing an exacerbation.

In some aspects, the reference level is a pre-assigned level of one or more of LPC, sphingomyelins, and ceramides.

In some aspects, the reference level is a level of one or more of LPC, sphingomyelins, and ceramides in a reference population, e.g., a reference population of patients having the inflammatory respiratory disease (e.g., COPD (e.g., stage II, stage III, or stage IV COPD), IPF, or asthma). In some aspects, the patient has experienced at least one exacerbation in the prior 12 months.

In some aspects, the reference level for LPC (e.g., LPC(16:0) or LPC(18:2)) is between about 227 nmol/mL to about 277 nmol/mL. In some aspects, the reference level for LPC (e.g., LPC(16:0) or LPC(18:2)), is about 252 nmol/mL.

In some aspects, the reference level for sphingomyelins is between about 448 nmol/mL to about 548 nmol/mL. In some aspects, the reference level for sphingomyelins is about 498 nmol/mL.

In some aspects, the ceramide is hexosylceramide (HCER). In some aspects, the reference level for HCER is between about 6.1 nmol/mL to about 7.5 nmol/mL. In some aspects, the reference level for HCER is about 6.8 nmol/mL.

In some aspects, the ceramide is lactosylceramide (LCER). In some aspects, the reference level for LCER is between about 4.3 nmol/mL to about 5.3 nmol/mL. In some aspects, the reference level for LCER is about 4.8 nmol/mL.

In some aspects, the reference level of LPC (e.g., LPC(16:0) or LPC(18:2)) is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th}percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPC (e.g., LPC(16:0) or LPC(18:2)), sphingomyelins, or ceramides levels, respectively, in the reference population.

In some aspects, the reference level of LPC (e.g., LPC(16:0) or LPC(18:2)) is the 33^{rd} percentile of LPC (e.g., LPC(16:0) or LPC(18:2)) levels in the reference population.

In some aspects, the reference level of sphingomyelins or ceramides (e.g., HCER or LCER) is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels, respectively, in the reference population.

C. Diagnostic Methods and Methods of Treatment for IPF

i. LPA Biomarkers for Increased Risk of Exacerbation or Respiratory Hospitalization

In one aspect, the disclosure features a method for identifying, diagnosing, and/or predicting whether a patient having idiopathic pulmonary fibrosis (IPF) may have an increased risk for an exacerbation of IPF (e.g., an exacerbation of IPF as described in Section IIIE herein) or respiratory hospitalization, the method comprising measuring a level of one or more of lysophosphatidic acid (LPA)16:0, LPA18:1, LPA18:2, and LPA20:4 (e.g., measuring a level of one, two, three, or all four of LPA16:0, LPA18:1, LPA18:2, and LPA20:4) in a sample from the patient, wherein a level of one or more of LPA16:0, LPA18:1, LPA18:2, and LPA20:4 in the sample that is at or above a reference level identifies the patient as one who is at an increased risk for an exacerbation or respiratory hospitalization; diagnoses the patient as one who is at an increased risk for an exacerbation or respiratory hospitalization; or predicts that the patient is one who is at an increased risk for an exacerbation or respiratory hospitalization.

In another aspect, the disclosure features a method for identifying, diagnosing, and/or predicting whether a patient having IPF may benefit from a treatment comprising an agent that reduces exacerbations, the method comprising measuring a level of one or more of LPA16:0, LPA18:1, LPA18:2, and LPA20:4 (e.g., measuring a level of one, two, three, or all four of LPA16:0, LPA18:1, LPA18:2, and LPA20:4) in a sample from the patient, wherein a level of one or more of LPA16:0, LPA18:1, LPA18:2, and LPA20:4 in the sample that is at or above a reference level identifies, diagnoses, and/or predicts the patient as one who may benefit from a treatment comprising an agent that reduces exacerbations.

In another aspect, the disclosure features a method of selecting a therapy for a patient having IPF, the method comprising measuring a level of one or more of LPA16:0, LPA18:1, LPA18:2, and LPA20:4 (e.g., measuring a level of one, two, three, or all four of LPA16:0, LPA18:1, LPA18:2, and LPA20:4) in a sample from the patient, wherein a level of one or more of LPA16:0, LPA18:1, LPA18:2, and LPA20:4 in the sample that is at or above a reference level identifies the patient as one who may benefit from a treatment comprising an agent that reduces exacerbations (e.g., an agent as described in Section IIIG herein).

In some aspects, the patient has a level of one or more of LPA16:0, LPA18:0, LPA18:2, and LPA20:4 (e.g., a level of one, two, three, or all four of LPA16:0, LPA18:1, LPA18:2, and LPA20:4) in the sample that is at or above a reference level and the method further comprises administering to the patient an effective amount of an agent that reduces exacerbations.

In another aspect, the disclosure features a method of treating a patient having IPF, the method comprising (a) measuring a level of one or more of LPA16:0, LPA18:1, LPA18:2, and LPA20:4 (e.g., measuring a level of one, two, three, or all four of LPA16:0, LPA18:1, LPA18:2, and LPA20:4) in a sample from the patient, wherein the level of one or more of LPA16:0, LPA18:1, LPA18:2, and LPA20:4 in the sample is at or above a reference level; and (b) administering an effective amount of an agent that reduces exacerbations to the patient.

In another aspect, the disclosure features a method of treating a patient having IPF and having a level of one or more of LPA16:0, LPA18:1, LPA18:2, and LPA20:4 (e.g., a level of one, two, three, or all four of LPA16:0, LPA18:1, LPA18:2, and LPA20:4) in a sample from the patient that is at or above a reference level comprising administering an effective amount of an agent that reduces exacerbations to the patient.

In another aspect, the disclosure features a method of treating a patient having IPF, the method comprising administering to the patient an effective amount of an agent that reduces exacerbations, wherein the level of one or more of LPA16:0, LPA18:1, LPA18:2, and LPA20:4 (e.g., a level of one, two, three, or all four of LPA16:0, LPA18:1, LPA18:2, and LPA20:4) in a sample from the patient has been determined to be at or above a reference level.

In some aspects, the sample is a whole blood sample, a plasma sample, a serum sample, or a combination thereof. In some aspects, the sample is an archival sample, a fresh sample, or a frozen sample. In some aspects, the sample is a serum sample.

In some aspects, the level of one or more of LPA16:0, LPA18:1, LPA18:2, and LPA20:4 is a baseline level of one or more of LPA16:0, LPA18:1, LPA18:2, and LPA20:4.

In some aspects, the reference level is a pre-assigned level of one or more of LPA16:0, LPA18:1, LPA18:2, and LPA20:4.

In some aspects, (a) the patient is female and the reference level for LPA16:0 is between about 0.207 μM to about 0.247 μM; or (b) the patient is male and the reference level for LPA16:0 is between about 0.153 μM to about 0.193 μM. In some aspects, (a) the patient is female and the reference level for LPA16:0 is about 0.227 μM; or (b) the patient is male and the reference level for LPA16:0 is about 0.173 μM.

In some aspects, (a) the patient is female and the reference level for LPA18:1 is between about 0.082 μM to about 0.122 μM; or (b) the patient is male and the reference level for LPA18:1 is between about 0.078 μM to about 0.118 μM. In some aspects, (a) the patient is female and the reference level for LPA18:1 is about 0.102 μM; or (b) the patient is male and the reference level for LPA18:1 is about 0.098 μM.

In some aspects, (a) the patient is female and the reference level for LPA18:2 is between about 0.388 μM to about 0.428 μM; or (b) the patient is male and the reference level for LPA18:2 is between about 0.339 μM to about 0.379 μM. In some aspects, (a) the patient is female and the reference level for LPA18:2 is about 0.408 μM; or (b) the patient is male and the reference level for LPA18:2 is about 0.359 μM.

In some aspects, (a) the patient is female and the reference level for LPA20:4 is between about 0.100 μM to about 0.140 μM; or (b) the patient is male and the reference level for LPA20:4 is between about 0.110 μM to about 0.150 μM. In some aspects, (a) the patient is female and the reference level for LPA20:4 is about 0.120 μM; or (b) the patient is male and the reference level for LPA20:4 is about 0.130 μM.

In some aspects, the reference level is a level (e.g., a median level) of one or more of LPA16:0, LPA18:1, LPA18:2, and LPA20:4 in a reference population.

In some aspects, the level of one or more of LPA16:0, LPA18:1, and LPA18:2 in the sample is at or above the median of levels of LPA16:0, LPA18:1, or LPA18:2, respectively, in the reference population.

In some aspects, the reference level of LPA16:0, LPA18:1, LPA18:2, or LPA20:4 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:1, LPA18:2, or LPA20:4 levels, respectively, in the reference population.

In some aspects, the reference population is a population of patients having IPF.

In some aspects, the reference population is a population of patients not having IPF. In some aspects. the level of one or more of LPA16:0, LPA18:1, LPA18:2, and LPA20:4 in the sample is at least two-fold greater than the reference level, e.g., the reference level in the population of patients not having IPF.

In some aspects, the benefit comprises an extension in the patient's time to an exacerbation compared to treatment without the agent that reduces exacerbations, e.g., an extension of at least one day, at least two days, at least three days, at least four days, at least five days, at least six days, at least one week, at least two weeks, at least three weeks, at least one month, at least two months, at least three months, at least four months, at least five months, at least six months, at least one year, or more than one year in the patient's time to a first exacerbation or respiratory hospitalization or time to a next exacerbation or respiratory hospitalization.

In some aspects, the exacerbation is an acute respiratory deterioration. In some aspects, the acute respiratory deterioration is dyspnea. In some aspects, the acute respiratory deterioration is not caused by pneumothorax, cancer, heart failure, fluid overload, or pulmonary embolism.

In some aspects, the acute respiratory deterioration is associated with a new radiologic abnormality. In some aspects, the radiologic abnormality is bilateral ground-glass opacification/consolidation.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof.

In some aspects, the agent that reduces exacerbations is nintedanib or pirfenidone.

In some aspects, the agent that reduces exacerbations is approved by a regulatory health agency for reducing, controlling, or maintaining exacerbations. In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

ii. TG Biomarkers for Increased Risk of Death

In another aspect, the disclosure features a method for identifying, diagnosing, and/or predicting whether a patient having idiopathic pulmonary fibrosis (IPF) may have an increased risk of death, the method comprising measuring a level of one or both of triglyceride (TG)48:4-FA12:0 and TG48:4-FA18:2 in a sample from the patient, wherein a level of one or both of TG48:4-FA12:0 and TG48:4-FA18:2 in the sample that is below a reference level identifies the patient as one who is at an increased risk of death; diagnoses the patient as one who is at an increased risk of death; or predicts that the patient is one who is at an increased risk of death.

In another aspect, the disclosure features a method for identifying, diagnosing, and/or predicting whether a patient having IPF may benefit from a treatment comprising an agent that reduces exacerbations, the method comprising measuring a level of one or both of TG48:4-FA12:0 and TG48:4-FA18:2 in a sample from the patient, wherein a level of one or both of TG48:4-FA12:0 and TG48:4-FA18:2 in the sample that is below a reference level identifies, diagnoses, and/or predicts the patient as one who may benefit from a treatment comprising an agent that reduces exacerbations.

In another aspect, the disclosure features a method of selecting a therapy for a patient having IPF, the method comprising measuring a level of one or both of TG48:4-FA12:0 and TG48:4-FA18:2 in a sample from the patient, wherein a level of one or both of TG48:4-FA12:0 and TG48:4-FA18:2 in the sample that is below a reference level identifies the patient as one who may benefit from a treatment comprising an agent that reduces exacerbations (e.g., an agent as described in Section IIIG herein).

In some aspects, the patient has a level of one or both of TG48:4-FA12:0 and TG48:4-FA18:2 in the sample that is below a reference level and the method further comprises administering to the patient an effective amount of an agent that reduces exacerbations.

In another aspect, the disclosure features a method of treating a patient having IPF, the method comprising: (a) measuring a level of one or both of TG48:4-FA12:0 and TG48:4-FA18:2 in a sample from the patient, wherein the level of one or both of TG48:4-FA12:0 and TG48:4-FA18:2 in the sample is below a reference level; and (b) administering an effective amount of an agent that reduces exacerbations to the patient.

In another aspect, the disclosure features a method of treating a patient having IPF and having a level of one or both of TG48:4-FA12:0 and TG48:4-FA18:2 in a sample from the patient that is below a reference level comprising administering an effective amount of an agent that reduces exacerbations to the patient.

In another aspect, the disclosure features a method of treating a patient having IPF, the method comprising administering to the patient an effective amount of an agent that reduces exacerbations, wherein the level of one or both of TG48:4-FA12:0 and TG48:4-FA18:2 in a sample from the patient has been determined to be below a reference level.

In some aspects, the sample is a whole blood sample, a plasma sample, a serum sample, or a combination thereof.

In some aspects, the sample is an archival sample, a fresh sample, or a frozen sample.

In some aspects, the sample is a serum sample.

In some aspects, the level of one or both of TG48:4-FA12:0 and TG48:4-FA18:2 is a baseline level of one or both of TG48:4-FA12:0 and TG48:4-FA18:2.

In some aspects, the reference level is a pre-assigned level of one or both of TG48:4-FA12:0 and TG48:4-FA18:2.

In some aspects, (a) the patient is female and the reference level for TG48:4-FA12:0 is between about 0.800 μM to about 0.840 μM; or (b) the patient is male and the reference level for TG48:4-FA12:0 is between about 1.166 μM to about 1.206 μM. In some aspects, (a) the patient is female and the reference level for TG48:4-FA12:0 is about 0.820 μM; or (b) the patient is male and the reference level for TG48:4-FA12:0 is about 1.186 μM.

In some aspects, (a) the patient is female and the reference level for TG48:4-FA18:2 (μM) is between about 1.587 μM to about 1.627 μM; or (b) the patient is male and the reference level for TG48:4-FA18:2 (μM) is between about 2.153 μM to about 2.193 μM. In some aspects, (a) the patient is female and the reference level for TG48:4-FA18:2 (μM) is about 1.607 μM; or (b) the patient is male and the reference level for TG48:4-FA18:2 is about 2.173 μM.

In some aspects, the reference level is a level (e.g., a median level) of one or both of TG48:4-FA12:0 and TG48:4-FA18:2 in a reference population.

In some aspects, the level of one or both of TG48:4-FA12:0 and TG48:4-FA18:2 in the sample is below the median of levels of TG48:4-FA12:0 or TG48:4-FA18:2, respectively, in the reference population.

In some aspects, the reference level of TG48:4-FA12:0 or TG48:4-FA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of TG48:4-FA12:0 or TG48:4-FA18:2 levels, respectively, in the reference population.

In some aspects, the reference population is a population of patients having IPF.

In some aspects, the reference population is a population of patients not having IPF. In some aspects, the level of one or both of TG48:4-FA12:0 or TG48:4-FA18:2 in the sample is at least two-fold less than the reference level, e.g., the reference level in the population of patients not having IPF.

In some aspects, the benefit comprises an extension in the patient's time to death compared to treatment without the agent that reduces exacerbations, e.g., an extension of at least one week, at least two weeks, at least three weeks, at least one month, at least two months, at least three months, at least four months, at least five months, at least six months, at least one year, or more than one year in the patient's time to death.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof.

In some aspects, the agent that reduces exacerbations is nintedanib or pirfenidone.

In some aspects, the agent that reduces exacerbations is approved by a regulatory health agency for reducing, controlling, or maintaining exacerbations.

In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

iii. LPA and TG Biomarkers for Decreased Time to Exacerbation or Respiratory Hospitalization

In another aspect, the disclosure features a method for predicting the time to exacerbation (e.g., an exacerbation of IPF as described in Section IIIE herein) or respiratory hospitalization for a patient having IPF, the method comprising measuring a level of one or more of LPA16:0, LPA18:1, LPA20:4, LPA22:4, TG48:4-FA12:0, and TG48:4-FA18:2 (e.g., one, two, three, four, five, or all six of LPA16:0, LPA18:1, LPA20:4, LPA22:4, TG48:4-FA12:0, and TG48:4-FA18:2) in a sample from the patient, wherein (a) a level of one or more of LPA16:0, LPA18:1, LPA20:4, and LPA22:4 in the sample (e.g., a level of one, two, three, or all four of LPA16:0, LPA18:1, LPA20:4, and LPA22:4) that is at or above a reference level or (b) a level of one or both of TG48:4-FA12:0 and TG48:4-FA18:2 in the sample that is below a reference level identifies the patient as one who may have a decreased time to exacerbation or respiratory hospitalization.

In some aspects, the patient has (a) a level of one or more of LPA16:0, LPA18:1, LPA20:4, and LPA22:4 in the sample that is at or above a reference level or (b) a level of one or both of TG48:4-FA12:0 and TG48:4-FA18:2 in the sample that is below a reference level and the method further comprises administering to the patient an effective amount of an agent that reduces exacerbations (e.g., an agent as described in Section IIIG herein).

In some aspects, the sample is a whole blood sample, a plasma sample, a serum sample, or a combination thereof.

In some aspects, the sample is an archival sample, a fresh sample, or a frozen sample.

In some aspects the sample is a serum sample.

In some aspects, the level of one or more of LPA16:0, LPA18:1, LPA20:4, LPA22:4, TG48:4-FA12:0, and TG48:4-FA18:2 is a baseline level of one or more of LPA16:0, LPA18:1, LPA20:4, LPA22:4, TG48:4-FA12:0, and TG48:4-FA18:2. In some aspects, the reference level is a pre-assigned level of one or more of LPA16:0, LPA18:1, LPA20:4, LPA22:4, TG48:4-FA12:0, and TG48:4-FA18:2.

In some aspects, (a) the patient is female and the reference level for LPA16:0 is between about 0.207 μM to about 0.247 μM; or (b) the patient is male and the reference level for LPA16:0 is between about 0.153 μM to about 0.193 μM. In some aspects, (a) the patient is female and the reference level for LPA16:0 is about 0.227 μM; or (b) the patient is male and the reference level for LPA16:0 is about 0.173 μM.

In some aspects, (a) the patient is female and the reference level for LPA18:1 is between about 0.082 μM to about 0.122 μM; or (b) the patient is male and the reference level for LPA18:1 is between about 0.078 μM to about 0.118 μM. In some aspects, (a) the patient is female and the reference level for LPA18:1 is about 0.102 μM; or (b) the patient is male and the reference level for LPA18:1 is about 0.098 μM.

In some aspects, (a) the patient is female and the reference level for LPA20:4 is between about 0.100 μM to about 0.140 μM; or (b) the patient is male and the reference level for LPA20:4 is between about 0.110 μM to about 0.150 μM. In some aspects, (a) the patient is female and the reference level for LPA20:4 is about 0.120 μM; or (b) the patient is male and the reference level for LPA20:4 is about 0.130 μM.

In some aspects, (a) the patient is female and the reference level for LPA22:4 is between about 0.100 μM to about 0.140 μM; or (b) the patient is male and the reference level for LPA22:4 is between about 0.110 μM to about 0.150 μM. In some aspects, (a) the patient is female and the reference level for LPA22:4 is about 0.120 μM; or (b) the patient is male and the reference level for LPA22:4 is about 0.130 μM.

In some aspects, (a) the patient is female and the reference level for TG48:4-FA12:0 is between about 0.800 μM to about 0.840 μM; or (b) the patient is male and the reference level for TG48:4-FA12:0 is between about 1.166 μM to about 1.206 μM. In some aspects, (a) the patient is female and the reference level for TG48:4-FA12:0 is about 0.820 μM; or (b) the patient is male and the reference level for TG48:4-FA12:0 is about 1.186 μM.

In some aspects, (a) the patient is female and the reference level for TG48:4-FA18:2 (μM) is between about 1.587 μM to about 1.627 μM; or (b) the patient is male and the reference level for TG48:4-FA18:2 (μM) is between about 2.153 μM to about 2.193 μM. In some aspects, (a) the patient is female and the reference level for TG48:4-FA18:2 (μM) is about 1.607 μM; or (b) the patient is male and the reference level for TG48:4-FA18:2 is about 2.173 μM.

In some aspects, the reference level is a level (e.g., a median level) of one or more of LPA16:0, LPA18:1, LPA20:4, LPA22:4, TG48:4-FA12:0, and TG48:4-FA18:2 in a reference population.

In some aspects, (a) the level of one or more of LPA16:0, LPA18:1, LPA20:4, and LPA22:4 in the sample is at or above the median of levels of LPA16:0, LPA18:1, LPA20:4, or LPA22:4, respectively, in the reference population or (b) the level of one or both of TG48:4-FA12:0 and TG48:4-FA18:2 in the sample is at or below the median of levels of TG48:4-FA12:0 or TG48:4-FA18:2, respectively, in the reference population.

In some aspects, the reference level of LPA16:0, LPA18:1, LPA20:4, LPA22:4, TG48:4-FA12:0, or TG48:4-FA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:1, LPA20:4, LPA22:4, TG48:4-FA12:0, orTG48:4-FA18:2 levels, respectively, in the reference population.

In some aspects, the reference population is a population of patients having IPF.

In some aspects, the reference population is a population of patients not having IPF. In some aspects, (a) the level of one or more of LPA16:0, LPA18:1, LPA20:4, and LPA22:4 in the sample is at least two-fold greater than the reference level or (b) the level of one or both of TG48:4-FA12:0 or TG48:4-FA18:2 in the sample is at least two-fold less than the reference level, e.g., the reference level in the population of patients not having IPF.

In some aspects, the exacerbation is an acute respiratory deterioration.

In some aspects, the acute respiratory deterioration is dyspnea.

In some aspects, the acute respiratory deterioration is not caused by pneumothorax, cancer, heart failure, fluid overload, or pulmonary embolism.

In some aspects, the acute respiratory deterioration is associated with a new radiologic abnormality.

In some aspects, the radiologic abnormality is bilateral ground-glass opacification/consolidation.

In some aspects, the exacerbation is a severe exacerbation.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid, an antibiotic, an althpa-1 antitrypsin augmentation therapy, mepolizumab, benralizumab, or a combination thereof.

In some aspects, the corticosteroid is an inhaled corticosteroid (ICS) or an oral corticosteroid (OCS).

In some aspects, the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof.

In some aspects, the agent that reduces exacerbations is nintedanib or pirfenidone.

In some aspects, the agent that reduces exacerbations is approved by a regulatory health agency for reducing, controlling, or maintaining exacerbations.

In some aspects, the regulatory health agency is the U.S. Food & Drug Administration (FDA), the European Medicines Agency (EMA), the Pharmaceuticals and Medical Devices Agency (PMDA), or the National Medical Products Administration (NMPA).

iv. LPA and TG Biomarkers for Risk of Deterioration in Measures of Lung Health

In another aspect, the disclosure features a method for identifying, diagnosing, and/or predicting whether a patient having idiopathic pulmonary fibrosis (IPF) may have an increased risk for deterioration in a measure of lung health, the method comprising: (a) measuring a level of one or more of LPA16:0, LPA16:1, LPA18:1, LPA18:2, and LPA20:4 in a sample from the patient, wherein the measure of lung health is diffusing capacity of carbon monoxide (DLCO) and a level of one or more of LPA16:0, LPA16:1, LPA18:1, LPA18:2, and LPA20:4 in the sample that is at or above a reference level identifies, diagnoses, and/or predicts the patient as one who is at an increased risk for deterioration of DLCO; (b) measuring a level of LPA22:4 in a sample from the patient, wherein the measure of lung health is ground glass opacity in the whole lung and a level of LPA22:4 in the sample that is at or above a reference level identifies, diagnoses, and/or predicts the patient as one who is at an increased risk for increased ground glass opacity in the whole lung; or (c) measuring a level of one or more of LPA16:0, LPA16:1, LPA18:0, LPA18:1, LPA18:2, and LPA20:4 in a sample from the patient, wherein the measure of lung health is fibrosis in lower zones of the lung and a level of one or more of LPA16:0, LPA16:1, LPA18:0, LPA18:1, LPA18:2, and LPA20:4 in the sample that is at or above a reference level identifies, diagnoses, and/or predicts the patient as one who is at an increased risk for fibrosis in lower zones of the lung.

In some aspects, (a) the patient is female and the reference level for LPA16:0 is between about 0.207 μM to about 0.247 μM; or (b) the patient is male and the reference level for LPA16:0 is between about 0.153 μM to about 0.193 μM. In some aspects, (a) the patient is female and the reference level for LPA16:0 is about 0.227 μM; or (b) the patient is male and the reference level for LPA16:0 is about 0.173 μM.

In some aspects, (a) the patient is female and the reference level for LPA16:1 is between about 0.101 ratio-to-standard (rts) to about 0.141 rts; or (b) the patient is male and the reference level for LPA16:1 is between about 0.058 rts to about 0.098 rts. In some aspects, (a) the patient is female and the reference level for LPA16:1 is about 0.121 rts; or (b) the patient is male and the reference level for LPA16:1 is about 0.078 rts.

In some aspects, (a) the patient is female and the reference level for LPA18:0 is between about 0.007 μM to about 0.047 μM; or (b) the patient is male and the reference level for LPA18:0 is between about 0.003 μM to about 0.043 μM. In some aspects, (a) the patient is female and the reference level for LPA18:0 is about 0.027 μM; or (b) the patient is male and the reference level for LPA18:0 is about 0.023 μM.

In some aspects, (a) the patient is female and the reference level for LPA18:1 is between about 0.082 μM to about 0.122 μM; or (b) the patient is male and the reference level for LPA18:1 is between about 0.078 μM to about 0.118 μM. In some aspects, (a) the patient is female and the reference level for LPA18:1 is about 0.102 μM; or (b) the patient is male and the reference level for LPA18:1 is about 0.098 μM.

In some aspects, (a) the patient is female and the reference level for LPA18:2 is between about 0.388 μM to about 0.428 μM; or (b) the patient is male and the reference level for LPA18:2 is between about 0.339 μM to about 0.379 μM. In some aspects, (a) the patient is female and the reference level for LPA18:2 is about 0.408 μM; or (b) the patient is male and the reference level for LPA18:2 is about 0.359 μM.

In some aspects, (a) the patient is female and the reference level for LPA20:4 is between about 0.100 μM to about 0.140 μM; or (b) the patient is male and the reference level for LPA20:4 is between about 0.110 μM to about 0.150 μM. In some aspects, (a) the patient is female and the reference level for LPA20:4 is about 0.120 μM; or (b) the patient is male and the reference level for LPA20:4 is about 0.130 μM.

In some aspects, (a) the patient is female and the reference level for LPA22:4 is between about 0.009 rts to about 0.049 rts; or (b) the patient is male and the reference level for LPA22:4 is between about 0.011 rts to about 0.051 rts. In some aspects, (a) the patient is female and the reference level for LPA22:4 is about 0.029 rts; or (b) the patient is male and the reference level for LPA22:4 is about 0.031 rts.

D. Methods for Measuring LPA Levels

The level of one or more of LPA16:0, LPA18:0, LPA18:1, LPA18:2, LPA20:4, and LPA22:4 in the sample from the patient may be assessed using any of the methods described herein, e.g., may be assessed using a method described in Section II herein, e.g., may be assessed using a method comprising (a) providing a sample from the patient and (b) extracting LPA from the sample in (a) using an extraction buffer comprising citric acid and disodium phosphate, wherein the extraction buffer does not result in the hydrolysis of the choline group from other lysophospholipids in the serum sample.

In some aspects, the level of one, two, three, four, or all five of LPA16:0, LPA18:0, LPA18:1, LPA18:2, and LPA20:4 is assessed in the sample from the patient. In some aspects, the level of one, two, three, four, five, or all six of LPA16:0, LPA18:0, LPA18:1, LPA18:2, LPA20:4, and LPA22:4 is assessed in the sample from the patient.

E. Inflammatory Respiratory Diseases

Inflammatory respiratory diseases include any disease, disorder, or condition associated with inflammation in the respiratory tract.

In some aspects, inflammatory respiratory diseases include respiratory diseases associated with fibrosis (e.g., idiopathic pulmonary fibrosis (IPF) and interstitial lung disease (ILD)).

In some aspects, the inflammatory respiratory disease is chronic obstructive pulmonary disease (COPD). COPD is a progressive, chronic inflammatory lung disease that is caused by smoking or exposure to other substances that irritate and damage the lungs.

The COPD may be stage I, stage II, stage III, or stage IV according to the GLOBAL INITIATIVE FOR CHRONIC OBSTRUCTIVE LUNG DISEASE™ (GOLD) staging system. In some aspects, the COPD is stage II, stage III, or stage IV.

In some aspects, the inflammatory respiratory disease is idiopathic pulmonary fibrosis (IPF).

In some aspects, the inflammatory respiratory disease is asthma.

In some aspects, the inflammatory respiratory disease is interstitial lung disease (ILD).

In some aspects, the patient having the inflammatory respiratory disease is male.

F. Exacerbations

An exacerbation of an inflammatory respiratory disease (e.g., COPD, IPF, or asthma) may be any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics.

Duration of an exacerbation may be defined as, e.g., the duration for which the patient experiences symptoms and/or the number of days for which the patient is on systemic corticosteroids and/or antibiotics to treat the exacerbation. The exacerbation may be a severe exacerbation, e.g., an exacerbation requiring hospitalization.

An increased risk of exacerbation may be, e.g., a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% increase in the risk of an exacerbation, e.g., an increase in the risk that an exacerbation will occur within a period of time, e.g., within about one week, two weeks, three weeks, one month, two months, three months, four months, five months, six months, seven months, eight months, nine months, ten months, eleven months, one year, two years, three years, four years, five years, six years, seven years, eight years, nine years, or ten years.

i. Exacerbations of COPD

An exacerbation of COPD may be one or more new or increased symptoms of COPD, e.g., an increase in breathlessness (dyspnea), cough, sputum volume, sputum purulence, fatigue, trouble sleeping, headache when waking up, confusion, or reduced oxygen level (hypoxemia) e.g., a new or increased symptom that lasts for at least 2 consecutive days and/or that leads to hospitalization and/or treatment with systemic corticosteroids and/or antibiotics. Exacerbations of COPD are described in GLOBAL INITIATIVE FOR CHRONIC OBSTRUCTIVE LUNG DISEASE™ (GOLD) Pocket Guide to COPD Diagnosis, Management, and Prevention (2020 Edition) and in Lareu et al., Am J Respir Crit Care Med, 198: 21-22, 2018, which are incorporated herein by reference in their entirety.

ii. Exacerbations of IPF

An exacerbation of IPF may be one or more new or increased symptoms of the IPF, e.g., acute respiratory deterioration (e.g., dyspnea), wherein the acute respiratory deterioration is not caused by pneumothorax, cancer, heart failure, fluid overload, or pulmonary embolism. The exacerbation of IPF may be associated with a new radiologic abnormality, e.g., bilateral ground-glass opacification/consolidation. Exacerbations of IPF are described in Collard et al., Am J Respir Crit Care Med, 194(3): 265-275, 2016, which is incorporated herein by reference in its entirety.

iii. Exacerbations of Asthma

An exacerbation of asthma may be an episode of one or more of progressively worsening shortness of breath, coughing, wheezing, and chest tightness. The episode may be acute or subacute. Exacerbations of asthma are described in Camargo et al., Proc Am Thorac Soc, 6(4): 357-366, 2009, which is incorporated herein by reference in its entirety.

G. Agents that Reduce Exacerbations

In some aspects, the methods of the invention include use of an agent that reduces exacerbations. An agent that reduces exacerbations may be any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease. Such agents include agents that are used for the treatment of an inflammatory respiratory disease (e.g., maintenance medications), e.g., agents used for the treatment of COPD, IPF, and/or asthma, and agents that are used for the treatment of exacerbations of an inflammatory respiratory disease.

Agents that reduce exacerbations include agents that have been approved by a regulatory health agency (e.g., the U.S. Food & Drug Administration (FDA), the European Medicines Agency (EMA), the Pharmaceuticals and Medical Devices Agency (PMDA), or the National Medical Products Administration (NMPA)) for reducing, controlling, or maintaining exacerbations.

In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid (e.g., an inhaled corticosteroid (ICS) or an oral corticosteroid (OCS)), an antibiotic, an althpa-1 antitrypsin augmentation therapy, or a combination thereof. In some aspects, the agent that reduces exacerbations is an agent that has been approved for the treatment of asthma, e.g., mepolizumab or benralizumab.

In some aspects, the agent that reduces exacerbations reduces the rate of exacerbations, e.g., reduces the rate of exacerbations by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some aspects, the agent that reduces exacerbations extends the time to exacerbation (e.g., increases the time to first exacerbation or increases the duration of time between exacerbations, e.g., increases the duration of time to the next exacerbation), e.g., extends the time to exacerbation by at least one week, at least two weeks, at least three weeks, at least one month, at least two months, at least three months, at least four months, at least five months, at least six months, at least seven months, at least eight months, at least nine months, at least ten months, at least 11 months, at least one year, at least two years, at least three years, at least four years, at least five years, at least six years, at least seven years, at least eight years, at least nine years, at least ten years, or more than ten years.

In some aspects, the agent that reduces exacerbations reduces the duration of an exacerbation, e.g., reduces the duration of an exacerbation by at least one day, two days, three days, four days, five days, six days, one week, two weeks, three weeks, one month, or more than one month.

In some aspects, the agent that reduces exacerbations reduces the severity of an exacerbation, e.g., reduces the severity of an exacerbation by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

i. Agents that Reduce Exacerbations of COPD

In some aspects, the patient has COPD and the agent that reduces exacerbations is an agent disclosed in the GLOBAL INITIATIVE FOR CHRONIC OBSTRUCTIVE LUNG DISEASE™ (GOLD) Pocket Guide to COPD Diagnosis, Management, and Prevention (2020 Edition). In some aspects, the agent that reduces exacerbations is an influenza vaccination, a pneumococcal vaccination, supplemental oxygen, a short-acting bronchodilator (SABD), a long-acting bronchodilator, a dual-acting bronchodilator, a short-acting anti-cholinergic, a long-acting anticholinergic, a short-acting anti-muscarinic antagonist (SAMA), a long-acting muscarinic antagonist (LAMA), a short-acting beta₂-agonist (SABA), a long-acting beta₂-agonist (LABA), a PDE4 inhibitor, a methylxanthine, a phosphodiesterase-4 inhibitor, a mucolytic agent, a mucoregulator, an antioxidant agent, an anti-inflammatory agent, a corticosteroid (e.g., an inhaled corticosteroid (ICS) or an oral corticosteroid (OCS)), an antibiotic, an althpa-1 antitrypsin augmentation therapy, or a combination thereof.

ii. Agents that Reduce Exacerbations of IPF

In some aspects, the patient has IPF and the agent that reduces exacerbations is nintedanib, pirfenidone, procalcitonin, cyclosporine, rituximab combined with plasma exchange and intravenous immunoglobulin, tacrolimus, thrombomodulin, anti-acid therapy, a corticosteroid, cyclophosphamide, or a combination thereof. In some aspects, the agent that reduces exacerbations is nintedanib or pirfenidone. In some aspects, the agent that reduces exacerbations is nintedanib. In some aspects, the agent that reduces exacerbations is pirfenidone.

iii. Agents that Reduce Exacerbations of Asthma

In some aspects, the patient has asthma and the agent that reduces exacerbations is an inhaled short-acting beta₂-agonist (SABA), albuterol, bitolterol, levalbuterol, pirbuterol, a systemic SABA (e.g., epinephrine or terbutaline), an anticholinergic (e.g., ipratropium bromide), a systemic corticosteroid (e.g., prednisone, methylprednisolone, or prednisolone), mepolizumab, or benralizumab. In some aspects, the agent that reduces exacerbations is mepolizumab or benralizumab. In some aspects, the agent that reduces exacerbations is mepolizumab. In some aspects, the agent that reduces exacerbations is benralizumab.

H. Methods of Delivery

The compositions utilized in the methods described herein (e.g., agents that reduce exacerbations) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated).

In some aspects, an agent that reduces exacerbations is administered by inhalation, intravenously, intramuscularly, subcutaneously, topically, orally, transdermally, intraperitoneally, intraorbitally, by implantation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Agents that reduce exacerbations, as described herein (and any additional therapeutic agent), may be formulated, dosed, and administered in a fashion consistent with good medical practice. Factors for consideration in this context include the particular disorder being treated, the particular mammal being treated, the clinical condition of the patient, the cause of the disorder, the site of delivery of the agent, the method of administration, the scheduling of administration, and other factors known to medical practitioners. The agent that reduces exacerbations need not be, but is optionally formulated with and/or administered concurrently with, one or more agents currently used to prevent or treat the inflammatory respiratory disease (e.g., COPD, IPF, or asthma). The effective amount of such other agents depends on the amount of the agent that reduces exacerbations present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of an inflammatory respiratory disease, e.g., a respiratory disease described in Section IIIE herein, e.g., COPD, IPF, or asthma, the appropriate dosage of an agent that reduces exacerbations described herein (e.g., an agent described in Section IIIG herein) (when used alone or in combination with one or more other additional therapeutic agents) will depend on the type of disease to be treated, the severity and course of the disease, whether the agent that reduces exacerbations is administered for preventive or therapeutic purposes, previous therapy, the patient's clinical history and response to the agent that reduces exacerbations, and the discretion of the attending physician. The agent that reduces exacerbations is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every day, every week, or every month. An initial higher loading dose, followed by one or more lower doses, may be administered. However, other dosage regimens may be useful. The progress of this therapy is easily monitored by conventional techniques and assays.

### IV. Methods of Monitoring Response to a Treatment

A. LPA Biomarkers for COPD and Asthma

In some aspects, the disclosure features a method of monitoring the response of a patient having an inflammatory respiratory disease (e.g., a respiratory disease described in Section IIIE herein, e.g., COPD or asthma) to a treatment comprising an agent that reduces exacerbations, the method comprising (a) measuring the level of one or more of LPA16:0, LPA18:0, LPA18:1, LPA18:2, and LPA20:4 in a sample obtained from the patient at a time point following the administration of a first dose of the treatment comprising the agent that reduces exacerbations; and (b) comparing the level of one or more of LPA16:0, LPA18:0, LPA18:1, LPA18:2, and LPA20:4 in the sample to a reference level, thereby monitoring the response of the patient to the treatment comprising an agent that reduces exacerbations. In some aspects, the inflammatory respiratory disease is asthma and the method comprises (a) measuring the level of one or more of LPA16:0, LPA18:0, and LPA18:2 in a sample obtained from the patient at a time point following the administration of a first dose of the treatment comprising the agent that reduces exacerbations; and (b) comparing the level of one or more of LPA16:0, LPA18:0, and LPA18:2 in the sample to a reference level, thereby monitoring the response of the patient to the treatment comprising an agent that reduces exacerbations.

In some aspects, a level of one or more of LPA16:0, LPA18:0, LPA18:1, LPA18:2, and LPA20:4 in a sample that is above a reference level indicates that the patient is responding to the agent that reduces exacerbations. In some aspects, the inflammatory respiratory disease is asthma and a level of one or more of LPA16:0, LPA18:0, and LPA18:2 in a sample that is above a reference level indicates that the patient is responding to the agent that reduces exacerbations.

In some aspects, the method further comprises administering at least a second dose (e.g., a second dose and one, two, three, four five, six, seven, eight nine, ten, or more than ten additional doses) of the agent that reduces exacerbations to a patient for whom a level of one or more of LPA16:0, LPA18:0, LPA18:1, LPA18:2, and LPA20:4 in the sample is above a reference level. In some aspects, the inflammatory respiratory disease is asthma and the method further comprises administering at least a second dose (e.g., a second dose and one, two, three, four five, six, seven, eight nine, ten, or more than ten additional doses) of the agent that reduces exacerbations to a patient for whom a level of one or more of LPA16:0, LPA18:0, and LPA18:2 in the sample is above a reference level.

In some aspects, the time point following the administration of the first dose of the treatment comprising an agent that reduces exacerbations is about one hour, about two hours, about three hours, about four hours, about five hours, about six hours, about seven hours, about eight hours, about 12 hours, about 1 day, about 2 days, about 3 days, about 4 days, about 5 days, about 6 days, about 1 week, about 2 weeks, about three weeks, about one month, or more than one month after the administration of the first dose of the treatment.

The sample may be, e.g., a whole blood sample, a plasma sample, a serum sample, or a combination thereof. In some aspects, the sample is a bronchoalveolar lavage fluid (BALF) sample or a urine sample. The sample may be an archival sample, a fresh sample, or a frozen sample. In some aspects, the sample is a serum sample.

The exacerbation may be an exacerbation as described in Section IIIF herein, e.g., any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics, e.g., an exacerbation of COPD or asthma.

The agent that reduces exacerbations may be an agent described in Section IIIG herein, e.g., any worsening of one or more symptoms of an inflammatory respiratory disease, e.g., any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease, e.g., an agent that reduces exacerbations of COPD or asthma.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4, e.g., a level that is measured when the patient is not experiencing an exacerbation.

In some aspects, the level of one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4) in the sample from the patient is below a reference level.

In some aspects, the reference level is a pre-assigned level of one or more of LPA16:0, LPA18:0, LPA18:1, LPA18:2, and LPA20:4.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level of LPA16:0, LPA18:0, LPA18:1, or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:0, LPA18:1, or LPA18:2 levels, respectively, in the reference population.

In some aspects, the reference level of LPA16:0, LPA18:0, LPA18:1, or LPA18:2 is the 33^{rd} percentile of LPA16:0, LPA18:0, LPA18:1, or LPA18:2 levels, respectively, in the reference population.

In some aspects, the reference level of LPA20:4 is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA20:4 levels, in the reference population.

In some aspects, the reference level of LPA20:4 is the 67^{th} percentile of LPA20:4 levels in the reference population.

B. Lipid Biomarkers for COPD and Asthma

In some aspects, the disclosure features a method of monitoring the response of a patient having an inflammatory respiratory disease (e.g., a respiratory disease described in Section IIIE herein, e.g., COPD or asthma) to a treatment comprising an agent that reduces exacerbations, the method comprising (a) measuring the level of one or more of LPC, sphingomyelins, and ceramides in a sample obtained from the patient at a time point following the administration of a first dose of the treatment comprising the agent that reduces exacerbations; and (b) comparing the level of one or more of LPC, sphingomyelins, and ceramides in the sample to a reference level, thereby monitoring the response of the patient to the treatment comprising an agent that reduces exacerbations. In some aspects, the ceramide is a hexosylceramide (HCER), a lactosylceramide (LCER), or a dihydroceramide (DCER). In some aspects, the patient is female and the ceramide is a DCER.

In some aspects, a level of LPC in the sample that is above a reference level and/or a level of one or both of sphingomyelins and ceramides in the sample that is below a reference level indicates that the patient is responding to the agent that reduces exacerbations.

In some aspects, the method further comprises administering at least a second dose (e.g., a second dose and one, two, three, four five, six, seven, eight nine, ten, or more than ten additional doses) of the agent that reduces exacerbations to a patient for whom a level of LPC in the sample is above a reference level and/or a level of one or both of sphingomyelins and ceramides in the sample is below a reference level.

In some aspects, the time point following the administration of the first dose of the treatment comprising an agent that reduces exacerbations is about one hour, about two hours, about three hours, about four hours, about five hours, about six hours, about seven hours, about eight hours, about 12 hours, about 1 day, about 2 days, about 3 days, about 4 days, about 5 days, about 6 days, about 1 week, about 2 weeks, about three weeks, about one month, or more than one month after the administration of the first dose of the treatment.

The sample may be, e.g., a whole blood sample, a plasma sample, a serum sample, or a combination thereof. In some aspects, the sample is a bronchoalveolar lavage fluid (BALF) sample or a urine sample. The sample may be an archival sample, a fresh sample, or a frozen sample. In some aspects, the sample is a serum sample.

The exacerbation may be an exacerbation as described in Section IIIF herein, e.g., any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics, e.g., an exacerbation of COPD or asthma.

The agent that reduces exacerbations may be an agent described in Section IIIG herein, e.g., any worsening of one or more symptoms of an inflammatory respiratory disease, e.g., any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease, e.g., an agent that reduces exacerbations of COPD or asthma.

In some aspects, the level of one or more of LPC, sphingomyelins, and ceramides is a baseline level of one or more of LPC, sphingomyelins, and ceramides, e.g., a level that is measured when the patient is not experiencing an exacerbation.

In some aspects, the reference level is a pre-assigned level of one or more of LPC, sphingomyelins, and ceramides.

In some aspects, the reference level is a level of one or more of LPC, sphingomyelins, and ceramides in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPC (e.g., LPC(16:0) or LPC(18:2)) is between about 227 nmol/mL to about 277 nmol/mL. In some aspects, the reference level for LPC (e.g., LPC(16:0) or LPC(18:2)), is about 252 nmol/mL.

In some aspects, the reference level for sphingomyelins is between about 448 nmol/mL to about 548 nmol/mL. In some aspects, the reference level for sphingomyelins is about 498 nmol/mL.

In some aspects, the ceramide is hexosylceramide (HCER). In some aspects, the reference level for HCER is between about 6.1 nmol/mL to about 7.5 nmol/mL. In some aspects, the reference level for HCER is about 6.8 nmol/mL.

In some aspects, the ceramide is lactosylceramide (LCER). In some aspects, the reference level for LCER is between about 4.3 nmol/mL to about 5.3 nmol/mL. In some aspects, the reference level for LCER is about 4.8 nmol/mL.

In some aspects, the reference level of LPC (e.g., LPC(16:0) or LPC(18:2)) is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPC (e.g., LPC(16:0) or LPC(18:2)), sphingomyelins, or ceramides levels, respectively, in the reference population.

In some aspects, the reference level of LPC (e.g., LPC(16:0) or LPC(18:2)) is the 33^{rd} percentile of LPC (e.g., LPC(16:0) or LPC(18:2)) levels in the reference population.

In some aspects, the reference level of sphingomyelins or ceramides (e.g., HCER or LCER) is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels, respectively, in the reference population.

In some aspects, the reference level of sphingomyelins or ceramides (e.g., HCER and/or LCER) is the 67^{th} percentile of sphingomyelins or ceramides levels, respectively, in the reference population.

### V. Agents for Use in Treatment of Inflammatory Respiratory Diseases

A. LPA Biomarkers for COPD and Asthma

In some aspects, the disclosure features an agent that reduces exacerbations for use in the treatment of a patient having an inflammatory respiratory disease (e.g., a respiratory disease described in Section IIIE herein, e.g., COPD or asthma) and having a level of one or more of LPA16:0, LPA18:0, LPA18:1, LPA18:2, and LPA20:4 in a sample from the patient that is below a reference level. In some aspects, the inflammatory respiratory disease is asthma and the patient has a level of one or more of LPA16:0, LPA18:0, and LPA18:2 in a sample from the patient that is below a reference level.

In some aspects, the disclosure features an agent that reduces exacerbations for use in the treatment of a patient having an inflammatory respiratory disease (e.g., a respiratory disease described in Section IIIE herein, e.g., COPD or asthma), wherein the level of one or more of LPA16:0, LPA18:0, LPA18:1, LPA18:2, and LPA20:4 in a sample from the patient has been determined to be below a reference level. In some aspects, the inflammatory respiratory disease is asthma and the level of one or more of LPA16:0, LPA18:0, and LPA18:2 in a sample from the patient has been determined to be below a reference level.

The sample may be, e.g., a whole blood sample, a plasma sample, a serum sample, or a combination thereof. In some aspects, the sample is a bronchoalveolar lavage fluid (BALF) sample or a urine sample. The sample may be an archival sample, a fresh sample, or a frozen sample. In some aspects, the sample is a serum sample.

The exacerbation may be an exacerbation as described in Section IIIF herein, e.g., any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics, e.g., an exacerbation of COPD or asthma.

The agent that reduces exacerbations may be an agent described in Section IIIG herein, e.g., any worsening of one or more symptoms of an inflammatory respiratory disease, e.g., any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease, e.g., an agent that reduces exacerbations of COPD or asthma.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4, e.g., a level that is measured when the patient is not experiencing an exacerbation.

In some aspects, the level of one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4) in the sample from the patient is below a reference level.

In some aspects, the reference level is a pre-assigned level of one or more of LPA16:0, LPA18:0, LPA18:1, LPA18:2, and LPA20:4.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level of LPA16:0, LPA18:0, LPA18:1, or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:0, LPA18:1, or LPA18:2 levels, respectively, in the reference population.

In some aspects, the reference level of LPA16:0, LPA18:0, LPA18:1, or LPA18:2 is the 33^{rd} percentile of LPA16:0, LPA18:0, LPA18:1, or LPA18:2 levels, respectively, in the reference population.

In some aspects, the reference level of LPA20:4 is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th}percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA20:4 levels, in the reference population.

In some aspects, the reference level of LPA20:4 is the 67^{th} percentile of LPA20:4 levels in the reference population.

B. Lipid Biomarkers for COPD and Asthma

In some aspects, the disclosure features an agent that reduces exacerbations for use in the treatment of a patient having an inflammatory respiratory disease (e.g., a respiratory disease described in Section IIIE herein, e.g., COPD or asthma) and having a level of LPC in the sample that is below a reference level and/or a level of one or both of sphingomyelins and ceramides in the sample that is above a reference level.

In some aspects, the disclosure features an agent that reduces exacerbations for use in the treatment of a patient having an inflammatory respiratory disease, wherein the level of LPC in a sample from the patient has been determined to be below a reference level and/or a level of one or both of sphingomyelins and ceramides in a sample from the patient has been determined to be above a reference level.

The sample may be, e.g., a whole blood sample, a plasma sample, a serum sample, or a combination thereof. In some aspects, the sample is a bronchoalveolar lavage fluid (BALF) sample or a urine sample. The sample may be an archival sample, a fresh sample, or a frozen sample. In some aspects, the sample is a serum sample.

The exacerbation may be an exacerbation as described in Section IIIF herein, e.g., any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics, e.g., an exacerbation of COPD or asthma.

The agent that reduces exacerbations may be an agent described in Section IIIG herein, e.g., any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease, e.g., an agent that reduces exacerbations of COPD or asthma.

In some aspects, the level of one or more of LPC, sphingomyelins, and ceramides is a baseline level of one or more of LPC, sphingomyelins, and ceramides, e.g., a level that is measured when the patient is not experiencing an exacerbation.

In some aspects, the reference level is a pre-assigned level of one or more of LPC, sphingomyelins, and ceramides.

In some aspects, the reference level is a level of one or more of LPC, sphingomyelins, and ceramides in a reference population, e.g., a reference population of patients having the inflammatory respiratory disease (e.g., COPD (e.g., stage II, stage III, or stage IV COPD), IPF, or asthma). In some aspects, the patient has experienced at least one exacerbation in the prior 12 months.

In some aspects, the reference level for LPC (e.g., LPC(16:0) or LPC(18:2)) is between about 227 nmol/mL to about 277 nmol/mL. In some aspects, the reference level for LPC (e.g., LPC(16:0) or LPC(18:2)), is about 252 nmol/mL.

In some aspects, the reference level for sphingomyelins is between about 448 nmol/mL to about 548 nmol/mL. In some aspects, the reference level for sphingomyelins is about 498 nmol/mL.

In some aspects, the ceramide is hexosylceramide (HCER). In some aspects, the reference level for HCER is between about 6.1 nmol/mL to about 7.5 nmol/mL. In some aspects, the reference level for HCER is about 6.8 nmol/mL.

In some aspects, the ceramide is lactosylceramide (LCER). In some aspects, the reference level for LCER is between about 4.3 nmol/mL to about 5.3 nmol/mL. In some aspects, the reference level for LCER is about 4.8 nmol/mL.

In some aspects, the reference level of LPC (e.g., LPC(16:0) or LPC(18:2)) is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPC (e.g., LPC(16:0) or LPC(18:2)), sphingomyelins, or ceramides levels, respectively, in the reference population.

In some aspects, the reference level of LPC (e.g., LPC(16:0) or LPC(18:2)) is the 33^{rd} percentile of LPC (e.g., LPC(16:0) or LPC(18:2)) levels in the reference population.

In some aspects, the reference level of sphingomyelins or ceramides (e.g., HCER or LCER) is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels, respectively, in the reference population.

In some aspects, the reference level of sphingomyelins or ceramides (e.g., HCER and/or LCER) is the 67^{th} percentile of sphingomyelins or ceramides levels, respectively, in the reference population.

In some aspects, the level of LPC (e.g., LPC(16:0) or LPC(18:2)) (e.g., the baseline level of LPC (e.g., LPC(16:0) or LPC(18:2)) in the patient) is at or below the 33^{rd} percentile of LPC (e.g., LPC(16:0) or LPC(18:2)) levels in the reference population, e.g., is at or below the 32^{nd} percentile, 31^{st} percentile, 30^{th} percentile, 29^{th} percentile, 28^{th} percentile, 27^{th} percentile, 26^{th} percentile, 25^{th} percentile, 24^{th} percentile, 23^{rd} percentile, 22^{nd} percentile, 21^{st} percentile, 20^{th} percentile, 19^{th} percentile, 18^{th} percentile, 17^{th} percentile, 16^{th} percentile, 15^{th} percentile, 14^{th} percentile, 13^{th} percentile, 12^{th} percentile, 11^{th} percentile, 10^{th} percentile, 9^{th} percentile, 8^{th} percentile, 7^{th} percentile, 6^{th} percentile, 5^{th} percentile, 4^{th} percentile, 3^{rd} percentile, 2^{nd} percentile, or 1^{st} percentile of LPC (e.g., LPC(16:0) or LPC(18:2)) levels in the reference population.

In some aspects, the level of sphingomyelins or ceramides (e.g., HCER and/or LCER) (e.g., the baseline level of sphingomyelins or ceramides in the patient) is at or above the 67^{th} percentile of sphingomyelins or ceramides levels in the reference population, e.g., is at or above the 68^{th} percentile, 69^{th} percentile, 70^{th} percentile, 71^{st} percentile, 72^{nd} percentile, 73^{rd} percentile, 74^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels in the reference population.

### VI. Uses of Agents in the Manufacture of Medicaments

A. LPA Biomarkers for Use in the Manufacture of Medicaments for COPD or Asthma

In some aspects, the disclosure features the use of an agent that reduces exacerbations in a patient having a level of one or more of LPA16:0, LPA18:0, LPA18:1, LPA18:2, and LPA20:4 in a sample from the patient that is below a reference level in the manufacture of a medicament for the treatment of an inflammatory respiratory disease (e.g., a respiratory disease described in Section IIIE herein, e.g., COPD or asthma). In some aspects, the inflammatory respiratory disease is asthma and the patient has a level of one or more of LPA16:0, LPA18:0, and LPA18:2 in a sample from the patient that is below a reference level.

In some aspects, the disclosure features the use of an agent that reduces exacerbations in a patient having a level of one or more of LPA16:0, LPA18:0, LPA18:1, LPA18:2, and LPA20:4 in a sample from the patient that is below a reference level in the manufacture of a medicament for reducing exacerbations of an inflammatory respiratory disease (e.g., a respiratory disease described in Section IIIE herein, e.g., COPD or asthma). In some aspects, the inflammatory respiratory disease is asthma and the patient has a level of one or more of LPA16:0, LPA18:0, and LPA18:2 in a sample from the patient that is below a reference level.

The sample may be, e.g., a whole blood sample, a plasma sample, a serum sample, or a combination thereof. In some aspects, the sample is a bronchoalveolar lavage fluid (BALF) sample or a urine sample. The sample may be an archival sample, a fresh sample, or a frozen sample. In some aspects, the sample is a serum sample.

The exacerbation may be an exacerbation as described in Section IIIF herein, e.g., any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics, e.g., an exacerbation of COPD or asthma.

The agent that reduces exacerbations may be an agent described in Section IIIG herein, e.g., any worsening of one or more symptoms of an inflammatory respiratory disease, e.g., any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease, e.g., an agent that reduces exacerbations of COPD or asthma.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4, e.g., a level that is measured when the patient is not experiencing an exacerbation.

In some aspects, the level of one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4) in the sample from the patient is below a reference level.

In some aspects, the reference level is a pre-assigned level of one or more of LPA16:0, LPA18:0, LPA18:1, LPA18:2, and LPA20:4.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level of LPA16:0, LPA18:0, LPA18:1, or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:0, LPA18:1, or LPA18:2 levels, respectively, in the reference population.

In some aspects, the reference level of LPA16:0, LPA18:0, LPA18:1, or LPA18:2 is the 33^{rd} percentile of LPA16:0, LPA18:0, LPA18:1, or LPA18:2 levels, respectively, in the reference population.

In some aspects, the reference level of LPA20:4 is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA20:4 levels, in the reference population.

In some aspects, the reference level of LPA20:4 is the 67^{th} percentile of LPA20:4 levels in the reference population.

B. Lipid Biomarkers for Use in the Manufacture of Medicaments for COPD or Asthma

In some aspects, the disclosure features the use of an agent that reduces exacerbations in a patient having a level of LPC in a sample from the patient that is below a reference level and/or a level of one or both of sphingomyelins and ceramides in a sample from the patient that is above a reference level in the manufacture of a medicament for the treatment of an inflammatory respiratory disease (e.g., a respiratory disease described in Section IIIE herein, e.g., COPD or asthma).

In some aspects, the disclosure features the use of an agent that reduces exacerbations in a patient having a level of LPC in the sample that is below a reference level and/or a level of one or both of sphingomyelins and ceramides in the sample that is above a reference level in the manufacture of a medicament for reducing exacerbations of an inflammatory respiratory disease (e.g., a respiratory disease described in Section IIIE herein, e.g., COPD or asthma).

The sample may be, e.g., a whole blood sample, a plasma sample, a serum sample, or a combination thereof. In some aspects, the sample is a bronchoalveolar lavage fluid (BALF) sample or a urine sample. The sample may be an archival sample, a fresh sample, or a frozen sample. In some aspects, the sample is a serum sample.

The exacerbation may be an exacerbation as described in Section IIIF herein, e.g., any worsening of one or more symptoms of an inflammatory respiratory disease, e.g., a worsening of the disease that lasts for at least two consecutive days, requires medical attention, and/or leads to hospitalization and/or treatment with systemic corticosteroids or antibiotics, e.g., an exacerbation of COPD or asthma.

The agent that reduces exacerbations may be an agent described in Section IIIG herein, e.g., any worsening of one or more symptoms of an inflammatory respiratory disease, e.g., any agent that reduces the rate of exacerbations, increases the time to exacerbation (e.g., increases the time to first exacerbation or increases the duration of time between exacerbations, e.g., increases the duration of time to the next exacerbation), reduces the duration of exacerbations, and/or reduces the severity of exacerbations in a patient having an inflammatory respiratory disease, e.g., an agent that reduces exacerbations of COPD or asthma.

In some aspects, the level of one or more of LPC, sphingomyelins, and ceramides is a baseline level of one or more of LPC, sphingomyelins, and ceramides, e.g., a level that is measured when the patient is not experiencing an exacerbation.

In some aspects, the reference level is a pre-assigned level of one or more of LPC, sphingomyelins, and ceramides.

In some aspects, the reference level is a level of one or more of LPC, sphingomyelins, and ceramides in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the level of one or more of LPC, sphingomyelins, and ceramides (e.g., HCER or LCER) is a baseline level of one or more of LPC (e.g., LPC(16:0) or LPC(18:2)), sphingomyelins, and ceramides, e.g., a level that is measured when the patient is not experiencing an exacerbation.

In some aspects, the reference level is a pre-assigned level of one or more of LPC (e.g., LPC(16:0) or LPC(18:2)), sphingomyelins, and ceramides.

In some aspects, the reference level is a level of one or more of LPC (e.g., LPC(16:0) or LPC(18:2)), sphingomyelins, and ceramides (e.g., HCER or LCER) in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPC (e.g., LPC(16:0) or LPC(18:2)) is between about 227 nmol/mL to about 277 nmol/mL. In some aspects, the reference level for LPC (e.g., LPC(16:0) or LPC(18:2)), is about 252 nmol/mL.

In some aspects, the reference level for sphingomyelins is between about 448 nmol/mL to about 548 nmol/mL. In some aspects, the reference level for sphingomyelins is about 498 nmol/mL.

In some aspects, the ceramide is hexosylceramide (HCER). In some aspects, the reference level for HCER is between about 6.1 nmol/mL to about 7.5 nmol/mL. In some aspects, the reference level for HCER is about 6.8 nmol/mL.

In some aspects, the ceramide is lactosylceramide (LCER). In some aspects, the reference level for LCER is between about 4.3 nmol/mL to about 5.3 nmol/mL. In some aspects, the reference level for LCER is about 4.8 nmol/mL.

In some aspects, the reference level of LPC (e.g., LPC(16:0) or LPC(18:2)) is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPC (e.g., LPC(16:0) or LPC(18:2)), sphingomyelins, or ceramides levels, respectively, in the reference population.

In some aspects, the reference level of LPC (e.g., LPC(16:0) or LPC(18:2)) is the 33^{rd} percentile of LPC (e.g., LPC(16:0) or LPC(18:2)) levels in the reference population.

In some aspects, the reference level of sphingomyelins or ceramides (e.g., HCER or LCER) is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels, respectively, in the reference population.

In some aspects, the reference level of sphingomyelins or ceramides (e.g., HCER and/or LCER) is the 67^{th} percentile of sphingomyelins or ceramides levels, respectively, in the reference population.

In some aspects, the level of LPC (e.g., LPC(16:0) or LPC(18:2)) (e.g., the baseline level of LPC (e.g., LPC(16:0) or LPC(18:2)) in the patient) is at or below the 33^{rd} percentile of LPC (e.g., LPC(16:0) or LPC(18:2)) levels in the reference population, e.g., is at or below the 32^{nd} percentile, 31^{st} percentile, 30^{th} percentile, 29^{th} percentile, 28^{th} percentile, 27^{th} percentile, 26^{th} percentile, 25^{th} percentile, 24^{th} percentile, 23^{rd} percentile, 22^{nd} percentile, 21^{st} percentile, 20^{th} percentile, 19^{th} percentile, 18^{th} percentile, 17^{th} percentile, 16^{th} percentile, 15^{th} percentile, 14^{th} percentile, 13^{th} percentile, 12^{th} percentile, 11^{th} percentile, 10^{th} percentile, 9^{th} percentile, 8^{th} percentile, 7^{th} percentile, 6^{th} percentile, 5^{th} percentile, 4^{th} percentile, 3^{rd} percentile, 2^{nd} percentile, or 1^{st} percentile of LPC (e.g., LPC(16:0) or LPC(18:2)) levels in the reference population.

In some aspects, the level of sphingomyelins or ceramides (e.g., HCER and/or LCER) (e.g., the baseline level of sphingomyelins or ceramides in the patient) is at or above the 67^{th} percentile of sphingomyelins or ceramides levels in the reference population, e.g., is at or above the 68^{th} percentile, 69^{th} percentile, 70^{th} percentile, 71^{st} percentile, 72^{nd} percentile, 73^{rd} percentile, 74^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of sphingomyelins or ceramides levels in the reference population.

### VII. Methods for Enrolling Patients in Clinical Studies

In some aspects, the disclosure features a method of enrolling a patient suitable for a clinical study, the method comprising measuring a level of one or more of LPA16:0, LPA18:0, LPA18:1, LPA18:2, and LPA20:4 in a sample from the patient, wherein a level of one or more of LPA16:0, LPA18:0, LPA18:1, LPA18:2, and LPA20:4 in the sample that is below a reference level (e.g., a reference level as described in Sections IIIA and IIIB herein) identifies the patient as one who is suitable for the clinical study. In some aspects, the patient has an inflammatory respiratory disease, e.g., a respiratory disease as described in Section IIIE herein, e.g., COPD or asthma. In some aspects, the inflammatory respiratory disease is asthma and a level of one or more of LPA16:0, LPA18:0, and LPA18:2 in the sample that is below a reference level (e.g., a reference level as described in Sections IIIA and IIIB herein) identifies the patient as one who is suitable for the clinical study.

In some aspects, the method further comprises enrolling the patient who has been identified as suitable for the clinical study in the clinical study.

In some aspects, the level of one or more of LPA16:0, LPA18:0, LPA18:1, LPA18:2, and LPA20:4 is a baseline level of one or more of LPA16:0, LPA18:0, LPA18:1, LPA18:2, and LPA20:4, e.g., a level that is measured when the patient is not experiencing an exacerbation.

In some aspects, the level of one or more of LPA14:0, LPA16:0, LPA16:1, LPA18:0, LPA18:1, LPA18:2, LPA20:4, and LPA 22:4 (e.g., one or more of LPA16:0, LPA18:0, LPA18:1, LPA18:2, and LPA20:4), e.g., one, two three, four, five, six, seven, or all eight of LPA14:0, LPA16:0, LPA16:1, LPA18:0, LPA18:1, LPA18:2, LPA20:4, and LPA 22:4 or one, two, three, four, or all five of LPA16:0, LPA18:0, LPA18:1, LPA18:2, and LPA20:4) in the sample from the patient is below a reference level.

In some aspects, the reference level is a pre-assigned level of one or more of LPA16:0, LPA18:0, LPA18:1, LPA18:2, and LPA20:4.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) asthma). In some aspects, the patient has experienced at least one exacerbation in the prior 12 months.

In some aspects, the reference level is a level of one or more of LPA16:0, LPA18:0, LPA18:1, LPA18:2, and LPA20:4 in a reference population, e.g., a reference population of patients having the inflammatory respiratory disease (e.g., COPD (e.g., stage II, stage III, or stage IV COPD) or asthma). In some aspects, the patient has experienced at least one exacerbation in the prior 12 months.

In some aspects, the reference level for LPA16:0 is between about 0.12 μM to about 0.16 μM. In some aspects, the reference level for LPA16:0 is about 0.14 μM.

In some aspects, the reference level for LPA18:0 is between about 0.01 μM to about 0.035 μM.

In some aspects, the reference level for LPA18:0 is about 0.025 μM.

In some aspects, the reference level for LPA18:1 is between about 0.10 μM to about 0.14 μM. In some aspects, the reference level for LPA18:1 is about 0.12 μM.

In some aspects, the reference level for LPA18:2 is between about 0.42 μM to about 0.53 μM. In some aspects, the reference level for LPA18:2 is about 0.48 μM.

In some aspects, the reference level for LPA20:4 is between about 9 μM to about 13 μM. In some aspects, the reference level for LPA20:4 is about 10.9 μM.

In some aspects, the reference level of LPA16:0, LPA18:0, LPA18:1, or LPA18:2 is the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of LPA16:0, LPA18:0, LPA18:1, or LPA18:2 levels, respectively, in the reference population.

In some aspects, the reference level of LPA16:0, LPA18:0, LPA18:1, or LPA18:2 is the 33^{rd} percentile of LPA16:0, LPA18:0, LPA18:1, or LPA18:2 levels, respectively, in the reference population.

In some aspects, the reference level of LPA20:4 is the 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of LPA20:4 levels, in the reference population.

In some aspects, the reference level of LPA20:4 is the 67^{th} percentile of LPA20:4 levels in the reference population.

### VIII. Examples

The following are examples of methods, uses, and compositions of the invention. It is understood that various other aspects may be practiced, given the general description provided above, and the examples are not intended to limit the scope of the claims.

### Example 1. Methods for Extraction of Lysophosphatidic Acid Species

A. LPA Species and Disease

The present study compared sample extraction processes for LPAs and found that sample preparations using very low concentrations of HCl in the extraction buffer can cause the overestimation of lipid recovery. We optimized LC-MS/MS parameters, and validated an alternative method using a citric acid and disodium phosphate extraction buffer based on sample matrix effects, linearity, accuracy, precision and stability.

B. Chemicals and Standards

HPLC-grade methanol and water were obtained from Fisher Chemical (Pittsburgh, PA). Dichloromethane (DCM, >99.5%) was purchased from Honeywell Burdick & Jackson (Muskegon, MI).

LPA and other lysophospholipid (lysophosphatidylglycerol, lysophosphatidylserine, lysophosphatidylinositol, lysophosphatidylethanolamine and lysophosphatidylcholine) standards were purchased from Avanti Polar Lipids, Inc (Alabaster, AL). HCl was obtained from Thermo Scientific™ (Rockford, IL). Citric acid and disodium phosphate were obtained from Sigma-Aldrich (St. Louis, MO).

C. Sample Collection

Two cohorts of samples were collected. For the small cohort study, serum samples from healthy controls (n=10) and COPD (n=11) patients were obtained from an internal biobank. To investigate LPA species associations with demographics and COPD metrics, patient baseline serum samples were collected from a COPD randomized controlled trial (NCT02546700, n=268; the large cohort study). Clinical characteristics of the patients are shown in Table 1.

TABLE 1
Clinical characteristics of patients

| | Female | | Male | |
|---|---|---|---|---|
| | (n = 105) | | (n = 163) | |
| Age (SD) | 64 | (7) | 65 | (6) |
| Race | | | | |
| White (%) | 97 | (92) | 154 | (94) |
| Black or African American (%) | 3 | (3) | 6 | (4) |
| Others (%) | 5 | (5) | 3 | (2) |
| BMI (SD) | 27.1 | (5.6) | 26.8 | (4.8) |
| Former smokers (%) | 53 | (50.5) | 103 | (63) |
| Current smokers (%) | 52 | (49.5) | 60 | (37) |
| Post-bronchodilator FEV1 (SD) | 1.2 | (0.4) | 1.4 | (0.5) |
| Post-bronchodilator FEV1/FVC ratio | 0.52 | (0.13) | 0.45 | (0.13) |
| Region | | | | |
| North & South America (%) | 49 | (47) | 40 | (25) |
| Others (%) | 56 | (53) | 123 | (75) |
| Patients with chronic bronchitis (%) | 74 | (70) | 127 | (78) |

SD: standard deviation; percentages (%) were calculated within female or male group.

D. Lipid Extraction

i. Citric Acid and Disodium Phosphate Method

Serum (20 μl) was spiked with 8 ng of LPA 17:0, and citric acid and disodium phosphate acidification buffer (0.5 ml; 30 mM citric acid, 40 mM disodium phosphate; pH=4.0) was added, followed by butanol (2 ml), similar to the method described previously (Baker et al., Anal Biochem. 292: 287-295, 2001). The solution was vortexed and centrifuged at 1000×g for 10 minutes. After centrifugation, the top layer was transferred to a clean glass tube. The bottom layer was extracted a second time with 1 ml water-saturated butanol. The sample extracts were pooled, dried under a gentle nitrogen stream and reconstituted in methanol (50 μl). Reconstituted samples were transferred into HPLC vials and analyzed by LC-MS/MS.

ii. HCl Method

For comparison to the previously used method, the citric acid and disodium phosphate buffer was replaced by 0.1N HCl acidification buffer (0.5 ml) in the above-described protocol. Serum samples from healthy donors were used in the experiments comparing the two acidification buffers (disodium phosphate/citric acid or 0.1 N HCl).

E. Liquid Chromatography-Mass Spectrometry

Chromatographic separation of LPAs was performed on a reverse-phase column (Luna Omega C18 1.6 μm, 100×2.1 mm). The temperatures of the column oven and auto sampler were set at 40° C. and 15° C., respectively. The injection volume was 5 μl. The LC flow rate was set at 0.2 ml/min. Gradient initial conditions were 80% mobile phase A (95:5 water:methanol, 5 mM ammonium acetate, 0.1% formic acid) and 20% mobile phase B (5:95 water:methanol, 5 mM ammonium acetate, 0.1% formic acid). After 2 minutes at initial conditions, mobile phase B was increased to 85% within 1 minute, further increased to 95% over 12 minutes, and then to 100% in 1 minute. Mobile B was held at 100% for 5 minutes and returned to the initial conditions for a 5-minute re-equilibration before the next injection.

The liquid chromatograph was coupled to a 6500+QTRAP mass spectrometer (Sciex, Redwood City, CA). The parameters for mass spectrometry were optimized by direct infusion of individual standard to obtain the highest signal intensities for all analytes. LC-MS/MS was operated under negative ionization mode with the following post-optimization source settings: turbo-ion-spray source at 300° C., N2 nebulization at 16 psi, N2 heater gas at 10 psi, curtain gas at 35 psi, collision-activated dissociation gas pressure was held at medium, turbo ion-spray voltage at −4500 V, declustering potential (DP) at −70V, entrance potential (EP) at −10V, and collision cell exit potential (CXP) at −10 V. Sample analysis was performed in multiple reactions monitoring (MRM) mode. The collision energy (CE) and transitions monitored for LPA species and other lysophospholipids (lysophosphatidylglycerol (LPG), lysophosphatidylserine (LPS), lysophosphatidylinositol (LPI), lysophosphatidylethanolamine (LPE) and lysophosphatidylcholine (LPC)) are listed in Table 2.

TABLE 2
MRM transitions and CE parameters of lysophospholipids

| Compound name | Q1 | Q3 | CE |
|---|---|---|---|
| LPA14:0 | 381.1 | 153.0 | −28 |
| LPA16:1 | 406.9 | 153.0 | −28 |
| LPA22:4 | 485.6 | 153.0 | −28 |
| LPA16:0 | 408.9 | 153.0 | −28 |
| LPA17:0 | 423.0 | 153.0 | −28 |
| LPA18:0 | 437.1 | 153.0 | −28 |
| LPA18:1 | 435.3 | 153.0 | −28 |
| LPA18:2 | 433.3 | 153.0 | −28 |
| LPA20:4 | 457.1 | 153.0 | −28 |
| LPC16:0 | 554.3 | 255.2 | −43 |
| LPC18:0 | 582.4 | 283.3 | −43 |
| LPC18:1 | 580.4 | 281.2 | −43 |
| LPC18:2 | 578.3 | 279.2 | −43 |
| LPC20:4 | 602.4 | 303.2 | −43 |
| LPE16:0 | 452.3 | 255.2 | −50 |
| LPE18:0 | 480.3 | 283.3 | −50 |
| LPE18:1 | 478.3 | 281.2 | −50 |
| LPE18:2 | 476.3 | 279.2 | −50 |
| LPE20:4 | 500.3 | 303.2 | −50 |
| LPG16:0 | 483.3 | 255.2 | −50 |
| LPG18:0 | 511.3 | 283.3 | −50 |
| LPG18:1 | 509.3 | 281.2 | −50 |
| LPG18:2 | 507.3 | 279.2 | −50 |
| LPG20:4 | 531.3 | 303.2 | −50 |
| LPI16:0 | 571.3 | 255.2 | −50 |
| LPI18:0 | 599.3 | 283.3 | −50 |
| LPI18:1 | 597.3 | 281.2 | −50 |
| LPI18:2 | 595.3 | 279.2 | −50 |
| LPI20:4 | 619.3 | 303.2 | −50 |
| LPS16:0 | 496.3 | 153.0 | −50 |
| LPS18:0 | 524.3 | 153.0 | −50 |
| LPS18:1 | 522.3 | 153.0 | −50 |
| LPS18:2 | 520.3 | 153.0 | −50 |
| LPS20:4 | 544.3 | 153.0 | −50 |

Q1: precursor ion; Q3: product ion; CE: collision energy; DP: declustering potential; EP: entrance potential; CXP: collision cell exit potential.

### Example 2. Analysis of Extraction Methods

A. Sample Matrix Effect and Linearity

Linearity of the method was evaluated by analyzing six different concentrations of standards prepared from a stock solution in DCM:methanol (1:1, v:v) (Different volumes of stock or diluted standards were spiked in the solvent). The concentrations of calibration curves are in the range of 0.01-14 μM for different LPA species. To investigate the sample matrix effects, LPA standards were spiked into serum sample extracts. The slopes were compared with the standard curves prepared in DCM:methanol (1:1) solvent.

B. Sensitivity, Accuracy, and Precision

Sensitivity of the assays was determined by limits of detection (LOD) and limits of quantitation (LOQ), which were evaluated by serial dilution of standards. LOD and LOQ were determined as the concentration levels that yielded the peaks with 3 S/N (signal/noise) and 10 S/N, respectively. Accuracy was determined by spiking analytes into serum samples (n=3) and comparing the recovered quantities with actual spiked quantities. Three different levels (low, medium and high) were spiked and covered the concentration range of LPAs in healthy and patient samples.

C. Evaluation of LPA stability

LPA levels in a sample can increase during storage in a freezer (−80° C.), presumably through enzymatic conversion. Large-scale sample analysis may require weeks for sample preparation. To monitor sample stability during storage and analysis between different batches, 10 μL of serum from each patient sample was pooled and aliquoted into separate glass vials as an internal quality control (QC) sample. A fresh aliquot of QC pooled sample was thawed and analyzed along with patient samples for each batch to monitor sample stability and analysis reproducibility across batches

To evaluate LPA stability in extract solvent, extracted samples were placed in a 15° C. autosampler and injected successively for a total of five injections over 55 hours.

D. Statistical Analysis

Statistical analyses were performed using nonparametric Mann-Whitney u test, one-way ANOVA, Pearson correlation analysis, logistic regression, multivariable linear regression, and false-discovery-rate (FDR) adjustment. The relationships between LPA levels and baseline demographics and clinical metrics were assessed on a logarithmic scale of LPA concentrations. For the comparison between healthy donors and COPD patients, logistic regression followed by FDR adjustment was performed to generate q values. LPA species were also assessed by partial least-squares discriminant analysis (PLS-DA) validated using 7-fold internal cross-validation. The quality of the statistical model was evaluated by R2Y and Q2Y scores. The permutation test was conducted to further validate the PLS-DA model. For the data from the baseline patient samples (n=268), the association of LPA with each parameter was first analyzed using univariate analysis to assess statistical significance of any correlations. The confounding factors that could affect LPA levels were then incorporated into a multivariable linear regression model for multivariate analysis to adjust for associations with LPAs. P values from multivariable linear regression were also adjusted by FDR to generate q values. P values less than 0.05 and q values less than 0.1 were considered statistically significant.

### Example 3. Method Comparison and Validation

A. Method Comparison

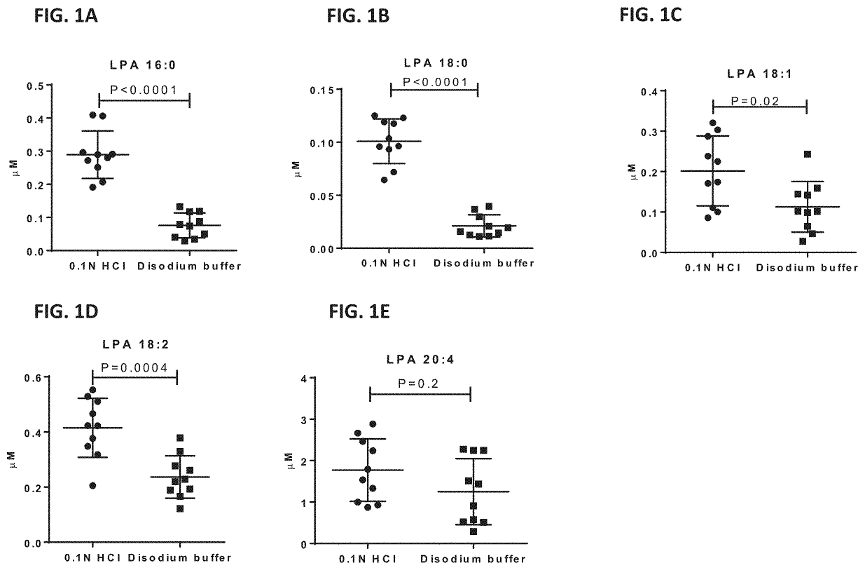

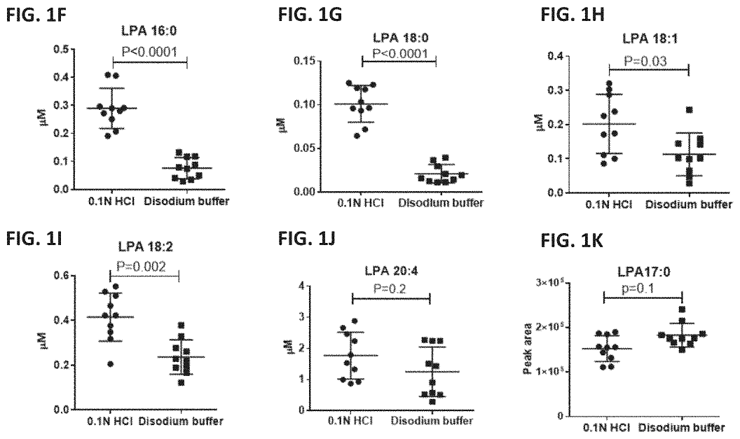

LPA concentration measurements in healthy donor serum samples were compared between samples processed using a 0.1 N HCl buffer and samples processed using a citric acid and disodium phosphate buffer. Five LPA species were compared: LPA16:0, LPA18:0, LPA18:1, LPA18:2 and LPA20:4. The method using the 0.1 N HCl buffer resulted in LPA species levels two- to three-fold higher than the citric acid and disodium phosphate buffer (FIGS. 1A-1E).

Due to ambiguity in previous publications regarding best practices for LPA quantification, LPA species were measured from serum or plasma samples using a variety of sample preparation methods for extraction. Compared to the traditional Bligh & Dyer method (Dayanjan et al., Analytical Methods, 3: 2822-2828, 2011), the addition of acid can greatly improve extraction efficiency. The most commonly used additives to acidify extraction buffer are HCl and disodium phosphate buffer (30 mM citric acid and 40 mM Na2HPO4) (Dayanjan et al., Analytical Methods, 3: 2822-2828, 2011; Baker et al., Anal Biochem, 292: 287-295, 2001; Onorato et al., J Lipid Res, 55: 1784-1796, 2014; Aoki et al., The Journal of Biological Chemistry, 277; 48737-48744, 2002). High concentrations of HCL have been reported to cause the overestimation of LPA species (Onorato et al., J Lipid Res, 55: 1784-1796, 2014; Scherer et al., Clin Chem, 55: 1218-1222, 2009). Acid increases hydrolysis of the choline group from other lysophospholipids, such as LPC, rendering LPA-like moieties. Extraction using a non-acidic buffer avoids hydrolysis, but compromises extraction efficiency. Consequently, a larger volume of biofluidic samples would be required. Alternatively, 0.1N HCl has been reported to improve extraction efficiency when used as acidification buffer (Montesi et al., BMC Pulmonary Medicine, 14: 5, 2014). To investigate whether lower concentrations of HCl also possess the ability to cause hydrolysis, we compared LPA extraction using either 0.1 N HCl or disodium phosphate buffer. Our data showed that 0.1 N HCl resulted in LPA levels two- to three-fold higher compared to disodium phosphate buffer (FIGS. 1F-1K). The lower concentrations of LPAs extracted using disodium phosphate buffer are not likely an underestimation. The intensity of the internal standard LPA17:0, which cannot undergo acid hydrolysis, were higher (but not significant) in samples extracted using disodium phosphate buffer than in 0.1N HCl extracted samples, suggesting the extraction efficiency is similar between the two methods, and furthermore the higher concentrations of detected LPA species from 0.1 N HCl extraction is due to the hydrolysis of other lysophospholipids (FIGS. 1F-1K). Due to potential hydrolysis and subsequent moiety overestimation resulting from even small amounts of HCl, we concluded that the disodium phosphate buffer was the preferential additive to improve LPA extraction efficiency and minimize hydrolysis. The sample preparation method was subsequently validated using 40 mM Na₂HPO₄ and 30 mM citric acid as an acidification buffer.

B. Liquid Chromatographic Separation of LPA in Serum

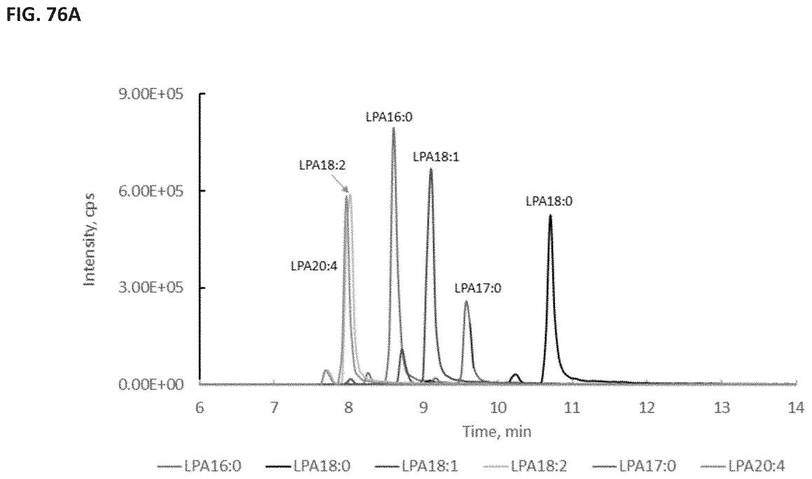

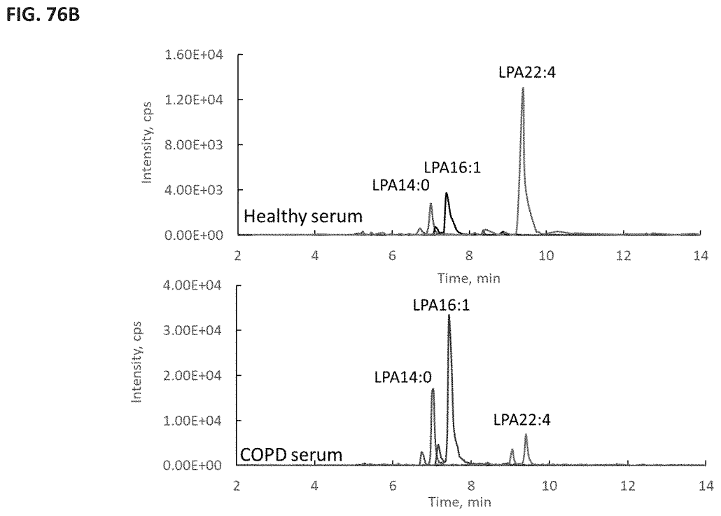

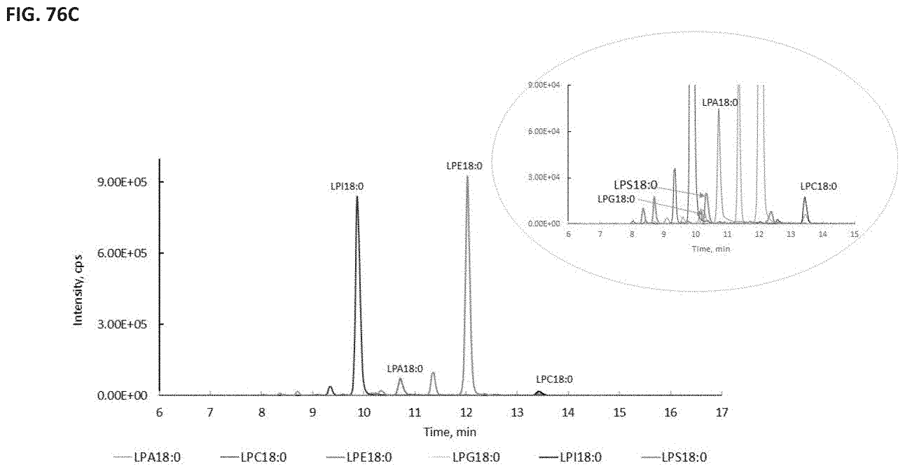

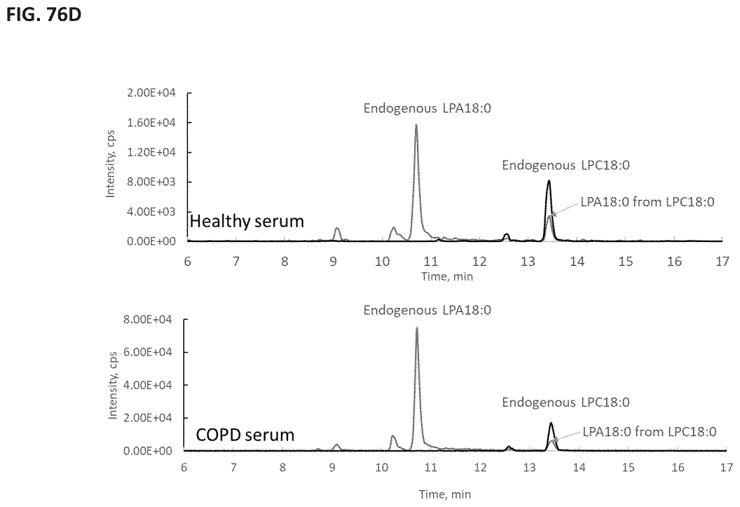

Five of the most abundant LPA species (LPA16:0, 18:0, 18:1, 18:2 and 20:4) were separated based on retention times and MRM transitions (Table 2 and FIG. 76A). LPA14:0, LPA16:1 and LPA22:4 peaks were also detected in healthy and COPD serum using theoretical transitions but were not validated nor used for quantitative analysis due to the lack of available standards (FIG. 76B). Other lysophospholipids, LPE, LPG, LPI, LPC and LPS, may be co-extracted from serum and converted into LPA during ionization (Onorato et al., J Lipid Res, 55: 1784-1796, 2014; Zhao et al., J. Chromatogr. B Analyt. Technol. Biomed. Life Sci, 877: 3739-3742, 2009). The chromatographic gradient was thus optimized to separate LPA from other lysophospholipids. The relative intensity ratios between LPA and other lysophospholipids vary in individual serum samples, but in general, LPA, LPC, LPE and LPI are abundant while LPG and LPS are at low level. As exemplified by LPA18:0 in FIG. 76C, LPA was completely separated from other endogenous lysophospholipids that contain the same acyl chain length. The extracted ion chromatograms (FIG. 76D, LPA18:0 and LPC18:0, as an example) from healthy and COPD sera showed that only LPC converted LPA was detected in serum, indicating the importance of the separation of LPA from LPC. The small peaks eluting right before all lysophospholipids represent the sn-2 isomer and were also noted in Onorato et al., J Lipid Res, 55: 1784-1796, 2014. We noticed that the peak shape of LPA varied on different batches of columns. LPA peaks also have a tendency to tail on C18 columns. Increasing the composition of ammonium acetate or formic acid in buffers can optimize the peak shapes to minimize or abrogate these issues. We also found that the retention time could shift if the instrument “environment” was altered by running long term strong buffers such as 0.1% heptafluorobutyric acid. This issue was resolved by a thorough cleaning of the LC and mass spectrometer system. The MRM transitions of lysophospholipids are usually generated by the loss of fatty acids or the glycerol-3-phosphate ion with loss of H₂O (fragments of 152.9958). The transitions listed in Table 2 were found to generate higher intensities than other commonly used fragment ions under our chromatographic and mass spectrometric conditions.

C. Sample Matrix Effect, Linearity, Sensitivities, Accuracy and Precision

The slope of the calibration curve reflects the response of the instrument to analyte concentration. We calculated the standard curves prepared from sample extract matrix as well as pure preparations in solvent (Table 3). Ratios of the slopes of the dilution curves ranged from 0.98-1.12. Therefore, the sample matrix did not affect the instrument response to LPA. To preserve limited patient samples, subsequent solutions for calibration curves were prepared in DCM:methanol (1:1, v:v). All LPA species showed good linear signal response across the assessed concentration ranges of the LPAs (R²>0.990).

The sensitivity (LOD and LOQ), accuracy and precision of all LPAs are shown in Table 3 and Table 4. The LODs ranged from 0.002 to 0.008 μM, indicating that the multiple reactions monitoring (MRM) method was sufficiently sensitive for LPA detection in serum. LPA recoveries ranged from 87.8 to 109.5% at low level (lowest tertile of levels), from 82.4 to 102.1% at middle level (middle tertile), and from 82.0 to 100.0% at high level (highest tertile) concentrations. The relative standard deviations (RSDs) ranged from 3.1 to 26.6%. These results showed that the method had the desired accuracy and precision for the detection of LPAs from serum samples having a small volume.

The optimized instrument parameters and the application of MRM scan mode taken together with the modified extraction protocol significantly increased sensitivity of LPA detection compared to previous reports. The optimized workflow utilizes only 20 μL serum, representing a significant reduction in sample volume compared to previously reported methods, which required 100-600 μl samples (Dayanjan et al., Analytical Methods, 3: 2822-2828, 2011; Baker et al., Anal Biochem, 292: 287-295, 2001; Tokumura et al., Biology of Reproduction, 67: 1386-1392, 2002; Wang Jialu, Facile Methods for the Analysis of Lysophosphatidic Acids in Human Plasma. Dissertations and Theses, Paper 2235, 2015). A further minimization of the reconstitution volume from 50 μL to 20 μL or less may further benefit samples with limited volume.

TABLE 3
LOD, LOQ, and sample matrix effects of LPA detection
via LC-MS/MS in human serum samples

| | standard curves | standard curves from | LOD(μM | LOQ(μM | |
|---|---|---|---|---|---|
| | from solvent | sample matrix | serum) | serum) | ratio |
| LPA16:0 | y = 1.2477x + 0.0845 | y = 1.3618x + 0.3391 | 0.008 | 0.03 | 1.09 |
| LPA18:0 | y = 0.2387x + 0.0844 | y = 0.2664x + 0.0864 | 0.002 | 0.007 | 1.12 |
| LPA18:1 | y = 1.1067x + 0.0046 | y = 1.163x + 0.2904 | 0.008 | 0.03 | 1.05 |
| LPA18:2 | y = 6.3609x + 0.1857 | y = 6.3771x + 1.346 | 0.008 | 0.03 | 1.00 |
| LPA20:4 | y = 0.5775x − 0.0067 | y = 0.5665x + 0.688 | 0.004 | 0.01 | 0.98 |

TABLE 4
Accuracy and precision of LPA detection via LCMS/MS in human serum

| | Spike 0.32 ng | | Spike 1.6 ng | | Spike 8 ng | |
|---|---|---|---|---|---|---|
| | Recovery | RSD* | Recovery | RSD* | Recovery | RSD* |
| LPA16:0 | 95.2 | 10.4 | 85.4 | 12.7 | 100.0 | 3.2 |
| LPA18:0 | 87.8 | 20.7 | 83.2 | 13.8 | 99.5 | 3.1 |
| LPA18:1 | 102.1 | 11.9 | 82.4 | 10.8 | 99.7 | 4.3 |
| LPA18:2 | 109.5 | 11.7 | 93.5 | 17.4 | 82.0 | 26.6 |
| LPA20:4 | NA | NA | 102.1 | 25.0 | 90.3 | 15.5 |

*RSD: relative standard deviation

D. LPA Stability

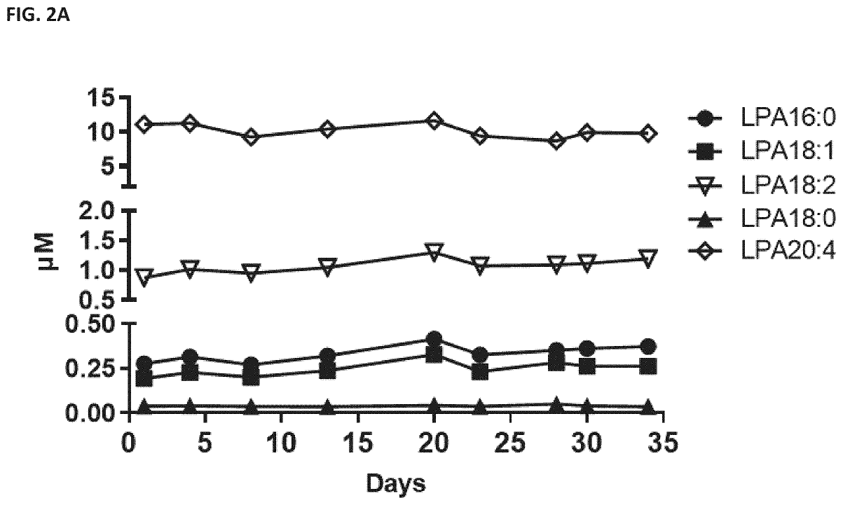

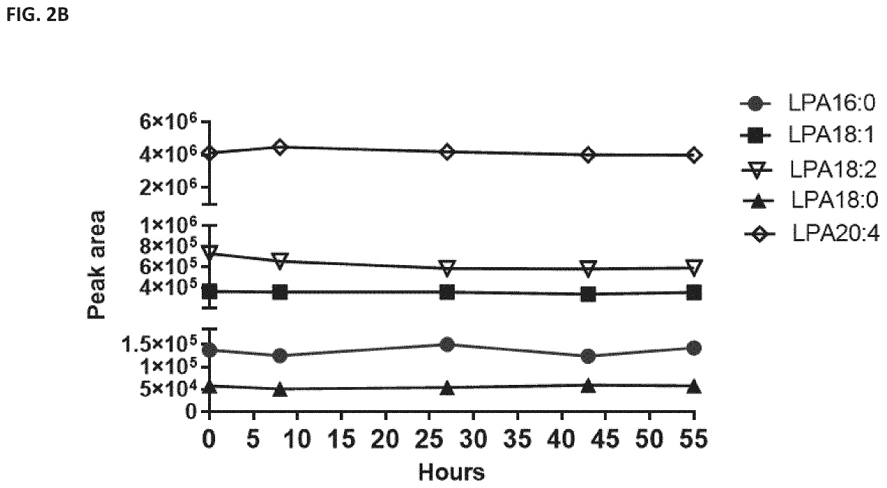

A pooled sample aliquoted and used as an internal QC was analyzed along with each sample batch. Stability curves demonstrated that there was no significant elevation of LPAs across the analysis interval of 35 days. While signals fluctuated slightly across the analysis interval, overall relative standard deviations (RSDs) of the QC sample results were within 20% (FIG. 2A). Therefore, LPAs in serum samples were deemed stable during storage at −80° C., and the quantification of LPAs across different batches or different days was reproducible. There was no observable degradation of LPAs within 55 hours during repeated injections from the autosampler, indicating that LPA levels were stable at 15° C. in organic solvent after extraction (FIG. 2B). Reducing temperature in the autosampler was not attempted due to the possibility of LPA precipitation.

### Example 4. Evaluation of LPA Levels in Healthy Donors and COPD Patients

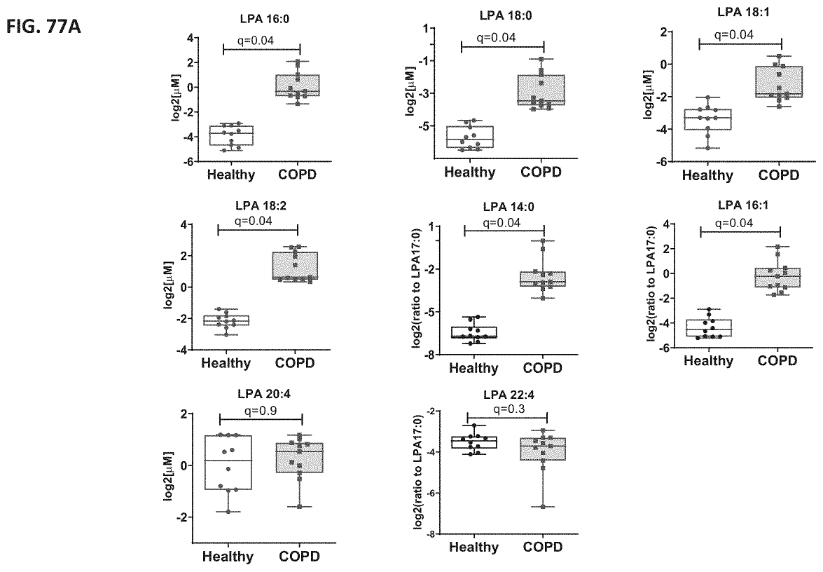

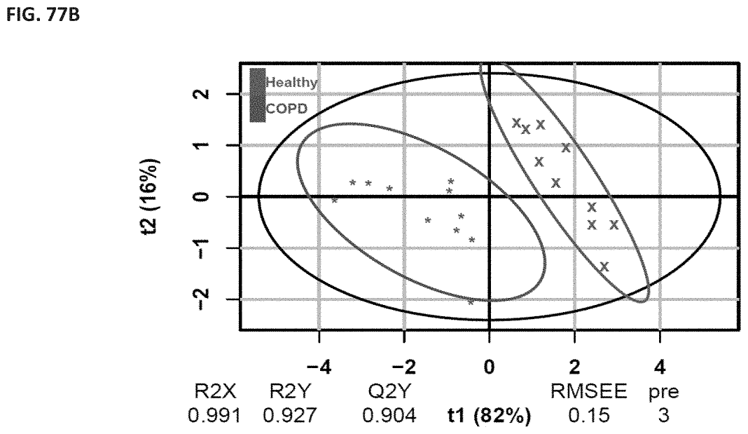

A. LPA levels in healthy versus COPD patient samples from a small cohort study Using the citric acid and disodium phosphate extraction buffer method established in Examples 1-3, serum samples from 10 heathy donors and 11 COPD patients were analyzed. Single injections were made for each sample. The concentrations of LPA species detected from 20 μl serum samples were all above LOQ. The average concentrations of LPA 16:0, 18:0, 18:1, 18:2 and 20:4 (±SD) were 0.08±0.04, 0.02±0.01, 0.11±0.06, 0.24±0.08 and 1.25±0.8 μM, respectively, in heathy donors, versus 1.45±1.28, 0.17±0.15, 0.52±0.41, 2.87±1.89 and 1.36±0.61 μM, respectively, in COPD patient samples. Other LPA species, such as LPA14:0, LPA16:1 and LPA22:4, were also identified using their theoretical MRM transitions. Intensities of LPA14:0 and LPA16:1 were from LOD to LOQ level in healthy control and increased significantly in COPD patient serum (FIG. 77). LPA22:4 ranged from LOD to 3 times LOQ level in healthy and COPD serum, while the intensity did not change between healthy and COPD groups (FIG. 77A). Though these three LPA analytes were not quantified, their relative changes of intensity are nonetheless valuable for exploratory/discovery study for disease.

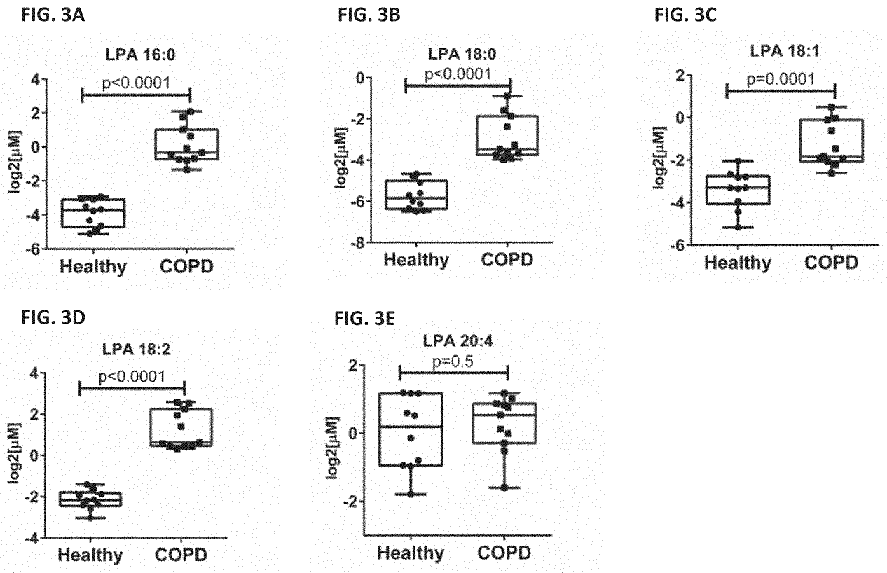

From univariate analysis, concentrations of the quantified LPA species were significantly higher in COPD patient serum compared to healthy donors (p≤0.0001), with the exception of LPA 20:4 (p=0.5) (FIGS. 3A-3E).

The comparison was performed using logistic regression adjusted for the confounding factors age and gender, followed by adjustment by false-discovery-rate. After adjustment, LPA levels (except for LPA20:4) remained significantly higher in COPD (FIG. 77A, p<0.05, q<0.05). The odd ratios from the logistic regression were 5.4, 22.0, 6.3, 6.4 and 0.94 for LPA16:0, LPA18:0, LPA18:1, LPA18:2 and LPA20:4, respectively. The LPA species levels measured in healthy controls are similar to those reported by Baker et al. (Anal Biochem, 292: 287-295, 2001), who also used the disodium phosphate buffer for sample extraction, but were but lower than those of Tokumura et al., (Biology of Reproduction, 67: 1386-1392, 2002), in which 1N HCl was used for the sample extraction.

With the exception of LPA 20:4, the average concentration of LPA species measured in COPD patients was 4.6- to 22.4-fold higher than healthy controls. After logistic regression and FDR adjustment, LPA species were significantly higher in COPD than in healthy donors. Healthy donors and COPD patients were completely separated by supervised PLS-DA score plot (FIG. 77B). Q2Y and R2Y measures (R2Y=0.927; Q2Y=0.904; p=0.01) indicated a good fitting and high predictive accuracy of the model based on the reported criteria (Chin, The partial least squares approach for structural equation modeling. In G. A. Marcoulides (Ed.), Methodology for business and management. Modern methods for business research. Lawrence Erlbaum Associates Publishers, 295-336, 1998; Westerhuis et al., Metabolomics, 4: 81-89, 2008; Triba et al., Mol Biosyst, 11: 13-19, 2015; Moltu et al., Nutrients, 8: 3-16, 2012; Szymanska et al., Metabolomics, 8: 3-16, 2012). These results suggest that activity of the LPA-ATX pathway is significantly upregulated in COPD disease, with the caveat that the age and sex of the healthy donors is unknown and so cannot be ruled out as a possible influence on the LPA values observed. Previous literature also reported higher levels of two LPA species, LPA16:0 and LPA18:2, in COPD smokers compared to smokers with normal lung function (Naz et al., Eur Respir J, 49: 1602322, 2017). Inhibition of ATX was proposed as a new potential treatment of COPD (Blanque et al., Eur Respir J, 46: PA2129, 2015). Therefore, LPAs can potentially act as a tool to characterize COPD patient subgroups. Our study is the first to detect eight LPA species from COPD patients, and to compare the concentrations of the five most abundant LPA species from COPD serum to healthy donors. These LPA species with various acyl chains are generated by different pathways and exhibit different binding affinity to the LPA receptors (Choi et al., Annu Rev Pharmacol Toxicol, 50: 157-186, 2010; Aoki et al., Seminars in Cell and Developmental Biology, 50: 157-186, 2010). Investigating the changes of LPA species with various fatty acyl chain length or saturation could assist the further study of disease mechanisms.

B. LPA Levels in COPD Patient Baseline Samples from a Large Cohort Study

The investigation of LPA changes on the ATX-LPA pathway is important for the diagnosis and treatment of COPD disease. Understanding the effects of demographics and/or clinical known data on LPA levels ensures the accurate evaluation of the changes in the ATX-LPA pathway. To investigate the correlation of LPA levels with demographic parameters and clinical metrics in the COPD patient population, 268 baseline samples from 105 female and 163 male patients covering different ages and geographic regions were tested. Patients were randomly assigned to batches by rejection sampling, which attempted to ensure an even distribution of the patients per group (age, gender, region, etc.) in each run to reduce the effect of confounding factors in the analysis. To assess the associations of LPAs with disease severity, it is important to understand how demographics affect LPA levels. Therefore, the effects of gender, age, region, and other known clinical data on LPA were investigated with both univariate and multivariable analysis.

i. Association of LPA Levels with Demographic Parameters

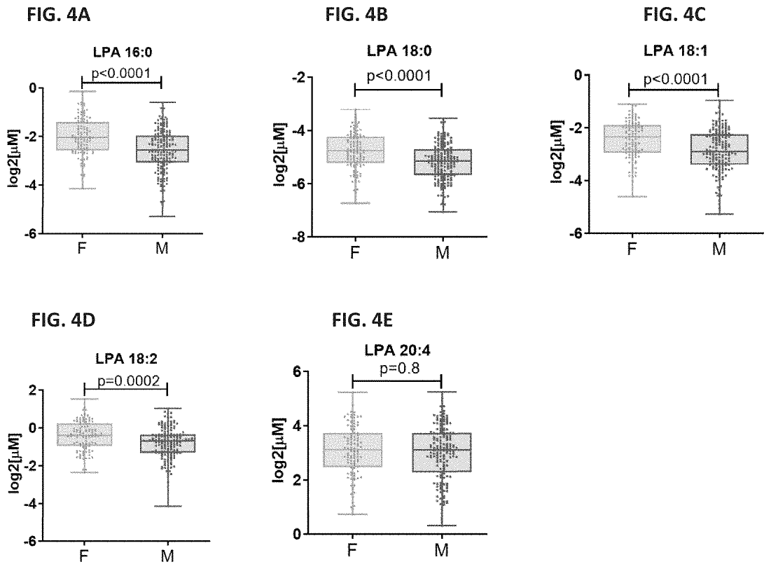

Results from univariate (FIGS. 4A-4E) and multivariate (Table 5) analyses showed significantly higher serum LPA levels in female patients (p≤0.01, q≤0.01) compared to male patients, with the exception of LPA 20:4 (p=0.5, q=0.5). Previous studies have also reported higher LPA levels in healthy females (Michalczyk et al., Lipids in Health and Disease, 16: 140, 2017; Hosogaya et al., Ann Clin Biochem, 45: 364-368, 2008; Knowlden et al., J Immunol., 192: 851-857, 2014), and serum ATX activity was found to be higher in women than in men (Michalczyk et al., Lipids in Health and Disease, 16: 140, 2017). These findings indicated potential interactions between sex hormones and the ATX-LPA pathway. Zhang et. al. (Mol Med Rep., 17: 4245-4252, 2018) reported that ATX mRNA levels are upregulated by estrogen and that estrogen may participate in regulating ATX generation and secretion. Autotaxin (Enpp2) expression was found to be upregulated in the hippocampus of ovariectomized rats when treated with estrogen (Takeo et al., Endocr J. 2008, 56, 113-120). Our findings validated the sex-based differences in LPA species levels in COPD patients and emphasized the importance of sex stratification in COPD biomarker studies.

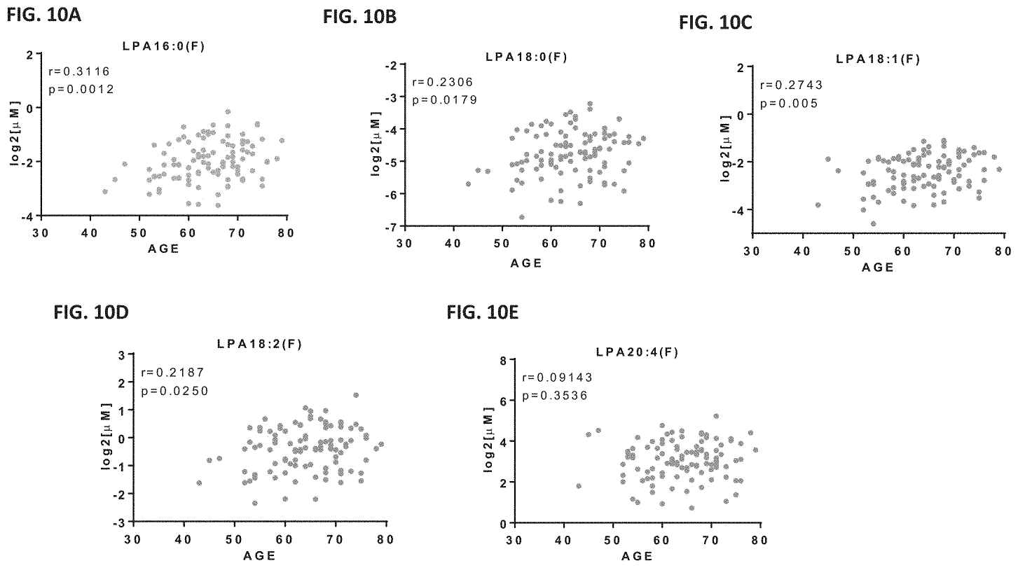

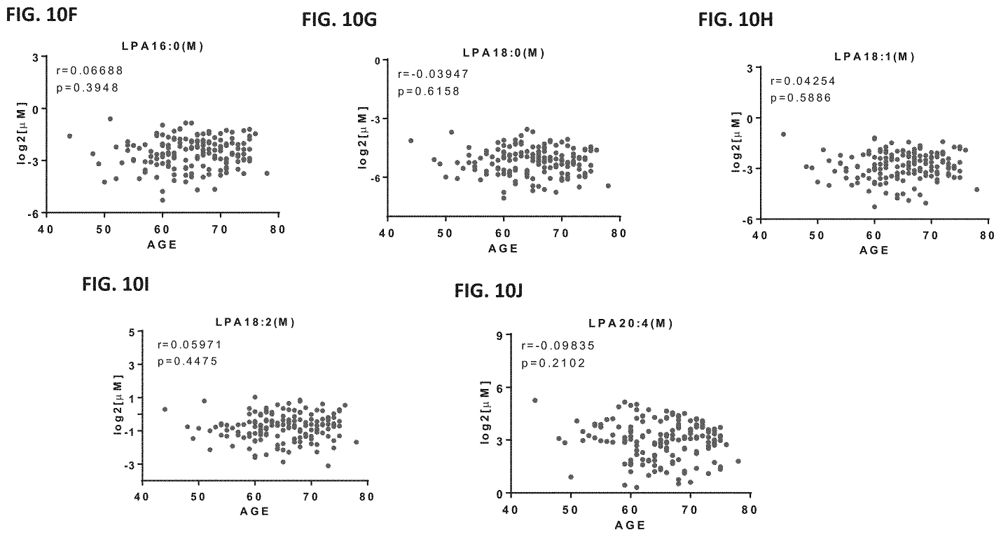

The subsequent correlations of LPA levels with other demographic parameters were therefore assessed in male and female patient groups separately. We plotted the correlation of LPA species concentrations with age (FIGS. 10A-10J). Univariate analysis results showed that increased age correlated significantly with increased LPA16:0 (r=0.3116, p=0.0012), LPA18:0 (r=0.2306, p=0.0179), LPA18:1 (r=0.2743, p=0.005) and LPA18:2 (r=0.2187, p=0.0250) in the female patient group. There was no significant correlation between age and LPA levels in the male patient group (p>0.1). After multivariable analysis (Table 5), LPA16:0 (p=0.03, q=0.1), LPA18:0 (p=0.1, q=0.1), LPA18:1 (p=0.05, q=0.1) and LPA18:2 (p=0.09, q=0.1) showed significant or moderate correlations with age in the female patient group.

TABLE 5
P values of LPA correlations with demographics and COPD
metrics after multivariable analysis and FDR adjustment

| | LPA levels | | Correlation | | Correlation | | | | | |
|---|---|---|---|---|---|---|---|---|---|---|
| | comparison in | | between LPA | | between LPA and | | | | Effect of region | |
| | male CB and | | and FVC in | | Age in female | | | | P ^{a} | q |
| | non-CB patients | | female patients | | patients | | Effect of gender | | Female | Female |
| | P ^{a} | q | P ^{a} | q | P ^{a} | q | P ^{b} | Q | (male) | (male) |
| LPA16:0 | 0.02 | 0.03 | 0.5 | 0.8 | 0.03 | 0.1 | 0.00002 | 0.0001 | 0.2 (0.9) | 0.2 (0.9) |
| LPA18:0 | 0.01 | 0.03 | 0.1 | 0.5 | 0.1 | 0.1 | 0.0003 | 0.0007 | 0.1 (0.8) | 0.2 (0.9) |
| LPA18:1 | 0.06 | 0.08 | 0.8 | 0.8 | 0.05 | 0.1 | 0.0004 | 0.0007 | 0.2 (0.5) | 0.2 (0.8) |
| LPA18:2 | 0.02 | 0.03 | 0.4 | 0.8 | 0.09 | 0.1 | 0.01 | 0.01 | 0.2 (0.3) | 0.2 (0.8) |
| LPA20:4 | 1 | 1 | 0.7 | 0.8 | 1 | 1 | 0.5 | 0.5 | 0.1 (0.03) | 0.1 (0.2) |

Multivariable analysis was performed by a multivariable linear regression model. The confounding factors that could affect LPA levels were adjusted.
^{a} the multivariable model adjusted LPA, age, FEV1, FVC, BMI, CB, smoking status and region (Gender-stratification was applied);
^{b} the multivariable model adjusted LPA, age, gender, smoking status, BMI, FEV1, FVC, CB and region.

Prior to our analysis, the correlation between age and LPA levels in COPD patients had not been discussed. COPD is a slow-developing disease that does not become prevalent in adults until after middle age. In the current study, the patient age ranged from 50 to 79 years. The significant positive correlation between age and levels of certain serum LPAs demonstrated that age in female patients could be an independent factor influencing LPA levels. Michalczyk et al. (Lipids in Health and Disease, 16: 140, 2017) reported a significant influence of age and sex on plasma LPA. Hosogaya et al. (Ann Clin Biochem, 45: 364-368, 2008) observed a weak but significant negative correlation between plasma LPAs concentrations and age in men, but not women. Both of these earlier studies were carried out on healthy subjects and not COPD patients. Due to the varying correlations observed across different studies and subject health and treatment conditions, the investigation of age effects is advisable when using LPAs species levels as potential prognostic/diagnostic markers in different patient cohorts.

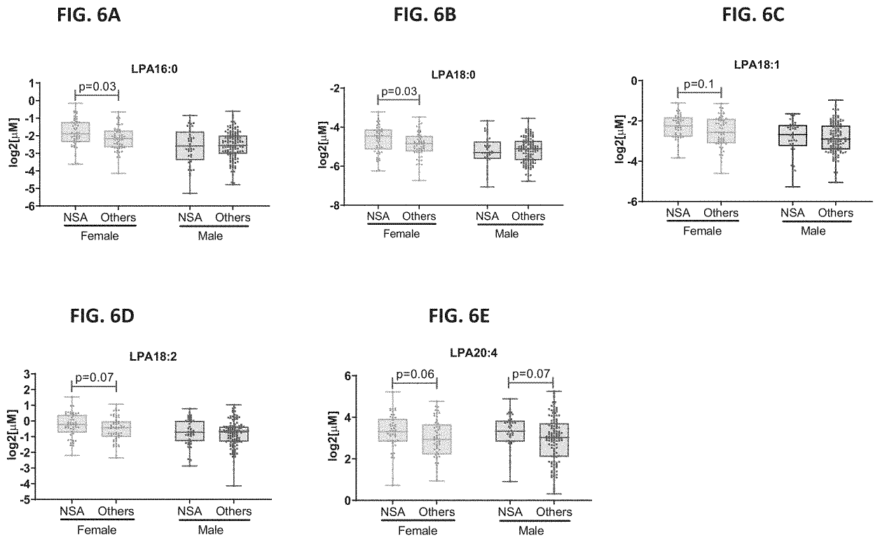

After univariate analysis, LPA levels were found to be higher in serum samples from women in North and South America compared to those in other regions (p=0.03 for LPA 16:0 and 18:0; p=0.1 for LPA 18:1, p=0.07 for LPA 18:2, p=0.06 for LPA20:4; FIGS. 6A-6E). No significant difference was detected in male patients, except for LPA 20:4 (p=0.04; FIGS. 6A-6E). However, after adjusting for age and gender with multivariate analysis, LPA levels were not significantly different across different regions (p>0.03, q>0.1, Table 5) in either female or male patients, indicating that demographic region in this cohort was not a major factor influencing LPA species levels.

ii. Association of LPA Levels with Smoking Status and BMI

(a) Smoking

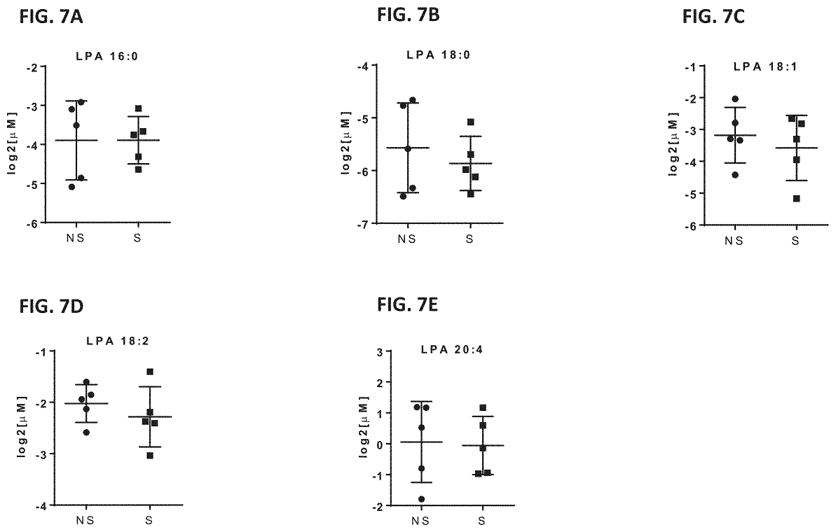

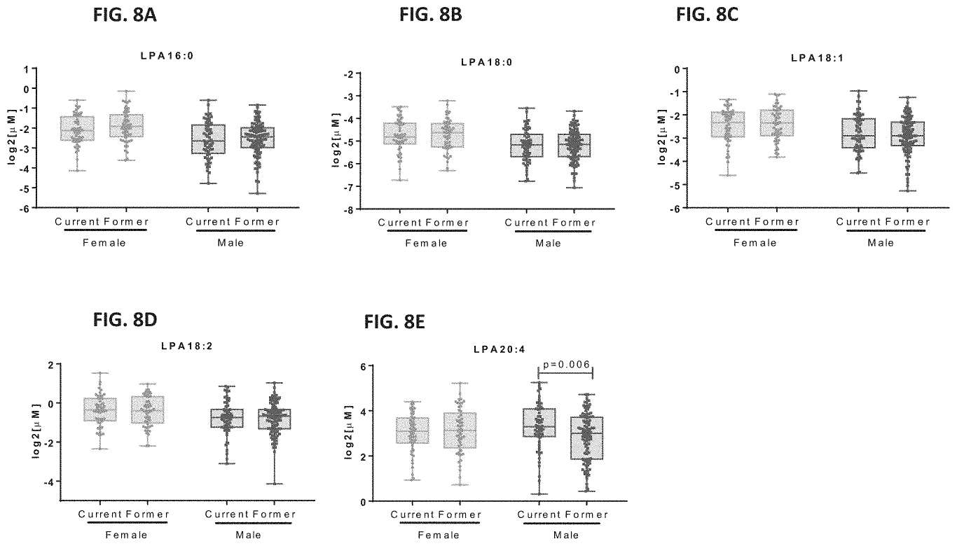

Smoking is the leading trigger for COPD. The prevalence of COPD is 15% in smokers, 12.8% in ex-smokers, and 4.1% in nonsmokers (Pena et al., Chest, 118: 981-989, 2000). LPA levels were similar between smokers and non-smokers in the heathy group (p>0.05) from the small cohort study (FIGS. 7A-7E). LPA concentrations were also similar between former and current smokers among COPD baseline patients (p>0.05; FIGS. 8A-8E), except for LPA 20:4, which was significantly lower in male former smokers compared to current smokers (FIG. 8E). One reason for which a difference was not observed between serum samples from healthy smokers and non-smokers could be related to the lack of lung inflmmation in either group. A lack of difference in the majority of LPA species between former and current smokers may indicate that smoking is not a major driving factor for LPA production in COPD patients, or that the inflammation status is not reversible for former smokers.

(b) Body Mass Index (BMI)

In healthy subjects, serum ATX has been reported to correlate with multiple measures of adiposity and glucose homeostasis (Reeves et al., Obesity (Silver Spring), 23: 2371-2376, 2015). ATX can be secreted by adipocytes (Rancoule et al., Biochimie, 96: 140-143, 2014). These authors suggested that ATX expression was up-regulated in obese patients who had insulin resistance. LPAs had the effect of tonic inhibition on adipose tissue expansion via LPAR1 to LPAR6 and were involved in obesity (Rancoule et al., Biochimie, 96: 140-143, 2014). Significantly, increased LPA concentrations were observed in human plasma of obese (BMI>30.0) individuals compared to individuals with normal BMI (BMI 18.5-25.0) (Fayyaz et al., Cell Physiol Biochem., 43: 445-456, 2017). Michalczyk's study also concluded that obesity was associated with significantly higher plasma LPA (Michalczyk et al., Lipids in Health and Disease, 16: 140, 2017).

In order to test whether body mass index (BMI) could affect LPA levels in COPD patient serum, we compared LPA levels in normal (15<BMI<25), overweight (25<BMI<30), and obese (BMI>30) patients.

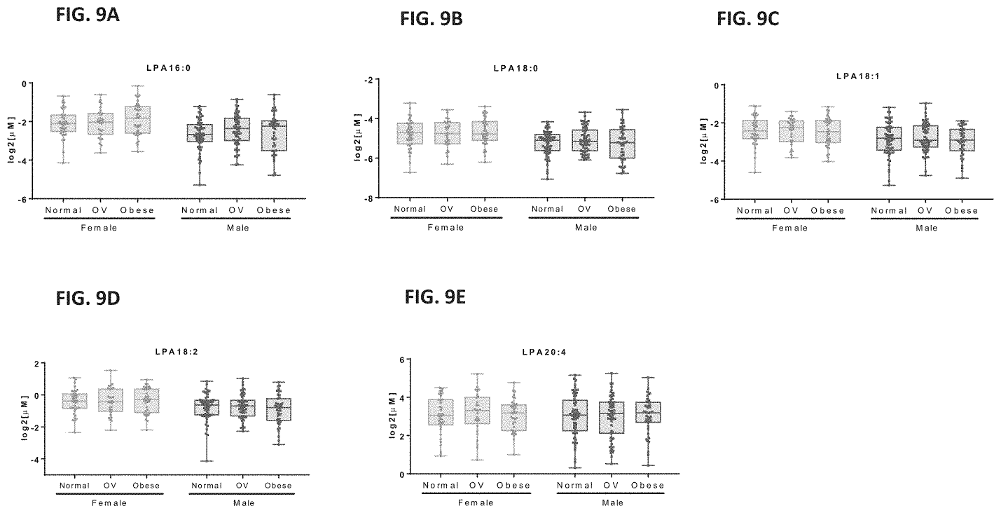

Our results showed no significant difference in LPA levels across the three groups in either male or female patients (FIGS. 9A-9E). This may be due to the previous studies' focus on healthy subjects. However, in COPD patients, the contribution of BMI to LPA production differences was minor compared with the effect of the disease and its associated inflammation.

iii. Association of LPA Levels with COPD Metrics

(a) Lung Functions

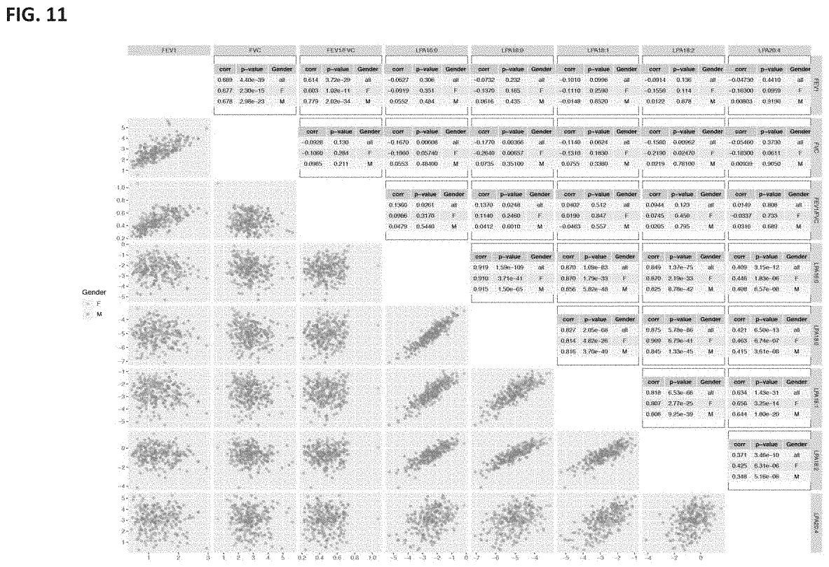

Although the ATX-LPA pathway has been suggested to be involved in COPD disease, the association of LPA with COPD metrics has not been fully explored. The correlations between LPA species levels and lung functions were calculated using Pearson correlation analysis and multivariable linear regression. LPA concentrations had significant or moderate negative correlation with forced vital capacity (FVC) values in female patients [LPA16:0 (r=−0.1860, p=0.06), LPA18:0 (r=−0.2640, p=0.007), LPA18:2 (r=−0.2190, p=0.02), and LPA20:4 (r=−0.18300, p=0.06)], but no significant correlation was detected in male patient samples (p>0.1) (FIG. 11). After adjusting for the effects of confounding factors, FVCs in women did not correlate with LPA species (p>0.1, q>0.5) (Table 5). The correlation was not significant between LPAs and FEV₁, or LPAs and post-bronchodilator ratio FEV₁/FVC in either female or male patients (FIG. 11). Overall, LPAs did not demonstrate strong correlation with lung function.

(b) Chronic Bronchitis

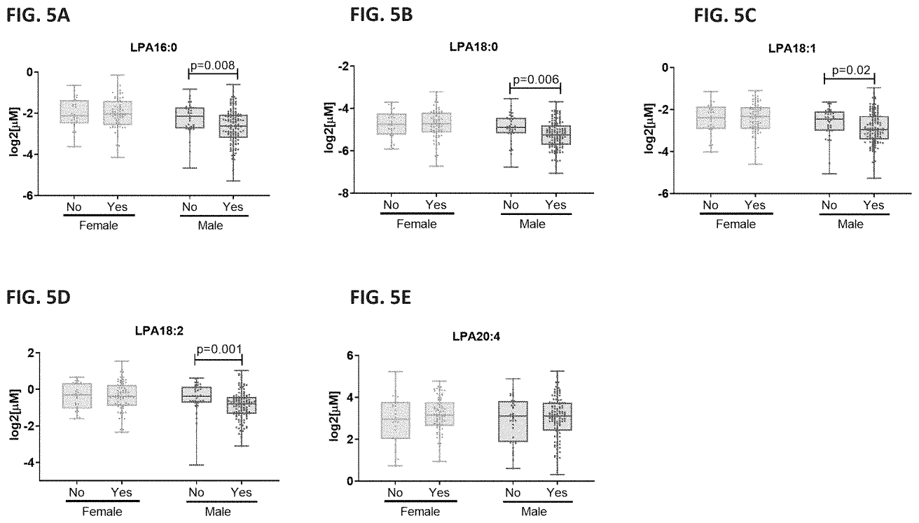

Chronic bronchitis (CB) is one of the most common conditions that can exacerbate COPD. LPA levels from female and male patients with and without chronic bronchitis (CB) were investigated (FIGS. 5A-5E). With univariate analysis, LPA 16:0 (p=0.01), LPA 18:0 (p=0.02), LPA 18:1 (p=0.06), LPA 18:2 (p=0.02) were significantly lower in male CB patients compared to male non-CB patients, but not LPA20:4. LPA levels were not significantly different between CB and non-CB female patients. With multivariate analysis, LPA 16:0 (p=0.02, q=0.03), LPA 18:0 (p=0.01, q=0.03), and LPA 18:2 (p=0.02, q=0.03) remained significantly lower in male CB patients. Because of the limited scientific studies, the mechanism causing lower LPA in male CB patients is unclear. Further investigation is needed to validate the correlation between LPAs and chronic bronchitis observed in male COPD patients in our study population.

C. Conclusions

We optimized and validated an MRM-based LC-MS/MS method for the accurate measurement of LPA species. The results emphasized that even low concentrations of HCl were not suitable for LPA extraction. Importantly, LPA species were separated from other interfering lysophospholipids. Stability of LPA during storage and analysis, which is critical for medium- to large-scale clinical sample analyses, was analyzed. The method was successfully applied in healthy donors and COPD patients to compare LPA species levels. With low sample volume, eight LPA species levels, instead of the 2-5 commonly analyzed species, were detected in COPD patient sera and compared with healthy donors. For the first time, this study defined the associations of demographics and clinical metrics with LPA species levels in COPD patients. Our results indicate that LPAs can act as a tool to facilitate COPD patient subgroup profiling. Several demographic parameters were determined to affect LPA concentrations within the COPD patient population. The majority of the observed alterations differed between males and females, providing further evidence of a gender-driven sub-phenotype of COPD. The LPA results from the study are not only helpful for researchers who study the involvement of ATX-LPA pathway in COPD disease, but also for researchers who are working on inflammatory-related diseases.

### Example 5. Lysophosphatidic Acid (LPA) Levels as Biomarkers for Exacerbation in Chronic Obstructive Pulmonary Disease

Currently used diagnostic methods for COPD include lung function tests such as FEVᵢ (forced expiratory volume in one second); however, a single lung function measure is not an adequate predictor of phenotypes of COPD, such as exacerbations (Singh et al., Am J Respir Crit Care Med, 194: 541-549, 2016). Exacerbations are heterogenous events, as the interactions between exacerbation triggers and host inflammatory responses are complex. Accordingly, studies have failed to identify consistent blood biomarkers associated with COPD exacerbation.

This study focused on the major LPA species (LPA16:0, 18:0, 18:1, 18:2, and 20:4) and assessed the relationship between circulating LPA levels and exacerbation risk, frequency, and severity in COPD patients.

A. Patient Cohort

Baseline serum samples from the placebo arm (n=136) of a global COPD randomized controlled trial (NCT02546700) were used for LPA and lipid measurements. The study included GLOBAL INITIATIVE FOR CHRONIC OBSTRUCTIVE LUNG DISEASE™ (GOLD) stage II to IV patients having a history of at least one exacerbation in the past 12 months and a smoking history of at least 10 pack-years. Patients with a current diagnosis of asthma were excluded. Clinical measures were collected at baseline and every 4 to 12 weeks thereafter during a 24-week placebo-controlled period. Chronic bronchitis was defined using St George's Respiratory Questionnaire For COPD Patients (SGRQ-C) cough and phlegm questions: patients were categorized as having chronic bronchitis if the cough was “most days a week” or “several days a week” and phlegm was “most days a week” or “several days a week.”

An exacerbation was defined as new or increased COPD symptoms (e.g., dyspnea, sputum volume, and sputum purulence) for at least 2 consecutive days that led to treatment with systemic corticosteroids and/or antibiotics, or hospitalization. Exacerbation duration corresponded to the number of days patients were on systemic corticosteroids and/or antibiotics.

Complete blood cell counts were measured by routine clinical laboratory tests. Serum immunoglobulin E (IgE) levels were measured using the ImmunoCAP® Specific IgE Blood Test (Viracor-Eurofin Laboratories). Plasma was collected in sodium citrate tubes for fibrinogen measurement using the Clauss method (Siemens BCS® XP system).

B. Mass Spectrometry LPA Assays

The technical details of the LPA assays are described in Examples 1-4. Briefly, 10 μl of serum from each sample was pooled together as quality control samples. 500 μl disodium phosphate buffer (30 mM citric acid and 40 mM disodium phosphate) was added to 20 μl serum to extract lipids. The extracted samples were reconstituted in methanol and analyzed by liquid chromatography-mass spectrometry (LC-MS/MS). LC coupling to a QTRAP® mass spectrometer (SCIEX) was employed under negative ionization mode. HPLC separation of LPAs was optimized on a C18 column to separate LPA from other lipids. Instrument analyses were performed in multiple reaction monitoring mode with dwell time of 0.10 seconds.

C. Lipidomic Profiling

Lipidomic measurement was carried out using a modified method derived from Contrepois et al., Sci Rep, 8: 17747, 2018. Patients with sufficient remaining serum volume (n=134) were used for lipidomic profiling. Briefly, lipids were purified using dichloromethane, methanol, and water two phase extraction. After direct infusion, lipid species were analyzed on a SELEXION™ enabled QTRAP® 6500 mass spectrometer (SCIEX, Redwood City, CA) in multiple reaction monitoring mode. Lipid species were identified and quantified on the basis of characteristic mass spectrometry transitions.

D. Statistical Analysis

Statistical analyses were performed using R (version 3.5.3). LPA species and lipid concentrations were log₂ transformed. The relationship between LPA species levels, baseline demographics, and other biomarkers were assessed using univariate linear regression or Spearman's rank order method. Tertile levels of each LPA species were used to assign patients into biomarker high (highest tertile), mid (middle tertile), and low (lowest tertile) subgroups for each LPA species. Comparisons among subgroups were assessed using ANOVA with Tukey HSD test, Student t-test, or Kruskal-Wallis test for continuous measures and Fisher's Exact test for categorical measures. Logistic regression and a Quasi-Poisson model were used to estimate exacerbation risk and rate, respectively. Cox proportional hazards regression was used to compare the time to first exacerbation. Covariates pre-specified as stratification factors in the study protocol (exacerbation history, smoking status, geographical region, bronchodilator response, inhaler use) were included in the exacerbation models. P-value <0.05 was considered to be statistically significant. Lipid species detectable in at least 90% of patients were included in the analyses. Lipid concentrations were compared among LPA subgroups, using Kruskal-Wallis test followed by Benjamini-Hochberg correction (FDR) for multiple comparisons. For this exploratory analysis, false discovery rate (FDR)<0.1 was considered to be statistically significant.

### Example 6. Baseline Demographics and Clinical Metrics

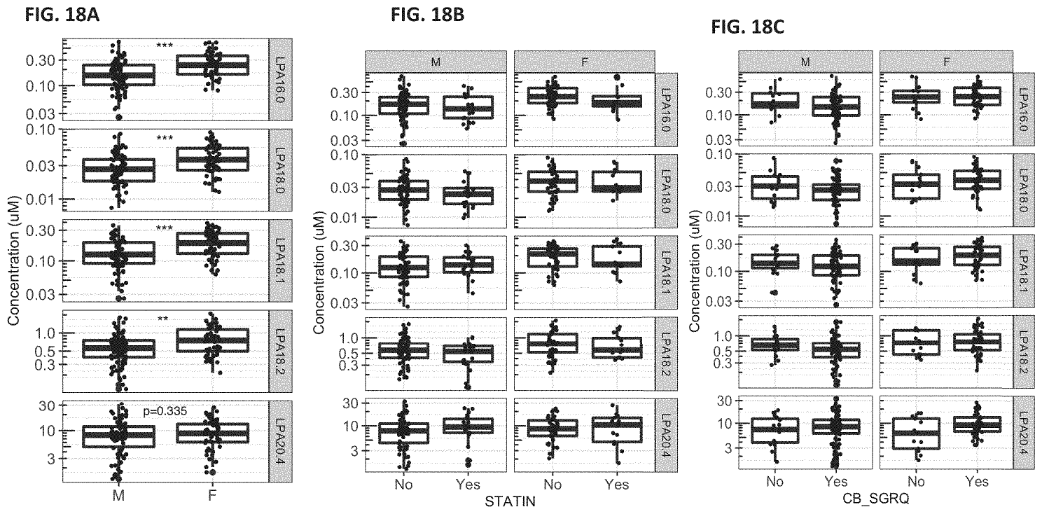

Baseline characteristics of the patients of Example 5 (i.e., patients from the NCT02546700 study) are shown in Table 6. A higher proportion of men than women had more severe disease, with 24% of men classified as GOLD stage IV, compared to 7% of women (p=0.011). LPA species concentrations, except for the concentration of LPA20:4, were significantly lower in men compared to women (FIG. 18A). LPA species concentrations were not significantly different between patients with or without statin use (FIG. 18B), with or without chronic bronchitis (FIG. 18C), or according to smoking status.

TABLE 6
Patient baseline characteristics

| | Male | | Female | | |
|---|---|---|---|---|---|
| | N = 82 | | N = 54 | | p-value |
| Age (years) | 65.2 | (6.8) | 63.6 | (8.1) | 0.24 |
| Race | | | | | |
| White, % | 77 | (94) | 50 | (93) | |
| Black or African American, % | 3 | (4) | 2 | (4) | 0.84 |
| Others, % | 2 | (2) | 2 | (4) | |
| Body mass index | 27.2 | (4.9) | 27.2 | (5.5) | 0.98 |
| Former smokers, % | 50 | (61) | 26 | (48) | 0.16 |
| Post-bronchodilator FEV₁ | | | | | |
| Absolute (L) | 1.4 | (0.5) | 1.2 | (0.4) | 0.0036 |
| % predicted | 43.8 | (15.5) | 51.3 | (13.6) | 0.0043 |
| Post-bronchodilator FEV₁/FVC ratio | 0.45 | (0.1) | 0.52 | (0.1) | 0.00024 |
| SGRQ-C score | 57.9 | (17.3) | 57.1 | (18.3) | 0.80 |
| GOLD, % | | | | | |
| Stage II | 31 | (38) | 32 | (59) | |
| Stage III | 31 | (38) | 18 | (33) | 0.011 |
| Stage IV | 20 | (24) | 4 | (7) | |
| Patients with bronchodilator reversibility, % | 14 | (17) | 6 | (11) | 0.46 |
| Patients with chronic bronchitis, % | 67 | (82) | 41 | (76) | 0.52 |
| Patients with severe exacerbation in previous 12 | 26 | (32) | 11 | (20) | 0.17 |
| months, % | | | | | |
| Median blood eosinophil count (cells per μL) | 180 | (130) | 175 | (183) | 0.89 |
| Median fibrinogen (g/L) | 3.8 | (1.4) | 3.5 | (0.9) | 0.037 |

Data are n (%), mean (SD), or median (IQR). FEV₁, forced expiratory volume in 1 second; FVC, forced vital capacity; SGRQ-C, St. George's Respiratory Questionnaire COPD.

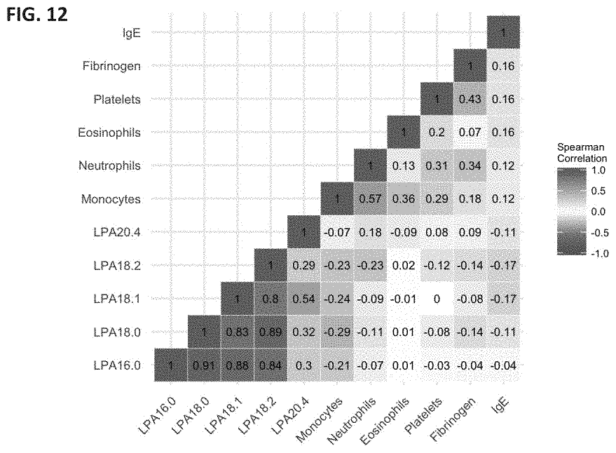

The levels of LPA species 16:0, 18:0, 18:1 and 18:2 were highly correlated with each other (rho 0.80-0.91), but exhibited modest correlation with LPA20:4 (rho 0.29-0.54) (FIG. 12). LPA species levels had no significant correlation with levels of blood eosinophils, platelets, plasma fibrinogen or serum IgE. Specific LPA species (16:0, 18:0, 18:1, and 18:2) showed modest negative correlation with levels of blood monocytes (rho 0.21-0.29). LPA18:2 showed modest negative correlation with level of neutrophils (rho 0.23) (FIG. 12). The correlations of these biomarkers were very similar between men and women.

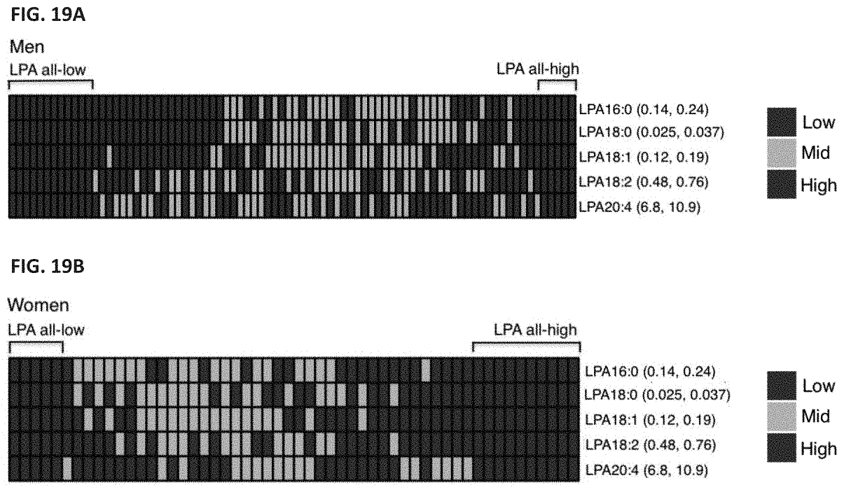

Tertile levels of each LPA species measured in all patients, regardless of gender, were used to categorize patients into biomarker high (highest tertile), mid (middle tertile), and low (lowest tertile) subgroups for each LPA species. There was overlap of these LPA species subgroups, but only a small proportion of patients were low (in the lowest tertile) in all LPA species: 15% and 9% of men and women, respectively (FIGS. 19A and 19B and Table 7).

TABLE 7
Distribution of patients having low,
mid, and high levels of LPA species

| | | |
|---|---|---|
| | Men | Women |
| LPA subgroup | N = 82 | N = 54 |

| | | | | |
|---|---|---|---|---|
| LPA all-low, n (%) | 12 | (15) | 5 | (9) |
| LPA all-mid, n (%) | 3 | (4) | 1 | (2) |
| LPA all-high, n (%) | 5 | (6) | 10 | (19) |
| LPA mix, n (%) | 62 | (76) | 38 | (70) |

Patient baseline characteristics in LPA16:0 and LPA18:0 subgroups are shown in Tables 8 and 9, respectively. Since LPA16 and LPA18 concentrations were significantly lower in men than women, the number of men in the low LPA subgroups was greater than the number of women. There was a significant difference in the distribution of GOLD stages among LPA16:0 subgroups in women: 88% of female LPA16:0 low patients were GOLD stage II, compared to 35% and 69% in mid and high LPA16:0 subgroups, respectively (p=0.0063) (Table 8). This may be influenced by the small number of women in the low subgroup, as this statistical difference was not consistently observed across other LPA18 subgroups (Tables 8, 9, and 10). A higher proportion of LPA18:0 low women (56%) had severe exacerbations in the past 12 months, as compared to 11% and 15% in LPA18:0 mid and high subgroups, respectively (p=0.025) (Table 8). This statistical difference was not consistent across other LPA18 subgroups (Tables 10 and 11). There were no significant differences in baseline characteristics among LPA20:4 subgroups (Table 12).

TABLE 8
Patient baseline characteristics by LPA16:0 subgroups and gender

| | Male | | | | | | | Female | | | | | | |
|---|---|---|---|---|---|---|---|---|---|---|---|---|---|---|
| | Low | | Mid | | High | | p- | Low | | Mid | | High | | P- |
| LPA16:0 | N = 36 | | N = 27 | | N = 19 | | value | N = 8 | | N = 20 | | N = 26 | | value |
| Age (years) | 63.3 | (6.9) | 67.2 | (6.2) | 65.9 | (6.5) | 0.064 | 59.8 | (9.9) | 61.8 | (8.2) | 66.2 | (6.7) | 0.058 |
| Body mass index | 27.1 | (5.4) | 26.2 | (4.5) | 28.8 | (4.1) | 0.20 | 28.3 | (4.9) | 26.1 | (5.5) | 27.6 | (5.7) | 0.54 |
| Former smokers % | 22 | (61) | 16 | (59) | 12 | (63) | 1.0 | 3 | (37.5) | 10 | (50) | 13 | (50) | 0.87 |
| Post-bronchodilator FEV₁ | | | | | | | | | | | | | | |
| Absolute (L) | 1.4 | (0.6) | 1.5 | (0.6) | 1.4 | (0.3) | 0.83 | 1.4 | (0.5) | 1.1 | (0.4) | 1.2 | (0.3) | 0.13 |
| % predicted | 41.7 | (16.6) | 46.3 | (14.9) | 44.8 | (13.9) | 0.50 | 59.2 | [symbol] * | 45.1 | (13.3)* | 53.6 | (10.9) | 0.0019 |
| Post-bronchodilator | 0.44 | (0.1) | 0.46 | (0.1) | 0.45 | (0.1) | 0.90 | 0.59 | (0.1)* | 0.47 | (0.1)* | 0.54 | (0.1) | 0.0076 |
| FEV₁/FVC ratio | | | | | | | | | | | | | | |
| SGRQ-C score | 60.1 | (16.7) | 53.7 | (16.3) | 59.4 | (19.6) | 0.33 | 57.3 | (14.3) | 57.4 | (19.1) | 65.8 | (19.3) | 1.0 |
| GOLD, % | | | | | | | | | | | | | | |
| Stage II | 12 | (33.3) | 11 | (41) | 8 | (42) | | 7 | (87.5) | 7 | (35) | 18 | (69.2) | |
| Stage III | 12 | (33.3) | 12 | (44) | 7 | (37) | 0.56 | 0 | | 10 | (50) | 8 | (30.8) | 0.0063 |
| Stage IV | 12 | (33.3) | 4 | (15) | 4 | (21) | | 1 | (12.5) | 3 | (15) | 0 | | |
| Patients with | 10 | (28) | 2 | (7) | 2 | (11) | 0.082 | 1 | (12.5) | 1 | (5) | 4 | (15.4) | 0.51 |
| bronchodilator | | | | | | | | | | | | | | |
| Patients with chronic | 33 | (92) | 21 | (78) | 13 | (68) | 0.081 | 5 | (62.5) | 16 | (80) | 20 | (76.9) | 0.63 |
| bronchitis, % | | | | | | | | | | | | | | |
| Patients with severe | 11 | (31) | 9 | (33) | 6 | (32) | 1.0 | 4 | (50) | 4 | (20) | 3 | (11.5) | 0.089 |
| exacerbation in previous | | | | | | | | | | | | | | |
| Median blood eosinophil | 190 | (95) | 170 | (145) | 165 | (243) | 0.71 | 145 | (113) | 180 | (98) | 170 | (350 | 0.76 |
| count (cells/ [symbol] ) | | | | | | | | | | | | | | |
| Media fibrinogen (g/L) | 3.7 | (1.2) | 3.8 | (1.3) | 3.8 | (1.2) | 0.90 | 3.5 | (0.3) | 3.5 | (1) | 3.4 | (0.7) | 0.80 |

[symbol]

indicates data missing or illegible when filed

TABLE 9
Patient baseline characteristics by LPA18:0 subgroups and gender

| | Male | | | | | | | Female | | | | | | |
|---|---|---|---|---|---|---|---|---|---|---|---|---|---|---|
| | Low | | Mid | | High | | p- | Low | | Mid | | High | | P- |
| LPA18:0 | N = 35 | | N = 28 | | N = 19 | | value | N = 9 | | N = 19 | | N = 26 | | value |
| Age (years) | 64.3 | (6.8) | 66.2 | (6.7) | 65.2 | (6.9) | 0.57 | 62.9 | (10.4) | 60.5 | (8.3) | 66.1 | (6.3) | 0.067 |
| Body mass index | 27 | (5.2) | 25.9 | (4.7)* | 29.5 | (3.7)* | 0.040 | 17.8 | (5.5) | 27.7 | (5.4) | 26.6 | (5.7) | 0.76 |
| Former smokers % | 20 | (57.1) | 19 | (67.9) | 11 | (57.9) | 0.68 | 6 | (66.7) | 7 | (36.8) | 13 | (50) | 0.33 |
| Post-bronchodilator FEV₁ | | | | | | | | | | | | | | |
| Absolute (L) | 1.3 | (0.5) | 1.5 | (0.5) | 1.4 | (0.5) | 0.67 | 1.3 | (0.5) | 1.2 | (0.4) | 1.1 | (0.3) | 0.41 |
| % predicted | 41.6 | (15.4) | 46.7 | (16.2) | 44.1 | (14.3) | 0.44 | 56.7 | (14.7) | 48.9 | (14.3) | 51.1 | (12.7) | 0.37 |
| Post-bronchodilator | 0.45 | (0.1) | 0.45 | (0.1) | 0.45 | (0.1) | 0.97 | 0.57 | (0.1) | 0.5 | (0.1) | 0.52 | (0.1) | 0.33 |
| FEV₁/FVC ratio | | | | | | | | | | | | | | |
| SGRQ-C score | 59.3 | (16.8) | 58.8 | (17.3) | 54 | (18.5) | 0.53 | 57 | (13.2) | 59.6 | (19.8) | 55.3 | (19) | 0.74 |
| GOLD, % | | | | | | | | | | | | | | |
| Stage II | 12 | (34.3) | 13 | (46.4) | 6 | (31.6) | | 8 | (88.9) | 10 | (52.6) | 14 | (53.8) | |
| Stage III | 12 | (34.3) | 10 | (35.7) | 9 | (47.4) | 0.63 | 0 | | 7 | (36.8) | 11 | (42.3) | 0.10 |
| Stage IV | 11 | (31.4) | 5 | (17.9) | 4 | (51.1) | | 1 | (11.1) | 2 | (10.5) | 1 | (3.8) | |
| Patients with | 7 | (20) | 6 | (21.4) | 1 | (5.3) | 0.31 | 0 | | 3 | (15.8) | 3 | (11.5) | 0.54 |
| bronchodilator | | | | | | | | | | | | | | |
| Patients with chronic | 30 | (85.7) | 23 | (82.1) | 14 | (73.7) | 0.51 | 5 | (55.6) | 15 | (78.9) | 21 | (20.8) | 0.36 |
| bronchitis, % | | | | | | | | | | | | | | |
| Patients with severe | 12 | (34.3) | 8 | (28.6) | 6 | (31.6) | 0.95 | 5 | (55.6) | 2 | (10.5) | 4 | (15.4) | 0.025 |
| exacerbation in previous | | | | | | | | | | | | | | |
| Median blood eosinophil | 210 | (113) | 160 | (135) | 180 | (205) | 0.36 | 128 | (58) | 200 | (145) | 180 | (340) | 0.23 |
| count (cells/ [symbol] ) | | | | | | | | | | | | | | |
| Media fibrinogen (g/L) | 3.8 | (1.4) | 3.8 | (1.4) | 3.6 | (1.1) | 0.70 | 3.5 | (0.3) | 4 | (1.2) | 3.4 | (0.3 | 0.53 |

[symbol]

indicates data missing or illegible when filed

TABLE 10
Patient baseline characteristics by LPA18:1 subgroups and gender

| | Male | | | | | | | Female | | | | | | |
|---|---|---|---|---|---|---|---|---|---|---|---|---|---|---|
| | Low | | Mid | | High | | p- | Low | | Mid | | High | | p- |
| LPA18:1 | N = 34 | | N = 29 | | N = 19 | | value | N = 10 | | N = 18 | | N = 26 | | value |
| Age (years) | 64.2 | (6.5) | 65.3 | (7.3) | 66.8 | (6.3) | 0.40 | 62 | (10.7) | 63.2 | (7.2) | 64.5 | (7.7) | 0.68 |
| Body mass index | 27.2 | (5.2) | 27.1 | (4.9) | 27.3 | (4.5) | 0.99 | 28.6 | (5.5) | 27.2 | (5.2) | 26.6 | (5.7) | 0.64 |
| Former smokers, % | 21 | (61.8) | 19 | (65.5) | 10 | (52.6) | 0.65 | 5 | (50) | 10 | (55.6) | 11 | (42.3) | 0.68 |
| Post-bronchodilator FEV₁ | | | | | | | | | | | | | | |
| Absolute (L) | 1.3 | (0.6) | 1.5 | (0.5) | 1.4 | (0.4) | 0.36 | 1.2 | (0.4) | 1.2 | (0.4) | 1.2 | (0.3) | 0.97 |
| % predicted | 41.1 | (16.3 [symbol] | 46.8 | (16.6) | 44.7 | (11.2) | 0.34 | 52.2 | (15.1) | 49.7 | (15.3) | 52 | (12.2) | 0.83 |
| Post-bronchodilator | 0.44 | (0.1) | 0.48 | (0.1) | 0.43 | (0.1) | 0.20 | 0.53 | (0.1) | 0.51 | (0.1) | 0.52 | (0.1) | 0.78 |
| FEV₁/FVC ratio | | | | | | | | | | | | | | |
| SGRQ-C score | 58.9 | (17) | 57 | (16.1) | 57.4 | (20.1) | 0.90 | 48.4 | (14.9) | 59.7 | (17.1) | 58.6 | (19. [symbol] | 0.25 |
| GOLD, % | | | | | | | | | | | | | | |
| Stage II | 10 | (29.4) | 13 | (44.8) | 8 | (42.1) | | 6 | (60) | 11 | (61.1) | 15 | (57.7) | |
| Stage III | 12 | (35.3) | 11 | (37.9) | 8 | (42.1) | 0.43 | 3 | (30) | 5 | (27.8) | 10 | (38.5) | 0.82 |
| Stage IV | 12 | (35.3) | 5 | (17.2) | 3 | (15.8) | | 1 | (10) | 2 | (11.1) | 1 | (3.8) | |
| Patients with | 7 | (20.6) | 5 | (17.2) | 2 | (10.5) | 0.70 | 0 | (0) | 3 | (16.7) | 3 | (11.5) | 0.46 |
| bronchodilator | | | | | | | | | | | | | | |
| Patients with chronic | 30 | (88.2) | 21 | (72.4) | 16 | (84.2) | 0.29 | 7 | (70) | 14 | (77.8) | 20 | (76.9) | 0.83 |
| bronchitis, % | | | | | | | | | | | | | | |
| Patients with severe | 9 | (26.5) | 11 | (37.9) | 6 | (31.6) | 0.65 | 4 | (40) | 4 | (22.2) | 3 | (11.5) | 0.16 |
| exacerbation in previous | | | | | | | | | | | | | | |
| Median blood eosinophil | 210 | (110) | 190 | (170) | 140 | (120) | 0.11 | 125 | (63) | 205 | (223) | 170 | (170) | 0.21 |
| count (cells/ [symbol] ) | | | | | | | | | | | | | | |
| Median fibrinogen (g/L) | 4.1 | (1.7) | 3.5 | (1.3) | 3.8 | (1.1) | 0.21 | 3.5 | (0.6) | 4.2 | (1.2) | 3.4 | (0.4) | 0.63 |

[symbol]

indicates data missing or illegible when filed

TABLE 11
Patient baseline characteristics by LPA18:2 subgroups and gender

| | Male | | | | | | | Female | | | | | | |
|---|---|---|---|---|---|---|---|---|---|---|---|---|---|---|
| | Low | | Mid | | High | | p- | Low | | Mid | | High | | p- |
| LPA18:2 | N = 31 | | N = 32 | | N = 19 | | value | N = 13 | | N = 15 | | N = 26 | | value |
| Age (years) | 63.3 | (6.6 [symbol] | 66 | (6.5) | 66.8 | (7) | 0.13 | 62.1 | (10.1) | 62.3 | (8) | 65.1 | (7) | 0.43 |
| Body mass index | 27.4 | (5.3 [symbol] | 26.6 | (4.5) | 27.9 | (4.8) | 0.62 | 28.4 | (5.5) | 26.4 | (5) | 27 | (5.8) | 0.61 |
| Former smokers, % | 19 | (61.3) | 22 | (68.8) | 9 | (47.4) | 0.31 | 8 | (61.5) | 6 | (40) | 12 | (46.2) | 0.53 |
| Post-bronchodilator FEV₁ | | | | | | | | | | | | | | |
| Absolute (L) | 1.4 | (0.5) | 1.4 | (0.5) | 1.5 | (0.5) | 0.53 | 1.3 | (0.5) | 1.1 | (0.3) | 1.2 | (0.3) | 0.49 |
| % predicted | 40.9 | (16) | 44.7 | (14) | 47.7 | (16. [symbol] | 0.30 | 54.8 | (15.5) | 47.5 | (12.9) | 51.6 | (13) | 0.37 |
| Post-bronchodilator | 0.43 | (0.1 [symbol] | 0.45 | (0.1) | 0.48 | (0.1) | 0.25 | 0.56 | (0.1) | 0.47 | (0.1) | 0.53 | (0.1) | 0.091 |
| FEV₁/FVC ratio | | | | | | | | | | | | | | |
| SGRQ-C score | 59.2 | (14. [symbol] | 59 | (18.5) | 53.9 | (19. [symbol] | 0.52 | 55.3 | (17.8) | 58.1 | (18.6) | 57.4 | (19) | 0.91 |
| GOLD, % | | | | | | | | | | | | | | |
| Stage II | 9 | (29) | 13 | (40.6) | 9 | (47.4) | | 9 | (69.2) | 8 | (53.3) | 15 | (57.7) | |
| Stage III | 11 | (35.5) | 14 | (43.8) | 6 | (31.6) | 0.39 | 3 | (23.1) | 5 | (33.3) | 10 | (38.5) | 0.75 |
| Stage IV | 11 | (35.5) | 5 | (15.6) | 4 | (21.1) | | 1 | (7.7) | 2 | (13.3) | 1 | (3.8) | |
| Patients with bronchodilator | 9 | (29) | 4 | (12.5) | 1 | (5.3) | 0.079 | 1 | (7.7) | 1 | (6.7) | 4 | (15.4) | 0.75 |
| reversibility, % | | | | | | | | | | | | | | |
| Patients with chronic | 28 | (90.3) | 26 | (81.2) | 13 | (68.4) | 0.14 | 9 | (69.2) | 12 | (80) | 20 | (76.9) | 0.84 |
| bronchitis, % | | | | | | | | | | | | | | |
| Patients with severe | 11 | (35.5) | 8 | (25) | 7 | (36.8) | 0.60 | 5 | (38.5) | 2 | (13.3) | 4 | (15.4) | 0.19 |
| exacerbation in previous 12 | | | | | | | | | | | | | | |
| Median blood eosinophil count | 195 | (115) | 190 | (110) | 140 | (150) | 0.37 | 145 | (133) | 180 | (95) | 200 | (270) | 0.59 |
| (cells/ [symbol] ) | | | | | | | | | | | | | | |
| Median fibrinogen (g/L) | 3.7 | (1.6) | 4.1 | (1.6) | 3.5 | (0.9) | 0.26 | 3.5 | (0.7) | 3.5 | (1.7) | 3.4 | (0.6) | 0.90 |

[symbol]

indicates data missing or illegible when filed

TABLE 12
Patient baseline characteristics by LPA20:4 subgroups and gender

| | Male | | | | | | | Female | | | | | | |
|---|---|---|---|---|---|---|---|---|---|---|---|---|---|---|
| | Low | | Mid | | High | | p- | Low | | Mid | | High | | p- |
| LPA20:4 | N = 26 | | N = 30 | | N = 26 | | value | N = 18 | | N = 17 | | N = 19 | | value |
| Age (years) | 64.8 | (6) | 64.7 | (7.8) | 66 | (6.3) | 0.73 | 63.6 | (9.4) | 64 | (5.2) | 63.3 | (9.2) | 0.97 |
| Body mass index | 27.7 | (4.1) | 26.1 | (5.7) | 28 | (4.5) | 0.31 | 26.9 | (5.6) | 26.6 | (5.2) | 28 | (5.8) | 0.72 |
| Former smokers, % | 19 | (73.1) | 16 | (53.3) | 15 | (57.7) | 0.28 | 11 | (61.1) | 5 | (29.4) | 10 | (52.6) | 0.17 |
| Post-bronchodilator FEV₁ | | | | | | | | | | | | | | |
| Absolute (L) | 1.3 | (0.6) | 1.4 | (0.5) | 1.5 | (0.4) | 0.71 | 1.1 | (0.4) | 51.6 | (13.9) | 1.1 | (0.4) | 0.51 |
| % predicted | 41.2 | (15.3) | 44.1 | (17.2) | 46.5 | (13.4) | 0.46 | 50.2 | (12.6) | 1.3 | (0.4) | 52 | (14.8) | 0.92 |
| Post-bronchodilator | 0.44 | (0.1) | 0.47 | (0.1) | 0.44 | (0.1) | 0.35 | 0.50 | (0.1) | 0.52 | (0.1) | 0.54 | (0.1) | 0.55 |
| FEV₁/FVC ratio | | | | | | | | | | | | | | |
| SGRQ-C score | 58.1 | (17.9) | 61.5 | (15.9) | 53.4 | (18) | 0.22 | 56 | (20.7) | 52.9 | (18.6 | 62 | (15) | 0.32 |
| GOLD, % | | | | | | | | | | | | | | |
| Stage II | 7 | (26.9) | 11 | (36.7) | 13 | (50) | | 11 | (61.1) | 11 | (64.7) | 10 | (52.6) | |
| Stage III | 11 | (42.3) | 10 | (33.3) | 10 | (38.5) | 0.30 | 6 | (33.3) | 4 | (23.5) | 8 | (42.1) | 0.81 |
| Stage IV | 8 | (30.8) | 9 | (30) | 3 | (11.5) | | 1 | (5.6) | 2 | (11.8) | 1 | (5.3) | |
| Patients with bronchodilator | 2 | (7.7) | 9 | (30) | 3 | (11.5) | 0.078 | 3 | (16.7) | 1 | (5.9) | 2 | (10.5) | 0.77 |
| reversibility, % | | | | | | | | | | | | | | |
| Patients with chronic | 20 | (76.9) | 26 | (86.7) | 21 | (80.8) | 0.68 | 11 | (61.1) | 15 | (88.2) | 15 | (78.9) | 0.16 |
| bronchitis, % | | | | | | | | | | | | | | |
| Patients with severe | 10 | (38.5) | 8 | (26.7) | 8 | (30.8) | 0.63 | 7 | (38.9) | 2 | (11.8) | 2 | (10.5) | 0.079 |
| exacerbation in previous 12 | | | | | | | | | | | | | | |
| Median blood eosinophil | 220 | (130) | 165 | (98) | 170 | (118) | 0.37 | 180 | (140) | 175 | (118) | 170 | (250) | 0.63 |
| count (cells/μL) | | | | | | | | | | | | | | |
| Median fibrinogen (g/L) | 3.6 | (1.2) | 3.8 | (1.9) | 3.8 | (1.1) | 0.88 | 3.5 | (0.6) | 3.4 | (0.9) | 3.5 | (0.8) | 0.65 |

### Example 7. LPA Species and Exacerbation

A. LPA Species and Gender

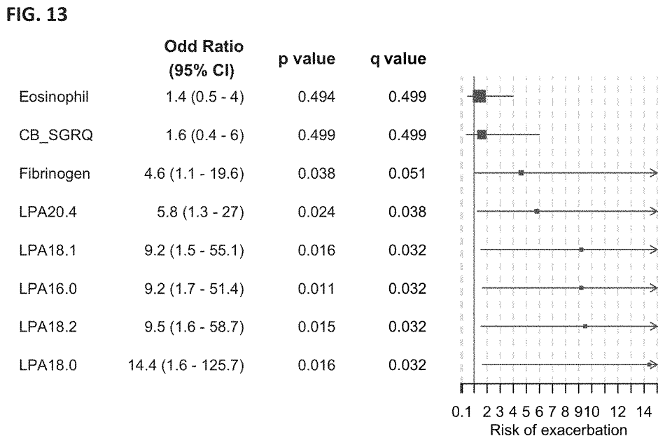

Since there were significant differences in LPA species levels between men and women, exacerbation analyses were stratified by gender. For each LPA species, men with low LPA (LPA in the lowest tertile) had significantly higher risk of having an exacerbation within the 24-week follow-up period (odds ratio (OR) (95% CI)): LPA16:0=9.2 (1.7-51.4); LPA18:0=14.4 (1.6-125.7); LPA18:1=9.2 (1.5-55.1); LPA18:2=9.5 (1.6-58.7); LPA20:4=5.8 (1.3-27), compared to those with high LPA (FIG. 13).

B. LPA Species and Blood Eosinophils, Fibrinogen, and Chronic Bronchitis

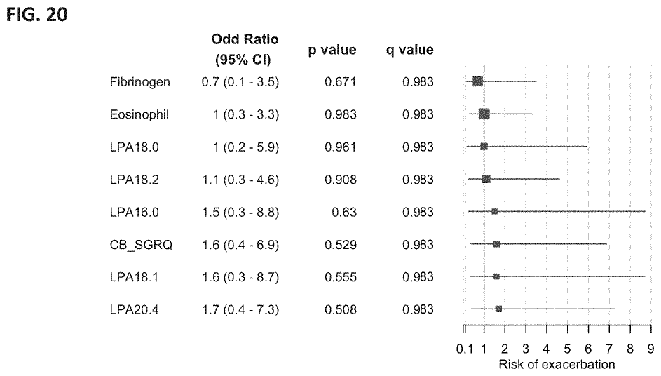

Blood eosinophils above 300 cells/μl (Yun et al., The Journal of Allergy and Clinical Immunology, 141: 2037-2047.e2010, 2018) and fibrinogen above 3.5 g/L (Mannino et al., Chronic Obstructive Pulmonary Diseases (Miami, Fla), 2: 23-34, 2015) have been reported to be associated with COPD exacerbation. In line with this, men with high fibrinogen (≥3.5 g/L) had increased risk of exacerbation (4.6 (1.1-19.6)) compared to those with low fibrinogen (<3.5 g/L) (p=0.038). Since there were very few patients with eosinophils at or above 300 cells/μl in this study, 200 cells/μl was used as cutoff to categorize patients. Men with high eosinophils (≥200 cells/μl) had no significant increase in risk (1.4 (0.5-4.0)) compared to those with low eosinophils (<200 cells/μl) (p=0.49). Similarly, men with chronic bronchitis had no significant increase in risk (1.6 (0.4-6.0)) compared to those without chronic bronchitis (p=0.50). There was no significant increase in risk for women by any of these biomarkers (FIG. 20).

C. LPA Species and Exacerbations

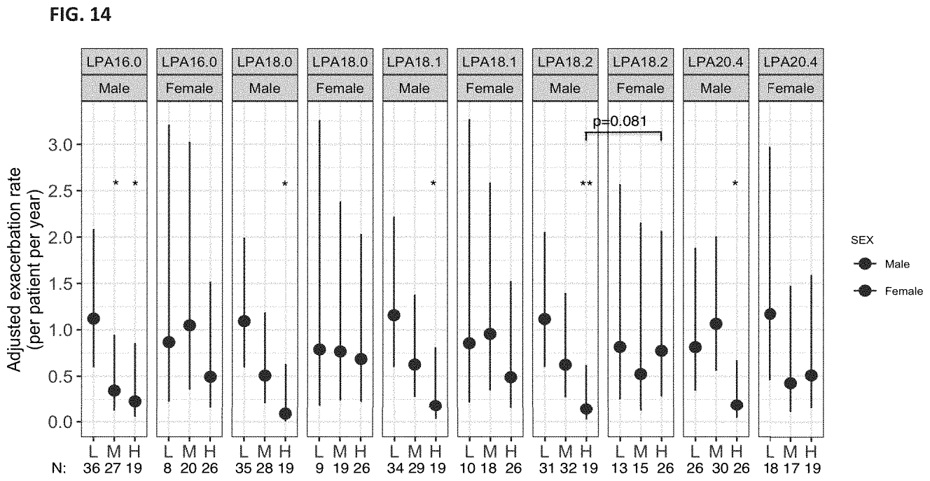

For each LPA species, exacerbation rate was significantly higher in men with low LPA (i.e., levels of LPA in the lowest tertile) (estimated rate (per patient per year) (95% CI)): LPA16:0=1.1 (0.6-2.1); LPA18:0=1.1 (0.6-2.0); LPA18:1=1.2 (0.6-2.2); LPA18:2=1.1 (0.6-2.0); LPA20:4=0.8 (0.4-1.9), compared to LPA-high men: LPA16:0=0.2 (0.06-0.9); LPA18:0=0.1 (0.01-0.6); LPA18:1=0.2 (0.04-0.8); LPA18:2=0.1 (0.03-0.6); LPA20:4=0.2 (0.05-0.7) (FIG. 14). Such trends were observed for some of the LPA species in women, but the differences were not significant. 25 of 82 male patients (30.5%) and 18 of 54 female patients (33%) had on-study exacerbation.

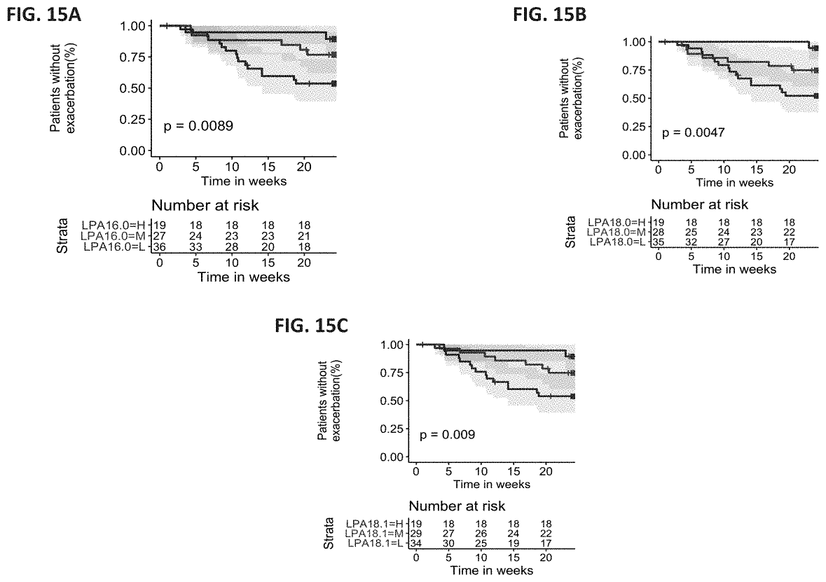

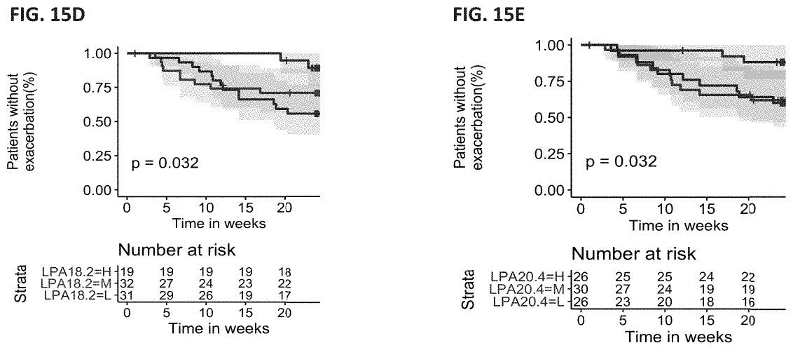

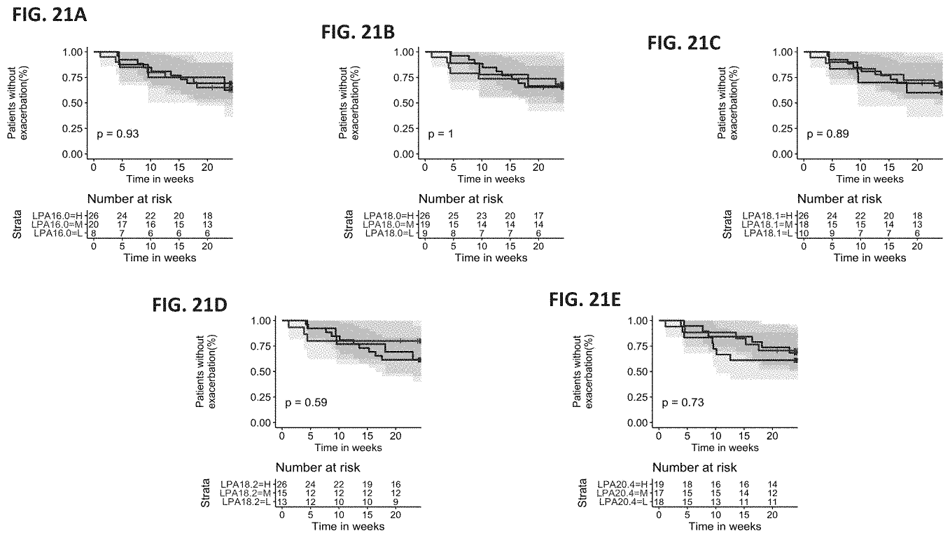

For each LPA species, time to first exacerbation was significantly shorter in men with low LPA compared to high LPA (hazard ratio (HR)): LPA16:0=HR 6.2, p=0.016; LPA18:0=HR 11.6, p=0.018; LPA18:1=HR 6.3, p=0.016; LPA18:2=HR 6.8, p=0.013; LPA20:4=HR 4.5, p=0.024 (FIGS. 15A-15E). Interestingly, LPA18:0 and LPA18:2 identified relatively homogenous groups of LPA-high patients in which the first exacerbation occurred after 160 days and 135 days of study entry, respectively, as compared to the rest of the subgroups, in which the first events occurred within 25 days of study entry. There were no differences in time to first exacerbation among LPA subgroups in women (FIGS. 21A-21E).

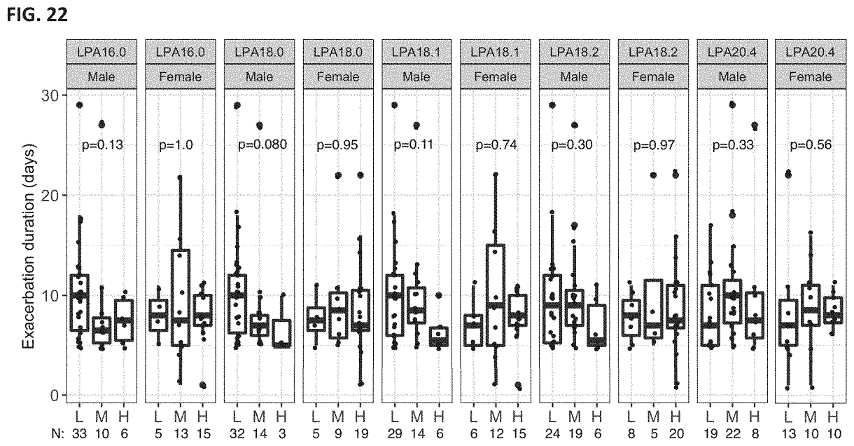

In patients with exacerbations, there was a trend for all LPA species except for LPA20:4 that the median exacerbation duration was longer in men with low LPA compared to high LPA, but the difference was not significant (FIG. 22). Women did not show such a trend of increased exacerbation duration. There was no significant difference in the proportion of hospitalized exacerbations among LPA species subgroups in either gender (Table 13).

TABLE 13
Hospitalized exacerbation by baseline biomarker profile and gender

| | | Hospitalized | | | | | Hospitalized | | |
|---|---|---|---|---|---|---|---|---|---|
| | Number of | exacerbation | | p- | | Number of | exacerbation | | p- |
| Male | exacerbation | N (%) | | value | Female | exacerbation | N (%) | | value |
| LPA16:0 | | | | | LPA16:0 | | | | |
| Low | 33 | 5 | (15.2) | | Low | 5 | 0 | | |
| Mid | 10 | 2 | (20.0) | 0.85 | Mid | 13 | 3 | (23.1) | 0.11 |
| High | 6 | 1 | (16.7) | | High | 15 | 0 | | |
| LPA18:0 | | | | | LPA18:0 | | | | |
| Low | 32 | 6 | (18.8) | | Low | 5 | 0 | | |
| Mid | 14 | 1 | (7.1) | 0.34 | Mid | 9 | 2 | (22.2) | 0.23 |
| High | 3 | 1 | (33.3) | | High | 19 | 1 | (5.3) | |
| LPA18:1 | | | | | LPA18:1 | | | | |
| Low | 29 | 4 | (13.8) | | Low | 6 | 0 | | |
| Mid | 14 | 3 | (21.4) | 0.86 | Mid | 12 | 3 | (25.0) | 0.077 |
| High | 6 | 1 | (16.7) | | High | 15 | 0 | | |
| LPA18:2 | | | | | LPA18:2 | | | | |
| Low | 24 | 3 | (12.5) | | Low | 8 | 0 | | |
| Mid | 19 | 3 | (15.8) | 0.48 | Mid | 5 | 2 | (40.0) | 0.089 |
| High | 6 | 2 | (33.3) | | High | 20 | 1 | (5.0) | |
| LPA20:4 | | | | | LPA20:4 | | | | |
| Low | 19 | 2 | (10.5) | | Low | 13 | 3 | (23.1) | |

| | | | | | | | | |
|---|---|---|---|---|---|---|---|---|
| Mid | 22 | 4 | (18:2) | 0.60 | Mid | 10 | 0 | 0.096 |
| High | 8 | 2 | (25.0) | | High | 10 | 0 | |

D. Discussion

This study identified serum LPA species as prognostic biomarkers of exacerbation. Men with low levels of LPA species had significantly higher risk and rate of exacerbation, and earlier time to first exacerbation, as compared to men with high levels of LPA species. There was no significant difference in exacerbation severity in terms of hospitalization or duration of systemic corticosteroids and/or antibiotic treatment. In this patient population, no other significant differences in baseline characteristics among LPA species subgroups were observed except for gender.

Gender differences in COPD disease susceptibility (Sorheim et al., Thorax, 65: 480-485, 2010), biomarkers (Gaggar et al., PLoS ONE, 6: 2011), and disease prognosis (Lisspers et al., NPJ Prim Care Respir Med, 29: 45, 2019) have been reported, wherein women had greater symptom burden (DeMeo et al., International Journal of Chronic Obstructive Pulmonary Disease, 13: 3021-3029, 2018), exacerbation rate, and mortality rate compared to men (Lisspers et al., NPJ Prim Care Respir Med, 29: 45, 2019). In this study, 33% of women had exacerbations compared to 31% of men, even though men had more severe disease at baseline. LPA-high men had very low exacerbation rate, despite the fact that all of them had had at least one exacerbation in the prior year.

In summary, a robust mass spectrometry method was used to measure the main serum LPA species in COPD and observed gender-associated differences in the ability of these biomarkers to identify patients at risk of exacerbation. Men with low LPA had increased risk and rate of exacerbation and earlier time to first exacerbation compared to LPA-high men. This study shows that LPA species may identify men with increased exacerbation risk. These biomarkers may aid in identifying high-risk patients to tailor treatment plans.

Consistent with Naz et al. (The European Respiratory Journal, 49, 2017), this study also noted gender-associated differences in these biomarkers, reiterating the importance of patient stratification by gender in biomarker and clinical outcome analysis in COPD.

### Example 8. LPA Species and Lipidomics

A. LPA Species and Lipidomics in Men

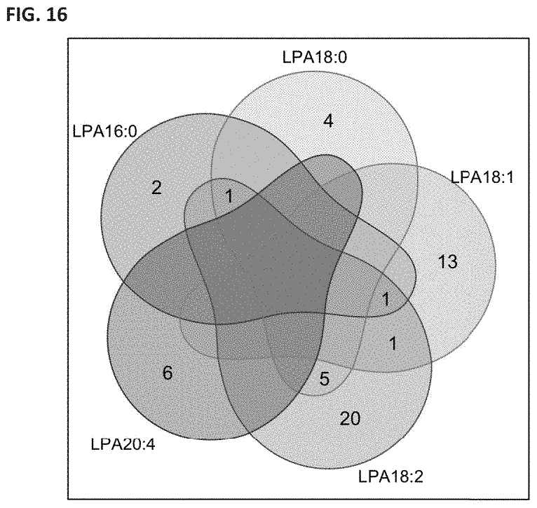

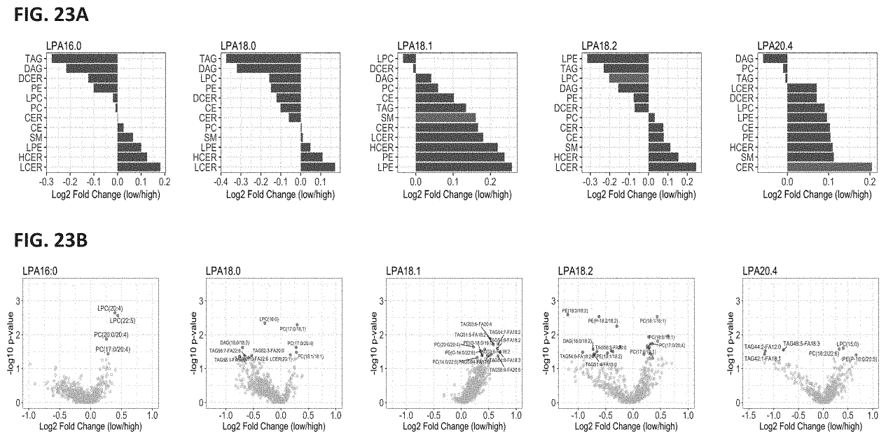

Lipidomics profiles were compared between LPA low subgroup and LPA high subgroup patients for each LPA species to characterize potential metabolic shifts underlying the observed increased exacerbation risk, since there were consistent differences in exacerbation metrics between these two extreme subgroups in men. LPC, the precursor of LPA, was lower in the male LPA18:2 low subgroup compared to the high subgroup (p=0.038) (FIGS. 23A and 23B). The relative abundance of sphingomyelins (SM) and ceramides (CER) was higher in the male LPA18:1 and LPA20:4 low subgroups, respectively, compared to the respective LPA high subgroups (SM p=0.041; CER p=0.040). Hexosylceramides (HCER) and lactosylceramides (LCER) showed trends of an increase in LPA low subgroups for all LPA species, but the differences were not significant. 507 lipid species were detectable in at least 90% of the patients. Comparison of these lipid species between LPA low and high subgroups showed some overlapping species, but also unique species; however, the changes were modest, as only a few lipid species showed differential expression (p<0.05) (FIGS. 23A and 23B). Table 14 shows the full list of differentially expressed lipid species between LPA subgroups in men. LPA18:0 and LPA18:2 showed the most overlap, with 6 lipid species commonly found in both subgroup comparisons (FIG. 16).

TABLE 14
Differential expression of lipid species
by baseline biomarker profile in men

| | LPA-low, | | | | Fold |
|---|---|---|---|---|---|
| | LPA-high | Lipid | | | Change |
| LPA | N | Species | p-value | FDR | Low/High |
| LPA16:0 | 35, 19 | LPC(20:4) | 0.002 | 0.695 | 1.32 |
| LPA16:0 | 35, 19 | LPC(22:5) | 0.003 | 0.695 | 1.36 |
| LPA16:0 | 35, 19 | PC(17:0/20:4) | 0.036 | 0.993 | 1.22 |
| LPA16:0 | 35, 19 | PC(20:0/20:4) | 0.013 | 0.993 | 1.19 |
| LPA18:0 | 34, 19 | DAG(16:0/18:2) | 0.024 | 0.828 | 0.62 |
| LPA18:0 | 34, 19 | LCER(20:1) | 0.039 | 0.828 | 1.13 |
| LPA18:0 | 34, 19 | LPC(16:0) | 0.005 | 0.828 | 0.82 |
| LPA18:0 | 34, 19 | PC(17:0/18:1) | 0.005 | 0.828 | 1.23 |
| LPA18:0 | 34, 19 | PC(17:0/20:4) | 0.024 | 0.828 | 1.21 |
| LPA18:0 | 34, 19 | PC(18:1/16:1) | 0.033 | 0.828 | 1.22 |
| LPA18:0 | 34, 19 | TAG52:3-FA20:0 | 0.041 | 0.828 | 0.69 |
| LPA18:0 | 34, 19 | TAG55:1-FA16:0 | 0.043 | 0.828 | 0.60 |
| LPA18:0 | 34, 19 | TAG56:7-FA22:6 | 0.041 | 0.828 | 0.63 |
| LPA18:0 | 34, 19 | TAG60:10-FA22:6 | 0.047 | 0.828 | 0.67 |
| LPA18:1 | 33, 19 | PC(14:0/22:5) | 0.048 | 0.682 | 1.22 |
| LPA18.:1 | 33, 19 | PC(20:0/20:4) | 0.023 | 0.682 | 1.17 |
| LPA18:1 | 33, 19 | PE(O-16:0/22:6) | 0.031 | 0.682 | 1.28 |
| LPA18:1 | 33, 19 | PE(O-18:0/16:0) | 0.041 | 0.682 | 1.30 |
| LPA18:1 | 33, 19 | TAG51:5-FA18:2 | 0.019 | 0.682 | 1.50 |
| LPA18:1 | 33, 19 | TAG52:6-FA16:1 | 0.041 | 0.682 | 1.39 |
| LPA18:1 | 33, 19 | TAG52:6-FA18:1 | 0.027 | 0.682 | 1.34 |
| LPA18:1 | 33, 19 | TAG52:6-FA18:2 | 0.037 | 0.682 | 1.47 |
| LPA18:1 | 33, 19 | TAG53:4-FA17:0 | 0.041 | 0.682 | 1.43 |
| LPA18:1 | 33, 19 | TAG53:6-FA20:4 | 0.013 | 0.682 | 1.45 |
| LPA18:1 | 33, 19 | TAG54:7-FA18:2 | 0.019 | 0.682 | 1.59 |
| LPA18:1 | 33, 19 | TAG54:8-FA18:2 | 0.026 | 0.682 | 1.57 |
| LPA18:1 | 33, 19 | TAG54:8-FA18:3 | 0.033 | 0.682 | 1.62 |
| LPA18:1 | 33, 19 | TAG56:9-FA20:5 | 0.042 | 0.682 | 1.59 |
| LPA18:1 | 33, 19 | TAG58:10-FA22:5 | 0.038 | 0.682 | 1.30 |
| LPA18:2 | 30, 19 | CE(18:3) | 0.049 | 0.713 | 1.28 |
| LPA18:2 | 30, 19 | CE(20:3) | 0.025 | 0.713 | 1.22 |
| LPA18:2 | 30, 19 | CE(22:4) | 0.036 | 0.713 | 1.23 |
| LPA18:2 | 30, 19 | DAG(16:0/18:2) | 0.027 | 0.713 | 0.60 |
| LPA18:2 | 30, 19 | HCER(24:1) | 0.038 | 0.713 | 1.27 |
| LPA18:2 | 30, 19 | LCER(20:1) | 0.038 | 0.713 | 1.15 |
| LPA18:2 | 30, 19 | LPC(16:0) | 0.023 | 0.713 | 0.85 |
| LPA18:2 | 30, 19 | LPC(18:2) | 0.031 | 0.713 | 0.77 |
| LPA18:2 | 30, 19 | PC(14:0/22:5) | 0.049 | 0.713 | 1.23 |
| LPA18:2 | 30, 19 | PC(17:0/18:1) | 0.019 | 0.713 | 1.24 |
| LPA18:2 | 30, 19 | PC(17:0/20:4) | 0.018 | 0.713 | 1.27 |
| LPA18:2 | 30, 19 | PC(18:0/16:1) | 0.015 | 0.713 | 1.36 |
| LPA18:2 | 30, 19 | PC(18:0/18:0) | 0.012 | 0.713 | 1.22 |
| LPA18:2 | 30, 19 | PC(18:0/22:5) | 0.024 | 0.713 | 1.23 |
| LPA18:2 | 30, 19 | PC(18:1/16:1) | 0.003 | 0.495 | 1.35 |
| LPA18:2 | 30, 19 | PC(20:0/18:2) | 0.006 | 0.685 | 0.81 |
| LPA18:2 | 30, 19 | PC(20:0/20:4) | 0.022 | 0.713 | 1.19 |
| LPA18:2 | 30, 19 | PE(18:0/18:2) | 0.035 | 0.713 | 0.72 |
| LPA18:2 | 30, 19 | PE(18:1/18:2) | 0.037 | 0.713 | 0.62 |
| LPA18:2 | 30, 19 | PE(18:2/18:2) | 0.003 | 0.495 | 0.44 |
| LPA18:2 | 30, 19 | PE(O-18:0/18:2) | 0.030 | 0.713 | 0.76 |
| LPA18:2 | 30, 19 | PE(P-18:2/18:2) | 0.003 | 0.495 | 0.65 |
| LPA18:2 | 30, 19 | TAG46:4-FA18:2 | 0.007 | 0.685 | 0.41 |
| LPA18:2 | 30, 19 | TAG51:4-FA15:0 | 0.038 | 0.713 | 0.61 |
| LPA18:2 | 30, 19 | TAG52:4-FA20:0 | 0.044 | 0.713 | 0.60 |
| LPA18:2 | 30, 19 | TAG54:6-FA18:2 | 0.033 | 0.713 | 0.53 |
| LPA18:2 | 30, 19 | TAG56:3-FA20:0 | 0.031 | 0.713 | 0.63 |
| LPA18:2 | 30, 19 | TAG58:8-FA20:3 | 0.011 | 0.713 | 1.55 |
| LPA20:4 | 26, 25 | LPC(15:0) | 0.025 | 0.996 | 1.32 |
| LPA20:4 | 26, 25 | PC(18:2/22:6) | 0.026 | 0.996 | 1.25 |
| LPA20:4 | 26, 25 | PE(P-16:0/20:5) | 0.039 | 0.996 | 1.59 |
| LPA20:4 | 26, 25 | TAG42:1-FA18:1 | 0.037 | 0.996 | 0.44 |
| LPA20:4 | 26, 25 | TAG44:2-FA12:0 | 0.030 | 0.996 | 0.44 |
| LPA20:4 | 26, 25 | TAG48:5-FA18:3 | 0.028 | 0.996 | 0.57 |

B. LPA Species and Lipidomics in Women

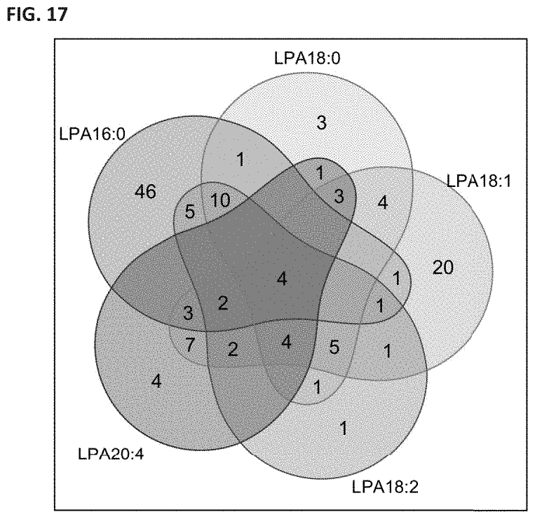

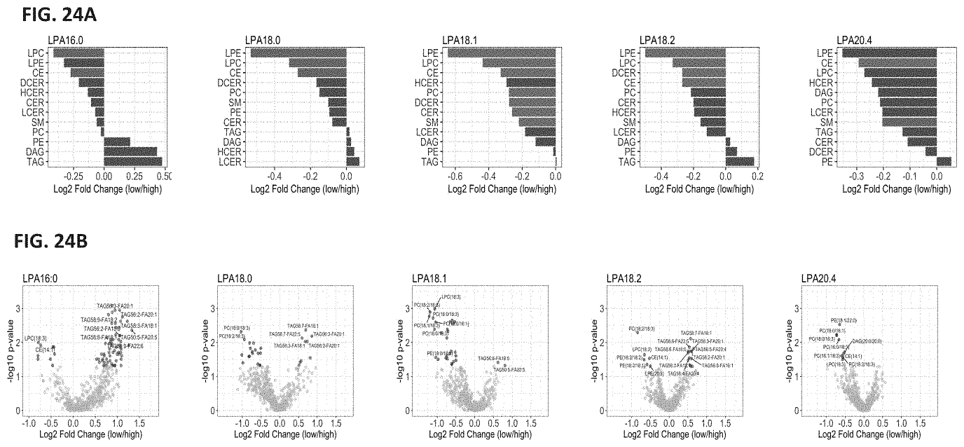

Despite the lack of significant differences in exacerbations among LPA species subgroups in women, more pronounced alterations in lipid classes were observed (FIGS. 24A and 24B). Cholesteryl esters (CE) were lower in all LPA low subgroups (p<0.05, FDR<0.1); dihydroceramides (DCER) were lower in LPA18:1 and LPA18:2 low subgroups compared to high subgroups (FDR<0.1); and sphingomyelins (SM) were lower in LPA18:1 and LPA20:4 low subgroups compared to the corresponding high subgroups (FDR<0.1, p<0.05). Accordingly, many differentially expressed lipid species were identified, with changes greater than one-fold (p<0.05) (FIGS. 17, 24A, and 24B). Table 15 contains the full list of differentially expressed lipid species between LPA species subgroups in women.

TABLE 15
Differential expression of lipid species
by baseline biomarker profile in women

| | LPA-low, | | | | Fold |
|---|---|---|---|---|---|
| | LPA-high | Lipid | | | Change |
| LPA | N | Species | p-value | FDR | Low/High |
| LPA16:0 | 7, 26 | CE(14:1) | 0.012 | 0.204 | 0.60 |
| LPA16:0 | 7, 26 | LPC(16:0) | 0.014 | 0.204 | 0.74 |
| LPA16:0 | 7, 26 | LPC(16:1) | 0.048 | 0.330 | 0.72 |
| LPA16:0 | 7, 26 | LPC(18:1) | 0.014 | 0.204 | 0.73 |
| LPA16:0 | 7, 26 | LPC(18:3) | 0.010 | 0.204 | 0.58 |
| LPA16:0 | 7, 26 | LPC(20:2) | 0.022 | 0.243 | 0.75 |
| LPA16:0 | 7, 26 | LPC(20:3) | 0.031 | 0.275 | 0.70 |
| LPA16:0 | 7, 26 | PC(18:1/18:3) | 0.025 | 0.251 | 0.58 |
| LPA16:0 | 7, 26 | PC(18:2/18:3) | 0.031 | 0.275 | 0.58 |
| LPA16:0 | 7, 26 | TAG50:5-FA20:5 | 0.004 | 0.171 | 2.51 |
| LPA16:0 | 7, 26 | TAG52:3-FA14:0 | 0.048 | 0.330 | 1.69 |
| LPA16:0 | 7, 26 | TAG52:6-FA14:0 | 0.022 | 0.243 | 1.62 |
| LPA16:0 | 7, 26 | TAG52:6-FA18:1 | 0.022 | 0.243 | 1.56 |
| LPA16:0 | 7, 26 | TAG52:7-FA20:5 | 0.025 | 0.251 | 1.63 |
| LPA16:0 | 7, 26 | TAG54:3-FA16:1 | 0.035 | 0.292 | 1.58 |
| LPA16:0 | 7, 26 | TAG54:5-FA20:5 | 0.048 | 0.330 | 1.61 |
| LPA16:0 | 7, 26 | TAG54:6-FA20:5 | 0.048 | 0.330 | 1.65 |
| LPA16:0 | 7, 26 | TAG54:7-FA16:1 | 0.031 | 0.275 | 1.69 |
| LPA16:0 | 7, 26 | TAG54:7-FA18:1 | 0.031 | 0.275 | 1.49 |
| LPA16:0 | 7, 26 | TAG54:7-FA22:5 | 0.038 | 0.310 | 1.72 |
| LPA16:0 | 7, 26 | TAG55:1-FA16:0 | 0.008 | 0.204 | 2.04 |
| LPA16:0 | 7, 26 | TAG55:1-FA18:1 | 0.020 | 0.242 | 1.75 |
| LPA16:0 | 7, 26 | TAG55:2-FA18:2 | 0.048 | 0.330 | 1.86 |
| LPA16:0 | 7, 26 | TAG56:2-FA16:0 | 0.022 | 0.243 | 1.99 |
| LPA16:0 | 7, 26 | TAG56:2-FA18:0 | 0.006 | 0.201 | 2.08 |
| LPA16:0 | 7, 26 | TAG56:2-FA20:1 | 0.002 | 0.139 | 2.16 |
| LPA16:0 | 7, 26 | TAG56:3-FA16:0 | 0.043 | 0.330 | 1.83 |
| LPA16:0 | 7, 26 | TAG56:3-FA18:0 | 0.006 | 0.201 | 1.95 |
| LPA16:0 | 7, 26 | TAG56:3-FA18:1 | 0.001 | 0.139 | 1.85 |
| LPA16:0 | 7, 26 | TAG56:3-FA18:2 | 0.022 | 0.243 | 2.05 |
| LPA16:0 | 7, 26 | TAG56:3-FA20:1 | 0.001 | 0.139 | 2.07 |
| LPA16:0 | 7, 26 | TAG56:4-FA18:0 | 0.038 | 0.310 | 1.63 |
| LPA16:0 | 7, 26 | TAG56:4-FA20:3 | 0.038 | 0.310 | 1.51 |
| LPA16:0 | 7, 26 | TAG56:4-FA20:4 | 0.009 | 0.204 | 1.80 |
| LPA16:0 | 7, 26 | TAG56:5-FA18:0 | 0.011 | 0.204 | 1.77 |
| LPA16:0 | 7, 26 | TAG56:5-FA18:2 | 0.035 | 0.292 | 1.96 |
| LPA16:0 | 7, 26 | TAG56:5-FA20:1 | 0.020 | 0.242 | 2.03 |
| LPA16:0 | 7, 26 | TAG56:5-FA20:4 | 0.008 | 0.204 | 1.81 |
| LPA16:0 | 7, 26 | TAG56:6-FA18:0 | 0.003 | 0.139 | 1.93 |
| LPA16:0 | 7, 26 | TAG56:6-FA22:6 | 0.048 | 0.330 | 1.86 |
| LPA16:0 | 7, 26 | TAG56:7-FA16:0 | 0.012 | 0.204 | 1.82 |
| LPA16:0 | 7, 26 | TAG56:7-FA18:0 | 0.015 | 0.217 | 1.77 |
| LPA16:0 | 7, 26 | TAG56:7-FA22:5 | 0.035 | 0.292 | 1.78 |
| LPA16:0 | 7, 26 | TAG56:7-FA22:6 | 0.048 | 0.330 | 1.92 |
| LPA16:0 | 7, 26 | TAG56:8-FA16:1 | 0.020 | 0.242 | 1.87 |
| LPA16:0 | 7, 26 | TAG56:8-FA18:2 | 0.014 | 0.204 | 1.97 |
| LPA16:0 | 7, 26 | TAG56:9-FA22:6 | 0.048 | 0.330 | 1.92 |
| LPA16:0 | 7, 26 | TAG57:3-FA18:2 | 0.012 | 0.204 | 2.07 |
| LPA16:0 | 7, 26 | TAG58:10-FA18:2 | 0.014 | 0.204 | 2.08 |
| LPA16:0 | 7, 26 | TAG58:10-FA20:4 | 0.031 | 0.275 | 2.12 |
| LPA16:0 | 7, 26 | TAG58:10-FA22:5 | 0.031 | 0.275 | 1.44 |
| LPA16:0 | 7, 26 | TAG58:10-FA22:6 | 0.015 | 0.217 | 2.12 |
| LPA16:0 | 7, 26 | TAG58:3-FA18:1 | 0.002 | 0.139 | 2.34 |
| LPA16:0 | 7, 26 | TAG58:6-FA18:0 | 0.003 | 0.139 | 1.83 |
| LPA16:0 | 7, 26 | TAG58:6-FA18:1 | 0.031 | 0.275 | 1.59 |
| LPA16:0 | 7, 26 | TAG58:6-FA20:4 | 0.001 | 0.139 | 1.93 |
| LPA16:0 | 7, 26 | TAG58:6-FA22:5 | 0.011 | 0.204 | 1.66 |
| LPA16:0 | 7, 26 | TAG58:7-FA18:0 | 0.004 | 0.154 | 2.06 |
| LPA16:0 | 7, 26 | TAG58:7-FA18:1 | 0.001 | 0.139 | 1.84 |
| LPA16:0 | 7, 26 | TAG58:7-FA18:2 | 0.025 | 0.251 | 1.78 |
| LPA16:0 | 7, 26 | TAG58:7-FA20:4 | 0.017 | 0.239 | 1.91 |
| LPA16:0 | 7, 26 | TAG58:7-FA22:5 | 0.002 | 0.139 | 1.74 |
| LPA16:0 | 7, 26 | TAG58:7-FA22:6 | 0.020 | 0.242 | 2.11 |
| LPA16:0 | 7, 26 | TAG58:8-FA18:1 | 0.006 | 0.201 | 2.07 |
| LPA16:0 | 7, 26 | TAG58:8-FA18:2 | 0.004 | 0.154 | 1.97 |
| LPA16:0 | 7, 26 | TAG58:8-FA22:5 | 0.011 | 0.204 | 1.84 |
| LPA16:0 | 7, 26 | TAG58:8-FA22:6 | 0.025 | 0.251 | 2.14 |
| LPA16:0 | 7, 26 | TAG58:9-FA18:2 | 0.002 | 0.139 | 2.10 |
| LPA16:0 | 7, 26 | TAG58:9-FA22:6 | 0.009 | 0.204 | 2.11 |
| LPA16:0 | 7, 26 | TAG60:10-FA22:5 | 0.007 | 0.204 | 1.70 |
| LPA16:0 | 7, 26 | TAG60:10-FA22:6 | 0.012 | 0.204 | 2.39 |
| LPA16:0 | 7, 26 | TAG60:11-FA22:5 | 0.008 | 0.204 | 1.87 |
| LPA16:0 | 7, 26 | TAG60:11-FA22:6 | 0.048 | 0.330 | 2.12 |
| LPA18:0 | 8, 26 | CE(14:1) | 0.010 | 0.527 | 0.62 |
| LPA18:0 | 8, 26 | CE(18:3) | 0.021 | 0.542 | 0.60 |
| LPA18:0 | 8, 26 | CE(20:2) | 0.026 | 0.542 | 0.80 |
| LPA18:0 | 8, 26 | CE(20:3) | 0.029 | 0.542 | 0.78 |
| LPA18:0 | 8, 26 | LPC(18:2) | 0.047 | 0.542 | 0.76 |
| LPA18:0 | 8, 26 | LPC(18:3) | 0.015 | 0.542 | 0.58 |
| LPA18:0 | 8, 26 | LPC(20:3) | 0.021 | 0.542 | 0.70 |
| LPA18:0 | 8, 26 | LPE(18:0) | 0.009 | 0.527 | 0.72 |
| LPA18:0 | 8, 26 | LPE(18:1) | 0.047 | 0.542 | 0.69 |
| LPA18:0 | 8, 26 | PC(16:0/18:3) | 0.026 | 0.542 | 0.56 |
| LPA18:0 | 8, 26 | PC(18:0/18:3) | 0.005 | 0.527 | 0.49 |
| LPA18:0 | 8, 26 | PC(18:2/18:3) | 0.008 | 0.527 | 0.52 |
| LPA18:0 | 8, 26 | PC(20:0/18:2) | 0.021 | 0.542 | 0.80 |
| LPA18:0 | 8, 26 | PE(18:0/18:2) | 0.029 | 0.542 | 0.77 |
| LPA18:0 | 8, 26 | PE(18:0/18:3) | 0.023 | 0.542 | 0.49 |
| LPA18:0 | 8, 26 | PE(18:1/18:2) | 0.044 | 0.542 | 0.75 |
| LPA18:0 | 8, 26 | PE(18:2/16:1) | 0.010 | 0.527 | 0.57 |
| LPA18:0 | 8, 26 | PE(18:2/18:2) | 0.019 | 0.542 | 0.60 |
| LPA18:0 | 8, 26 | PE(O-18:0/18:2) | 0.015 | 0.542 | 0.70 |
| LPA18:0 | 8, 26 | PE(P-16:0/18:2) | 0.021 | 0.542 | 0.67 |
| LPA18:0 | 8, 26 | PE(P-18:0/18:2) | 0.017 | 0.542 | 0.64 |
| LPA18:0 | 8, 26 | TAG46:3-FA14:1 | 0.040 | 0.542 | 0.50 |
| LPA18:0 | 8, 26 | TAG47:1-FA16:1 | 0.026 | 0.542 | 0.57 |
| LPA18:0 | 8, 26 | TAG52:4-FA20:4 | 0.044 | 0.542 | 0.67 |
| LPA18:0 | 8, 26 | TAG52:6-FA20:4 | 0.047 | 0.542 | 0.69 |
| LPA18:0 | 8, 26 | TAG56:2-FA18:0 | 0.038 | 0.542 | 1.46 |
| LPA18:0 | 8, 26 | TAG56:2-FA20:1 | 0.009 | 0.527 | 1.66 |
| LPA18:0 | 8, 26 | TAG56:3-FA18:1 | 0.009 | 0.527 | 1.60 |
| LPA18:0 | 8, 26 | TAG56:3-FA20:1 | 0.007 | 0.527 | 1.81 |
| LPA18:0 | 8, 26 | TAG56:5-FA20:4 | 0.047 | 0.542 | 1.42 |
| LPA18:0 | 8, 26 | TAG58:3-FA18:1 | 0.035 | 0.542 | 1.48 |
| LPA18:0 | 8, 26 | TAG58:6-FA18:0 | 0.038 | 0.542 | 1.40 |
| LPA18:0 | 8, 26 | TAG58:6-FA20:4 | 0.028 | 0.542 | 1.35 |
| LPA18:0 | 8, 26 | TAG58:7-FA18:1 | 0.004 | 0.527 | 1.62 |
| LPA18:0 | 8, 26 | TAG58:7-FA22:5 | 0.007 | 0.527 | 1.51 |
| LPA18:0 | 8, 26 | TAG58:8-FA18:1 | 0.028 | 0.542 | 1.75 |
| LPA18:1 | 9, 26 | CE(14:1) | 0.007 | 0.197 | 0.60 |
| LPA18:1 | 9, 26 | CE(18:3) | 0.021 | 0.409 | 0.63 |
| LPA18:1 | 9, 26 | CE(20:2) | 0.008 | 0.201 | 0.75 |
| LPA18:1 | 9, 26 | CE(22:0) | 0.039 | 0.418 | 0.73 |
| LPA18:1 | 9, 26 | DAG(16:0/18:0) | 0.042 | 0.425 | 0.65 |
| LPA18:1 | 9, 26 | DCER(24:0) | 0.040 | 0.418 | 0.71 |
| LPA18:1 | 9, 26 | HCER(18:0) | 0.047 | 0.433 | 0.73 |
| LPA18:1 | 9, 26 | LCER(18:0) | 0.023 | 0.409 | 0.83 |
| LPA18:1 | 9, 26 | LPC(16:0) | 0.001 | 0.128 | 0.73 |
| LPA18:1 | 9, 26 | LPC(16:1) | 0.003 | 0.130 | 0.66 |
| LPA18:1 | 9, 26 | LPC(17:0) | 0.042 | 0.425 | 0.76 |
| LPA18:1 | 9, 26 | LPC(18:1) | 0.000 | 0.110 | 0.65 |
| LPA18:1 | 9, 26 | LPC(18:2) | 0.026 | 0.409 | 0.72 |
| LPA18:1 | 9, 26 | LPC(18:3) | 0.001 | 0.128 | 0.48 |
| LPA18:1 | 9, 26 | LPC(20:1) | 0.035 | 0.417 | 0.73 |
| LPA18:1 | 9, 26 | LPC(20:2) | 0.007 | 0.196 | 0.71 |
| LPA18:1 | 9, 26 | LPC(20:3) | 0.002 | 0.128 | 0.66 |
| LPA18:1 | 9, 26 | LPE(16:1) | 0.005 | 0.166 | 0.54 |
| LPA18:1 | 9, 26 | LPE(18:0) | 0.002 | 0.128 | 0.69 |
| LPA18:1 | 9, 26 | LPE(18:1) | 0.005 | 0.157 | 0.61 |
| LPA18:1 | 9, 26 | LPE(20:3) | 0.004 | 0.149 | 0.60 |
| LPA18:1 | 9, 26 | PC(14:0/18:1) | 0.025 | 0.409 | 0.60 |
| LPA18:1 | 9, 26 | PC(14:0/20:2) | 0.025 | 0.409 | 0.70 |
| LPA18:1 | 9, 26 | PC(15:0/18:1) | 0.019 | 0.391 | 0.70 |
| LPA18:1 | 9, 26 | PC(16:0/14:0) | 0.030 | 0.409 | 0.60 |
| LPA18:1 | 9, 26 | PC(16:0/18:3) | 0.002 | 0.128 | 0.46 |
| LPA18:1 | 9, 26 | PC(16:0/20:2) | 0.008 | 0.201 | 0.77 |
| LPA18:1 | 9, 26 | PC(16:0/20:3) | 0.025 | 0.409 | 0.76 |
| LPA18:1 | 9, 26 | PC(17:0/18:1) | 0.027 | 0.409 | 0.73 |
| LPA18:1 | 9, 26 | PC(18:0/14:0) | 0.029 | 0.409 | 0.59 |
| LPA18:1 | 9, 26 | PC(18:0/16:1) | 0.003 | 0.128 | 0.49 |
| LPA18:1 | 9, 26 | PC(18:0/18:0) | 0.003 | 0.137 | 0.72 |
| LPA18:1 | 9, 26 | PC(18:0/18:3) | 0.004 | 0.149 | 0.48 |
| LPA18:1 | 9, 26 | PC(18:1/16:1) | 0.002 | 0.128 | 0.63 |
| LPA18:1 | 9, 26 | PC(18:1/18:3) | 0.002 | 0.128 | 0.43 |
| LPA18:1 | 9, 26 | PC(18:1/20:2) | 0.038 | 0.418 | 0.75 |
| LPA18:1 | 9, 26 | PC(18:2/16:1) | 0.033 | 0.413 | 0.77 |
| LPA18:1 | 9, 26 | PC(18:2/18:3) | 0.001 | 0.128 | 0.44 |
| LPA18:1 | 9, 26 | PC(18:2/20:3) | 0.033 | 0.413 | 0.71 |
| LPA18:1 | 9, 26 | PC(20:0/14:1) | 0.049 | 0.433 | 0.81 |
| LPA18:1 | 9, 26 | PC(20:0/18:1) | 0.012 | 0.260 | 0.71 |
| LPA18:1 | 9, 26 | PC(20:0/18:2) | 0.007 | 0.197 | 0.74 |
| LPA18:1 | 9, 26 | PC(20:0/20:3) | 0.012 | 0.260 | 0.78 |
| LPA18:1 | 9, 26 | PE(16:0/16:0) | 0.036 | 0.418 | 0.72 |
| LPA18:1 | 9, 26 | PE(18:0/18:2) | 0.044 | 0.425 | 0.79 |
| LPA18:1 | 9, 26 | PE(18:0/18:3) | 0.027 | 0.409 | 0.49 |
| LPA18:1 | 9, 26 | PE(18:1/16:1) | 0.030 | 0.409 | 0.72 |
| LPA18:1 | 9, 26 | PE(18:1/18:2) | 0.049 | 0.433 | 0.78 |
| LPA18:1 | 9, 26 | PE(18:2/16:1) | 0.018 | 0.384 | 0.65 |
| LPA18:1 | 9, 26 | PE(18:2/18:2) | 0.044 | 0.425 | 0.65 |
| LPA18:1 | 9, 26 | PE(P-16:0/18:1) | 0.031 | 0.409 | 0.75 |
| LPA18:1 | 9, 26 | PE(P-16:0/18:2) | 0.044 | 0.425 | 0.76 |
| LPA18:1 | 9, 26 | PE(P-18:0/16:1) | 0.037 | 0.418 | 0.68 |
| LPA18:1 | 9, 26 | PE(P-18:0/18:2) | 0.030 | 0.409 | 0.73 |
| LPA18:1 | 9, 26 | TAG47:1-FA16:1 | 0.031 | 0.409 | 0.51 |
| LPA18:1 | 9, 26 | TAG50:5-FA20:5 | 0.048 | 0.433 | 1.75 |
| LPA18:1 | 9, 26 | TAG56:6-FA18:0 | 0.038 | 0.418 | 1.54 |
| LPA18:2 | 12, 26 | CE(14:1) | 0.029 | 0.646 | 0.69 |
| LPA18:2 | 12, 26 | CE(20:2) | 0.006 | 0.646 | 0.78 |
| LPA18:2 | 12, 26 | DCER(22:1) | 0.035 | 0.651 | 0.74 |
| LPA18:2 | 12, 26 | DCER(24:0) | 0.028 | 0.646 | 0.74 |
| LPA18:2 | 12, 26 | LPC(16:0) | 0.030 | 0.646 | 0.80 |
| LPA18:2 | 12, 26 | LPC(18:1) | 0.031 | 0.646 | 0.80 |
| LPA18:2 | 12, 26 | LPC(18:2) | 0.012 | 0.646 | 0.73 |
| LPA18:2 | 12, 26 | LPC(18:3) | 0.023 | 0.646 | 0.63 |
| LPA18:2 | 12, 26 | LPC(20:3) | 0.039 | 0.666 | 0.74 |
| LPA18:2 | 12, 26 | LPE(18:0) | 0.038 | 0.666 | 0.79 |
| LPA18:2 | 12, 26 | LPE(18:1) | 0.030 | 0.646 | 0.72 |
| LPA18:2 | 12, 26 | LPE(20:3) | 0.050 | 0.698 | 0.71 |
| LPA18:2 | 12, 26 | PC(18:0/18:0) | 0.030 | 0.646 | 0.81 |
| LPA18:2 | 12, 26 | PC(18:2/18:3) | 0.005 | 0.646 | 0.56 |
| LPA18:2 | 12, 26 | PC(20:0/18:2) | 0.002 | 0.646 | 0.74 |
| LPA18:2 | 12, 26 | PE(18:2/16:1) | 0.045 | 0.698 | 0.66 |
| LPA18:2 | 12, 26 | PE(18:2/18:2) | 0.032 | 0.646 | 0.63 |
| LPA18:2 | 12, 26 | PE(O-18:0/18:2) | 0.030 | 0.646 | 0.81 |
| LPA18:2 | 12, 26 | PE(P-16:0/18:2) | 0.048 | 0.698 | 0.76 |
| LPA18:2 | 12, 26 | PE(P-18:0/18:2) | 0.023 | 0.646 | 0.72 |
| LPA18:2 | 12, 26 | TAG56:2-FA18:0 | 0.039 | 0.666 | 1.40 |
| LPA18:2 | 12, 26 | TAG56:2-FA20:1 | 0.018 | 0.646 | 1.52 |
| LPA18:2 | 12, 26 | TAG56:3-FA18:1 | 0.020 | 0.646 | 1.42 |
| LPA18:2 | 12, 26 | TAG56:3-FA20:1 | 0.015 | 0.646 | 1.57 |
| LPA18:2 | 12, 26 | TAG56:4-FA20:4 | 0.028 | 0.646 | 1.45 |
| LPA18:2 | 12, 26 | TAG56:5-FA18:0 | 0.033 | 0.651 | 1.42 |
| LPA18:2 | 12, 26 | TAG56:5-FA20:4 | 0.018 | 0.646 | 1.46 |
| LPA18:2 | 12, 26 | TAG56:6-FA18:0 | 0.046 | 0.698 | 1.46 |
| LPA18:2 | 12, 26 | TAG56:8-FA16:1 | 0.030 | 0.646 | 1.51 |
| LPA18:2 | 12, 26 | TAG58:6-FA18:0 | 0.018 | 0.646 | 1.42 |
| LPA18:2 | 12, 26 | TAG58:6-FA20:4 | 0.017 | 0.646 | 1.37 |
| LPA18:2 | 12, 26 | TAG58:6-FA22:5 | 0.014 | 0.646 | 1.45 |
| LPA18:2 | 12, 26 | TAG58:7-FA18:0 | 0.050 | 0.698 | 1.49 |
| LPA18:2 | 12, 26 | TAG58:7-FA18:1 | 0.008 | 0.646 | 1.49 |
| LPA18:2 | 12, 26 | TAG58:7-FA22:5 | 0.014 | 0.646 | 1.40 |
| LPA18:2 | 12, 26 | TAG58:8-FA18:1 | 0.050 | 0.698 | 1.53 |
| LPA20:4 | 18, 19 | CE(14:1) | 0.022 | 0.768 | 0.68 |
| LPA20:4 | 18, 19 | CE(20:2) | 0.048 | 0.796 | 0.82 |
| LPA20:4 | 18, 19 | CE(20:3) | 0.048 | 0.796 | 0.83 |
| LPA20:4 | 18, 19 | CE(22:0) | 0.008 | 0.420 | 0.72 |
| LPA20:4 | 18, 19 | DAG(16:0/18:0) | 0.037 | 0.796 | 0.73 |
| LPA20:4 | 18, 19 | DAG(16:1/18:2) | 0.049 | 0.796 | 0.73 |
| LPA20:4 | 18, 19 | DAG(20:0/20:0) | 0.019 | 0.768 | 0.70 |
| LPA20:4 | 18, 19 | LPC(16:0) | 0.043 | 0.796 | 0.80 |
| LPA20:4 | 18, 19 | LPC(16:1) | 0.021 | 0.768 | 0.76 |
| LPA20:4 | 18, 19 | LPC(18:1) | 0.034 | 0.796 | 0.78 |
| LPA20:4 | 18, 19 | LPC(18:3) | 0.031 | 0.796 | 0.67 |
| LPA20:4 | 18, 19 | LPC(20:2) | 0.046 | 0.796 | 0.80 |
| LPA20:4 | 18, 19 | LPC(20:3) | 0.043 | 0.796 | 0.77 |
| LPA20:4 | 18, 19 | LPE(18:0) | 0.041 | 0.796 | 0.78 |
| LPA20:4 | 18, 19 | LPE(20:3) | 0.040 | 0.796 | 0.73 |
| LPA20:4 | 18, 19 | PC(16:0/18:3) | 0.008 | 0.420 | 0.63 |
| LPA20:4 | 18, 19 | PC(16:0/20:2) | 0.023 | 0.768 | 0.82 |
| LPA20:4 | 18, 19 | PC(18:0/16:1) | 0.006 | 0.420 | 0.60 |
| LPA20:4 | 18, 19 | PC(18:0/18:0) | 0.016 | 0.755 | 0.83 |
| LPA20:4 | 18, 19 | PC(18:0/18:3) | 0.006 | 0.420 | 0.60 |
| LPA20:4 | 18, 19 | PC(18:0/20:2) | 0.005 | 0.420 | 0.81 |
| LPA20:4 | 18, 19 | PC(18:1/18:3) | 0.025 | 0.786 | 0.66 |
| LPA20:4 | 18, 19 | PC(18:2/16:1) | 0.005 | 0.420 | 0.77 |
| LPA20:4 | 18, 19 | PC(18:2/18:3) | 0.027 | 0.796 | 0.68 |
| LPA20:4 | 18, 19 | PC(20:0/14:1) | 0.008 | 0.420 | 0.78 |
| LPA20:4 | 18, 19 | PC(20:0/18:2) | 0.003 | 0.420 | 0.80 |
| LPA20:4 | 18, 19 | PC(20:0/20:3) | 0.006 | 0.420 | 0.84 |
| LPA20:4 | 18, 19 | PE(18:0/18:2) | 0.040 | 0.796 | 0.78 |
| LPA20:4 | 18, 19 | PE(18:1/22:0) | 0.003 | 0.420 | 0.64 |
| LPA20:4 | 18, 19 | PE(18:2/18:2) | 0.042 | 0.796 | 0.72 |

C. Discussion

There were modest changes in lipidomics among the male LPA subgroups, suggesting that LPA species could be the more sensitive biomarkers to identify these patients. There were pronounced shifts in lipidomics among female LPA subgroups, providing evidence of a gender-specific molecular phenotype in COPD.

Although levels of LPA species were correlated, there were differences in lipidomic profiles among the LPA species subgroups. Many of the shifts in lipid classes and species could be reflective of changes in pulmonary surfactant homeostasis. Pulmonary surfactant is made up of a complex mixture of lipids (phospholipids, triglycerides (TAG), fatty acids, cholesterol, sphingolipids, and others) and surfactant proteins (SP). Phosphatidylcholine (PC) is the predominant lipid class, making up to 50% of the phospholipids in pulmonary surfactant lipids. HCER and LCER are likely minor components of the lung lipids (Kyle et al., Sci Rep, 8: 13455, 2018), but increased accumulation of these glucosphingolipids in lung tissues has been observed in lung adenocarcinoma (Lemay et al., J Lipid Res, 60: 1776-1786, 2019) and emphysema (Bodas et al., Apoptosis, 20: 725-739, 2015), respectively. Lipids from the sphingolipid pathway, SM and CER, were upregulated in COPD compared to controls, wherein increasing sputum CER levels were linked to greater airflow obstruction and gas trapping (Telenga et al., Am J Resp Crit Care Med, 190: 155-164, 2014). Systemic changes in surfactant proteins have also been observed in COPD; of note, SP-D levels were increased in COPD patients and were associated with increased exacerbation risk (Agusti et al., Clinics In Chest Medicine, 35: 131-141, 2014). Reduction in serum SP-D levels by inhaled or systemic corticosteroids was associated with disease improvement (Agusti et al., Clinics In Chest Medicine, 35: 131-141, 2014). Although lipid levels could be influenced by lipid-lowering drugs such as statins, LPA species levels were not significantly different between patients with or without statin use in this study, and corticosteroid inhaler use was limited to one inhaler per patient; hence, the differences in lipidomics between LPA species subgroups were unlikely to be due to these concomitant medications. Moreover, the alterations in lipid species showed the most overlap between LPA18:0 and LPA18:2 in men, and male patients in the LPA18:0 high subgroup or the LPA18:2 high subgroup had the most extended period of time from baseline to next exacerbation, with the next event occurring after 135 days in these patients, as compared to within the first 25 days in the rest of the patient subgroups, suggesting that changes in these lipid species might be indicative of an imminent exacerbation.

LPA-high women had higher levels of lipid classes previously reported to be associated with emphysema and exacerbation, such as DCER and SM (Bowler et al., Am J Respir Crit Care Med, 191: 275-284, 2015), compared to LPA-low women. The increase in DCER was not significant in LPA-high men; in fact, SM levels were higher in LPA-low men compared to LPA-high men, indicating that increased levels of these sphingolipids might contribute to exacerbation in LPA-high women. Without being bound by theory, the changes in lipid species in women could also be linked to compromised lung surfactant phospholipid metabolism, as many PC and TAG family species were altered between the LPA subgroups.

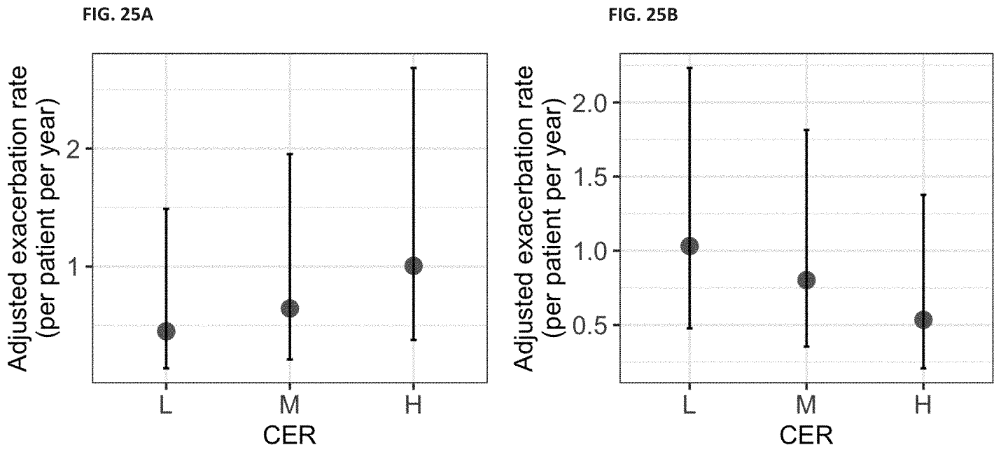

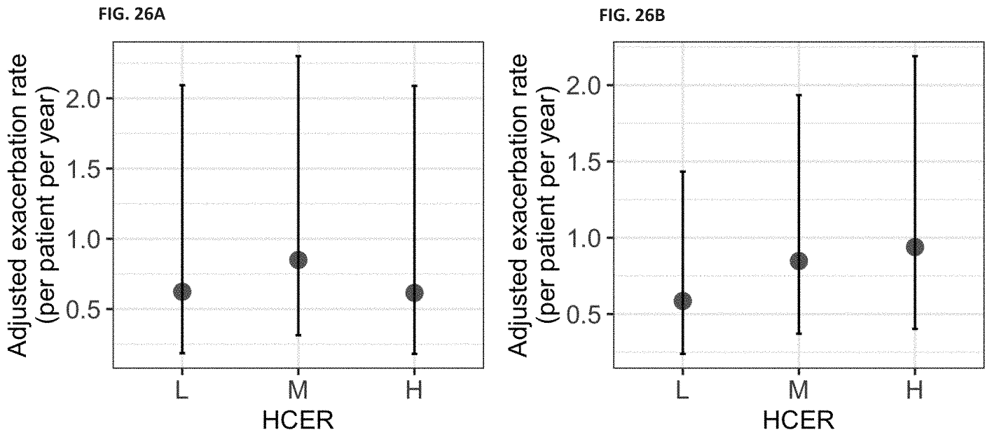

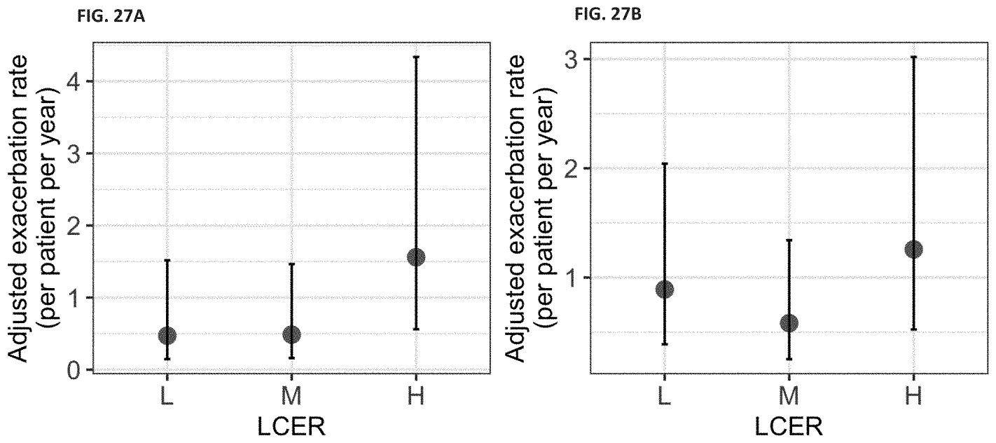

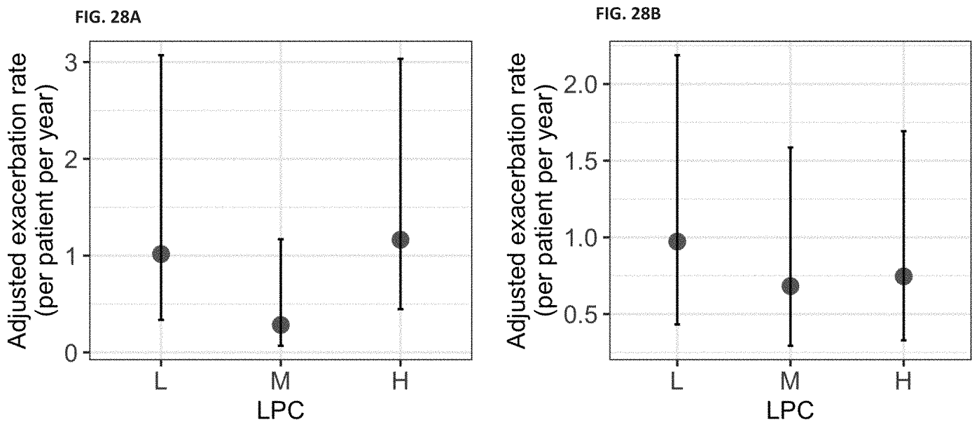

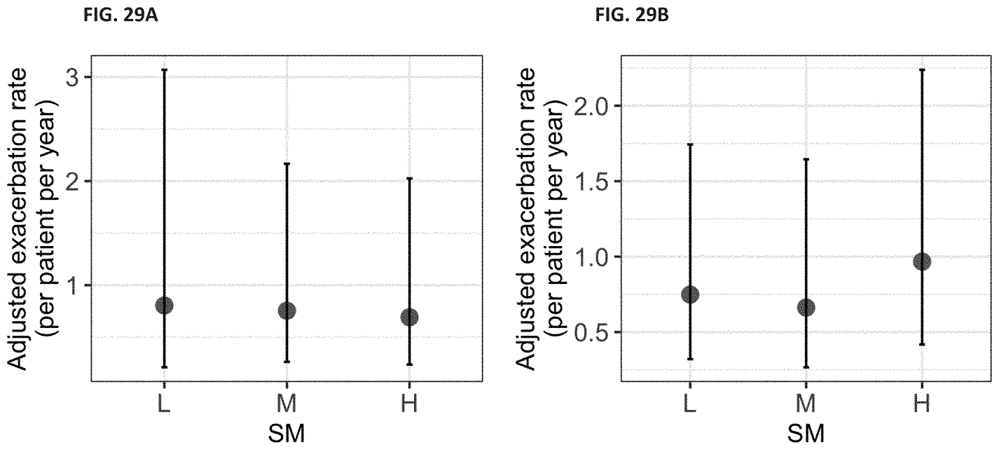

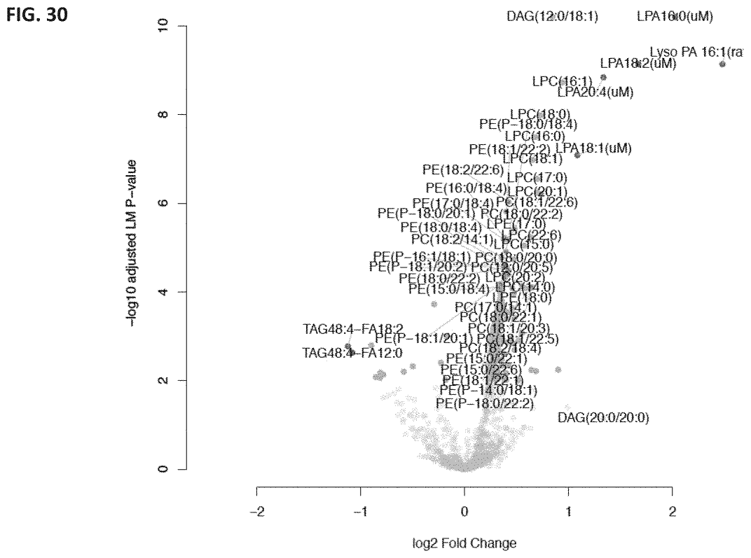

FIGS. 25A and 25B, 26A and 26B, 27A and 27B, 28A and 28B, and 29A and 29B show the adjusted exacerbation rate (per patient per year) stratified by L=lowest-; M=mid-; H=highest tertile of baseline ceramide (CER), hydroxyceramide (HCER), lactosylceramide (LCER), lysophosphatidylcholine (LPC), and sphingomyelin (SM) levels, respectively, in female and male COPD patients.

### Example 9. Plasma Lysophosphatidic Acid and Triglyceride Species are Prognostic of Disease Progression in Idiopathic Pulmonary Fibrosis

A. Background

Idiopathic pulmonary fibrosis (IPF) is a heterogenous disease of unknown etiology and high mortality with a median survival of about 3 years from the time of disease diagnosis (Adkins and Collard., Semin Respir Crit Care Med, 33(5): 433-439, 2012; Raghu et al., Am J Respir Crit Care Med, 192(2), e3-19, 2015. The rate of disease progression is highly variable among patients, but acute declines in lung function and respiratory failure occur in approximately 10% of patients every year (Ryerson et al., Eur Respir J, 46(2): 512-520, 2015). Acute exacerbations of IPF (AE-IPF) are the most common cause of death among IPF patients; their onset are unpredictable and can progress rapidly, with a 50% in-hospital mortality rate (Kulkarni and Duncan, Curr Pulmonol Rep, 8(4): 123-130, 2015). Biomarkers that could predict disease progression and AE-IPF are urgently needed to better manage the disease.

Autotaxin-lysophosphatidic acid (ATX-LPA) signaling pathway has been implicated in lung fibrosis (Shea and Tager, Proc Am Thorac Soc, 9(3), 102-110, 2012; Magkrioti et al., World Journal of Respirology, 3(3): 77, 2013). ATX generates the majority of the bioactive lipid LPA detected in blood and inflamed tissues (Knowlden and Georas, J Immunol, 192(3): 851-857, 2014; Valdes-Rives and Gonzalez-Arenas, Autotaxin-Lysophosphatidic Acid: From Inflammation to Cancer Development, p. 9173090, 2017). LPA signals through G protein-coupled LPA receptors (LPAR₁₋₆), expressed on many tissues and immune cells (Choi et al., Annu Rev Pharmacol Toxicol, 50: 157-186, 2010), to mediate lymphocyte homing (Magkrioti et al., World Journal of Respirology, 3(3): 77, 2013; Knowlden and Georas, J Immunol, 192(3): 851-857, 2014) and promote fibrosis and vascular leakage (Tager et al., Nat Med, 14(1): 45-54, 2008). LPA levels were increased in bronchoalveolar lavage fluid following lung injury in a mouse bleomycin model of pulmonary fibrosis, and deletion of one of its receptors, LPA₁, protected mice from fibrosis and mortality (Tager et al., Nat Med, 14(1): 45-54, 2008). Treatment of normal human bronchial epithelial cells with LPA caused stress fiber formation and integrin αvβ6 re-organization, leading to transforming growth factor beta (TGF-φ activation, linking LPA to TGFβ responses and establishing LPA as an important profibrotic factor (Magkrioti et al., J Autoimmun, 104: 102327, 2019).

Besides ATX, LPA can be produced by other lipid metabolism pathways. Phospholipids such as phosphatidylcholine (PC) and phosphatidylethanolamine (PE) can be converted to lysophosphatidylcholine (LPC) and lysophosphatidylethanolamine (LPE), respectively, and subsequently metabolized to LPA by phospholipases (Shea and Tager, Proc Am Thorac Soc, 9(3), 102-110, 2012). Some of these LPA precursors, as well as triglyceride (TG) species, were shown to be upregulated in IPF patients with progressive disease as compared to patients with stable disease (Nambiar et al., Respir Res, 22(1): 105, 2021).

LPA species vary in length and fatty acid saturation. In IPF patients, LPA 22:4 levels were elevated in the exhaled breath condensate compared to control (Montesi et al., Docosatetraenoyl LPA is elevated in exhaled breath condensate in idiopathic pulmonary fibrosis, 2014). LPC, a precursor of LPA, was also found to be higher in IPF patients compared to control serum in an independent study (Rindlisbacher et al., Respir Res, 19(1): 7, 2018). In a recent phase 2a study in IPF patients, an ATX inhibitor reduced plasma LPA18:2 levels by at least 50% over 12 weeks, and the reduction was accompanied by forced vital capacity (FVC) stabilization in the treated group while the placebo group showed a trend of FVC decline (Maher et al., The Lancet Respiratory Medicine, 6(8): 627-637, 2018). This further supports the idea of exploring LPA and its precursor lipids as disease biomarkers in IPF.

Because different LPA and lipid species have been reported in IPF and other respiratory diseases, we performed global lipidomic profiling as well as targeted assays that measured LPA species (LPA16:0, 16:1, 18:0, 18:1, 18:2, 20:4, and 22:4) to identify lipids that were dysregulated in IPF patients and assessed the relationship between the dysregulated lipids and disease progression.

B. Methods

Patient Cohort

Available baseline plasma samples from the placebo arm of the IPF randomized control trial CAPACITY-006 (NCT00287729) were used for LPA (n=102) and lipid (n=99) measurements. The design of the study has been described (Noble et al., The Lancet, 377(9779), 1760-1769, 2018). Briefly, patients aged 40-80 years with a diagnosis of IPF in the previous 48 months, with FVC % pred of 50-90%, diffusing capacity of carbon monoxide (DLCO) % pred of 35-90%, and 6-minute walk distance of at least 150 meters were enrolled and observed for 72 weeks. High-resolution computed topography (HRCT) was captured at screening and week 72 only. Details of other protein biomarker measurements have been described (Neighbors et al., The Lancet Respiratory Medicine, 6(8): 615-626, 2018). Age- and sex-matched healthy controls (n=30) from an internal biobank were used for comparison.

Mass Spectrometry LPA Assays

LPA species were measured using a targeted method as described above and in Li et al., J Am Soc Mass Spectrom, 2021). Briefly, 500 μl disodium phosphate buffer and 2 ml butanol were added to 20 μl plasma to extract lipids. The extracted samples were reconstituted in methanol and analyzed by liquid chromatography-mass spectrometry (LC-MS/MS), with LC coupling to a QTRAP mass spectrometer employed under negative ionization mode. HPLC separation of LPA was optimized on a C18 column to separate LPA from other lipids. Sample analysis was performed in multiple reactions monitoring mode. LPA species were identified and quantified on the basis of characteristic mass spectrometry transitions and internal standards. Additional LPA species (LPA16:0, 18:0, 18:1, 18:2, 20:4) standards (Avanti Polar Lipids, Alabaster, AL) were used to generate quantitative standard curves over a range of concentrations.

Lipidomic Profiling

Patients with sufficient remaining plasma volume (n=99) were used for lipidomic profiling. Lipidomic measurement was performed with a modified method derived from a previous study (Contrepois et al., Sci Rep, 8(1): 17747, 2018). Briefly, lipids were purified using dichloromethane, methanol and water in two extraction phases. After direct infusion, lipid species were analyzed on a SELEXION® enabled 6500 QTRAP mass spectrometer (Sciex, Redwood City, CA) in multiple reaction monitoring mode. Lipid species were identified and quantified on the basis of characteristic mass spectrometry transitions.

Statistical Analysis

Statistical analyses were performed using R (version 3.6.3). LPA and lipid concentrations were log₂ transformed when appropriate. LPA and lipid concentrations were compared between healthy controls and IPF patients, using multivariate regression adjusting for age and sex, followed by Benjamini-Hochberg correction (FDR) for multiple comparisons. FDR<0.05 was considered to be statistically significant. FVC and DLCO slope were calculated using linear regression in patients with at least three measurements within 52 weeks. The differences in HRCT indices between screening and week 72 were calculated. The relationship between LPA levels, baseline demographics, and biomarkers were assessed using univariate and multivariate linear regression adjusting for age and sex, and geographical regions (United States versus rest of the world). For FVC and DLCO decline, and changes in HRCT indices analyses, baseline FVC and DLCO were included as additional covariates. Sex-specific median levels of each LPA and TG species were used to assign patients into biomarker high (≥median) and low (<median) subgroups. Comparisons between the patient subgroups were assessed using Student t-test or Wilcoxon rank sum test for continuous measures, and Fisher's Exact test for categorical measures. Multivariate logistic regression was used to estimate the risk of exacerbation or respiratory hospitalization, or death. Cox proportional hazards regression was used to compare the time to first exacerbation or respiratory hospitalization, or death. The aforementioned covariates were included the logistic and Cox models. P-value <0.10 was considered as statistically significant.

C. Results

Differences in Lipids Between IPF and Healthy Controls

Baseline characteristics of IPF patients and the age- and gender-matched healthy controls are shown in Table 16. The available samples used in this study was representative of the overall placebo patients as there were no significant differences between the cohorts.

TABLE 16
Patient baseline characteristics

| | All | | LPA | | Global lipid | | P-value | Healthy | |
|---|---|---|---|---|---|---|---|---|---|
| | Placebo | | Placebo | | Placebo | | All vs LPA, | Controls | |
| | N = 344 | | N = 102 | | N = 99 | | All vs Global | N = 30 | |
| Age (years) | 66.9 | (7.8) | 67 | (8.0) | 66.7 | (8.0) | 0.88, 0.88 | 66..9 | (6.0) |
| Sex | | | | | | | | | |
| Female, % | 97 | (28.2%) | 28 | (27.5%) | 28 | (28.3%) | 0.98, 1.0 | 6 | (20%) |
| Male, % | 247 | (71.8%) | 74 | (72.5%) | 71 | (71.7%) | | 24 | (80%) |
| Race | | | | | | | | | |
| White, % | 340 | (98.8%) | 102 | (100%) | 99 | (100%) | 0.55, 0.56 | 27 | (90%) |
| Black or African | 3 | (0.9%) | 0 | | 0 | | | 1 | (3.3%) |
| American, % | | | | | | | | | |
| Others, % | 1 | (0.3%) | 0 | | 0 | | | 2 | (6.7%) |
| Ever smokers, % | 221 | (64.2%) | 64 | (62.7%) | 62 | (62.6%) | 0.89, 0.87 | NA | |
| Lung function | | | | | | | | | |
| FVC % predicted | 74 | (13.7) | 72.8 | (14.1) | 73 | (14.2) | 0.34, 0.41 | NA | |
| DLCO % predicted | 47.6 | (9.5) | 47.9 | (9.6) | 48.1 | (9.6) | 0.81, 0.60 | | |
| Six-minute walk | 388.5 | (86.6) | 406.4 | (95.9) | 407.1 | (96.5) | 0.10, 0.10 | NA | |
| distance (meter) | | | | | | | | | |

Data are n (%) or mean (SD). P-values compared all the placebo patients enrolled in the study versus the cohort of patients with available samples for LPA or global lipidomic profiling. FVC % predicated = percentage of predicted forced vital capacity; DLCO % predicted = percentage of predicted diffusion capacity of carbon monoxide; NA = not available.

A total of 235 lipid species were significantly upregulated in IPF patients, including many of the LPA precursors such as PC, PE, LPC and LPE species; a total of 28 lipid species were significantly downregulated in IPF compared to controls (FDR<0.05) (Table 17). Among these lipids, only seven species had a fold change greater than two: LPA16:0, 16:1, 18:1, 18:2, 20:4 and triglycerides (TG) species TG48:4:FA12:0 and TG48:4-FA18:2 (FIG. 28).

TABLE 17
Differential expression of lipid species
between IPF and healthy controls

| Upregulated | Fold Change | |
|---|---|---|
| Lipid species in IPF | IPF/control | −log10(FDR) |
| CE(20:0) | 1.984 | 1.394 |
| CE(20:1) | 1.444 | 1.944 |
| CE(22:2) | 1.866 | 2.243 |
| CE(22:6) | 1.232 | 2.008 |
| CER(24:1) | 1.196 | 1.611 |
| DAG(12:0/18:1) | 1.796 | 10.204 |
| LPC(14:0) | 1.497 | 4.086 |
| LPC(15:0) | 1.485 | 5.044 |
| LPC(16:0) | 1.600 | 7.490 |
| LPC(16:1) | 1.919 | 8.728 |
| LPC(17:0) | 1.623 | 6.551 |
| LPC(18:0) | 1.656 | 7.983 |
| LPC(18:1) | 1.574 | 6.979 |
| LPC(20:0) | 1.299 | 3.598 |
| LPC(20:1) | 1.627 | 6.239 |
| LPC(20:2) | 1.374 | 4.086 |
| LPC(20:3) | 1.223 | 1.904 |
| LPC(20:4) | 1.218 | 1.767 |
| LPC(20:5) | 1.608 | 2.212 |
| LPC(22:4) | 1.244 | 2.220 |
| LPC(22:5) | 1.284 | 2.977 |
| LPC(22:6) | 1.551 | 5.235 |
| LPE(14:0) | 1.177 | 2.243 |
| LPE(15:0) | 1.210 | 2.848 |
| LPE(16:0) | 1.325 | 3.397 |
| LPE(16:1) | 1.298 | 2.608 |
| LPE(17:0) | 1.338 | 5.182 |
| LPE(18:0) | 1.554 | 4.086 |
| LPE(20:0) | 1.289 | 3.504 |
| LPE(20:1) | 1.385 | 3.942 |
| LPE(20:2) | 1.217 | 2.237 |
| LPE(20:5) | 1.150 | 1.359 |
| LPE(22:6) | 1.268 | 3.985 |
| PC(12:0/14:1) | 1.176 | 1.753 |
| PC(12:0/16:1) | 1.247 | 2.839 |
| PC(12:0/18:1) | 1.364 | 3.519 |
| PC(12:0/18:3) | 1.320 | 2.167 |
| PC(12:0/18:4) | 1.176 | 1.838 |
| PC(12:0/20:1) | 1.282 | 3.610 |
| PC(12:0/20:2) | 1.202 | 2.247 |
| PC(12:0/20:3) | 1.299 | 3.696 |
| PC(12:0/20:4) | 1.266 | 3.222 |
| PC(12:0/20:5) | 1.338 | 4.391 |
| PC(12:0/22:2) | 1.212 | 2.340 |
| PC(12:0/22:4) | 1.176 | 1.669 |
| PC(12:0/22:5) | 1.291 | 3.635 |
| PC(12:0/22:6) | 1.190 | 2.116 |
| PC(14:0/14:1) | 1.162 | 1.455 |
| PC(14:0/18:4) | 1.202 | 2.158 |
| PC(14:0/20:1) | 1.251 | 3.074 |
| PC(14:0/20:5) | 1.308 | 2.015 |
| PC(14:0/22:1) | 1.179 | 2.167 |
| PC(14:0/22:2) | 1.188 | 1.932 |
| PC(14:0/22:6) | 1.285 | 2.085 |
| PC(15:0/14:1) | 1.261 | 3.546 |
| PC(15:0/16:1) | 1.213 | 1.655 |
| PC(15:0/18:3) | 1.179 | 1.663 |
| PC(15:0/20:5) | 1.280 | 2.340 |
| PC(15:0/22:4) | 1.230 | 2.758 |
| PC(15:0/22:5) | 1.161 | 1.304 |
| PC(15:0/22:6) | 1.218 | 1.389 |
| PC(16:0/22:1) | 1.328 | 2.858 |
| PC(16:0/22:2) | 1.380 | 2.838 |
| PC(16:0/22:6) | 1.318 | 2.015 |
| PC(17:0/14:1) | 1.282 | 4.148 |
| PC(17:0/16:1) | 1.240 | 2.167 |
| PC(17:0/18:3) | 1.253 | 2.958 |
| PC(17:0/20:5) | 1.394 | 1.475 |
| PC(17:0/22:4) | 1.198 | 2.624 |
| PC(17:0/22:6) | 1.281 | 1.932 |
| PC(18:0/14:1) | 1.215 | 2.797 |
| PC(18:0/18:1) | 1.215 | 2.102 |
| PC(18:0/20:0) | 1.384 | 4.738 |
| PC(18:0/20:1) | 1.215 | 3.126 |
| PC(18:0/22:1) | 1.303 | 4.391 |
| PC(18:0/22:2) | 1.394 | 5.429 |
| PC(18:0/22:6) | 1.394 | 2.689 |
| PC(18:1/14:1) | 1.247 | 3.133 |
| PC(18:1/16:1) | 1.340 | 2.340 |
| PC(18:1/18:1) | 1.361 | 2.954 |
| PC(18:1/18:4) | 1.235 | 3.179 |
| PC(18:1/20:1) | 1.249 | 2.708 |
| PC(18:1/20:2) | 1.372 | 3.635 |
| PC(18:1/20:3) | 1.465 | 4.139 |
| PC(18:1/20:4) | 1.282 | 1.806 |
| PC(18:1/20:5) | 1.573 | 1.802 |
| PC(18:1/22:1) | 1.270 | 3.536 |
| PC(18:1/22:2) | 1.260 | 3.610 |
| PC(18:1/22:4) | 1.464 | 3.074 |
| PC(18:1/22:5) | 1.562 | 4.112 |
| PC(18:1/22:6) | 1.667 | 6.207 |
| PC(18:2/14:1) | 1.330 | 4.497 |
| PC(18:2/18:4) | 1.272 | 4.139 |
| PC(18:2/22:1) | 1.257 | 2.977 |
| PC(18:2/22:2) | 1.180 | 1.849 |
| PC(18:2/22:6) | 1.200 | 2.251 |
| PC(20:0/16:1) | 1.280 | 2.475 |
| PC(20:0/18:1) | 1.245 | 2.063 |
| PC(20:0/18:4) | 1.215 | 2.517 |
| PC(20:0/20:2) | 1.228 | 2.688 |
| PC(20:0/20:5) | 1.260 | 1.591 |
| PC(20:0/22:2) | 1.233 | 2.599 |
| PC(20:0/22:4) | 1.257 | 2.858 |
| PC(20:0/22:5) | 1.180 | 1.715 |
| PC(20:0/22:6) | 1.322 | 3.546 |
| PE(14:0/14:0) | 1.167 | 1.805 |
| PE(14:0/14:1) | 1.279 | 3.808 |
| PE(14:0/16:1) | 1.272 | 3.506 |
| PE(14:0/18:1) | 1.277 | 1.359 |
| PE(14:0/18:2) | 1.197 | 1.846 |
| PE(14:0/18:3) | 1.237 | 3.043 |
| PE(14:0/18:4) | 1.236 | 3.470 |
| PE(14:0/20:1) | 1.147 | 1.339 |
| PE(14:0/20:2) | 1.193 | 2.213 |
| PE(14:0/20:3) | 1.214 | 2.853 |
| PE(14:0/20:4) | 1.192 | 2.340 |
| PE(14:0/20:5) | 1.200 | 2.774 |
| PE(14:0/22:1) | 1.206 | 2.573 |
| PE(14:0/22:2) | 1.182 | 2.082 |
| PE(14:0/22:4) | 1.233 | 3.443 |
| PE(14:0/22:6) | 1.163 | 1.764 |
| PE(15:0/14:1) | 1.204 | 2.714 |
| PE(15:0/16:1) | 1.201 | 2.689 |
| PE(15:0/18:1) | 1.259 | 3.706 |
| PE(15:0/18:2) | 1.196 | 2.356 |
| PE(15:0/18:3) | 1.231 | 3.352 |
| PE(15:0/18:4) | 1.268 | 4.112 |
| PE(15:0/20:3) | 1.225 | 2.912 |
| PE(15:0/20:4) | 1.175 | 2.246 |
| PE(15:0/20:5) | 1.164 | 1.803 |
| PE(15:0/22:1) | 1.306 | 4.315 |
| PE(15:0/22:2) | 1.194 | 2.805 |
| PE(15:0/22:4) | 1.198 | 3.003 |
| PE(15:0/22:5) | 1.237 | 3.230 |
| PE(15:0/22:6) | 1.266 | 4.112 |
| PE(16:0/14:0) | 1.281 | 1.753 |
| PE(16:0/14:1) | 1.224 | 2.813 |
| PE(16:0/15:0) | 1.206 | 2.477 |
| PE(16:0/16:0) | 1.197 | 1.747 |
| PE(16:0/17:0) | 1.211 | 2.620 |
| PE(16:0/18:4) | 1.305 | 5.221 |
| PE(16:0/20:1) | 1.218 | 2.617 |
| PE(16:0/22:1) | 1.247 | 3.294 |
| PE(16:0/22:2) | 1.251 | 3.621 |
| PE(17:0/14:1) | 1.247 | 3.444 |
| PE(17:0/16:1) | 1.265 | 3.635 |
| PE(17:0/18:3) | 1.220 | 2.774 |
| PE(17:0/18:4) | 1.299 | 5.179 |
| PE(17:0/20:3) | 1.234 | 3.397 |
| PE(17:0/20:5) | 1.254 | 3.397 |
| PE(17:0/22:1) | 1.173 | 1.955 |
| PE(17:0/22:2) | 1.209 | 3.054 |
| PE(17:0/22:5) | 1.261 | 3.164 |
| PE(17:0/22:6) | 1.268 | 2.885 |
| PE(18:0/14:0) | 1.220 | 2.174 |
| PE(18:0/15:0) | 1.223 | 2.488 |
| PE(18:0/16:0) | 1.165 | 1.481 |
| PE(18:0/17:0) | 1.244 | 2.794 |
| PE(18:0/18:4) | 1.305 | 4.630 |
| PE(18:0/20:1) | 1.165 | 1.820 |
| PE(18:0/22:2) | 1.323 | 4.557 |
| PE(18:1/14:1) | 1.241 | 2.184 |
| PE(18:1/16:1) | 1.245 | 2.135 |
| PE(18:1/18:4) | 1.242 | 3.546 |
| PE(18:1/20:1) | 1.287 | 2.912 |
| PE(18:1/20:2) | 1.252 | 2.624 |
| PE(18:1/20:5) | 1.320 | 1.471 |
| PE(18:1/22:0) | 1.432 | 2.904 |
| PE(18:1/22:1) | 1.271 | 4.177 |
| PE(18:1/22:2) | 1.320 | 5.813 |
| PE(18:1/22:4) | 1.428 | 2.010 |
| PE(18:1/22:5) | 1.271 | 1.342 |
| PE(18:1/22:6) | 1.362 | 1.499 |
| PE(18:2/18:4) | 1.235 | 3.368 |
| PE(18:2/20:2) | 1.159 | 1.342 |
| PE(18:2/20:3) | 1.217 | 2.608 |
| PE(18:2/20:5) | 1.245 | 3.413 |
| PE(18:2/22:1) | 1.143 | 1.368 |
| PE(18:2/22:4) | 1.276 | 3.352 |
| PE(18:2/22:5) | 1.236 | 3.137 |
| PE(18:2/22:6) | 1.339 | 6.029 |
| PE(O-16:0/14:1) | 1.181 | 2.049 |
| PE(O-16:0/16:1) | 1.261 | 3.263 |
| PE(O-16:0/18:0) | 1.232 | 2.617 |
| PE(O-16:0/18:4) | 1.210 | 2.807 |
| PE(O-16:0/20:1) | 1.209 | 3.026 |
| PE(O-16:0/20:2) | 1.192 | 2.402 |
| PE(O-16:0/22:2) | 1.177 | 2.225 |
| PE(O-18:0/14:1) | 1.197 | 2.354 |
| PE(O-18:0/16:1) | 1.200 | 2.254 |
| PE(O-18:0/18:4) | 1.184 | 2.370 |
| PE(O-18:0/20:1) | 1.206 | 2.976 |
| PE(O-18:0/20:2) | 1.207 | 2.997 |
| PE(O-18:0/20:5) | 1.319 | 1.455 |
| PE(O-18:0/22:2) | 1.204 | 2.807 |
| PE(P-14:0/18:0) | 1.211 | 2.354 |
| PE(P-14:0/18:1) | 1.269 | 4.069 |
| PE(P-14:1/18:1) | 1.229 | 2.997 |
| PE(P-16:0/14:1) | 1.208 | 2.652 |
| PE(P-16:0/16:0) | 1.156 | 1.383 |
| PE(P-16:0/16:1) | 1.239 | 1.403 |
| PE(P-16:0/18:0) | 1.189 | 2.885 |
| PE(P-16:0/18:4) | 1.208 | 3.182 |
| PE(P-16:0/20:1) | 1.163 | 1.934 |
| PE(P-16:0/20:2) | 1.221 | 2.410 |
| PE(P-16:0/22:1) | 1.221 | 2.971 |
| PE(P-16:0/22:2) | 1.229 | 3.282 |
| PE(P-16:1/18:1) | 1.334 | 4.497 |
| PE(P-18:0/14:1) | 1.259 | 3.213 |
| PE(P-18:0/16:1) | 1.280 | 1.714 |
| PE(P-18:0/18:0) | 1.240 | 3.702 |
| PE(P-18:0/18:4) | 1.316 | 4.912 |
| PE(P-18:0/20:1) | 1.282 | 4.776 |
| PE(P-18:0/20:2) | 1.260 | 2.853 |
| PE(P-18:0/22:1) | 1.152 | 1.753 |
| PE(P-18:0/22:2) | 1.298 | 4.557 |
| PE(P-18:1/14:1) | 1.183 | 2.049 |
| PE(P-18:1/16:1) | 1.392 | 3.413 |
| PE(P-18:1/18:0) | 1.196 | 2.624 |
| PE(P-18:1/18:1) | 1.217 | 1.310 |
| PE(P-18:1/18:4) | 1.184 | 2.258 |
| PE(P-18:1/20:1) | 1.268 | 4.177 |
| PE(P-18:1/20:2) | 1.304 | 4.630 |
| PE(P-18:1/20:5) | 1.575 | 1.383 |
| PE(P-18:1/22:1) | 1.228 | 2.968 |
| PE(P-18:1/22:2) | 1.150 | 1.672 |
| PE(P-18:1/22:4) | 1.562 | 2.239 |
| PE(P-18:1/22:6) | 1.315 | 2.354 |
| PE(P-18:2/22:6) | 1.206 | 2.688 |
| SM(18:1) | 1.234 | 2.504 |
| LPA16:0 | 4.070 | 10.204 |
| LPA18:1 | 2.120 | 7.085 |
| LPA18:2 | 3.186 | 9.138 |
| LPA20:4 | 2.523 | 8.841 |
| LPA 16:1 | 5.580 | 9.138 |
| Downregulated | Fold Change | |
| Lipid species in IPF | IPF/control | −log10(FDR) |
| DAG(18:1/18:2) | 0.854 | 2.402 |
| DAG(18:1/20:4) | 0.872 | 1.874 |
| DAG(18:2/18:3) | 0.887 | 2.996 |
| DAG(18:2/20:4) | 0.816 | 3.724 |
| PC(18:2/18:2) | 0.835 | 1.366 |
| PC(18:2/18:3) | 0.839 | 1.622 |
| PC(18:2/20:2) | 0.861 | 1.453 |
| PE(18:0/18:2) | 0.881 | 2.015 |
| PE(O-16:0/22:5) | 0.825 | 1.837 |
| TAG44:2-FA18:2 | 0.581 | 2.135 |
| TAG46:2-FA18:2 | 0.568 | 2.063 |
| TAG46:3-FA18:1 | 0.656 | 1.481 |
| TAG46:3-FA18:2 | 0.643 | 1.802 |
| TAG46:4-FA18:2 | 0.537 | 2.794 |
| TAG48:3-FA12:0 | 0.581 | 1.611 |
| TAG48:4-FA12:0 | 0.473 | 2.630 |
| TAG48:4-FA18:2 | 0.459 | 2.774 |
| TAG48:5-FA18:2 | 0.663 | 1.310 |
| TAG48:5-FA18:3 | 0.637 | 1.500 |
| TAG50:4-FA14:0 | 0.666 | 2.201 |
| TAG50:4-FA18:2 | 0.715 | 1.564 |
| TAG50:5-FA14:0 | 0.690 | 1.607 |
| TAG50:5-FA18:2 | 0.716 | 1.320 |
| TAG54:6-FA18:2 | 0.602 | 1.660 |
| TAG54:7-FA18:2 | 0.553 | 2.081 |
| TAG54:7-FA18:3 | 0.570 | 2.174 |
| TAG54:8-FA18:3 | 0.623 | 1.711 |
| TAG56:8-FA20:4 | 0.707 | 2.320 |

FDR = false discovery rate of multivariate regression adjusted for age and sex.

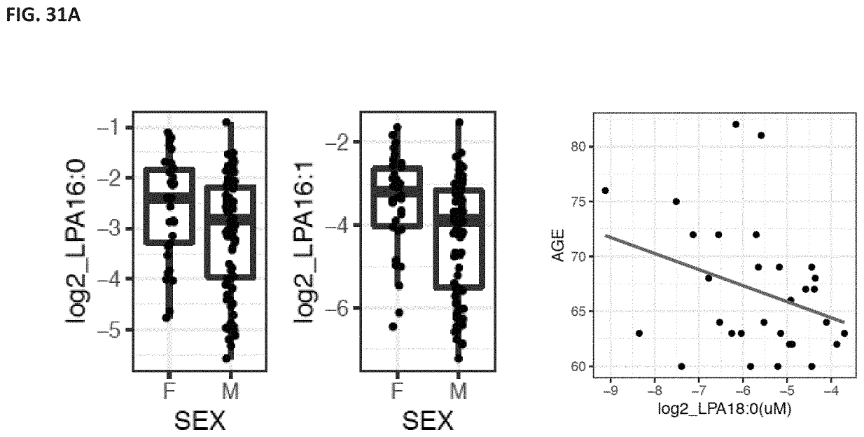

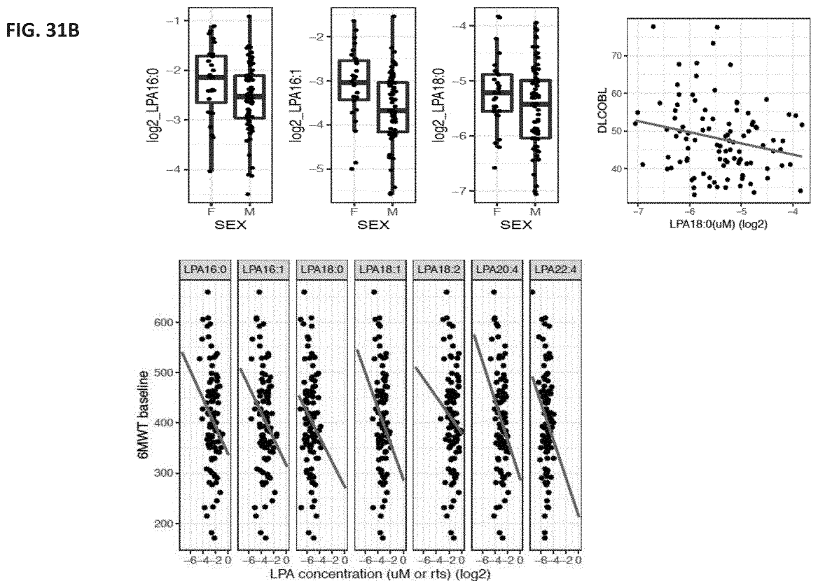

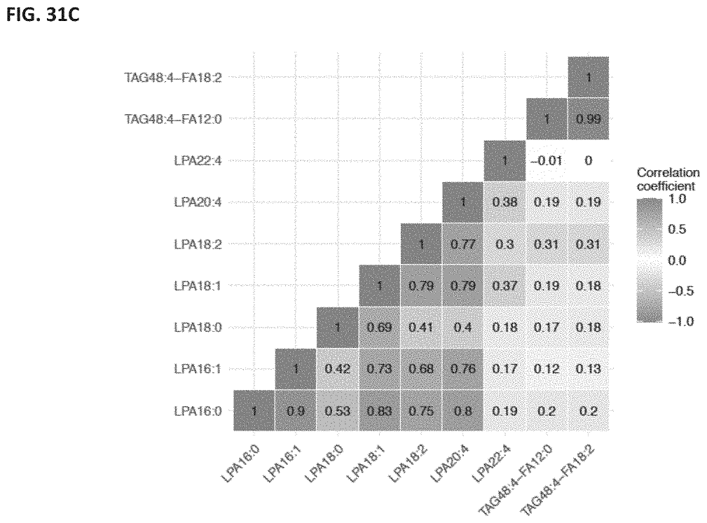

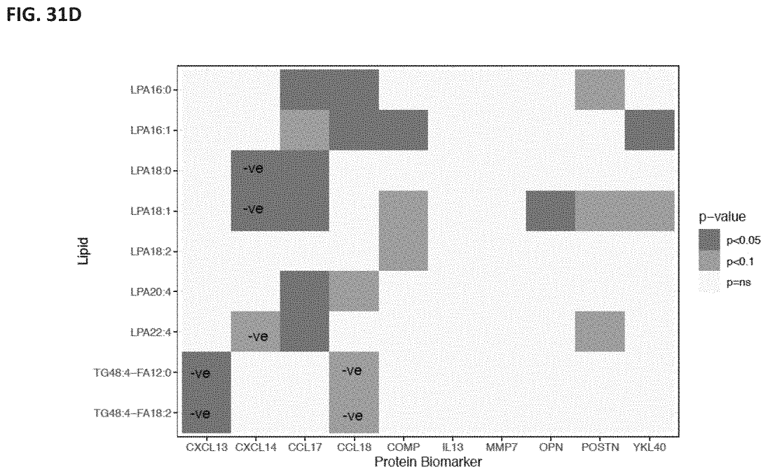

The baseline association of these seven lipids with demographic and clinical measures was assessed. In healthy controls, LPA16:0 and 16:1 were higher in females than males (p<0.05), and LPA18:0 was negatively associated with age (p<0.1) (FIG. 31A). In IPF patients, in addition to LPA16:0 and 16:1, LPA18:0 was also higher in females compared to males (p<0.1-p<0.01) and was negatively associated with baseline DLCO (p<0.05) (FIG. 31B). Except for LPA18:2, all the other LPA species were negatively associated with 6-minute walk distance (p<0.1-p<0.05) (FIG. 31B). There was no significant association between LPA levels and FVC % pred (not shown). There was no significant association between the triglyceride (TG) species and any of the above demographic or clinical measures (not shown).

In IPF patients, LPA species, except LPA22:4, were intercorrelated (rho 0.40-0.83) but showed no significant correlation with the two TG species (rho −0.01-0.31) (FIG. 31C). The TG species were highly correlated with each other (rho=0.99) (FIG. 31C). A number of LPA species were positively associated with reported prognostic biomarkers such as CCL17, CCL18, COMP, OPN, periostin, and YKL40 (p<0.05-p<0.1); while LPA18:0, 18:1 and 22:4 showed negative association with CXCL14 (p<0.05-p<0.1) (FIG. 31D). TG species had significant negative association with CXCL13 and CCL18 (p<0.05-p<0.1) (FIG. 31D).

Since some of the LPA levels were significantly different between female and male patients, sex-specific median levels of each LPA and TG species were used to categorize patients into biomarker high and low subgroups. The median cutoff concentrations or ratio-to-standards are shown in Table 18.

TABLE 18
Median cutoffs used to subgroup IPF patients
into biomarker high and low subgroups

| Lipid species | Female | Male |
|---|---|---|
| LPA16:0 (μM) | 0.227 | 0.173 |
| LPA16:1 (rts) | 0.121 | 0.078 |
| LPA18:0 (μM) | 0.027 | 0.023 |
| LPA18:1 (μM) | 0.102 | 0.098 |
| LPA18:2 (μM) | 0.408 | 0.359 |
| LPA20:4 (μM) | 0.120 | 0.130 |
| LPA22:4 (rts) | 0.029 | 0.031 |
| TG48:4-FA12:0 (μM) | 0.820 | 1.186 |
| TG48:4-FA18:2 (μM) | 1.607 | 2.173 |

μM = micromolar; rts = ratio-to-standard.

Prognostic Biomarkers of Clinical Outcomes

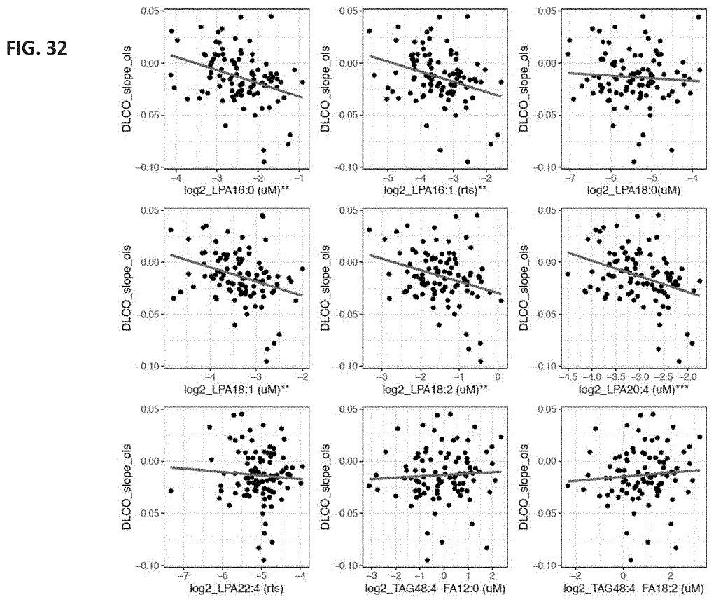

DLCO slope of decline was significantly associated with the five LPA levels that were upregulated in IPF, where patients with higher levels of LPA at baseline had greater decline in DLCO over week 52 (p<0.05-p<0.001) (FIG. 32). There was no association between TG species and DLCO decline.

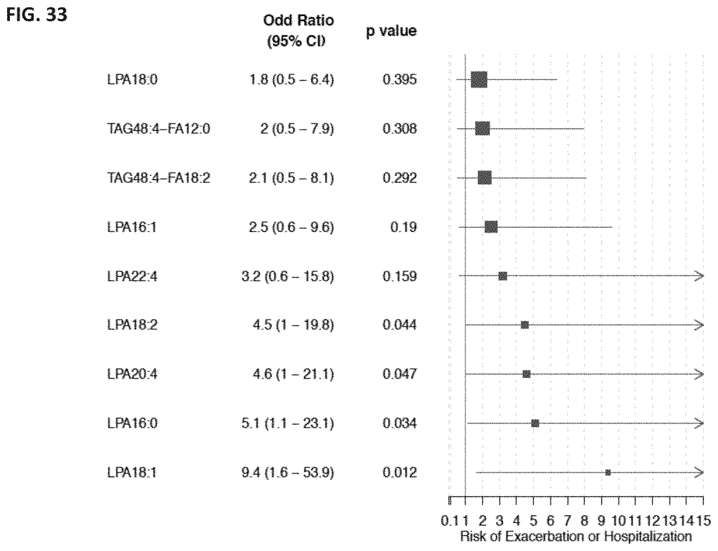

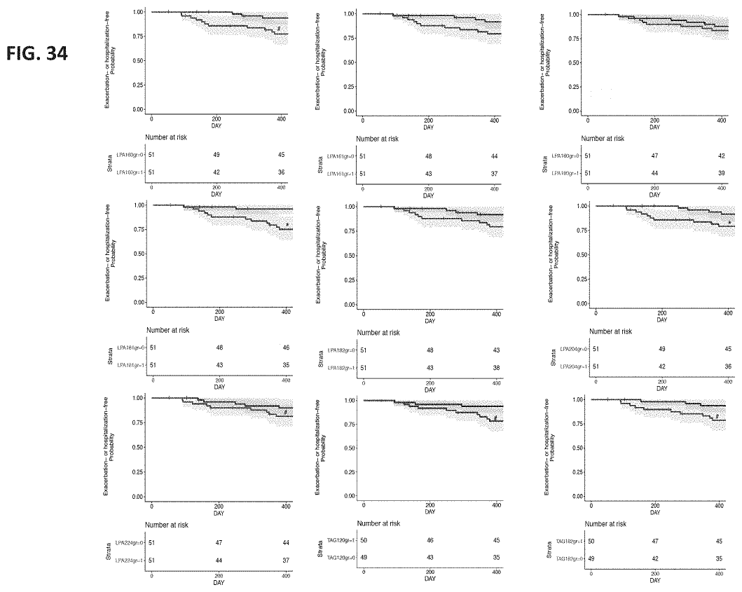

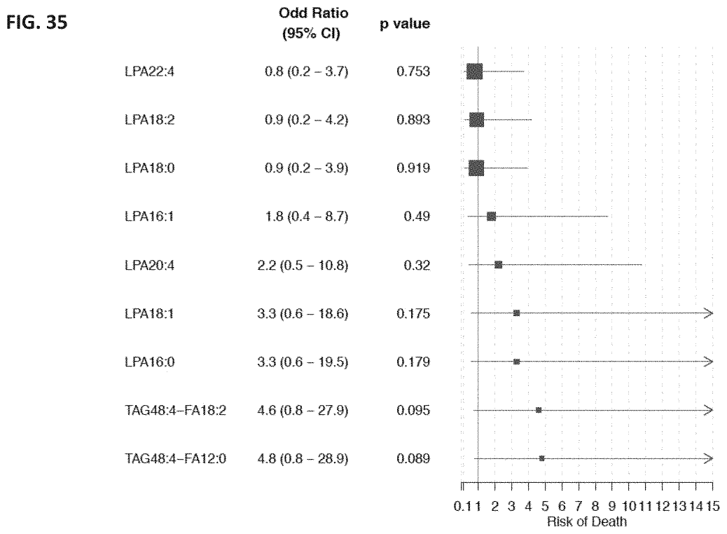

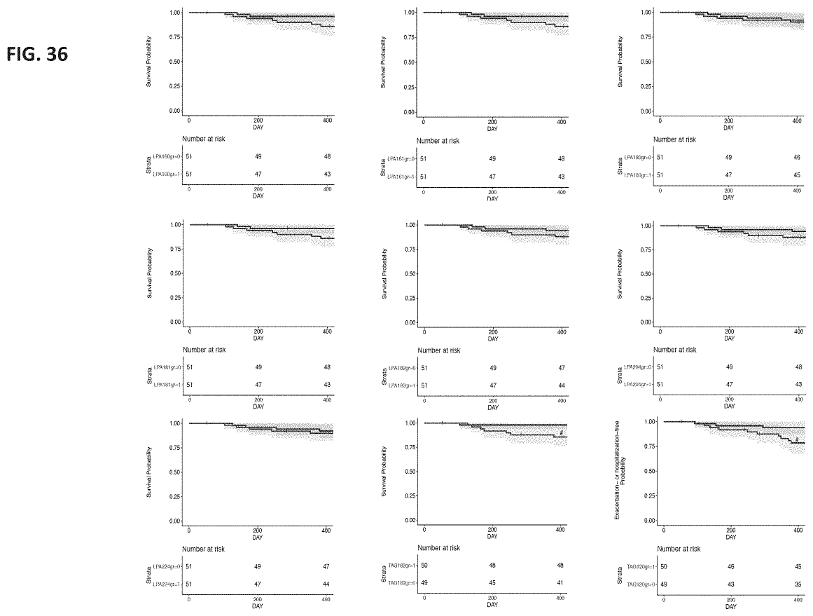

Four out of the five LPA species that were significantly upregulated in IPF-LPA16:0, 18:1, 18:2, 20:4—were prognostic of exacerbation or respiratory hospitalization, wherein patients with higher levels (≥median) of the biomarkers had increased risk of these events (odd ratio (95% CI)): LPA16:0-high=5.1 (1.1-23.1) (p=0.034); LPA18:1-high=9.4 (1.6-53.9) (p=0.012); LPA18:2-high=4.5 (1.0-19.8) (p=0.044); LPA20:4-high=4.6 (1.0-21.1) (p=0.047) (FIG. 33). Consistently, patients with higher levels of these LPA species (LPA16:0, 18:1, 20:4 and LPA22:4), or lower levels of the TG species, had earlier time to exacerbation or respiratory hospitalization compared to the respective subgroups (hazard ratio (95% CI)): LPA16:0-high=3.2 (0.9-11.7) (p=0.077); LPA18:1-high=5.2 (1.1-23.8) (p=0.034); LPA20:4-high=4.8 (1.3-18.4) (p=0.022); LPA22:4-high=4.6 (1.0-21.5) (p=0.050); TAG48:4-FA12:0-low=2.8 (0.8-9.4) (p=0.093); TAG48:4-FA18:2-low=3.0 (0.8-10.1) (p=0.079) (FIG. 34). The TG species were prognostic of mortality as patients with lower levels of the TG species had increased risk of mortality (odd ratio (95% CI)): TG48:4-FA12:0-low=4.8 (0.8-28.9) (p=0.089), TAG48:4-FA18:2-low=4.6 (0.8-27.9) (p=0.095) (FIG. 35) as well as earlier time to death within 52 weeks (hazard ratio (95% CI): TG48:4-FA12:0-low=4.4 (0.8-23.0) (p=0.081); TAG48:4-FA18:2-low=4.3 (0.8-22.5) (p=0.086) (FIG. 36).

Prognostic Biomarkers of Radiographic Changes

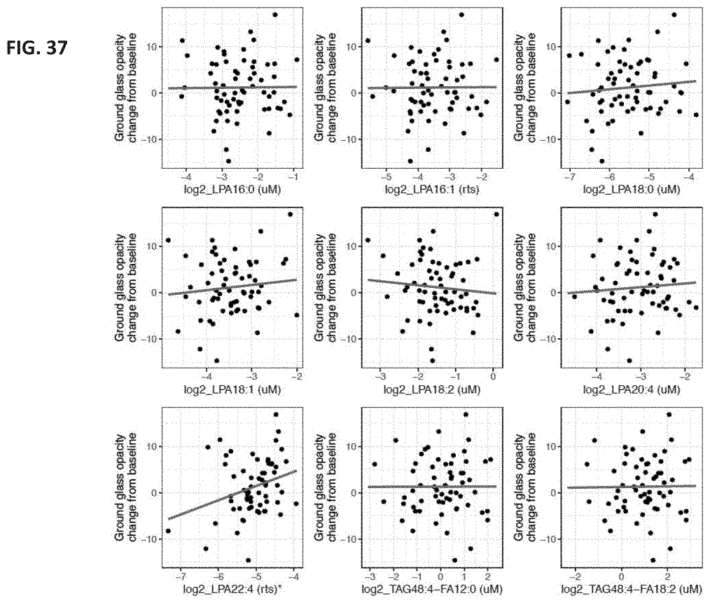

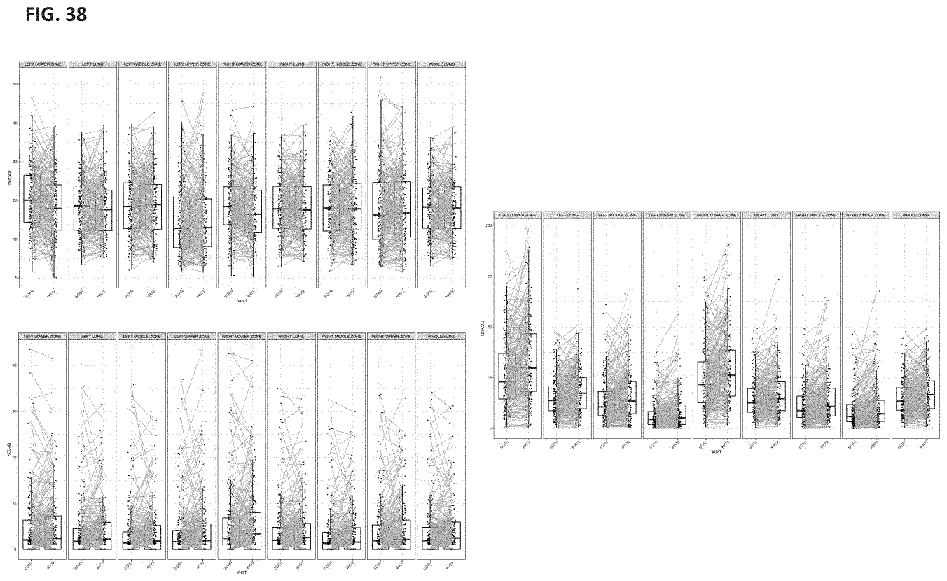

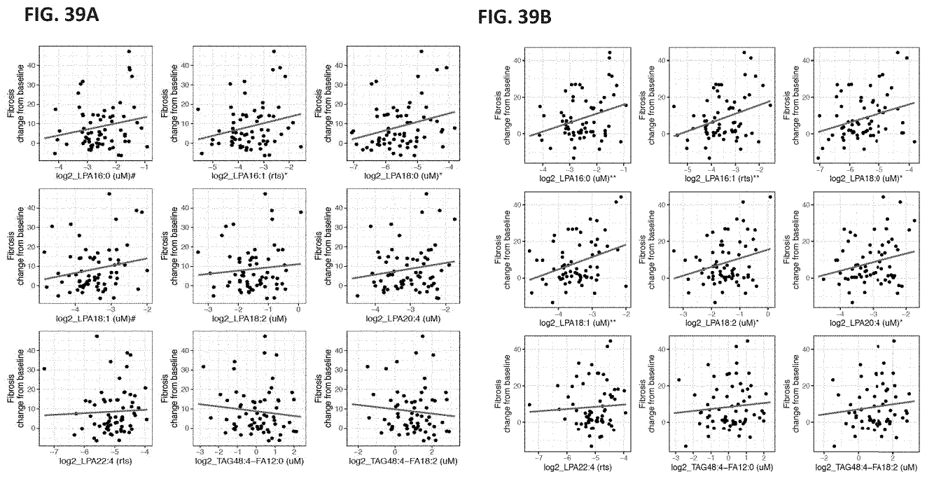

Patients with higher levels of LPA22:4 at baseline had greater increases in the overall ground glass opacity in both lungs at week 72 (p<0.05) (FIG. 37). IPF is recognized on HRCT by subpleural lower lobe reticular opacities and honeycombing (Contrepois et al., Sci Rep, 8(1): 17747, 2018). Interestingly, honeycombing progression is the fastest in the lower lobes of the lungs (Araawa et al., AJR AM J Roentgenol, 196(4): 773-782, 2011). Radiographic changes by lung regions (lower, middle, and upper) were investigated, and the increases in honeycombing and fibrosis at week 72 were observed to occur mostly in the lower lobes (FIG. 38). Honeycombing is considered the end-stage of fibrosis; the decreases observed in some patients could be within the noise of the HRCT measurements. All of the LPA species, except LPA22:4, were prognostic of fibrosis increase in the lower zones of the lungs at week 72, wherein patients with higher baseline levels of LPA had greater increase in fibrosis (p<0.1-p<0.5) (FIGS. 39A and 39B). TG species were not prognostic of radiographic changes (FIGS. 37, 39A, and 39B).

D. Discussion

In this study, a number of LPA and TG species that were significantly dysregulated in IPF patients were identified. Some of these lipid species were prognostic of clinical and radiographic outcomes including DLCO decline, exacerbation or respiratory hospitalization, mortality, and increases in ground glass opacity, and fibrosis in the lungs.

LPA in the systemic circulation is predominantly from activated platelets, but could also be produced by other cells including fibroblasts (Yang et al., World J Gastroenterol, 24: 4132-4151, 2018), macrophages, or inflammatory cells in the form of vesicles to be transported to the site of injury (Fourcade et al., Cell, 80(6): 919-927, 1995; Jethwa et al., J Cell Sci, 129(20): 3948-3957, 2016). LPA promotes monocyte migration (Takeda et al., Int J Mol Sci, 20(6), 2019) and mediates the differentiation of monocytes to macrophages (Ray and Rai, Blood, 129(9): 1177-1183, 2017). Interestingly, monocyte recruitment via LPA signaling has been shown to be crucial for the resolution of tissue inflammation (McArthur et al., J Immunol, 195(3): 1139-1151, 2015). In this study, a number of the LPA species (LPA16:0, 16:1 and 20:4) and both TG species showed significant association with inflammation related biomarkers CCL18 and CCL17; CCL18 is a biomarker of M2 macrophage with fibrogenic properties and has been shown to be a predictor of FVC decline and mortality (Neighbors et al., The Lancet Respiratory Medicine, 6(8): 615-626, 2018). Monocytes preferentially differentiate into M2 macrophages under Th2 inflammation, and Th2 cytokine such as CCL17 has been shown to contribute to the development of pulmonary fibrosis in bleomycin mouse model (Belperio et al., J Immunol, 173(7): 4692-4698, 2004). The association of LPA and TG species with CCL 8 and CCL17 in the systemic circulation suggest that these lipid species contribute to disease progression via M2 macrophage activity or Th2-mediated responses leading to fibrosis.

IPF lungs have elevated levels of palmitic acid (C16:0) compared with control subjects (Chu et al., Am J Respir Cell Mol Biol, 61(6): 737-746, 2019). Exogenous addition of palmitic acid to epithelial cells increased reactive oxygen species and apoptosis (Sunaga et al., Nat Commun, 4: 2563, 2013). Moreover, deficiency of stearoyl CoA desaturase-1 that catalyzes the conversion of saturated to monounsaturated fatty acid resulted in ER stress and fibrosis in mice (Romero et al., Am J Respir Cell Mol Biol, 59(2): 225-236, 2018). These data suggest that lipotoxicity due to the accumulation of saturated fatty acids may contribute to fibrosis by inducing apoptosis in epithelial cells and ER stress. Transcriptomic analyses corroborate the findings as genes involved in lipid metabolism were dysregulated in IPF lungs as well as the alveolar type 2 cells isolated from IPF patients (Reyfman et al., Am J Respir Crit Care Med, 199(12): 1517-1536, 2019). These findings are not unexpected as the pulmonary surfactant produced by alveolar type 2 cells is made up of a complex mixture of lipids (phospholipids, TG, others) and surfactant proteins. Changes in lipid metabolism in the lungs are reflected in the respiratory compartment and the systemic circulation; phospholipid levels in the bronchoalveolar lavage have been shown to correlate with disease severity (Suryadevara et al., Int J Mol Sci, 21(12), 2020, and systemic levels of surfactant protein A and D are found to be prognostic of mortality in IPF (Greene et al., Eur Respir J, 19(3): 439-446, 2002). Taken together, it is plausible that the increased levels of LPA and TG in the systemic circulation observed in this study are a reflection of the dysregulated lipid or surfactant metabolism in IPF lungs.

A growing body of evidence implicates the role of endothelial dysfunction and increased alveolar-capillary permeability in IPF pathogenesis (Probst et al., Eur RespirJ, 56(1), 2020). Pro-inflammatory and pro-fibrotic mediators including TGF-β1 and LPA activate Rho-kinase signaling in endothelium, leading to cytoskeletal reorganization and increased endothelial permeability (Probst et al., Eur Respir J, 56(1), 2020; van Nieuw Amerongen et al, Arterioscler Thromb Vasc Biol, 20(12): E127-133, 2000). Inhibiting Rho-kinase signaling by pharmacological inhibitors reduce LPA-induced increases in vascular permeability in mouse models of lung injury (van Nieuw Amerongen et al, Arterioscler Thromb Vasc Biol, 20(12): E127-133, 2000) and attenuate bleomycin-induced fibrotic response (Shimizu et al., Am J Resp Crit Care Med, 163(1): 210-217, 2001). Consistently, the deletion of LPA1 receptor reduced vascular leakage in bleomycin mouse model of fibrosis (Tager et al., Nat Med, 14(1): 45-54, 2008). Deterioration in endothelial dysfunction or vascular permeability could potentially manifest clinically as a decline in DLCO, as DLCO is a marker of alveolar-capillary interface integrity that measures the gas transfer capacity of the capillary interface and the volume of blood available for gas exchange (Roughton et al., J Appl Physiol, 11(2): 290-302, 1957). The link between DLCO and in endothelial dysfunction or vascular permeability could explain why patients with higher levels of LPA had greater decline in DLCO which was accompanied by increases in ground glass opacity and fibrosis in the lungs.

Ground glass opacity is associated with fibrotic thickening of alveolar septa and intra-alveolar granulation tissue, but it can also be associated with alveolar inflammation (American Thoracic Society, Am J Respir Crit Care Med, 161(2 Pt. 1), 646-664, 2000). The distribution of ground glass and fibrosis in IPF lungs has been linked to acute exacerbation and mortality (Tcherakian et al., Thorax, 66(3): 226-231, 2011; Sokai et al., ERJ Open Res, 3(2), 2017). Patients with asymmetrical disease, defined as having fibrosis in one lung that was 1.5 fold greater than that of another lung, had significantly higher rate of acute exacerbations compared to patients with symmetrical disease (Tcherakian et al., Thorax, 66(3): 226-231, 2011). Patients with both lungs affected symmetrically with ground glass during AE-IPF had higher mortality rate within 6 months (Sokai et al., ERJ Open Res, 3(2), 2017). We observed that HRCT changes were variable by lung zones. Importantly, LPA levels were associated with ground glass and fibrosis increase in whole lung and lower regions of the lungs, respectively, and these changes were accompanied by worse outcomes including an increase in the risk of AE-IPF or respiratory hospitalization, establishing evidence that the LPA signaling pathway plays an important role in these radiographic and clinical manifestations.

It is unclear why LPA and TG species were prognostic of some but not all of the clinical and radiographic outcomes in this study. LPA mediates downstream signaling through receptor activation. Six LPA receptors have been reported and are expressed at different levels on different cell types (Choi et al., Annu Rev Pharmacol Toxicol, 50: 157-186, 2010), and as a result, both the protective as well as pathogenic roles of LPA in the airway have been reported. LPA receptors are required to maintain epithelial barrier function, control allergic lung inflammation (He et al., J Biol Chem, 284(36): 24123-24132, 2009; Park et al., Am J Respir Crit Care Med, 188(8): 928-940, 2013), and support alveolarization (Funke et al., Am J Respir Cell Mol Biol, 55(1): 105-116, 2016). Related to lung pathology, LPA receptor activation leads to lung fibrosis (Tager et al., Nat Med, 14(1): 45-54, 2008; Gan et al., Bichem Biophys Res Commun, 409(1): 7-13, 2011), epithelial cell apoptosis (Funke et al., Am J Respir Cell Mol Biol, 46(3): 355-364, 2012), inflammatory cytokines production and neutrophilic infiltration (Cummings et al., J Biol Chem, 279(39): 41085-41094, 2004).

E. Conclusion

IPF has poor prognosis, and patients with AE-IPF may have increased mortality and morbidity.

Early identification of high-risk patients is imperative to prompt the initiation of appropriate treatments to preserve lung function. In this study, plasma LPA and TG species that were significantly dysregulated in IPF patients were identified, these LPA and TG species were prognostic of clinical outcomes, but only LPA species were prognostic of radiographic changes in the lung.

### Example 10. Plasma LPA, LPC, and LPE in Idiopathic Pulmonary Fibrosis Patients

A. Methods

Levels of lipid species were assessed in baseline plasma samples from the patient cohort described in Example 9 (n=97, 69 males and 28 females from the CAPACITY-006 study). 30 age- and sex-matched healthy controls were included for comparison as described above.

Levels of the LPA species LPA16:0, LPA16:1, LPA18:0, LPA18:1, LPA18:2, LPA20:4, and LPA22:4 were measured as described above and in Li et al., J Am Soc Mass Spectrom, 2021). The Lipidyzer platform was used for global lipidomic profiling.

Statistical analysis was performed as follows: LPA and lipid concentrations were log 2 transformed. The relationship between lipid levels and baseline characteristics were assessed using univariate or multivariate linear regression adjusting for age and sex. To assess the prognostic effects of lipids, DLCO (diffusing capacity of carbon monoxide) or FVC (forced vital capacity) change (as slope), and absolute change in HRCT (high-resolution computed tomography) quantified metrics (percentage of ground glass, honeycombing, fibrosis, and interstitial lung disease in lungs) were calculated and multivariate linear regression adjusting for age, sex, height, baseline DLCO or FVC, and geographic region was used. A Cox proportional hazards regression model adjusted for the aforementioned covariates was used for mortality analysis. Median levels of lipids were used as cutoffs to subgroup patients into biomarker-high (median) and biomarker-low (<median) subgroups. P-value <0.1 was deemed significant.

B. Results for LPA Species

LPA Baseline Characteristics

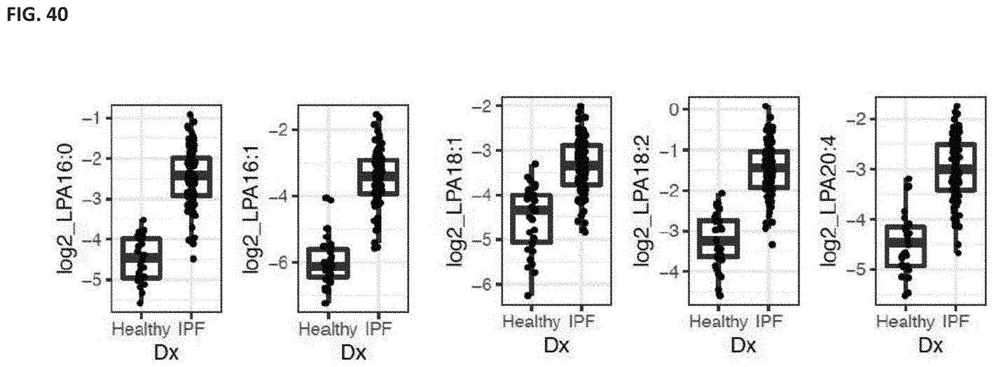

As discussed above, five LPA species were found to be significantly upregulated in IPF patients compared to controls, in univariate or multivariate regression adjusted for age and sex: LPA16:0, LPA16:1, LPA18:1, LPA18:2, and LPA20:4 (FIG. 40).

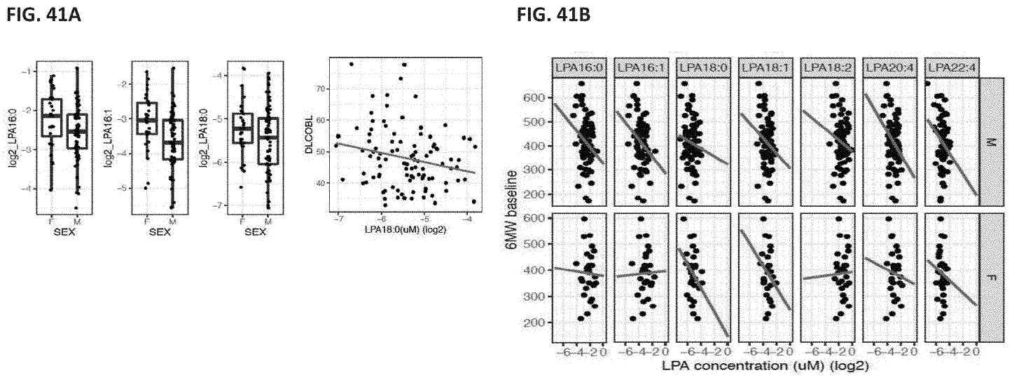

In IPF patients, LPA16:0, LPA16:1 and LPA18:0 were higher in females compared to males (FIG. 41A). LPA18:0 was negatively associated with DLCO, in univariate or multivariate regression adjusted for age and sex (FIG. 41A). Five LPA species were negatively associated with 6 MWD (6-minute walk distance), in univariate or multivariate regression adjusted for age and sex: LPA16:0, LPA16:1, LPA18:1, LPA20:4, and LPA22:4 (FIG. 41B).

Prognostic Effects in Male Patients

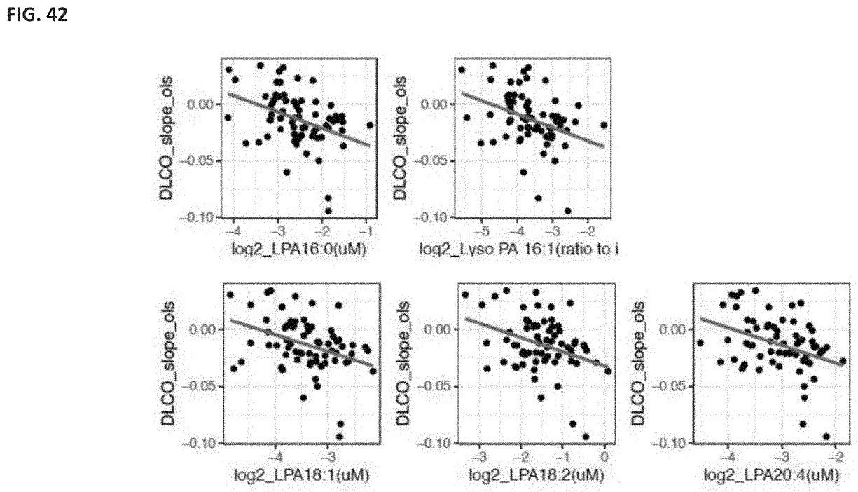

Over 48 weeks, the following LPA species were prognostic of DLCO decline in male patients: LPA16:0, LPA16:1, LPA18:1, LPA18:2, and LPA20:4 (FIG. 42).

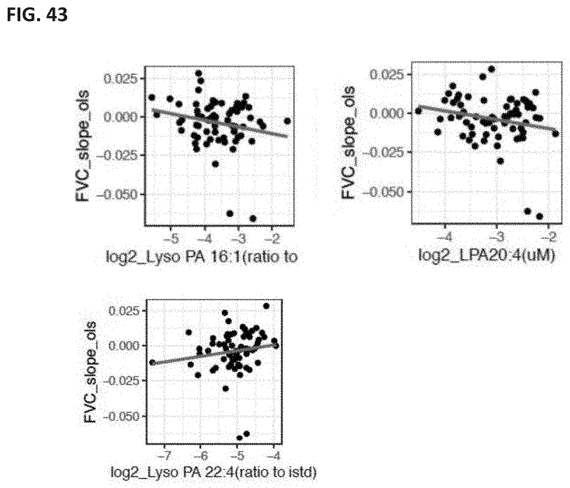

Over 48 weeks, the following LPA species were prognostic of FVC decline in male patients: LPA16:1, LPA18:1, LPA20:4, and LPA22:4 (FIG. 43). Patients with higher levels of LPA16:1 and LPA20:4 had greater decline in FVC over 48 weeks, whereas patients with lower levels of LPA22:4 had greater decline in FVC over 48 weeks.

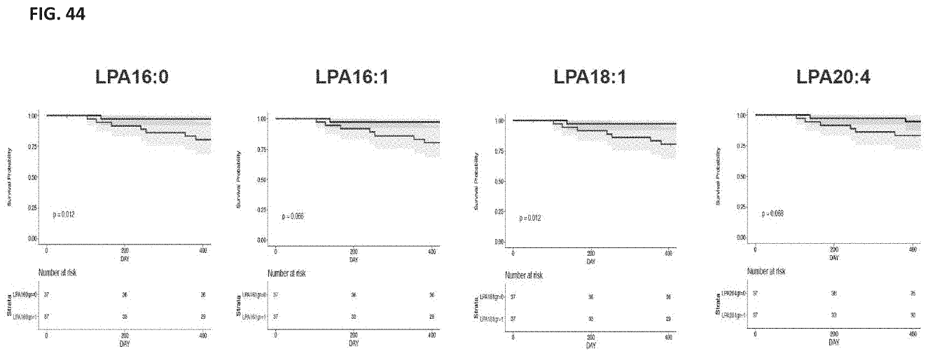

Over 48 weeks, the following LPA species were prognostic of mortality in male patients: LPA16:0, LPA16:1, LPA18:1, LPA20:4 (FIG. 44). Patients with higher levels of these species had earlier time to death.

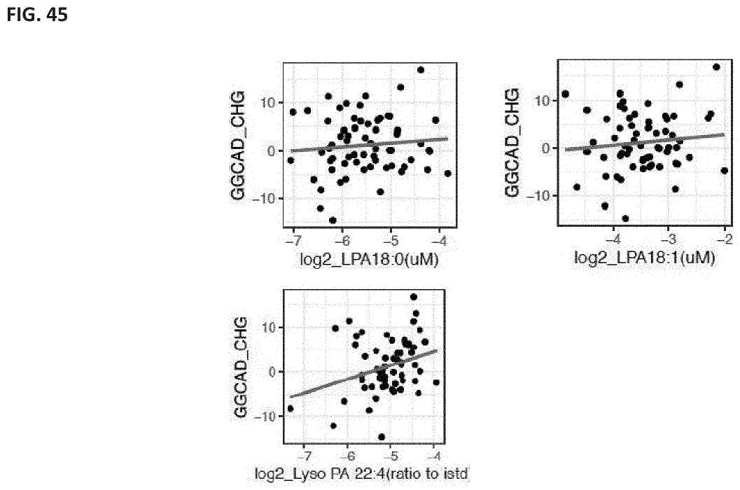

Over 72 weeks, the following LPA species were prognostic of increased ground glass opacity in male patients: LPA18:0, LPA18:1, and LPA22:4 (FIG. 45). Patients with higher levels of these LPA species had greater increase in ground glass opacity at week 72.

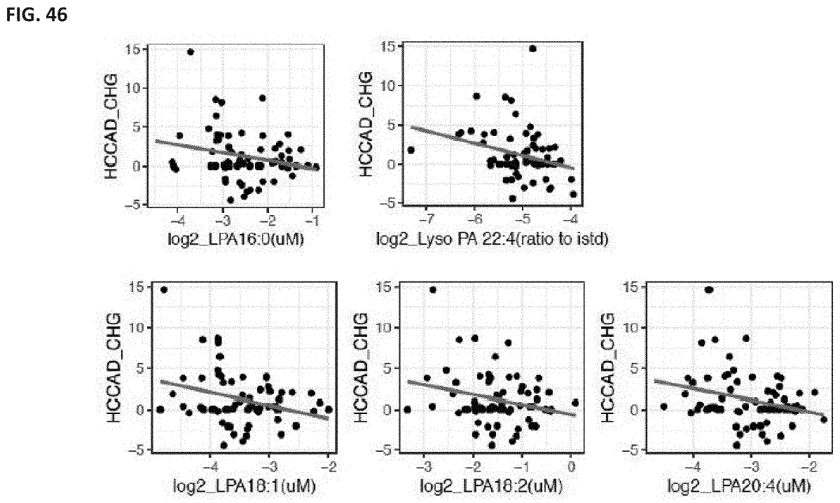

Over 72 weeks, the following LPA species were prognostic of increased honeycombing in male patients: LPA16:0, LPA16:1, LPA18:1, LPA18:2, and LPA20:4 (FIG. 46). Patients with lower levels of these LPA species had greater increase in honeycombing at week 72.

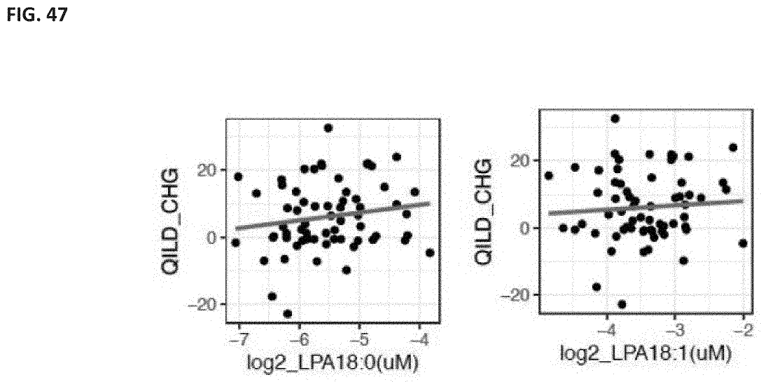

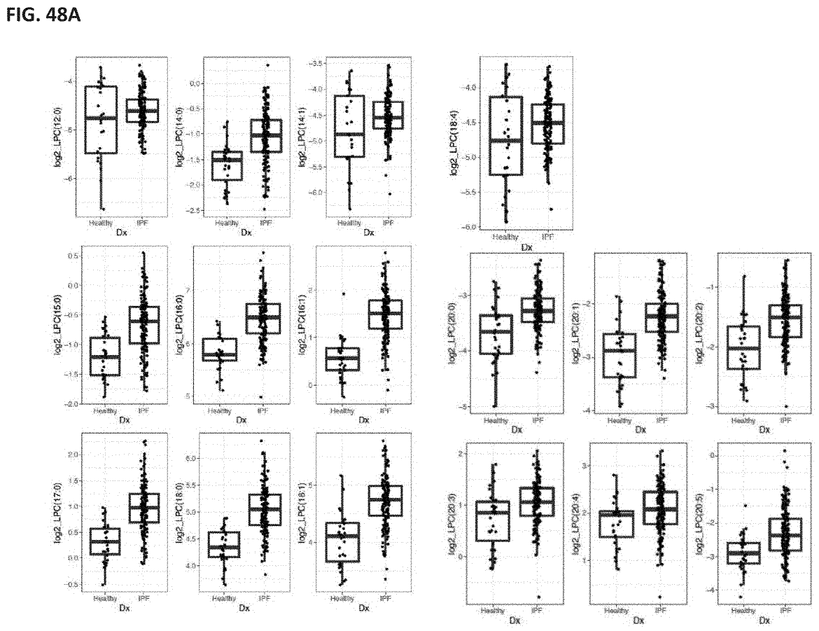

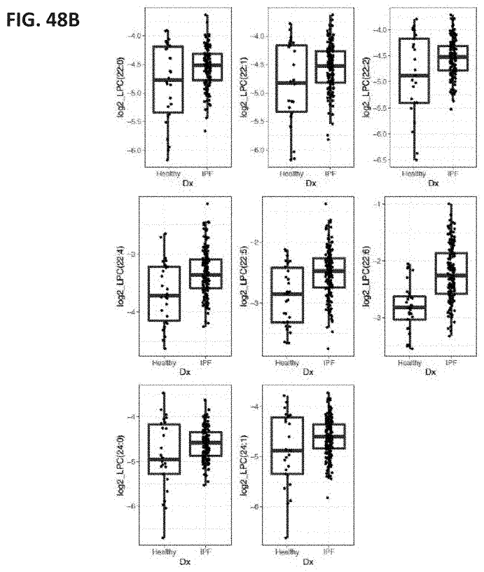

Over 72 weeks, the following LPA species were prognostic of interstitial lung disease (ILD) metric (ground glass opacity, honeycombing, and fibrosis) increase in male patients: LPA18:0 and LPA18:1 (FIG. 47). Patients with higher levels of these LPA species had greater increase in ILD metric at week 72.

C. Results for LPC Species

LPC Species Upregulated in IPF Patients

Twenty-four LPC species were found to be significantly upregulated in IPF patients compared to controls in univariate or multivariate regression adjusted for age and sex: LPC12:0, LPC14:0, LPC14:1, LPC15:0, LPC16:0, LPC16:1, LPC17:0, LPC18:0, LPC18:1, LPC18:4, LPC20:0, LPC20:1, LPC20:2, LPC20:3, LPC20:4, LPC20:5, LPC22:0, LPC22:1, LPC22:2, LPC22:4, LPC22:5, LPC22:6, LPC24:0, and LPC24:1 (FIGS. 48A and 48B).

Prognostic Effects of LPC Species in all Patients

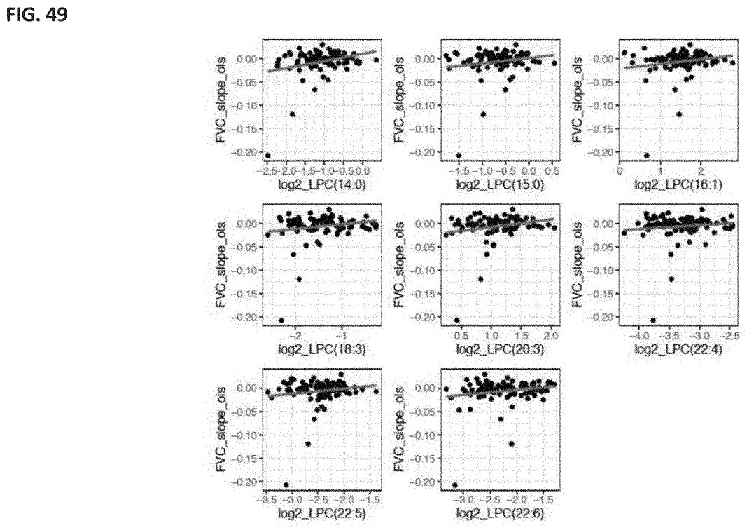

Over 48 weeks, the following LPC species were prognostic of FVC decline: LPC14:0, LPC15:0, LPC16:1, LPC18:3, LPC20:3, LPC22:4, LPC22:5, and LPC22:6 (FIG. 49). Patients with lower levels of these LPC species had greater decline in FVC over 48 weeks.

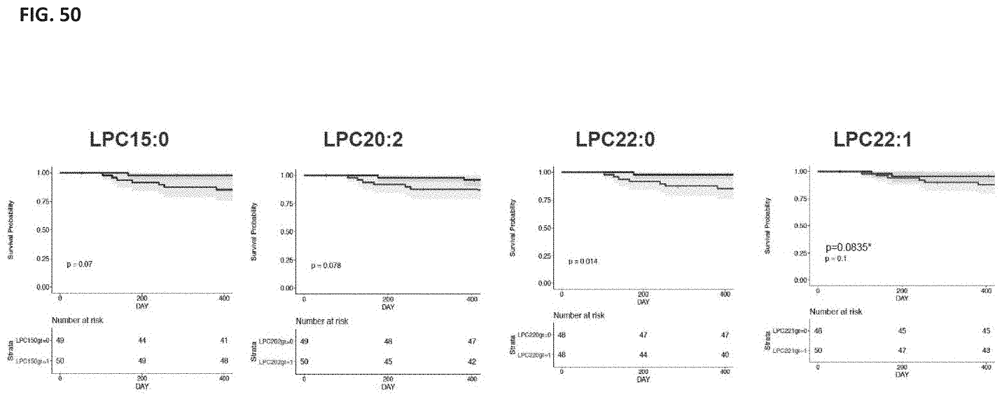

Over 48 weeks, the following LPC species were prognostic of mortality: LPC15:0, LPC20:2, LPC22:0, and LPC22:1 (FIG. 50). Patients with lower levels (<median) of LPC15:0 or higher levels (median) of LPC20:2 LPC22:0, or LPC22:1 had earlier time to death.

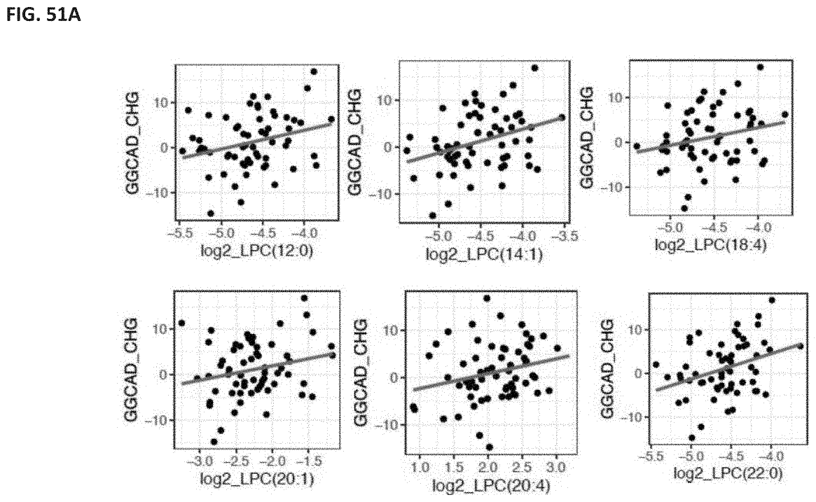

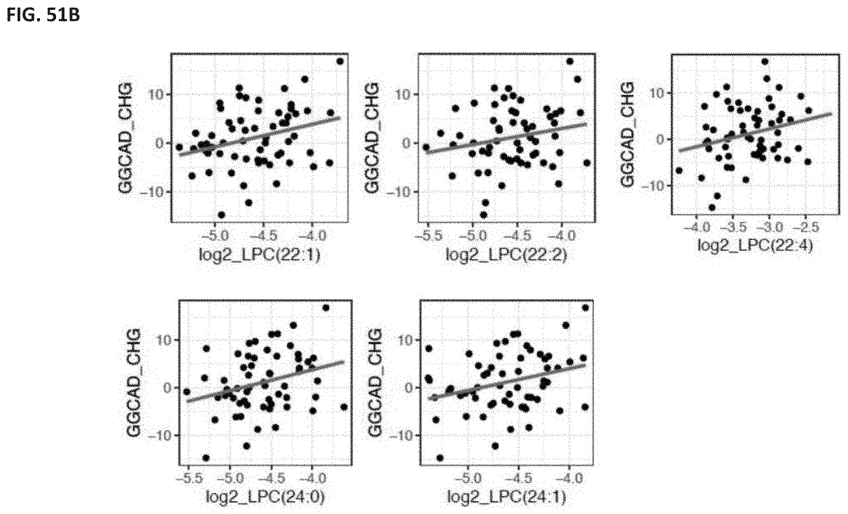

Over 72 weeks, the following LPC species were prognostic of increased ground glass opacity: LPC12:0, LPC14:1, LPC18:4, LPC20:1, LPC20:4, LPC22:0, LPC22:1, LPC22:2, LPC22:4, LPC24:0, and LPC24:1 (FIGS. 51A and 51B). Patients with higher levels of these LPC species had greater increase in ground glass opacity at week 72.

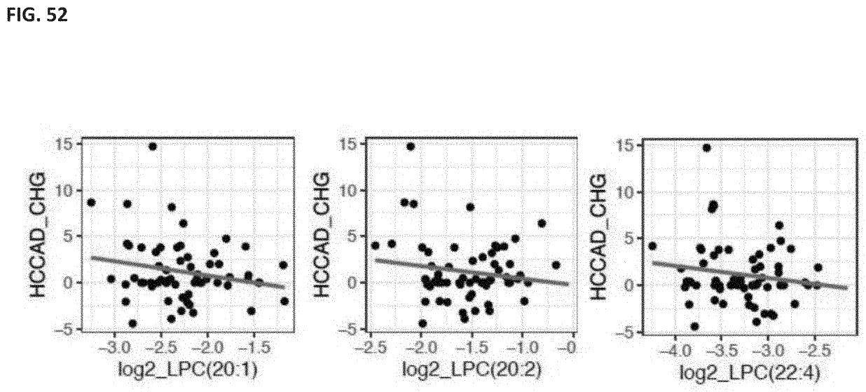

Over 72 weeks, the following LPC species were prognostic of increased honeycombing: LPC20:1, LPC20:2, and LPC22:4 (FIG. 52). Patients with lower levels of these LPC species had greater increase in honeycombing at week 72.

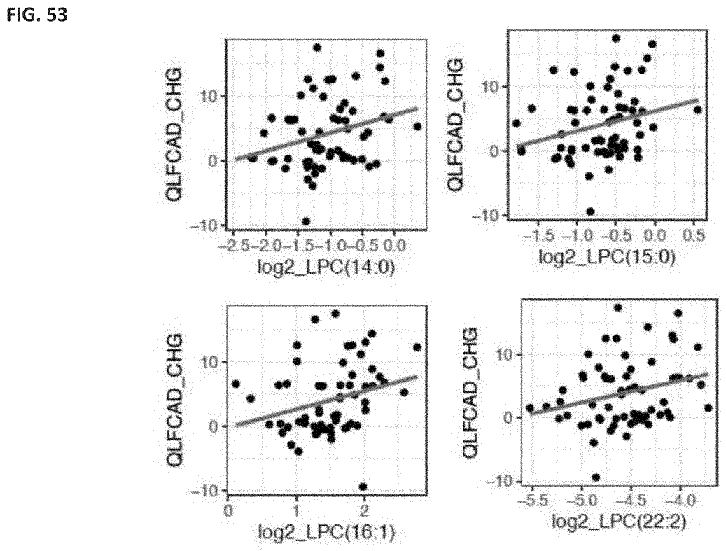

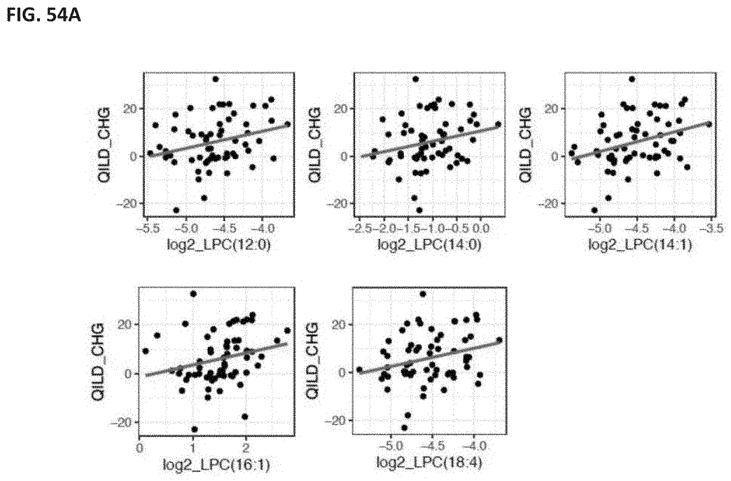

Over 72 weeks, the following LPC species were prognostic of increased fibrosis: LPC14:0, LPC15:0, LPC16:1, and LPC22:2 (FIG. 53). Patients with higher levels of these LPC species had greater increase in fibrosis at week 72.

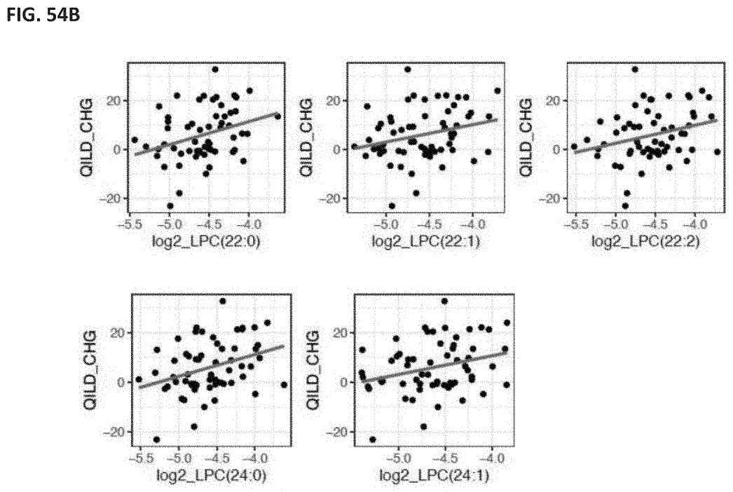

Over 72 weeks, the following LPC species were prognostic of ILD metric increase: LPC12:0, LPC14:0. LPC14:1, LPC16:1, LPC18:4, LPC22:0, LPC22:1, LPC22:2, LPC22:4, LPC24:0, and LPC24:1 (FIGS. 54A and 54B). Patients with higher levels of these LPC species had greater increase in ILC metric at week 72.

D. Results for LPE Species

LPE Species Upregulated in IPF Patients

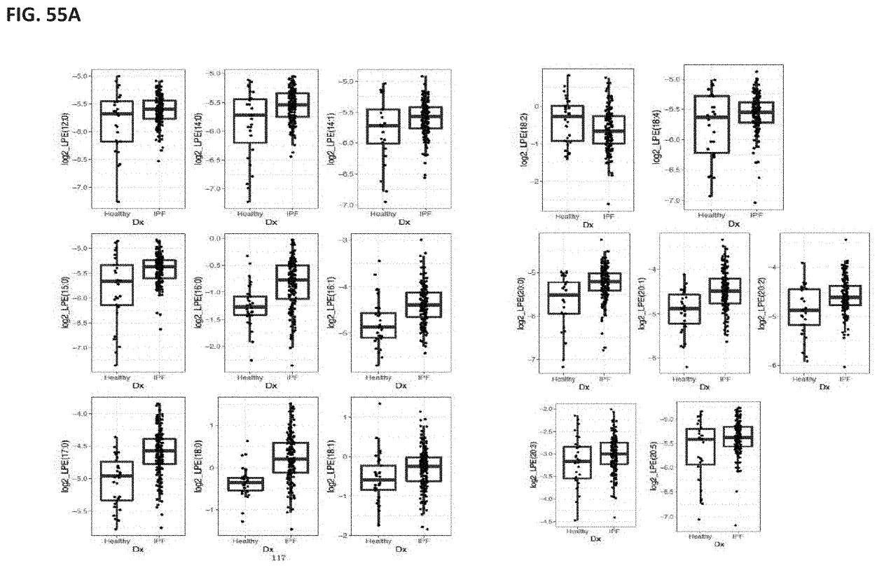

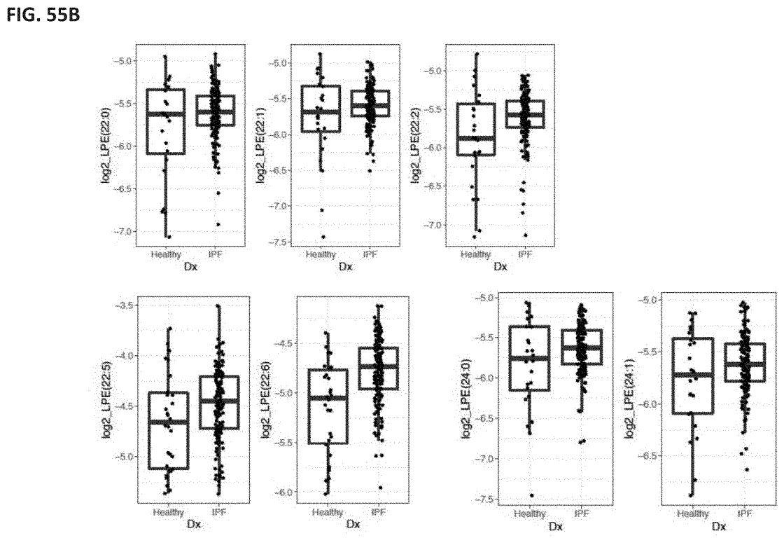

Twenty-three LPE species were found to be significantly upregulated in IPF patients compared to controls, in univariate or multivariate regression adjusted for age and sex: LPE12:0, LPE14:0, LPE14:1, LPE15:0, LPE16:0, LPE16:1, LPE17:0, LPE18:0, LPE18:1, LPE18:2, LPE18:4, LPE20:0, LPE20:1, LPE20:2, LPE20:3, LPE20:5, LPE22:0, LPE22:1, LPE22:2, LPE22:5, LPE22:6, LPE24:0, and LPE24:1 (FIGS. 55A and 55B).

Prognostic Effects of LPE Species in all Patients

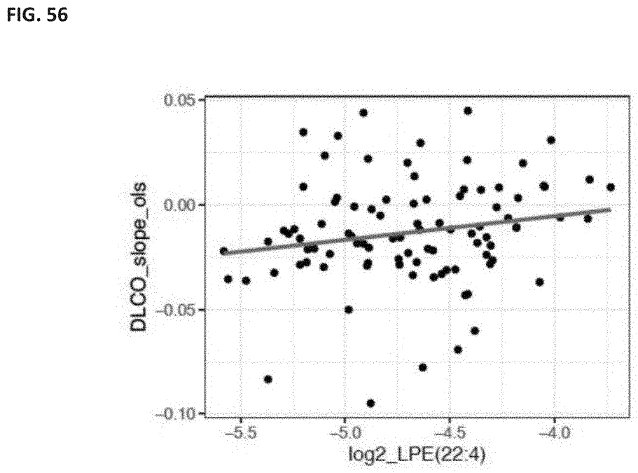

Over 48 weeks, the following LPE species was prognostic of DLCO decline: LPE22:4 (FIG. 56). Patients with lower levels of LPE22:4 had greater decline in DLCO over 48 weeks.

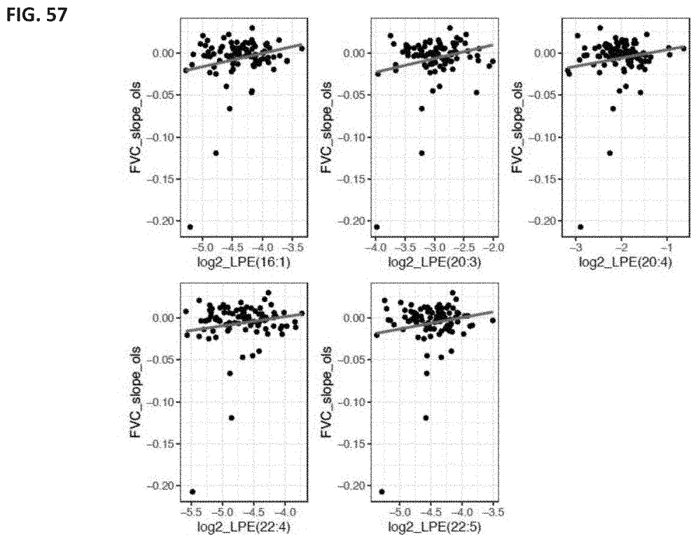

Over 48 weeks, the following LPE species were prognostic of FVC decline: LPE16:1, LPE20:3, LPE20:4, LPE22:4, and LPE22:5 (FIG. 57). Patients with lower levels of these LPE species had greater decline in FVC over 48 weeks.

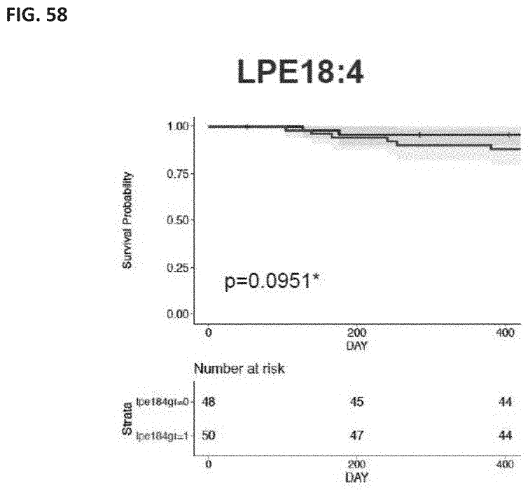

Over 48 weeks, the following LPE species was prognostic of mortality: LPE18:4 (FIG. 58). Patients with higher levels (median) of LPE18:4 had earlier time to death.

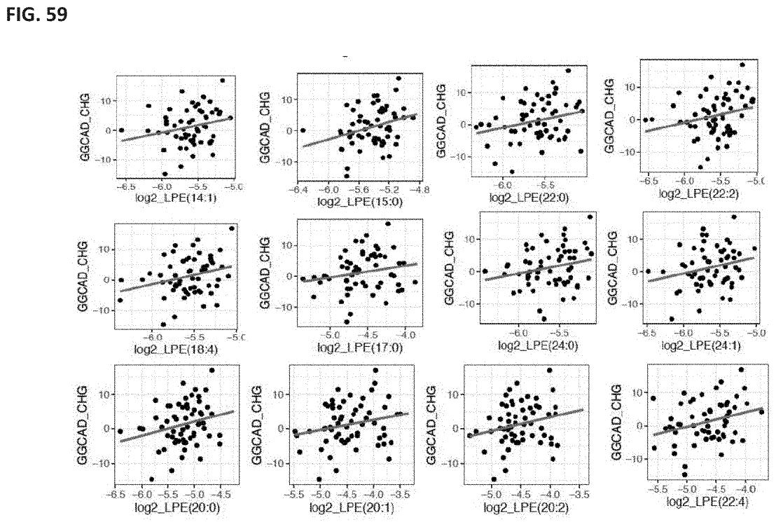

Over 72 weeks, the following LPE species were prognostic of increased ground glass opacity: LPE14:1, LPE15:0, LPE17:0, LPE18:4, LPE20:0, LPE20:1, LPE20:2, LPE22:0, LPE22:2, LPE22:4, LPE24:0, and LPE24:1 (FIG. 59). Patients with higher levels of these LPE species had greater increase in ground glass opacity at week 72.

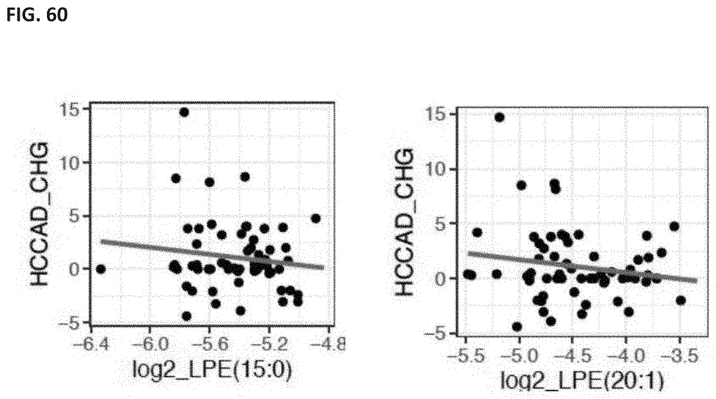

Over 72 weeks, the following LPE species were prognostic of increased honeycombing: LPE15:0 and LPE20:1 (FIG. 60). Patients with lower levels of these LPE species had greater increase in honeycombing at week 72.

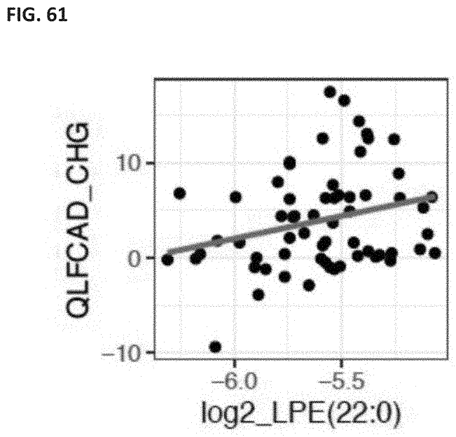

Over 72 weeks, the following LPE species was prognostic of fibrosis: LPE22:0 (FIG. 61). Patients with higher levels of LPE22:0 had greater increase in fibrosis at week 72.

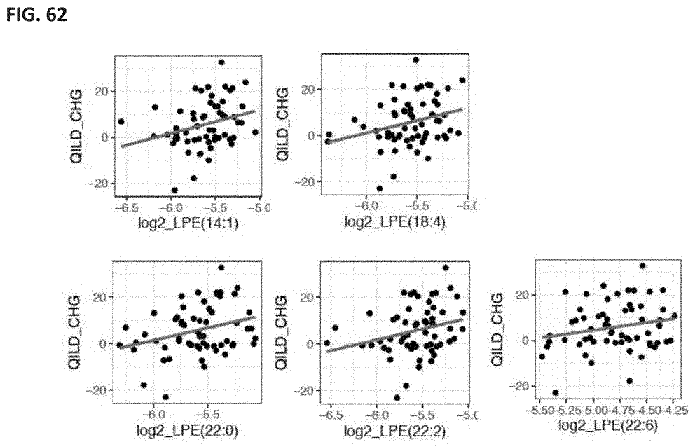

Over 72 weeks, the following LPE species were prognostic of ILD metric increase: LPE14:1, LPE18:4, LPE22:0, LPE22:2, and LPE22:6 (FIG. 62). Patients with higher levels of these LPE species had greater increase in ILD metric at week 72.

### Example 11. Plasma Lipidomics Results

A. Methods

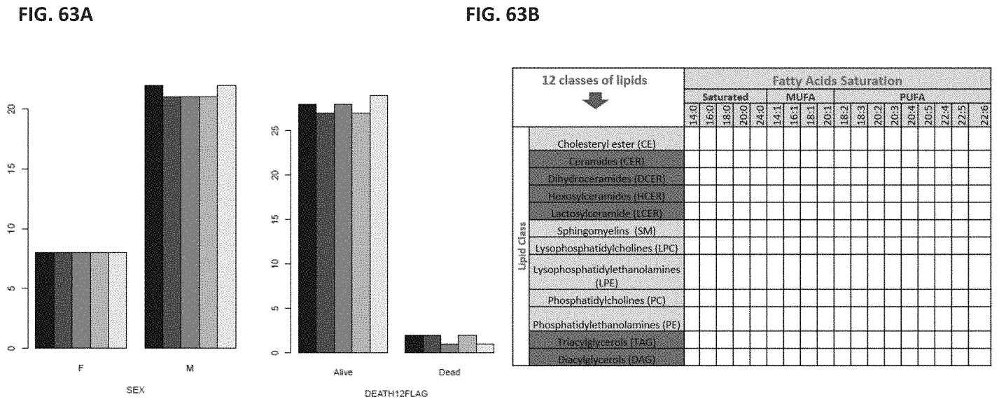

Levels of lipid species were assessed as described above for 151 IPF patient samples and 30 healthy patient samples. Characteristics of the sample population are provided in FIG. 63A. 20 μL of each plasma sample was used for the LPA assay, and 50 μL was used for global profiling of lipids, as summarized in FIG. 63B. The effects of demographics on lipid levels and the correlation of lipids with disease status, fibrosis and clinical biomarkers were assessed using univariate statistical analysis.

B. Results

Lipid Species Differing Between Control and Healthy Patients

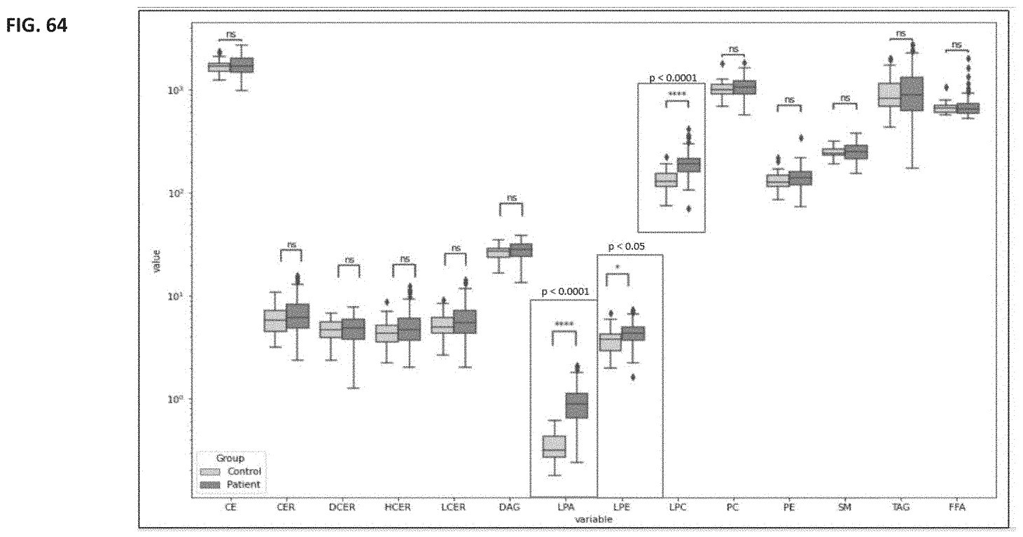

Lipid species that changed significantly in IPF plasma compared with a healthy control are shown in FIG. 64. Levels of LPA, LPC, and LPE were significantly increased; levels of these lipids increase during inflammation.

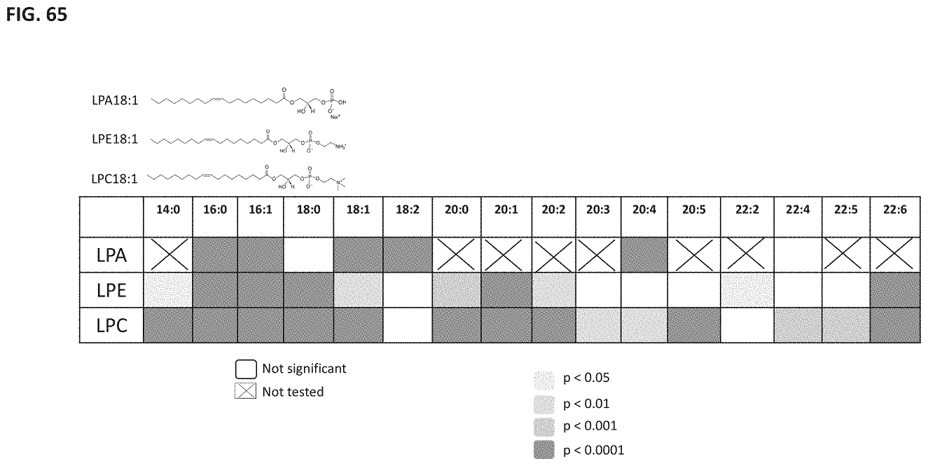

Specific fatty acid (FA) tails that were significantly increased in IPF patient samples relative to controls are shown in FIG. 65.

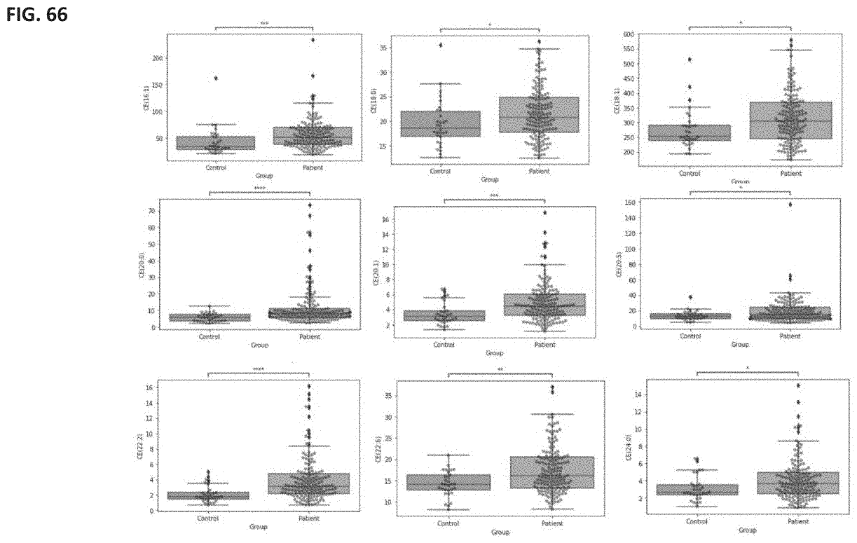

Certain ceramide (CE) species increased significantly in the IPF patient group: CE (16:1), CE (18:0), CE (18:1), CE (20:0), CE (20:1), CE (20:5), CE (22:2), CE (22:6), and CE (16:1) (FIG. 66).

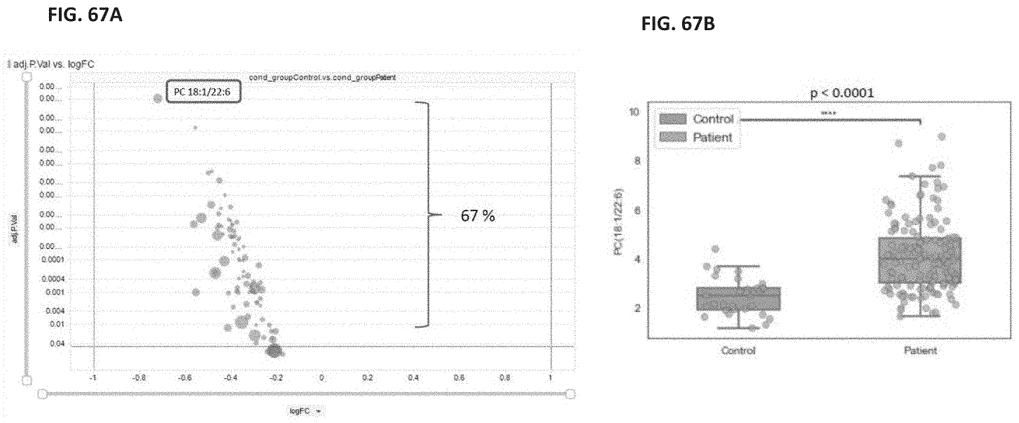

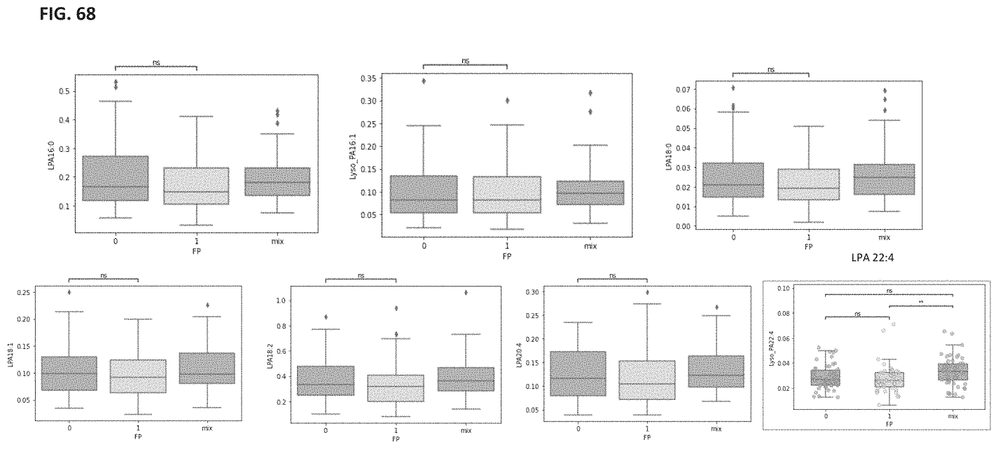

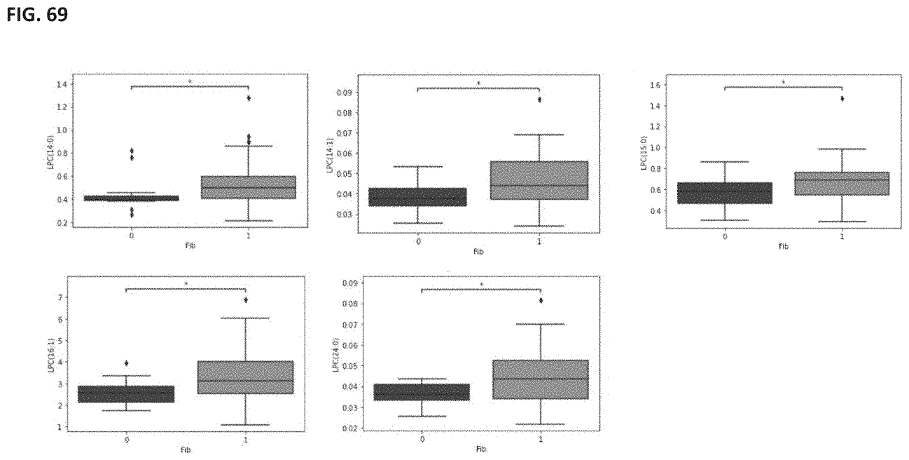

About 67% of phosphatidylcholine (PC) species were significantly increased in the IPF patient group, as shown in FIGS. 67A and 67B. The increased fold changes were less than 2. PC 18:1/22:6 had the most significant fold change and p value.

143 of the 216 phosphatidylethanolamine (PE) species assessed were significantly different between control patients and IPF patients.

Levels of LPA22:4 differed significantly between disease progressors (defined as ≥10% absolute FVC_{% pred} decline in 12 months, n˜31) and non-progressors (defined as no change or increase in absolute FVC_{% pred} in 12 months, n˜46) (FIG. 68).

The LPC species LPC14:0, LPC14:1, LPC15:0, LPC16:1, and LPC24:0 were significantly higher in patients who experienced fibrosis compared to the non-fibrosis group (FIG. 69).

Lipid Species Correlating with Biomarkers

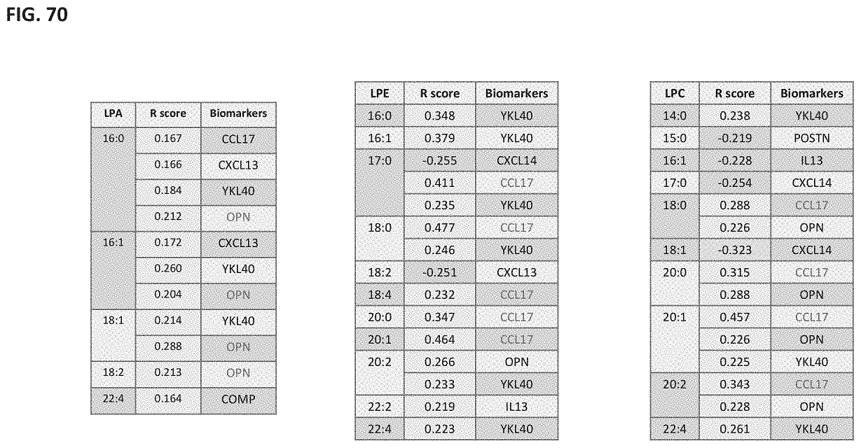

Some LPA, LPE, and LPC species correlated with clinical biomarkers for IPF, as shown in FIG. 70 (p<0.05).

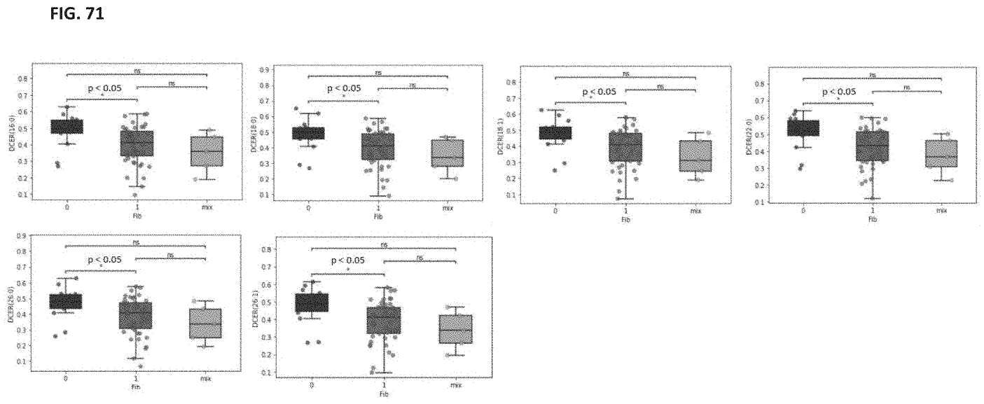

The dihydroceramide (DOER) species DOER (16:0), DOER (18:0), DOER (18:1), DOER (22:0), DOER (26:0), and DOER (26:1) differed significantly in patients who experienced fibrosis compared to the non-fibrosis group (FIG. 71).

Table 19 summarizes correlations between DOER species and biomarkers. CXCL14 had a positive correlation with DOER; IL13 and YKL4 had a negative correlation with DOER.

TABLE 19
Correlation between DCER and biomarkers

Lipid

| Species | Negative Correlation | | Positive Correlation | |
|---|---|---|---|---|
| DCER(14:0) | YKL40 | r = −0.240(p = 0.026) | | |
| DCER(16:0) | YKL40 | r = −0.234(p = 0.030) | CXCL14 | r = 0.215(p = 0.046) |
| DCER(18:0) | YKL40 | r = −0.234(p = 0.030) | CXCL14 | r = 0.222(p = 0.040) |
| DCER(18:1) | IL13 | r = −0.215(p = 0.047) | CXCL14 | r = 0.221(p = 0.040) |
| | YKL40 | r = −0.243(p = 0.030) | | |
| DCER(20:1) | IL13 | r = −0.226(p = 0.037) | CXCL14 | r = 0.222(p = 0.040) |
| | YKL40 | r = −0.228(p = 0.035) | | |
| DCER(22:0) | IL13 | r = −0.223(p = 0.039) | CXCL14 | r = 0.224(p = 0.038) |
| | YKL40 | r = −0.222(p = 0.040) | | |
| DCER(22:1) | IL13 | r = −0.259(p = 0.016) | CXCL14 | r = 0.232(p = 0.032) |
| DCER(24:0) | IL13 | r = −0.276(p = 0.010) | | |
| DCER(24:1) | IL13 | r = −0.276(p = 0.010) | CXCL14 | r = 0.219(p = 0.042) |
| DCER(26:0) | IL13 | r = −0.219(p = 0.043) | CXCL14 | r = 0.214(p = 0.048) |
| | YKL40 | r = −0.237(p = 0.028) | | |
| DCER(24:1) | IL13 | r = −0.217(p = 0.044) | | |
| | YKL40 | r = −0.236(p = 0.029) | | |

Table 20 summarizes correlations between hexosylceramide (HCER) species and biomarkers. CXCL13, CXCL14, and CCL18 showed a positive correlation with HOER. There was no negative correlation between HOER and biomarkers.

TABLE 20
Correlation between HCER and biomarkers

| | |
|---|---|
| Lipid Species | Positive Correlation |

| | | |
|---|---|---|
| HCER(14:0) | CXCL14 | r = 0.216(p = 0.046) |
| HCER(16:0) | CXCL13 | r = 0.308(p = 0.004) |
| HCER(20:1) | CXCL14 | r = 0.215(p = 0.047) |
| HCER(22:0) | CCL18 | r = 0.234(p = 0.030) |
| HCER(24:0) | CXCL13 | r = 0.241(p = 0.026) |
| | CCL18 | r = 0.231(p = 0.033) |
| HCER(24:1) | CXCL13 | r = 0.309(p = 0.004) |
| HCER(26:0) | CXCL14 | r = 0.221(p = 0.041) |
| HCER(26:1) | CXCL14 | r = 0.221(p = 0.041) |

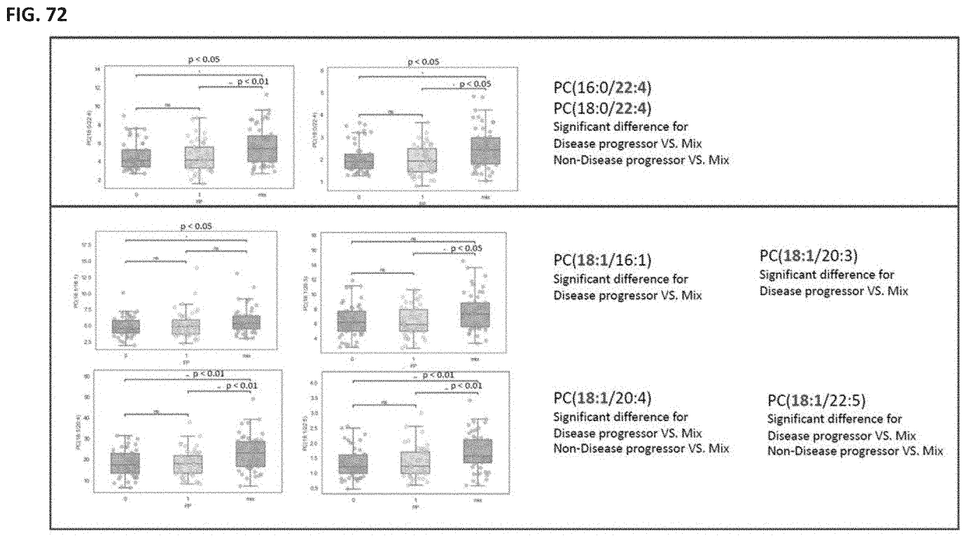

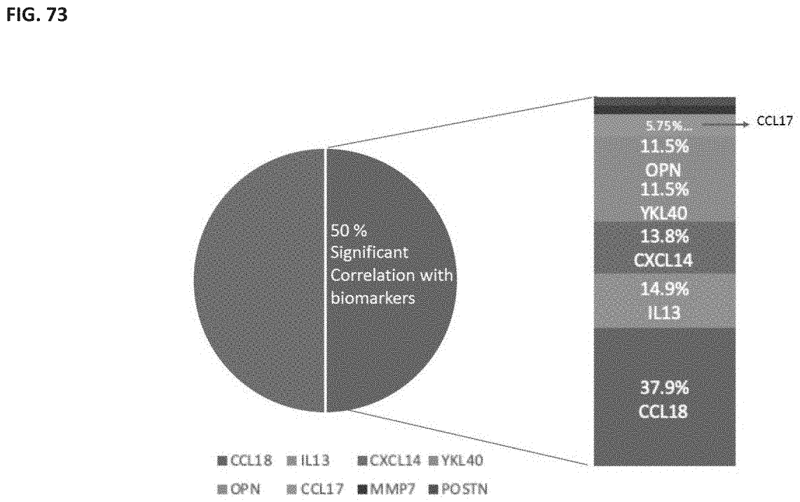

Certain phosphatidylcholine (PC) species with polyunsaturated fatty acids were significantly increased in the mixed group relative to disease progressors or non-disease progressors (FIG. 72).

About 50% of PC species showed significant correlation with biomarkers. CCL17, YKN40, OPN, and POSTN showed positive correlation with PC species; CCL18, IL13, and MMP7 showed negative correlation with PC species, and CXCL14 showed both negative and positive correlations with different PC species.

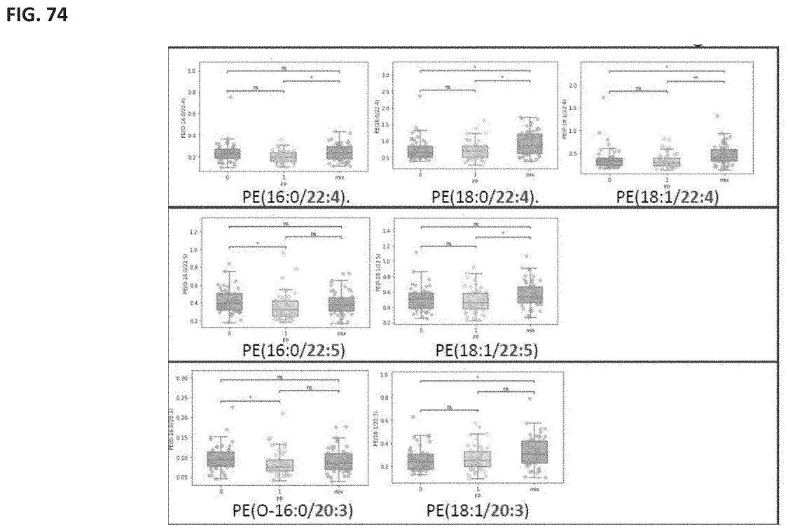

Certain phosphatidylethanolamine (PE) species were significantly increased in mixed or fibrosis progressor patients as compared to non-progressors (FIG. 74).

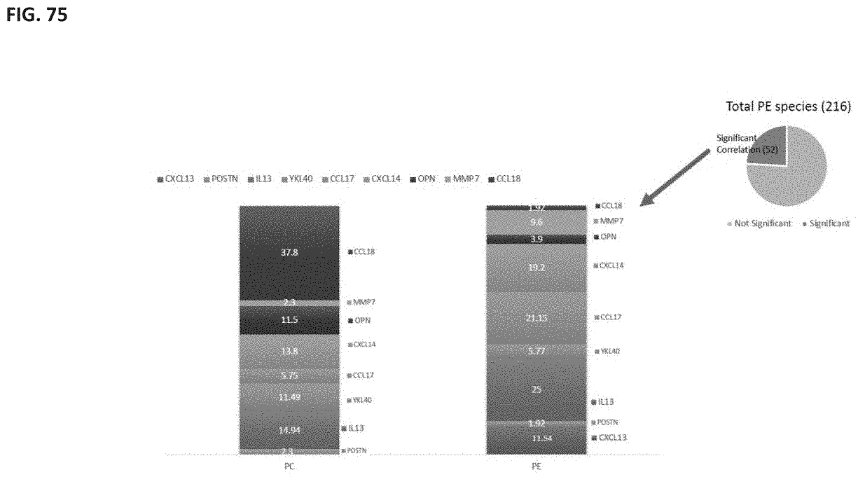

52 of the 216 PE species were significantly correlated with biomarkers (FIG. 75). YKN40, POSTN, CXCL13, CXCL14, and CCL17 showed positive correlation with PE species. CCL18, IL13, MMP7, and OPN showed negative correlation with PE species.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-308. (canceled)

309. A method of treating or reducing exacerbations in a patient having COPD, the method comprising administering to the patient an effective amount of an agent that reduces exacerbations, wherein the level of one or more of LPA16:0, LPA18:0, LPA18:1, LPA18:2, and LPA20:4 in a sample from the patient has been determined to be below a reference level.

310. A method of monitoring the response of a patient having COPD to a treatment comprising an agent that reduces exacerbations, the method comprising:
(a) measuring the level of one or more of LPA16:0, LPA18:0, LPA18:1, LPA18:2, and LPA20:4 in a sample obtained from the patient at a time point following the administration of a first dose of the treatment comprising the agent that reduces exacerbations; and
(b) comparing the level of one or more of LPA16:0, LPA18:0, LPA18:1, LPA18:2, and LPA20:4 in the sample to a reference level, thereby monitoring the response of the patient to the treatment comprising an agent that reduces exacerbations.

311. A method of treating a patient having COPD, wherein the patient has experienced at least one exacerbation in the prior 12 months, the method comprising:
(a) measuring a level of one or both of LPA18:0 and LPA18:2 in a sample from the patient, wherein a level of one or both of LPA18:0 and LPA18:2 in the sample that is above a reference level identifies the patient as one who may have an increased time to next exacerbation; and
(b) maintaining the treatment regimen of and/or reducing monitoring of a patient who has been determined to have a level of one or both of LPA18:0 and LPA18:2 in the sample that is above a reference level.

312. A method of treating a patient having IPF, the method comprising administering to the patient an effective amount of an agent that reduces exacerbations, wherein the level of one or more of LPA16:0, LPA18:1, LPA18:2, and LPA20:4 in a sample from the patient has been determined to be at or above a reference level.

313. A method of identifying and treating a patient having IPF, the method comprising:
(a) measuring a level of one or more of LPA16:0, LPA18:1, LPA20:4, and LPA22:4 in a sample from the patient, wherein a level of one or more of LPA16:0, LPA18:1, LPA20:4, and LPA22:4 in the sample that is at or above a reference level identifies the patient as one who may have a decreased time to exacerbation or respiratory hospitalization; and
(b) administering an effective amount of an agent that reduces exacerbations to a patient who has been determined to have a level of one or more of LPA16:0, LPA18:1, LPA20:4, and LPA22:4 in the sample that is at or above a reference level.

314. A method of treating a patient having IPF, the method comprising:
(i)
(a) measuring a level of one or more of LPA16:0, LPA16:1, LPA18:1, LPA18:2, and LPA20:4 in a sample from the patient, wherein a level of one or more of LPA16:0, LPA16:1, LPA18:1, LPA18:2, and LPA20:4 in the sample that is at or above a reference level identifies, diagnoses, and/or predicts the patient as one who is at an increased risk for deterioration of diffusing capacity of carbon monoxide (DLCO);
(b) measuring a level of LPA22:4 in a sample from the patient, wherein a level of LPA22:4 in the sample that is at or above a reference level identifies, diagnoses, and/or predicts the patient as one who is at an increased risk for increased ground glass opacity in the whole lung; or
(c) measuring a level of one or more of LPA16:0, LPA16:1, LPA18:0, LPA18:1, LPA18:2, and LPA20:4 in a sample from the patient, wherein a level of one or more of LPA16:0, LPA16:1, LPA18:0, LPA18:1, LPA18:2, and LPA20:4 in the sample that is at or above a reference level identifies, diagnoses, and/or predicts the patient as one who is at an increased risk for fibrosis in lower zones of the lung; and
(ii) administering an effective amount of an agent that reduces exacerbations to a patient who has been determined to have a level of one or more of LPA16:0, LPA16:1, LPA18:1, LPA18:2, and LPA20:4 in the sample that is at or above a reference level; a level of LPA22:4 in the sample that is at or above a reference level; or a level of one or more of LPA16:0, LPA16:1, LPA18:0, LPA18:1, LPA18:2, and LPA20:4 in the sample that is at or above a reference level.
